SEQUENCE LISTING (40 sequences)

SEQ ID NO: 1 - DNA, 1659 bp, Homo sapiens
atggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggagtttgtcaattttttcctcctccattataagaaggaagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgctcgtgtggcttggagtcatccgatacctcggtttctttgcaaagtacaacctcctcattttgacccttcaggcagcgctgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaag

SEQ ID NO: 2 - protein, 553 aa, Homo sapiens
MADPEVVVSSCSSHEEENRCNFNQQTSPSEELLLEDQMRRKLKFFFMNPCEKFWARGRKPWKLAIQILKIAMVTIQLVLFGLSNQMVVAFKEENTIAFKHLFLKGYMDRMDDTYAVYTQSDVYDQLIFAVNQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHVSGSIQKNTHYMMIFDAFVILTCLVSLILCIRSVIRGLQLQQEFVNFFLLHYKKEVSVSDQMEFVNGWYIMIIISDILTIIGSILKMEIQAKSLTSYDVCSILLGTSTMLVWLGVIRYLGFFAKYNLLILTLQAALPNVIRFCCCAAMIYLGYCFCGWIVLGPYHDKFRSLNMVSECLFSLINGDDMFATFAKMQQKSYLVWLFSRIYLYSFISLFIYMILSLFIALITDTYETIKQYQQDGFPETELRTFISECKDLPNSGKYRLEDDPPVSLFCCCKK

SEQ ID NO: 3 - DNA, 1659 bp, Homo sapiens
atggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggagtttgtcaattttttcctcctccattataagaaggaagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgctcgtgtggcttggagtcatccgatacctcggtttctttgcaaagtacaacctcctcattttgacccttcaggcagcgctgcccaatgtcatcaggttctgctgctgtccagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaag

SEQ ID NO: 4 - protein, 553 aa, Homo sapiens
MADPEVVVSSCSSHEEENRCNFNQQTSPSEELLLEDQMRRKLKFFFMNPCEKFWARGRKPWKLAIQILKIAMVTIQLVLFGLSNQMVVAFKEENTIAFKHLFLKGYMDRMDDTYAVYTQSDVYDQLIFAVNQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHVSGSIQKNTHYMMIFDAFVILTCLVSLILCIRSVIRGLQLQQEFVNFFLLHYKKEVSVSDQMEFVNGWYIMIIISDILTIIGSILKMEIQAKSLTSYDVCSILLGTSTMLVWLGVIRYLGFFAKYNLLILTLQAALPNVIRFCCCPAMIYLGYCFCGWIVLGPYHDKFRSLNMVSECLFSLINGDDMFATFAKMQQKSYLVWLFSRIYLYSFISLFIYMILSLFIALITDTYETIKQYQQDGFPETELRTFISECKDLPNSGKYRLEDDPPVSLFCCCKK

SEQ ID NO: 5 - DNA, 1740 bp, Homo sapiens
atgacagccccggcgggtccgcgcggctcagagaccgagcggcttctgacccccaaccccgggtatgggacccaggcggggccttcaccggcccctccgacacccccagaagaggaagaccttcgccgtcgtctcaaatactttttcatgagtccctgcgacaagtttcgagccaagggccgcaagccctgcaagctgatgctgcaagtggtcaagatcctggtggtcacggtgcagctcatcctgtttgggctcagtaatcagctggctgtgacattccgggaagagaacaccatcgccttccgacacctcttcctgctgggctactcggacggagcggatgacaccttcgcagcctacacgcgggagcagctgtaccaggccatcttccatgctgtggaccagtacctggcgttgcctgacgtgtcactgggccggtatgcgtatgtccgtggtgggggtgacccttggaccaatggctcagggcttgctctctgccagcggtactaccaccgaggccacgtggacccggccaacgacacatttgacattgatccgatggtggttactgactgcatccaggtggatccccccgagcggccccctccgccccccagcgacgatctcaccctcttggaaagcagctccagttacaagaacctcacgctcaaattccacaagctggtcaatgtcaccatccacttccggctgaagaccattaacctccagagcctcatcaataatgagatcccggactgctataccttcagcgtcctgatcacgtttgacaacaaagcacacagtgggcggatccccatcagcctggagacccaggcccacatccaggagtgtaagcaccccagtgtcttccagcacggagacaacagcttccggctcctgtttgacgtggtggtcatcctcacctgctccctgtccttcctcctctgcgcccgctcactccttcgaggcttcctgctgcagaacgagtttgtggggttcatgtggcggcagcggggacgggtcatcagcctgtgggagcggctggaatttgtcaatggctggtacatcctgctcgtcaccagcgatgtgctcaccatctcgggcaccatcatgaagatcggcatcgaggccaagaacttggcgagctacgacgtctgcagcatcctcctgggcacctcgacgctgctggtgtgggtgggcgtgatccgctacctgaccttcttccacaactacaatatcctcatcgccacactgcgggtggccctgcccagcgtcatgcgcttctgctgctgcgtggctgtcatctacctgggctactgcttctgtggctggatcgtgctggggccctatcatgtgaagttccgctcactctccatggtgtctgagtgcctgttctcgctcatcaatggggacgacatgtttgtgacgttcgccgccatgcaggcgcagcagggccgcagcagcctggtgtggctcttctcccagctctacctttactccttcatcagcctcttcatctacatggtgctcagcctcttcatcgcgctcatcaccggcgcctacgacaccatcaagcatcccggcggcgcaggcgcagaggagagcgagctgcaggcctacatcgcacagtgccaggacagccccacctccggcaagttccgccgcgggagcggctcggcctgcagccttctctgctgctgcggaagggacccctcggaggagcattcgctgctggtgaat

SEQ ID NO: 6 - protein, 580 aa, Homo sapiens
MTAPAGPRGSETERLLTPNPGYGTQAGPSPAPPTPPEEEDLRRRLKYFFMSPCDKFRAKGRKPCKLMLQVVKILVVTVQLILFGLSNQLAVTFREENTIAFRHLFLLGYSDGADDTFAAYTREQLYQAIFHAVDQYLALPDVSLGRYAYVRGGGDPWTNGSGLALCQRYYHRGHVDPANDTFDIDPMVVTDCIQVDPPERPPPPPSDDLTLLESSSSYKNLTLKFHKLVNVTIHFRLKTINLQSLINNEIPDCYTFSVLITFDNKAHSGRIPISLETQAHIQECKHPSVFQHGDNSFRLLFDVVVILTCSLSFLLCARSLLRGFLLQNEFVGFMWRQRGRVISLWERLEFVNGWYILLVTSDVLTISGTIMKIGIEAKNLASYDVCSILLGTSTLLVWVGVIRYLTFFHNYNILIATLRVALPSVMRFCCCVAVIYLGYCFCGWIVLGPYHVKFRSLSMVSECLFSLINGDDMFVTFAAMQAQQGRSSLVWLFSQLYLYSFISLFIYMVLSLFIALITGAYDTIKHPGGAGAEESELQAYIAQCQDSPTSGKFRRGSGSACSLLCCCGRDPSEEHSLLVN

SEQ ID NO: 7 - DNA, 1698 bp, Homo sapiens
atggcccggcagccttatcgttttccccaggcaaggattccggagagaggatcaggtgttttcaggttaaccgtcagaaacgcaatggcacatcgtgattctgagatgaaagaagaatgtctaagggaagacctgaagttttacttcatgagcccttgtgaaaaataccgagccagacgccagattccgtggaaactgggtttgcagattttgaagatagtcatggtcaccacacagcttgttcgttttggtttaagtaaccagctggtggttgctttcaaagaagataacactgttgcttttaagcacttgtttttgaaaggatattctggtacagatgaagatgactacagctgcagtgtatatactcaagaggatgcctatgagagcatcttttttgctattaatcagtatcatcagctaaaggacattaccctggggacccttggttatggagaaaatgaagacaatagaattggcttaaaagtctgtaagcagcattacaagaaagggaccatgtttccttctaatgagacactgaatattgacaacgacgttgagctcgattgtgttcaattagaccttcaggacctctccaagaagcctccggactggaagaactcatcattcttcagactggaattttatcggctcttacaggttgaaatctcctttcatcttaaaggcattgacctacagacaattcattcccgtgagttaccagactgttatgtctttcagaatacgattatctttgacaataaagctcacagtggcaaaatcaaaatctattttgacagtgatgccaaaattgaagaatgtaaagacttgaacatatttggatctactcagaaaaatgctcagtatgtcctggtgtttgatgcatttgtcattgtgatttgcttggcatctcttattctgtgtacaagatccattgttcttgctctaaggttacggaagagatttctaaatttcttcctggagaagtacaagcggcctgtgtgtgacaccgaccagtgggagttcatcaacggctggtatgtcctggtgattatcagcgacctaatgacaatcattggctccatattaaaaatggaaatcaaagcaaagaatctcacaaactatgatctctgcagcatttttcttggaacctctacgctcttggtttgggttggagtcatcagatacctgggttatttccaggcatataatgtgctgattttaacaatgcaggcctcactgccaaaagttcttcggttttgtgcttgtgctggtatgatttatctgggttacacattctgtggctggattgtcttaggaccataccatgacaagtttgaaaatctgaacacagttgctgagtgtctgttttctctggtcaacggtgatgacatgtttgcaacctttgcccaaatccagcagaagagcatcttggtgtggctgttcagtcgtctgtatttatattccttcatcagcctttttatatatatgattctcagtctttttattgcacttattacagattcttatgacaccattaagaaattccaacagaatgggtttcctgaaacggatttgcaggaattcctgaaggaatgcagtagcaaagaagagtatcagaaagagtcctcagccttcctgtcctgcatctgctgtcggaggaggaaaagaagtgatgatcacttgatacctattagc

SEQ ID NO: 8 - protein, 566 aa, Homo sapiens
MARQPYRFPQARIPERGSGVFRLTVRNAMAHRDSEMKEECLREDLKFYFMSPCEKYRARRQIPWKLGLQILKIVMVTTQLVRFGLSNQLVVAFKEDNTVAFKHLFLKGYSGTDEDDYSCSVYTQEDAYESIFFAINQYHQLKDITLGTLGYGENEDNRIGLKVCKQHYKKGTMFPSNETLNIDNDVELDCVQLDLQDLSKKPPDWKNSSFFRLEFYRLLQVEISFHLKGIDLQTIHSRELPDCYVFQNTIIFDNKAHSGKIKIYFDSDAKIEECKDLNIFGSTQKNAQYVLVFDAFVIVICLASLILCTRSIVLALRLRKRFLNFFLEKYKRPVCDTDQWEFINGWYVLVIISDLMTIIGSILKMEIKAKNLTNYDLCSIFLGTSTLLVWVGVIRYLGYFQAYNVLILTMQASLPKVLRFCACAGMIYLGYTFCGWIVLGPYHDKFENLNTVAECLFSLVNGDDMFATFAQIQQKSILVWLFSRLYLYSFISLFIYMILSLFIALITDSYDTIKKFQQNGFPETDLQEFLKECSSKEEYQKESSAFLSCICCRRRKRSDDHLIPIS

SEQ ID NO: 9 - DNA, 1659 bp, Mus musculus
atggcaaatcccgaggtgctggttagcagctgcagagctcgccaagatgaaagcccctgcactttccacccgagctcgtccccgtcagagcagcttctcttagaagaccagatgaggcggaaactcaagttcttttttatgaatccttgtgagaagttctgggctcggggtaggaagccatggaaacttgccatacagattctgaaaatcgcgatggtgactatccagctggttctgtttggactaagtaaccagatggtagtagctttcaaagaggagaacactatagccttcaaacacctcttcctaaagggctacatggatcgaatggacgacacctatgcagtgtacactcagagtgaagtgtatgaccagatcatctttgcagtgacccagtacttgcagcttcagaacatctccgtgggcaatcacgcttatgagaacaaggggactaagcagtcggcgatggcaatctgtcagcacttctacaggcaaggaaccatctgccccgggaacgacacctttgacatcgatccagaagttgaaacagaatgtttccttgtagagccagatgaagcttcccaccttggaacgcctggagaaaataaactcaacctgagcctggacttccacagacttctgacggtggagctccagtttaagctcaaagccatcaatctgcagacagttcgacaccaggagcttcctgactgttacgactttacgctgactataacattcgacaacaaggctcacagtggaagaatcaaaataagcttagacaacgacatttctatcaaagaatgcaaagactggcatgtgtctggatcaattcagaagaacacacactacatgatgatctttgatgcctttgtcattctgacctgcttggcctcactggtgctgtgtgccaggtctgtgattaggggtcttcagcttcagcaggagtttgtcaacttcttccttcttcactacaagaaggaagtttcggcctctgatcagatggagttcatcaacgggtggtacattatgatcatcattagtgacatattgacaatcgttggatcagttctgaaaatggaaatccaagccaagagtctcacaagctatgatgtctgcagcatacttctcgggacgtcaactatgctcgtgtggcttggagttatccgatacctgggtttctttgcgaagtacaatctccttattctgaccctccaggcagcgctgcccaacgtcatgaggttctgttgctgcgctgctatgatctatctaggctattgcttttgcggatggattgtgctgggcccttaccatgagaagttccgttccctgaacagggtctccgagtgcctgttctcgctgataaacggagacgatatgttttccacatttgcgaaaatgcagcagaagagttacctggtgtggctgttcagccgagtctacctgtactcgttcatcagcctcttcatttacatgattctgagccttttcatcgcgctcatcacagacacatacgaaacaattaagcactaccagcaagatggcttcccagagacggaacttcgaaagtttatagcggaatgcaaagacctccccaactccggaaaatacagattagaagatgaccctccgggttctttactctgctgctgcaaaaag

SEQ ID NO: 10 - protein, 553 aa, Mus musculus
MANPEVLVSSCRARQDESPCTFHPSSSPSEQLLLEDQMRRKLKFFFMNPCEKFWARGRKPWKLAIQILKIAMVTIQLVLFGLSNQMVVAFKEENTIAFKHLFLKGYMDRMDDTYAVYTQSEVYDQIIFAVTQYLQLQNISVGNHAYENKGTKQSAMAICQHFYRQGTICPGNDTFDIDPEVETECFLVEPDEASHLGTPGENKLNLSLDFHRLLTVELQFKLKAINLQTVRHQELPDCYDFTLTITFDNKAHSGRIKISLDNDISIKECKDWHVSGSIQKNTHYMMIFDAFVILTCLASLVLCARSVIRGLQLQQEFVNFFLLHYKKEVSASDQMEFINGWYIMIIISDILTIVGSVLKMEIQAKSLTSYDVCSILLGTSTMLVWLGVIRYLGFFAKYNLLILTLQAALPNVMRFCCCAAMIYLGYCFCGWIVLGPYHEKFRSLNRVSECLFSLINGDDMFSTFAKMQQKSYLVWLFSRVYLYSFISLFIYMILSLFIALITDTYETIKHYQQDGFPETELRKFIAECKDLPNSGKYRLEDDPPGSLLCCCKK

SEQ ID NO: 11 - DNA, 1773 bp, Gallus gallus
atgatcacccgtggtttccgttcaggcttttacggctgtagcaatcgatatagtggtgctaagtgccagttagttgctcagacgtttgtcatttcagcaatggagactcctgaagtggctgtaagcagctgcagtgctcgggatgacgaagggctctgcagctacggacaacacctattgctgccacaagagctggtggcggaagaccagctgaggaggaagctgaagttcttcttcatgaacccatgtgaaaaattctgggctcggggcagaaaaccttggaaacttgggattcagctgctcaaaatagcaatggttaccattcagctggtgctttttggattgagcaatcaaatggtggttgctttcaaagaagagaacactattgcattcaaacatctcttcttgaaagggtacatggacagaatggatgatacctatgcggtatacacacagacagatgtctatgaccaaatattctttgccatcaatcagtacttacagttgcccaacatttctgttggaaaccatgcttatgagaagaaaggagcagaagagacagctctggctgtatgtcaacagttctacaagcaaggaaccatctgtcctggaaatgacacctttgatatagacccagagattgtgactgactgcttgtacattgagccgatgatgtctttagacaacagaacagtgggaaagcacaatttgaatttcactctggatttccacaggctcgtggcagtgcaactcatgttcaatctgaaggcaatcaacctccagaccgtccgtcaccacgagctccctgactgttacgatttcaccctgacgatagtgtttgataataaagcccacagtggaagaatcaaaatcagtctagacaacgacatagagatcagggaatgtaaagactggcacgtttctggatcaatacagaagaatacgcattacatgatgatcttcgatgcttttgtcatactgatctgtctgagctcattgatcctttgcactcgatcagtagtcaaaggaattcggctccaaagagaatttgtaagttttttcctatattattacaagaaagaggtatcttacaatgatcagatggaatttgtcaatggctggtatatcctcattatggttagtgatgtcctcactatcgttggatcaactctcaaaatggagatacaggccaagagtctgacaagttacgacgtctgtagcatactcttaggaacatccactatgctggtgtggcttggagtcattcgctacctcggtttctttcagaagtataatcttctcattctaacgctgcgagcagcactacccaacgtcatgaggttctgctgttgtgctgctatgatctatctaggttattgtttctgcggatggattgtactggggccataccacgtgaagttccgttctctgaatgtggtttctgaatgcctcttttcattgataaatggagatgacatgtttgccacttttgcaaaaatgcagcagaaaagttacttggtttggttattcagtagaatctacctctactccttcatcagcctgttcatctacatggtgctaagtctcttcattgcactcattacagatacatatgaaactatcaagcactaccaacaagatggctttccagagacagaacttcagagatttatatcacagtgcaaagacttaccaaactctggaaggtacagattagaagaggaaggttctgtatctctcttctgttgttgcagtggtcctagtgaacatatc

SEQ ID NO: 12 - protein, 591 aa, Gallus gallus
MITRGFRSGFYGCSNRYSGAKCQLVAQTFVISAMETPEVAVSSCSARDDEGLCSYGQHLLLPQELVAEDQLRRKLKFFFMNPCEKFWARGRKPWKLGIQLLKIAMVTIQLVLFGLSNQMVVAFKEENTIAFKHLFLKGYMDRMDDTYAVYTQTDVYDQIFFAINQYLQLPNISVGNHAYEKKGAEETALAVCQQFYKQGTICPGNDTFDIDPEIVTDCLYIEPMMSLDNRTVGKHNLNFTLDFHRLVAVQLMFNLKAINLQTVRHHELPDCYDFTLTIVFDNKAHSGRIKISLDNDIEIRECKDWHVSGSIQKNTHYMMIFDAFVILICLSSLILCTRSVVKGIRLQREFVSFFLYYYKKEVSYNDQMEFVNGWYILIMVSDVLTIVGSTLKMEIQAKSLTSYDVCSILLGTSTMLVWLGVIRYLGFFQKYNLLILTLRAALPNVMRFCCCAAMIYLGYCFCGWIVLGPYHVKFRSLNVVSECLFSLINGDDMFATFAKMQQKSYLVWLFSRIYLYSFISLFIYMVLSLFIALITDTYETIKHYQQDGFPETELQRFISQCKDLPNSGRYRLEEEGSVSLFCCCSGPSEHI

SEQ ID NO: 13 - DNA, 2424 bp, Canis familiaris
atgaccccttttggcagcttggcttctgcaaaggcttcaaactcaagagctgcctggaagattgtggtggattcattcagctcgcagcacacgccccaggtgggcactggtggtccccaggaatcagacaaggcccttaccttcggagagttaacgttctcccactcatctccattcttcatctgcgccagcccttctctcccaccgttgcacagcagtgggctagacgatgagccatactgctggacaggttttcactgcatcaagtacctcgcgggcccagcgagtgtcccaaactcccttgaaagagggagtaagatattggtttcccaagcctcctttcccatccggacctccccttacctgacactgctaagccgaggggaaaagaagcctctctgcagttccgtggagaaaaggcctttgggggttttggagatgggaagtctgactctcctctcggaagagctcaaacggcagctccccggcacgctgttgctaggacaacctccgttgctaagggaaagaggcggctcctctgctgagattgacaagacgccgctaccaatagggcgtctactctccggccgcctcttcaaggccgcacttgtgattggctgctgtcgtgctgacgtcacgcaactcgaaccgccgagagatcctgggttttcgcccgctcggcaggaggtgtgcggtttgggttcccgcgtggagggtgctgctggctcgaatgtaaacaatctttttttttttctccccctagagatggcaaatcctacggttgttataagtagctgcagctctcatgaagaggaaaatcgttgcacttttagccagcacacatcgccctctgaggagcttctgttagaagaccagataaggcgaaaactcaaattttttttcatgaatccttgtgaaaagttctgggctcgaggtagaaaaccatggaagcttgccatacaaattctaaaaattgcaatggtgactatccagctggtcttttttgggctaagtaaccagatggttgtagctttcaaggaagaaaacactatagcattcaaacacctcttcttaaaaggatatatggaccgaatggatgacacatatgcagtgtacacacaacgtgatgtatatgatcagatcatctttgcagtgaaccagtacttgcttctacgcaatacctcggttgggaatcatgcttatgagaacaaggggacggaacagtctgctatggcaatctgtcagcacttctacaagcagggaaacatctgtcctggaaatgataccttcgacattgatccagagattgaaactgagtgtttctctgtagagccagctgagcctttccacgtcggaacactggaagaaaataaactcaacttaacgctggactttcacagactcctcacggtggacctgcagtttaagctgaaggccattaatctgcagaccattcggcatcacgagctccctgactgttatgactttactctcactataacatttgacaataaggcccatagtggaagaattaagataagtttagataatgatatttccatcagagaatgtaaagactggcatgtatcgggatcaattcagaagaacactcactacatgatgatctttgatgcctttgttattctgacatgcttggcttcactaaccctgtgccttcgatctgtaattagaggacttcagcttcaacaggaatttgtcaattttttcctcctccattataagaaggaagtttctgtttctgatcgaatggaatttgtcaatggatggtacattatgattattattagtgacatgttgacaattattggatcaattctgaaaatggaaattcaagctaagagtctaacaagttatgatgtttgtagcatacttcttgggacttccaccatgcttgtgtggcttggagttattcgatacctcggcttctttcagaagtacaatctccttattctgaccctgcaggcagcactgcccagtgtcatcaggttctgttgctgtgccgctatgatttatttaggctattgcttctgtggatggattgtgctggggccgtaccatgataagttccgttctctgaacatggtctctgagtgccttttctctctgataaatggagatgatatgtttgccacatttgcaaaaatgcaacaaaaaagttacttggtctggctgtttagcagaatttatctctactcattcatcagcctctttatatatatgattttaagtcttttcatcgcactgatcactgatacgtatgaaacaattaagcattaccaacaagatggctttccagagactgaacttcgtacatttatatcagagtgcaaagatctacccaattctggaaaatacagattagaagatgacactccaatatctatattctgctgttgtaaaaag

SEQ ID NO: 14 - protein, 808 aa, Canis familiaris
MTPFGSLASAKASNSRAAWKIVVDSFSSQHTPQVGTGGPQESDKALTFGELTFSHSSPFFICASPSLPPLHSSGLDDEPYCWTGFHCIKYLAGPASVPNSLERGSKILVSQASFPIRTSPYLTLLSRGEKKPLCSSVEKRPLGVLEMGSLTLLSEELKRQLPGTLLLGQPPLLRERGGSSAEIDKTPLPIGRLLSGRLFKAALVIGCCRADVTQLEPPRDPGFSPARQEVCGLGSRVEGAAGSNVNNLFFFLPLEMANPTVVISSCSSHEEENRCTFSQHTSPSEELLLEDQIRRKLKFFFMNPCEKFWARGRKPWKLAIQILKIAMVTIQLVFFGLSNQMVVAFKEENTIAFKHLFLKGYMDRMDDTYAVYTQRDVYDQIIFAVNQYLLLRNTSVGNHAYENKGTEQSAMAICQHFYKQGNICPGNDTFDIDPEIETECFSVEPAEPFHVGTLEENKLNLTLDFHRLLTVDLQFKLKAINLQTIRHHELPDCYDFTLTITFDNKAHSGRIKISLDNDISIRECKDWHVSGSIQKNTHYMMIFDAFVILTCLASLTLCLRSVIRGLQLQQEFVNFFLLHYKKEVSVSDRMEFVNGWYIMIIISDMLTIIGSILKMEIQAKSLTSYDVCSILLGTSTMLVWLGVIRYLGFFQKYNLLILTLQAALPSVIRFCCCAAMIYLGYCFCGWIVLGPYHDKFRSLNMVSECLFSLINGDDMFATFAKMQQKSYLVWLFSRIYLYSFISLFIYMILSLFIALITDTYETIKHYQQDGFPETELRTFISECKDLPNSGKYRLEDDTPISIFCCCKK

SEQ ID NO: 15 - DNA, 1518 bp, Danio rerio
atgtctgaccgagcgtcacacactcatgaaagcgcaacacttctggacccggagtgtgtggaaagcttaaggagaaaactcaagtatttcttcatgagtccgtgtcagaaatacagcactagaggacggataccatggaagatgatgcttcagatactcaagatttgtttagtattcatctacctggtctcttttggattgagcaacgagatgatggtgacgttcaaagaggaaaatctcatcgccttcaagcacttctttctgaaaaactacaaggacagcaataaacattacgccttgtacacaaaacatgaagttcacgaccacatcctctacaccatcagacggtatctacagctacaaaacctgacgattggcaatcaagcgctggagatgatcgatggtctggcgactcctctgtctctctgtcagcagttgtatcgacatgcgcgcgtcgtgccgtctaatgagacgtttgaaatcgatccacatgtagagacagagtgtgtttctgtgtatcccctttctcccatcacgactgacagtctggaaaactccctgaacttgactttagattttcaaaggttgttagcggtaaacatttatctgaagatcaaggctatcaacattcagacggttcgccatcaagagttaccagactgctacgacttcagcattaatatcatgtttgacaatcgtgcacacagcggacagatcaagatctctctcagcagcggcgtgcagataaacgtctgtaaggactggaacatttctggctcaagtaagttgaacagccactttgcgctgattgtggtgtttgactgtttgatcatctgcttctgtctgctgtcactcatcctctgcacgcgctcagtccacacaggatttctcctacagactgaatacagaagattcatgtccagtcagcacagtaaaagcgtctcatggtctgagaggctggagttcatcaacggctggtacatcctcatcatcatcagcgatgcgctgactattgcaggctcaatcctcaaaatctgcatacagagcaaagaactgacgagctatgacgtgtgcagtattctgctgggcactgcaacaatgctggtgtggattggagtaatgcgctacctcagtttcttccagaaatattatatcctcatcctcaccctgaaggctgcacttcccaatgtgattcgattctccatctgcgctgttatgatctacctgagttactgcttctgcggatggatcgttttggggccacaccatgaaaattttcgcacattcagtagggttgctggctgtcttttctccatgattaatggggatgaaatctactccacgttcaccaagctccgggaatacagcactctggtgtggctgttcagcagactctacgtctacagcttcatcccggtcttcacatacatggttctgagtgtcttcatcgccctcatcacagacacgtatgaaaccatcagggtgagttatttcagcttcagtgagagtagctgcaaa

SEQ ID NO: 16 - protein, 506 aa, Danio rerio
MSDRASHTHESATLLDPECVESLRRKLKYFFMSPCQKYSTRGRIPWKMMLQILKICLVFIYLVSFGLSNEMMVTFKEENLIAFKHFFLKNYKDSNKHYALYTKHEVHDHILYTIRRYLQLQNLTIGNQALEMIDGLATPLSLCQQLYRHARVVPSNETFEIDPHVETECVSVYPLSPITTDSLENSLNLTLDFQRLLAVNIYLKIKAINIQTVRHQELPDCYDFSINIMFDNRAHSGQIKISLSSGVQINVCKDWNISGSSKLNSHFALIVVFDCLIICFCLLSLILCTRSVHTGFLLQTEYRRFMSSQHSKSVSWSERLEFINGWYILIIISDALTIAGSILKICIQSKELTSYDVCSILLGTATMLVWIGVMRYLSFFQKYYILILTLKAALPNVIRFSICAVMIYLSYCFCGWIVLGPHHENFRTFSRVAGCLFSMINGDEIYSTFTKLREYSTLVWLFSRLYVYSFIPVFTYMVLSVFIALITDTYETIRVSYFSFSESSCK

SEQ ID NO: 17 - DNA, 1659 bp, Homo sapiens
atggccgaccctgaggtggtggtgtcctcctgctctagccacgaggaagagaaccggtgcaacttcaaccagcagaccagccccagcgaggaactgctgctggaagatcagatgcggcggaagctgaagttcttcttcatgaacccctgcgagaagttctgggccagaggccggaagccttggaagctggccatccagatcctgaagatcgccatggtgaccatccagctggtgctgttcggcctgagcaaccagatggtggtggccttcaaagaggaaaacacaatcgccttcaagcacctgtttctgaagggctacatggaccggatggacgacacctacgccgtgtacacccagagcgacgtgtacgaccagctgatcttcgccgtgaaccagtacctgcagctgtacaacgtgagcgtgggcaaccacgcctacgagaacaagggcaccaagcagagcgccatggccatctgccagcacttctacaagcggggcaacatctaccccggcaacgacaccttcgacatcgaccccgagatcgagacagagtgcttcttcgtggagcccgacgagcctttccacatcggcacccctgccgagaacaagctgaacctgaccctggacttccaccggctgctgaccgtggagctgcagttcaagctgaaggccatcaacctgcagaccgtgcggcaccaggaactgcccgactgctacgacttcaccctgaccatcaccttcgataacaaggcccacagcggccggatcaagatcagcctggacaacgacatcagcatccgggagtgcaaggactggcacgtgagcggcagcatccagaaaaacacccactacatgatgatcttcgacgccttcgtgatcctgacctgcctggtgtccctgatcctgtgcatcagaagcgtcatcaggggcctgcagctccagcaggaattcgtcaacttcttcctgctgcactacaagaaagaagtgtccgtcagcgaccagatggaatttgtgaacggctggtacatcatgatcatcatcagcgacatcctgacaatcatcggcagcattctgaagatggaaatccaggccaagagcctgaccagctacgacgtgtgcagcattctgctgggaacctccaccatgctggtctggctcggcgtgatcagatacctgggcttcttcgccaagtacaacctgctgattctgacactgcaggccgccctgcccaacgtgatccggttctgctgctgcgccgccatgatctacctgggctactgcttctgcggctggatcgtgctgggcccctaccacgacaagttccggtccctgaacatggtgtccgagtgcctgttcagcctgatcaacggcgacgacatgttcgccaccttcgccaagatgcagcagaaaagctacctggtctggctgttcagccggatctacctgtacagcttcatcagcctgttcatctacatgatcctgagcctgtttatcgccctgatcaccgatacctacgagacaatcaagcagtaccagcaggacggcttccccgagacagagctgcggaccttcatcagcgagtgtaaggacctgcccaacagcggcaagtaccggctggaagatgacccccccgtgtccctgttctgctgttgcaagaag

SEQ ID NO: 18 - DNA, 1659 bp, Homo sapiens
atggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggagtttgtcaattttttcctcctccattataagaaggaagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgcttgtgtggcttggagtcatccgatacctcggtttctttgcaaagtacaacctcctcattttgacccttcaggcagcgctgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaag

SEQ ID NO: 19 - DNA, 2000 bp, Rattus norvegicus
gttcgacagaagcttgtgatttatggtccaaggaatccagtgtcagatcaatagacaaaatgccccagggaagttgtgtgtgcattctactggacagatcagagactggtcagaacaggtgcttggctggcggtgcgtccaaacctcagagatggcaaatcctgaggtggtggtaagcagctgcagttctcaccaggatgaaagtccctgcactttctacccgagctcatcccagtccgagcagcttctcttagaagatcagatgaggcggaaactcaaattcttttttatgaatccttgcgagaagttctgggctcggggtaggaagccatggaaacttgccatacagattctgaaaatcgctatggtgactatccagctggttctgtttggactaagtaaccagatggtagtagctttcaaggaagagaacacgatagccttcaaacacctcttcctgaaaggctacatggaccgaatggacgacacctacgcggtgtacactcagaatgatgtgtacgaccagatcatctttgcagtgacccggtacttgcaacttcgaaacatctccgtcggcaaccatgcttatgagaacaaggggactaagcagtcagcaatggcagtctgtcagcacttctacaggcaaggcaccatctgccccgggaacgataccttcgacatcgatccagaagtcgaaacagactgtttccttatagagccagaggaagctttccacatgggaacacctggagaaaacaaactcaacctgaccctggacttccacagacttctgacagtggagctccaatttaagctcaaagccatcaacctgcagacagttcgccaccaggagcttcctgactgttacgactttaccctgactataacattcgacaacaaggcacacagtggaagaatcaaaataagtttagacaacgacatttctatcagagaatgcaaagattggcacgtgtctggatcaattcagaagaacacccactacatgatgatcttcgatgcctttgttatcctgacctgcttgtcctcgctggtgctctgcgccaggtctgtgattcggggtcttcagcttcagcaggagtttgtcaactttttccttcttcactacaagaaggaagtttcggcctctgatcagatggagttcatcaacgggtggtacattatgatcatcgttagtgacatactgacgatcgttggatcgattctgaaaatggaaatccaagccaagagtcttacaagctacgatgtctgtagcatacttcttgggacttccaccatgctcgtgtggcttggcgttatccgatacctgggtttctttgcgaagtacaatctccttatcctgaccctccaggcagcgctgcccaatgtcatcaggttctgttgctgtgcggctatgatctatcttgggtattgcttttgcggatggattgtgctgggcccttaccatgagaagttccgctctctgaacaaggtctctgagtgcctattctcactgataaatggagacgacatgttttccacgttcgcgaaaatgcagcagaaaagttacctggtgtggctgttcagccgcgtctacctgtactcgttcatcagcctcttcatctacatgatcttgagccttttcatcgcgctcatcacagacacgtacgaaaccattaagcactaccagcaagatggcttcccagagacggaacttcgaaagtttatcgctgaatgcaaagacctccccaactctggaaaatacagattggaagatgaccctccaggttctttattctgctgctgcaaaaagtaactgtcgggtttctctgtgcctgggaggaaaatacagtgtgtggatgagtcagagacaatatggattattggtaatcacgcaacagtgtgttcagatactagtgttctgagttaactcacagctatgactttgcggggcctgttaaatatatttttaaatattaaaaaaaaaaaaaaaaaaaaaaaaa

SEQ ID NO: 20 - protein, 553 aa, Rattus norvegicus
MANPEVVVSSCSSHQDESPCTFYPSSSQSEQLLLEDQMRRKLKFFFMNPCEKFWARGRKPWKLAIQILKIAMVTIQLVLFGLSNQMVVAFKEENTIAFKHLFLKGYMDRMDDTYAVYTQNDVYDQIIFAVTRYLQLRNISVGNHAYENKGTKQSAMAVCQHFYRQGTICPGNDTFDIDPEVETDCFLIEPEEAFHMGTPGENKLNLTLDFHRLLTVELQFKLKAINLQTVRHQELPDCYDFTLTITFDNKAHSGRIKISLDNDISIRECKDWHVSGSIQKNTHYMMIFDAFVILTCLSSLVLCARSVIRGLQLQQEFVNFFLLHYKKEVSASDQMEFINGWYIMIIVSDILTIVGSILKMEIQAKSLTSYDVCSILLGTSTMLVWLGVIRYLGFFAKYNLLILTLQAALPNVIRFCCCAAMIYLGYCFCGWIVLGPYHEKFRSLNKVSECLFSLINGDDMFSTFAKMQQKSYLVWLFSRVYLYSFISLFIYMILSLFIALITDTYETIKHYQQDGFPETELRKFIAECKDLPNSGKYRLEDDPPGSLFCCCKK

SEQ ID NO: 21 - DNA, 751 bp, Sus scrofa
gtgaaatggcagatcctgagcctgtcataagtagctgcagctctcgtgaagaggaaaatcgctgcacttttaaccagcacacatgtccctctgaggagcgtctattagaagaccagatgaggcgaaaactcaaattttttttcatgactccttgtgagaagttctggactcgaggtcgaaaaccatggaaacttgccatgcaagttctaaaaattgcgatggtgactatccagctgatctttttcgggctaagtaaccagatggtggtagctttcaaggaagagaacacgatagcatttaaacacctctttctaaagggctatgtggaccagatggatgacacatatgccgtgtacacccaaagcgacgtatacgatcggatcgtcttcgcagtgaaccagtacttgcagctacgcagcatctcggttgggaaccacgcttacgagaacaagggcgcggagcagtcggccatggcgatctgttggcacttctacaagcaaggaaacatctgtcctggaaatgacacctttgacgttgatccagaagtaaaaactgaatgtttctttgttgagccggatgaagctgttgacactggaacactggaggagaataagctcaacttaacccttgactttcacagactcctaacggtggagctgcagtttaaactcaaggccattaatctgcagacgattcgccatcacgaactccctgactgttatgacttcaccctgaccataacatttgacaa

SEQ ID NO: 22 - protein, 248 aa, Sus scrofa
MADPEPVISSCSSREEENRCTFNQHTCPSEERLLEDQMRRKLKFFFMTPCEKFWTRGRKPWKLAMQVLKIAMVTIQLIFFGLSNQMVVAFKEENTIAFKHLFLKGYVDQMDDTYAVYTQSDVYDRIVFAVNQYLQLRSISVGNHAYENKGAEQSAMAICWHFYKQGNICPGNDTFDVDPEVKTECFFVEPDEAVDTGTLEENKLNLTLDFHRLLTVELQFKLKAINLQTIRHHELPDCYDFTLTITFD

SEQ ID NO: 23 - DNA, 2880 bp, Pan troglodytes
cttactccaatcaagcctctgcccgccaggaataggtaacctgtgtgtgtccgtttgctccttctaagagcatgcctgatagatacttcggtagcctctccggatggccccttcgtcgggtagcctctcctgatggggtccttcgcccaccctgcctcccgcgccggcgctccgggtgaatgtcaagggtggctggctgcgaatacctccttcagctgctggggttcccgacagtttgcagtttttaaaagtgcaccctcggaagggcttttcagactgggtaaagctgacttttccaagagatggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggtagggaacgttgctttctaggaatgctactgacattttgattgacagagacattcactgtgcctcccctcttttccctaaaggagtttgtcaattttttcctcctccattataagaagggagtttctgtttctgatcaaatggaatttgtcaatggatggtacattatgattattattagtgacatattgacaatcattggatcaattctaaaaatggaaatccaagctaaggtaatttttttcctaatcatgctattgttagtgtcagatttgcactaatggtaatgtatttttccagaatgtaagaattttcagaatgaattgtttcttccaaactgtatatcaagtagacttgaaattggtaatggtaattttcttaaatctagtcaggaggtctcttaggcagagtttttcaaagtgtgatccacaaaccattgcatcagaatcattgggtgcctggtaaagtgtaccatgttagacctactgaattcagactcttcggcggggcctgtgaattcttacacacaccaaaattcatacacaaccaaggtaactaaggtaagagttttttttttttttaaatcttacaagaaatgctcaaatctttaacaaaaatgagtgggtctataggggaaagtgaggtcaaggcactatggtgtgcatgcttgcatttgtttcctccgtccattcaaagtgagaatgctcccattttcttactttaccattgatgtgctacaagcttatttattttaagactaacctagcctaaaaatcaactgtccccacaaaataaaaatcacattaaaaaaactaatagtgttcagactaatcttgctcaaacttatgtttccctagtcttgatgcgactgattgagtcacctggggagttggttataaacctgggcagagaccccaaatgcaatggctcagagaagataggagcttatttctgtcttatgcaatagtcagaatgggttttacagactggtgagtagctcaacatctcacagtcattcaggcacccatgttcctcccattttgtttctctgccatcccttaaggacttgccctgactgcatgattattgctgtgttgcctcaaacaggttgcagcttatgggaagcaaaaacacggtatggtggaagctctcccatagactgatggcttggctcaagagtggccgactttatttctgtacatatcccactggatagaatttagtcaatcctaactgcagagggagccagggaacacagcccaggcatgtgcctaggaaggggagaatgggtttaggttgacacttagcagctgccactatatgtggctatagtatgtatcattggaatagatgtttaactttagggacaaataaacaaaaaaccaaaacaaaaaaaggagtaaggggagagatttgcagcaaatctttatttttaccaacctcaactatcattaatttcagtgaaccctaaatggtgtccaacaaaatatctttctagaccattcaccgtctctgcctcatagatgatcatatcatgttttcttctcttctgaaacctctaatacccttgtcctatcctcattctaagctgatgaccttacttcctatttcacaaaaataatagaaaaaaaaa

SEQ ID NO: 24 - protein, 321 aa, Pan troglodytes
MADPEVVVSSCSSHEEENRCNFNQQTSPSEELLLEDQMRRKLKFFFMNPCEKFWARGRKPWKLAIQILKIAMVTIQLVLFGLSNQMVVAFKEENTIAFKHLFLKGYMDRMDDTYAVYTQSDVYDQLIFAVNQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHVSGSIQKNTHYMMIFDAFVILTCLVSLILCIRSVIRGLQLQQVGNVAF

SEQ ID NO: 25 - DNA, 1868 bp, Pan troglodytes
cctctagagatggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcattctccctccgtcgcccagcctggaaacactcattacatgatgatctttgatgcctttgtcattctgacttgcttggtttcattaatcctctgcattagatctgtgattagaggacttcagcttcagcaggagtttgtcaattttttcctcctccattataagaagggagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgcttgtgtggcttggagtcatccgatacctcggtttctttgcaaagtacaatctcctcattttgacccttcaggcagcactgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaagtagctatcaggtttatctgtactttagaggaaaatataatgtgtagctgagttagaacactgtggatattctgagatcagatgtagtatgtttgaagactgttattttgagctaattgagacctataattcaccaataactgtttatatttttaaaagcaatatttaatgtctttgcagctttatgctgggcttgtt

SEQ ID NO: 26 - protein, 554 aa, Pan troglodytes
MADPEVVVSSCSSHEEENRCNFNQQTSPSEELLLEDQMRRKLKFFFMNPCEKFWARGRKPWKLAIQILKIAMVTIQLVLFGLSNQMVVAFKEENTIAFKHLFLKGYMDRMDDTYAVYTQSDVYDQLIFAVNQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHSPSVAQPGNTHYMMIFDAFVILTCLVSLILCIRSVIRGLQLQQEFVNFFLLHYKKGVSVSDQMEFVNGWYIMIIISDILTIIGSILKMEIQAKSLTSYDVCSILLGTSTMLVWLGVIRYLGFFAKYNLLILTLQAALPNVIRFCCCAAMIYLGYCFCGWIVLGPYHDKFRSLNMVSECLFSLINGDDMFATFAKMQQKSYLVWLFSRIYLYSFISLFIYMILSLFIALITDTYETIKQYQQDGFPETELRTFISECKDLPNSGKYRLEDDPPVSLFCCCKK

SEQ ID NO: 27 - DNA, 2546 bp, Pan troglodytes
tcctctagagatggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggagtttgtcaattttttcctcctccattataagaagggagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgcttgtgtggcttggagtcatccgatacctcggtttctttgcaaagtacaatctcctcattttgacccttcaggcagcactgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaagtagctatcaggtttatctgtactttagaggaaaatataatgtgtagctgagttagaacactgtggatattctgagatcagatgtagtatgtttgaagactgttattttgagctaattgagacctataattcaccaataactgtttatatttttaaaagcaatatttaatgtctttgcagctttatgctgggcttgtttttaaaacaactttaatgaggaaagctattggattattattatttcttgtttattttgccatggctttagaatgtattctgtatgcctctcttttgctctgatactcttgcttcctgctattctgattgtgcagactgtgtaattagtggaaaacaatccttggtctgactgtgactttggacaactcagtaaccctggcttggaccactctcaggagtccatccttgagagagtgggtgtagttaccatttatacagtaatcattgcattttaaaatctgctcttgaaaggaagaataagagtgcaccagaataagagcgcaccagaataagagcgcaccagctaacaatgtgatacggccatatgtcacttaaggatagagatatgttctgagaaatgtgtcattaggcgattttgtcattaaacatcatagcatgtacttccacaaacctagatggtatagcctactacacacctaggctatttggtatagcctgttgatcctggggtacaaatctgtacaacatgttactgtattgaatacagtaggcaattgtaacacaatggtaagtatctaaacatagaaaagggacagtaaaaatatggttttataatcttctgggaccaccattgtatatgcggtacatcgttgaccaaaacatcgttatccagcatatgactgtatttggttatgaaagccaactgttacttgattctgcttttagttcttaagaggatcaggtttttaaatactcatttacaagttttctatcctccttcagtgttaaagtagaaagtaaaaagagtatcttatacatgcatgaaattaaagcatataccaaatgca

SEQ ID NO: 28 - protein, 497 aa, Pan troglodytes
MADPEVVVSSCSSHEEENRCNFNQQTSPSEELLLEDQMRRKLKFFFMNPCEKFWARGRKPWKLAIQILKIAMVTIQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHVSGSIQKNTHYMMIFDAFVILTCLVSLILCIRSVIRGLQLQQEFVNFFLLHYKKGVSVSDQMEFVNGWYIMIIISDILTIIGSILKMEIQAKSLTSYDVCSILLGTSTMLVWLGVIRYLGFFAKYNLLILTLQAALPNVIRFCCCAAMIYLGYCFCGWIVLGPYHDKFRSLNMVSECLFSLINGDDMFATFAKMQQKSYLVWLFSRIYLYSFISLFIYMILSLFIALITDTYETIKQYQQDGFPETELRTFISECKDLPNSGKYRLEDDPPVSLFCCCKK

SEQ ID NO: 29 - DNA, 2714 bp, Pan troglodytes
tcctctagagatggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggagtttgtcaattttttcctcctccattataagaagggagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgcttgtgtggcttggagtcatccgatacctcggtttctttgcaaagtacaatctcctcattttgacccttcaggcagcactgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaagtagctatcaggtttatctgtactttagaggaaaatataatgtgtagctgagttagaacactgtggatattctgagatcagatgtagtatgtttgaagactgttattttgagctaattgagacctataattcaccaataactgtttatatttttaaaagcaatatttaatgtctttgcagctttatgctgggcttgtttttaaaacaactttaatgaggaaagctattggattattattatttcttgtttattttgccatggctttagaatgtattctgtatgcctctcttttgctctgatactcttgcttcctgctattctgattgtgcagactgtgtaattagtggaaaacaatccttggtctgactgtgactttggacaactcagtaaccctggcttggaccactctcaggagtccatccttgagagagtgggtgtagttaccatttatacagtaatcattgcattttaaaatctgctcttgaaaggaagaataagagtgcaccagaataagagcgcaccagaataagagcgcaccagctaacaatgtgatacggccatatgtcacttaaggatagagatatgttctgagaaatgtgtcattaggcgattttgtcattaaacatcatagcatgtacttccacaaacctagatggtatagcctactacacacctaggctatttggtatagcctgttgatcctggggtacaaatctgtacaacatgttactgtattgaatacagtaggcaattgtaacacaatggtaagtatctaaacatagaaaagggacagtaaaaatatggttttataatcttctgggaccaccattgtatatgcggtacatcgttgaccaaaacatcgttatccagcatatgactgtatttggttatgaaagccaactgttacttgattctgcttttagttcttaagaggatcaggtttttaaatactcatttacaagttttctatcctccttcagtgttaaagtagaaagtaaaaagagtatcttatacatgcatgaaattaaagcatataccaaatgca

SEQ ID NO: 30 - protein, 553 aa, Pan troglodytes
MADPEVVVSSCSSHEEENRCNFNQQTSPSEELLLEDQMRRKLKFFFMNPCEKFWARGRKPWKLAIQILKIAMVTIQLVLFGLSNQMVVAFKEENTIAFKHLFLKGYMDRMDDTYAVYTQSDVYDQLIFAVNQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHVSGSIQKNTHYMMIFDAFVILTCLVSLILCIRSVIRGLQLQQEFVNFFLLHYKKGVSVSDQMEFVNGWYIMIIISDILTIIGSILKMEIQAKSLTSYDVCSILLGTSTMLVWLGVIRYLGFFAKYNLLILTLQAALPNVIRFCCCAAMIYLGYCFCGWIVLGPYHDKFRSLNMVSECLFSLINGDDMFATFAKMQQKSYLVWLFSRIYLYSFISLFIYMILSLFIALITDTYETIKQYQQDGFPETELRTFISECKDLPNSGKYRLEDDPPVSLFCCCKK

SEQ ID NO: 31 - DNA, 1896 bp, Silurana tropicalis
ttgcaactaggtctgacagtaggacaatgtggcaggtcacgtgacagcagtgctgatggtagagatgcgccagcattcaggtctgagagcagaaagaaaagctggccaaaacaaaggacattctctttgctgcttcgctagctgagacgctgctatagtatagcagacatggaaaacccagagctaataaagacatgcaactctttggatgaacatgatggtccctactgctgcaagcagtgccctatgactgatgagctacttatggaagaccagctacgaaggaaacttaaattctttttcatgaacccatgtgagaagttccgtgcccgtggacgaaagccttggaagctttgtattcaaattttaaaaattgcaatggtgacaatccaattagttttatttggactcagtaatgaaatggtagtcacctttaaagaggagaacactgtagcttttaagcatctgtttttgaaaggatataaggatggacatgatgacacatatgctatctacagtcaagaagatgttcatgctcatataaactttacaattaaaaggtacctagagctacaaaacatatctgttggaaatcatgcatatgaaagtaatggtaaaggtcaaactggaatgtcattatgtcaacattactataaacaagggagtatctttcctggaaatgaaacatttgaaattgacccacaaatagatactgaatgtttccatattgatccatcaactctgtgttctaatgacacacctgcagaatactactggtctaatatgacactagacttctatagacttgtttcagttgaaattatgtttaagcttaaagcaattaatcttcaaaccattcgtcatcatgaacttccagactgctatgacttcatggttataataacatttgataataaggcacacagtggaaggataaaaatcagcttagataatgatgttggaatccaggaatgcaaagactggcatgtgtctggatctattcaaaaaaatactcattacatgatgatttttgatgctgctgttattttggtctgcttatcttccataacactctgcattcgctccgtggttaaaggaattcacctacaaaaagaatatgtaaactttttccagcatcgttttgcaaggactgtgtcctcagctgatcgcatggaatttgtcaatggctggtacattatgataatcatcagtgatgttttgtcaattattggctcaatcttgaagatggagatccaagcaaagagcctcaccagttatgatgtctgcagtattctcttgggaacatccaccttattagtgtggcttggagttattcgctacttgggattttttaagaaatacaatcttctgatcctgacacttagggcagccttacctaatgtgatacgattctgctgttgtgctgctatgatctacctgggctactgcttctgtggctggattgttctggggccttaccatgtaaagttcaggtccctgaacatggtttcagagtgcctgttctcccttattaatggagacgatatgtttacaacgttttcaatcatgcaggagaagagctacttggtttggctgtttagtcgcatttatttgtattcctttataagtctcttcatatacatggttctgagtctcttcattgcacttattactgacacatacgatacaatcaagaattaccagatcgatggctttccagaatcagaacttcacacatttgtatccgagtgcaaagatttgccaacctctggtcgatatagggaacaagacgagacctcctgtttgtctatgctgtgttgtaatcggtaaaaaagaatcccagaagaagcactttatccatggcctttaaaaatctgcaaaaaaaaaaaaaaaaaa

SEQ ID NO: 32 - protein, 553 aa, Silurana tropicalis
MENPELIKTCNSLDEHDGPYCCKQCPMTDELLMEDQLRRKLKFFFMNPCEKFRARGRKPWKLCIQILKIAMVTIQLVLFGLSNEMVVTFKEENTVAFKHLFLKGYKDGHDDTYAIYSQEDVHAHINFTIKRYLELQNISVGNHAYESNGKGQTGMSLCQHYYKQGSIFPGNETFEIDPQIDTECFHIDPSTLCSNDTPAEYYWSNMTLDFYRLVSVEIMFKLKAINLQTIRHHELPDCYDFMVIITFDNKAHSGRIKISLDNDVGIQECKDWHVSGSIQKNTHYMMIFDAAVILVCLSSITLCIRSVVKGIHLQKEYVNFFQHRFARTVSSADRMEFVNGWYIMIIISDVLSIIGSILKMEIQAKSLTSYDVCSILLGTSTLLVWLGVIRYLGFFKKYNLLILTLRAALPNVIRFCCCAAMIYLGYCFCGWIVLGPYHVKFRSLNMVSECLFSLINGDDMFTTFSIMQEKSYLVWLFSRIYLYSFISLFIYMVLSLFIALITDTYDTIKNYQIDGFPESELHTFVSECKDLPTSGRYREQDETSCLSMLCCNR

SEQ ID NO: 33 - DNA, 1896 bp, Silurana tropicalis; identical to SEQ ID NO: 31

SEQ ID NO: 34 - protein, 553 aa, Silurana tropicalis; identical to SEQ ID NO: 32

SEQ ID NO: 35 - DNA, 6923 bp, Homo sapiens
cgcgctgcctgagctgagccgccgtaggtgaggggcccgcgtccccgcccgccctgggcgccgcgcctggcactgatcctgccggtcgcccactgtcgccgccgccgccgcccgcgggcaccatgacagctctgagcgctggggttacagactgtggttttgtgcttgctcaccaaagctaacctcagcatgctcaaaaggaagcagagttccagggtggaagcccagccagtcactgactttggtcctgatgagtctctgtcggataatgctgacatcctctggattaacaaaccatgggttcactctttgctgcgcatctgtgccatcatcagcgtcatttctgtttgtatgaatacgccaatgaccttcgagcactatcctccacttcagtatgtgaccttcactttggatacattattgatgtttctctacacggcagagatgatagcaaaaatgcacatccggggcattgtcaagggggatagttcctatgtgaaagatcgctggtgtgtttttgatggatttatggtcttttgcctttgggtttctttggtgctacaggtgtttgaaattgctgatatagttgatcagatgtcaccttggggcatgttgcggattccacggccactgattatgatccgagcattccggatttatttccgatttgaactgccaaggaccagaattacaaatattttaaagcgatcgggagaacaaatatggagtgtttccatttttctacttttctttctacttctttatggaattttaggagttcagatgtttggaacatttacttatcactgtgttgtaaatgacacaaagccagggaatgtaacctggaatagtttagctattccagacacacactgctcaccagagctagaagaaggctaccagtgcccacctggatttaaatgcatggaccttgaagatctgggacttagcaggcaagagctgggctacagtggctttaatgagataggaactagtatattcaccgtctatgaggccgcctcacaggaaggctgggtgttcctcatgtacagagcaattgacagctttccccgttggcgttcctacttctatttcatcactctcattttcttcctcgcctggcttgtgaagaacgtgtttattgctgttatcattgaaacatttgcagaaatcagagtacagtttcaacaaatgtggggatcgagaagcagcactacctcaacagccaccacccagatgtttcatgaagatgctgctggaggttggcagctggtagctgtggatgtcaacaagccccagggacgcgccccagcctgcctccagaaaatgatgcggtcatccgttttccacatgttcatcctgagcatggtgaccgtggacgtgatcgtggcggctagcaactactacaaaggagaaaacttcaggaggcagtacgacgagttctacctggcggaggtggcttttacagtactttttgatttggaagcacttctgaagatatggtgtttgggatttactggatatattagctcatctctccacaaattcgaactactactcgtaattggaactactcttcatgtatacccagatctttatcattcacaattcacgtactttcaggttctccgagtagttcggctgattaagatttcacctgcattagaagactttgtgtacaagatatttggtcctggaaaaaagcttgggagtttggttgtatttactgccagcctcttgattgttatgtcagcaattagtttgcagatgttctgctttgtcgaagaactggacagatttactacgtttccgagggcatttatgtccatgttccagatcctcacccaggaaggatgggtggacgtaatggaccaaactctaaatgctgtgggacatatgtgggcacccgtggttgccatctatttcattctctatcatctttttgccactctgatcctcctgagtttgtttgttgctgttattttggacaacttagaacttgatgaagacctaaagaagcttaaacaattaaagcaaagtgaagcaaatgcggacaccaaagaaaagctccctttacgcctgcgaatctttgaaaaatttccaaacagacctcaaatggtgaaaatctcaaagcttccttcagattttacagttcctaaaatcagggagagttttatgaagcagtttattgaccgccagcaacaggacacatgttgcctcctgagaagcctcccgaccacctcttcctcctcctgcgaccactccaaacgctcagcaattgaggacaacaaatacatcgaccaaaaacttcgcaagtctgttttcagcatcagggcaaggaaccttctggaaaaggagaccgcagtcactaaaatcttaagagcttgcacccgacagcgcatgctgagcggatcatttgaggggcagcccgcaaaggagaggtcaatcctcagcgtgcagcatcatatccgccaagagcgcaggtcactaagacatggatcaaacagccagaggatcagcaggggaaaatctcttgaaactttgactcaagatcattccaatacagtgagatatagaaatgcacaaagagaagacagtgaaataaagatgattcaggaaaaaaaggagcaagcagagatgaaaaggaaagtgcaagaagaggaactcagagagaaccacccatacttcgataagccactgttcattgtcgggcgagaacacaggttcagaaacttttgccgggtggtggtccgagcacgcttcaacgcatctaaaacagaccctgtcacaggagctgtgaaaaatacaaagtaccatcaactttatgatttgctgggattggtcacttacctggactgggtcatgatcatcgtaaccatctgctcttgcatttccatgatgtttgagtccccgtttcgaagagtcatgcatgcacctactttgcagattgctgagtatgtgtttgtgatattcatgagcattgagcttaatctgaagattatggcagatggcttatttttcactccaactgctgtcatcagggacttcggtggagtaatggacatatttatatatcttgtgagcttgatatttctttgttggatgcctcaaaatgtacctgctgaatcgggagctcagcttctaatggtccttcggtgcctgagacctctgcgcatattcaaactggtgccccagatgaggaaagttgttcgagaacttttcagcggcttcaaggaaatttttttggtctccattcttttgctgacattaatgctcgtttttgcaagctttggagttcagctttttgctggaaaactggccaagtgcaatgatcccaacattattagaagggaagattgcaatggcatattcagaattaatgtcagtgtgtcaaagaacttaaatttaaaattgaggcctggagagaaaaaacctggattttgggtgccccgtgtttgggcgaatcctcggaactttaatttcgacaatgtgggaaacgctatgctggcgttgtttgaagttctctccttgaaaggctgggtggaagtgagagatgttattattcatcgtgtggggccgatccatggaatctatattcatgtttttgtattcctgggttgcatgattggactgaccctttttgttggagtagttattgctaatttcaatgaaaacaaggggacggctttgctgaccgtcgatcagagaagatgggaagacctgaagagccgactgaagatcgcacagcctcttcatcttccgcctcgcccggataatgatggttttagagctaaaatgtatgacataacccagcatccattttttaagaggacaatcgcattactcgtcctggcccagtcggtgttgctctctgtcaagtgggacgtcgaggacccggtgaccgtacctttggcaacaatgtcagttgttttcaccttcatctttgttctggaggttaccatgaagatcatagcaatgtcgcctgctggcttctggcaaagcagaagaaaccgatacgatctcctggtgacgtcgcttggcgttgtatgggtggtgcttcactttgccctcctgaatgcatatacttacatgatgggcgcttgtgtgattgtatttaggtttttctccatctgtggaaaacatgtaacgctaaagatgctcctcttgacagtggtcgtcagcatgtacaagagcttctttatcatagtaggcatgtttctcttgctgctgtgttacgcttttgctggagttgttttatttggtactgtgaaatatggggagaatattaacaggcatgcaaatttttcttcggctggaaaagctattaccgtactgttccgaattgtcacaggtgaagactggaacaagattatgcatgactgtatggttcagcctccgttttgtactccagatgaatttacatactgggcaacagactgtggaaattatgctggggcacttatgtatttctgttcattttatgtcatcattgcctacatcatgctaaatctgcttgtagccataattgtggagaatttctccttgttttattccactgaggaggaccagcttttaagttacaatgatcttcgccactttcaaataatatggaacatggtggatgataaaagagagggggtgatccccacgttccgcgtcaagttcctgctgcggctactgcgtgggaggctggaggtggacctggacaaggacaagctcctgtttaagcacatgtgctacgaaatggagaggctccacaatggcggcgacgtcaccttccatgatgtcctgagcatgctttcataccggtccgtggacatccggaagagcttgcagctggaggaactcctggcgagggagcagctggagtacaccatagaggaggaggtggccaagcagaccatccgcatgtggctcaagaagtgcctgaagcgcatcagagctaaacagcagcagtcgtgcagtatcatccacagcctgagagagagtcagcagcaagagctgagccggtttctgaacccgcccagcatcgagaccacccagcccagtgaggacacgaatgccaacagtcaggacaacagcatgcaacctgagacaagcagccagcagcagctcctgagccccacgctgtcggatcgaggaggaagtcggcaagatgcagccgacgcagggaaaccccagaggaaatttgggcagtggcgtctgccctcagccccaaaaccaataagccattcagtgtcctcagtcaacttacggtttggaggaaggacaaccatgaaatctgtcgtgtgcaaaatgaaccccatgactgacgcggcttcctgcggttctgaagttaagaagtggtggacccggcagctgactgtggagagcgacgaaagtggggatgaccttctggatatttaggtggatgtcaatgtagatgaatttctagtggtggaaaccgttttctaataatgtccttgattgtccagtgagcaatctgtaattgatctataactgaattccagcttgtcacaagatgtttataaattgattttcatcctgccacagaaaggcataagctgcatgtatgatgggttactatcaatcattgctcaaaaaaatttttgtataatgacagtactgataatattagaaatgataccgcaagcaaatgtatatcacttaaaaatgtcatatattctgtctgcgtaaactaaggtatatattcatatgtgctctaatgcagtattatcaccgccccgcaaaagagtgctaagcccaaagtggctgatatttagggtacaggggttatagctttagttcacatctttcccatttccactagaaatatttctcttgagagaatttattatttatgattgatctgaaaaggtcagcactgaacttatgctaaaatgatagtagttttacaaactacagattctgaattttaaaaagtatcttctttttctcgtgttatatttttaaatatacacaagacatttggtgaccagaacaagttgatttctgtcctcagttatgttaatgaaactgttgcctccttctaagaaaattgtgtgtgcaagcaccaggcaaagaaatggactcaggatgcttagcggtttaaaacaaacctgtagataaatcacttgagtgacatagttgcgcaaagatgttaagtttcttaagaaaccttttaataactgagtttagcaaaaagaataaaactatatagctcaatttatttaaaaaaatctttgcatgtgtgatgttatcattggcttcatttcttacccaaggtatgtctgttttgccataaatcagcagagtcatttcattctgggtgatcctaacacaccattgctacgttagatttgaaatgacatctctgttaaaagaatcttctatggaaataatggtgccctgcaaaatcttcctttgaactcacaggttagggatcacacaacttacttaatcgttttttgtttttgttttttttccttatatgtcaatggcccatgtcctccgggaaaattagaaaagcaaaatgattacaaagtgctgttagatttcttgtgctgggccagccaagtagaagtggacttgacttggacctttaactattttattacagattggacatttgctgttcagatgttttttaacagagggattatctcagaatcctgtgacctccaggttgttttataatctatttttctctatttaacattcctcagatagataggcaaataggacattccttctgtgtcacagaagtatcgtggtagtggcagtctacagtttatatgattcattgtaactatgagataaagaacaaccagtcatgtggccaaaaggattagatttgatttgatgttcacttggagtttactttttgtacatacaagataaaataaatattggatttgtaaaat

SEQ ID NO: 36 - protein, 1738 aa, Homo sapiens
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 37 - DNA, 1566 bp, Homo sapiens
agccgcgagcggcggccgcggggccgaggagcctgggccgggccgggcggggactactccggagtcaggaggcagcagcggcggaggacgaggatctctggcagtcagcgccgctcggacgccgccggcaccatgggctgctgcaccggacgctgctcgctcatctgcctctgcgcgctgcagttggtctcagcattagagaggcagatctttgacttccttggtttccagtgggcgcctattcttggaaattttctacacataatagttgtcatattgggtttgtttgggaccattcagtacagacctcgatacataatggtgtatacagtgtggactgccctctgggtcacctggaatgtgttcattatctgcttttatttggaagtaggtggactctcaaaggacaccgatctaatgacattcaatatctctgtacatcggtcatggtggagagaacatgggcctggttgtgtcagaagagtgctgcctccctcagcccatggcatgatggacgattacacgtacgtctctgtcacaggctgcatcgttgacttccagtacctggaggtcatccacagtgctgtccaaatactactctctttggtgggttttgtgtatgcctgttatgtgatcagtatttccatggaagaagaagacacatattcatgtgatctgcaagtatgcaaacatctttttatccagatgctgcaaattattgaataagcaagaattagtaagatattatcaccaaattgtcacatcagtcaagcctcatgtgcttcctaagaactgaggtgatgcattattttagagtgtcattctaaaccccagattcaacatcttcctaatctttctagtgcagtctaatatataaattttatgaaaagcataggtttttttttaaccagcagtgctctttgagaatttacattgattcctaaagattgccattgctttgtataaaatgttataaattatcttagcatcttacctggaatttccactaaattcaccaatttatgatttgtgaaatctgattttactttttgaaaattttcatgtgaatttcccattttcagtgttgtagcacctctctcttcctctaagatcctccaagctcatcaaaagccatgatcttattataccagcagttttatttattcaatctttcaacaagtagttattgaacttctataatgtgccaggctctggagctcgccttacaccaaacagacacaatcgatccattcgaagtgtcgtaattacacattgagggaccaactagaccttttctcattgtaaacttggagcaaaagtaaattcattaaaataaatttacattatagtgccacaaaaaaatgaacagaaccagaaagcattttttacaaaaattaacagaacagtgtgatagaggggaaaaggatgtgagatcatggtgccctaccttcaatagggtggccagaaaacacctctctgaagaagcagcatttgagctgagacctgaagaacgaggagtcagtgatgcagagaacctcaggagatgccttccaatctgagaaaaga

SEQ ID NO: 38 - protein, 197 aa, Homo sapiens
MGCCTGRCSLICLCALQLVSALERQIFDFLGFQWAPILGNFLHIIVVILGLFGTIQYRPRYIMVYTVWTALWVTWNVFIICFYLEVGGLSKDTDLMTFNISVHRSWWREHGPGCVRRVLPPSAHGMMDDYTYVSVTGCIVDFQYLEVIHSAVQILLSLVGFVYACYVISISMEEEDTYSCDLQVCKHLFIQMLQIIE

SEQ ID NO: 39 - DNA, 2958 bp, Homo sapiens
tggagtcgctcgctgactcgccctgcgccctcgccgcggacaccggagctgcggccgctccccgctgtcccccagcttactccaatcaagcctctgcccgccaggaacaggtaacctgtgtgtgtccgtttgctccttctaagagcatgcctgatagatacttcggtagcctctccggatggccccttcgtcgggtagcctctcctgatggggtccttcgcccaccctgcctcccgcgccggcgctccgggtgaatgtcaagggtggctggctgcgaatacctccttcagctgctggggttcccgacagtttgcagtttttaaaagtgcaccctcggaagggcttttcagactgggtaaacctgacttttccaagagatggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttatacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggtagggaacgttgctttctaggaatgctactgacattttgattgacagagacattcactgtgcctcccctcttttccctaaaggagtttgtcaattttttcctcctccattataagaaggaagtttctgtttctgatcaaatggaatttgtcaatggatggtacattatgattattattagtgacatattgacaatcattggatcaattctaaaaatggaaatccaagctaaggtaatttttttcctaatcatgctattgttagtgtcagatttgcactaatggtaatgtatttttccagaatgtaagaattttcagaatgaattgtttcttccaaactgtatatcaagtagacttgaaattggtaatggtaattttcttaaatctagtcaggaggtctcttaggcagagtttttcaaagtgtgatccacaaaccattgcatcagaatcattgggtgcctggtaaagtgtaccatgttagacctactgaattcagactcttcggcggggcctgtgaattcttacacacaccaaaattcatacacaaccaaggtaactaaggtaagagtttttttttttttttaatcttacaagaaatgctcgaatctttaacaaaaatgagtggggctataggggaaagtgaggtcaaggcactatggtgtgcatgcttgcatttgtttcctccgtccattcaaagtgagaatgctcccattttcttactttaccattgatgtgctacaagcttatttattttaagactaacctagcctaaaaatcaactgtccccacaaaataaaaatcacattaaaaaaactaatagtgttcagactaatcttgctcaaacttatgtttccctagtcttgatgcaactgattgagtcacctggggagttggttataaacctgggcagagaccccaaatgcaatggctcagagaagataggagcttatttctgtcttatgcaatagtcagaatgggttttacagactggtgagtagctcaacatctcacagtcattcaggcacccatgttcctcccattttgtttctctgccaccccttaaggacttgccctgactgcatgattattgccgtgttgcctcaaacaggttgcagcttatgggaagcaaaaacacggtatggtagaagctctcccatagactgatggcttggctcaagagtggccgactttatttctgtacatatcccactggatagaatttagtcaatcctaactgcagagggagccagggaacacagcccaggcatgtgcctaggaaggggagaatgggtttaggttgacacttagcagctgccactatatgtggctatagtatgtatcattggaatagatgtttaactttagggacaaataaaaaaccaaaacaaaaaaaggagtaaggggagagatttgcagcaaatctttatttttaccaacctcaactatcattaatttcagtgaaccctaaatggtatccaacaaaatatctttctagaccattcaccgtctctgcctcatagatgatcatatcatgttttcttctcttctgaaacctctaatacccttgtcctatcctcattctaagctgatgaccttacttcctatttcacaaaaataatagaaaaaaaaaaaaaaaa

SEQ ID NO: 40 - DNA, 2727 bp, Homo sapiens
atagcctttcaaatttcggttaatggtaactctcatcagttactcaagccaaaaatcttggattcatcacagactcctctctttcactaatctcttcttccccacatctaatccaagaggaaatcctattgttctaccttcattggccaggcactgtgactcatataatcccagcacattgggaggctgaggcgggaggatggcttgagcccaggagttccagaccagcctgggaaacatagtgaggccccatctctacaaaaaattaaaaaaattagcagtcaaggtggcatgtgtctgtaatccaagctacttggggggctgaggtgggaggatttcttgagcccgggaagtcgaggctgcattgagccacggttgtgccacggcactccagcctgggtgacagagtgagacccctgtttccaaaaaaaaaaaaaaaaaaaaagaataaaaatcacctggtcaactccactattaccagcctggtccaagctactatctctcatttatattattgcaatagcctcctcactcctccaacaacctgctgtcaaccacagcagccaacatctgatcatatcacttctgtttgtggttctcaaatctccccaattgagttacagtaaaagacaaacttggtgagtgccaccttatctctataactgtataccctttctattgctcactccagccagatgcaatatccttgccaagcaccctcctgtctcagggcctttgcacttgccagtccctgtgcctggaaggcttctcccctagatttttgcatgacttctccctccctcccttcagatctttgctcaaatgccttctttttagtgtatgtaaaatgacaaacccatacccattccttatcccctcctctgaattttctcttcagcaattatcagcagcaagtgtcccaaagtttctattaacttatttctgttgtctctttcttccctccactagaatgtaagctttatgagagcagagacttttgtttgttcactgctttatccttagcacctaaaacagtgccttactcatagttacctcaatatttattgccaaatgaatttctgctttataatctgattatatttttccactctctcttagagtctaactagttatgatgtctgtagcatacttcttgggacttctaccatgcttgtgtggcttggagtcatccgatacctcggtttctttgcaaagtacaacctcctcattttgacccttcaggcagcgctgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtaactttattctgctgttgtaaaaagtagctatcaggtttatctgtactttagaggaaaatataatgtgtagctgagttggaacactgtggatattctgagatcagatgtagtatgtttgaagactgttattttgagctaattgagacctataattcaccaataactgtttatatttttaaaagcaatatttaatgtctttgcaactttatgctgggattgtttttaaaaaaaactttaatgaggaaagctattggattattattatttcttgtttattttgccatggctttagaatgtattctgtatgcctctcttttgctctgatactgttgctcctgctattctgattgtgcagactgtataattagtggaaaacaatccttggtctgactgtgactttggacaactcagtaaccctggcttggaccactctcaggagtccatccttgagagagtgggtgtagttaccatttatacagtaatcattgcattttaaaatcttctcttgaaaggaagaataagagtgcaccagaataagagcgcaccagaataagagcacaccagctaacaatgtgatacggccatatgtcacttaaggatggagatatgttctgagaaatgtgtcattaggcgattttgtcattaaacatcatagcatgtacttccacaaacctagatggtatagcctactacacacctaggctatttggtatagcctgttggtcctggggtacaaatctgtacaacatgttactgtattgaatacagtaggcaattgtaactcaatggtaagtatctaaacatagaaaagggacagtaaaaatatggttttataatcttctgggaccaccattgtatatgcggtacatcattgaccaaaacatcgttatccagcatatgactgtatttggttatgaaagccaactgttacttgattctgcttttagttcttaagaggatcaggcttttaaatactcatttacaagctttctatcctccttcagtgttaaagtagaaagtaaaaagagtatcttatacatgcatgaaattaaagcatataccaaatgc

(19) United States
(12) Patent Application Publication
(10) Pub. No.: US 2009/0210953 A1
(43) Pub. Date: Aug. 20, 2009

(54) Identification of TRPML3 (MCOLN3) as a salty taste receptor and use in assays for identifying taste (salty) modulators and/or therapeutics that modulate sodium transport, absorption or excretion and/or aldosterone, and/or vasopressin production or release

(76) Inventors: Bryan Moyer, San Diego, CA (US); Albert Zlotnik, San Diego, CA (US); Peter Hevezi, Encinitas, CA (US); Hortensia Soto, San Diego, CA (US); Dalia Kalabat, El Cajon, CA (US); Min Lu, San Diego, CA (US); Na Gao, San Diego, CA (US); Guy Servant, San Diego, CA (US); Evan Carl White, Fair Oaks, CA (US); Paul Brust, San Diego, CA (US); Mark Williams, Carlsbad, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/328,887

(22) Filed: Dec. 5, 2008

Related U.S. Application Data
(63) Continuation-in-part of application No. 12/134,390, filed on Jun. 6, 2008.
(60) Provisional application No. 60/929,017, filed on Jun. 8, 2007.
(60) Provisional application No. 60/929,007, filed on Jun. 8, 2007.
(60) Provisional application No. 60/947,052, filed on Jun. 29, 2007.
(60) Provisional application No. 60/935,297, filed on Aug. 3, 2007.
(60) Provisional application No. 60/987,611, filed on Nov. 13, 2007.
(60) Provisional application No. 60/988,938, filed on Nov. 19, 2007.
(60) Provisional application No. 60/991,274, filed on Nov. 30, 2007.
(60) Provisional application No. 60/991,289, filed on Nov. 30, 2007.
(60) Provisional application No. 60/992,502, filed on Dec. 5, 2007.
(60) Provisional application No. 60/992,517, filed on Dec. 5, 2007.
(60) Provisional application No. 61/017,244, filed on Dec. 28, 2007.
(60) Provisional application No. 61/021,437, filed on Jan. 16, 2008.
(60) Provisional application No. 61/043,257, filed on Apr. 8, 2008.
(60) Provisional application No. 61/053,310, filed on May 15, 2008.

(51) Int. Cl. A01K 67/00; C12Q 1/68; C12Q 1/02; C12N 5/06; C12N 5/10; C12N 1/00

(52) U.S. Cl. 800/13; 435/6; 435/29; 435/325; 435/349; 435/350; 435/351; 435/352; 435/363; 435/366; 435/317.1; 800/14; 800/19; 800/20

(57) ABSTRACT

The present invention relates to the elucidation that TRPML3 is involved in salty taste perception in primates including humans and likely other mammals and based thereon high-throughput mammalian and medium-throughput oocyte-based electrophysiological assays for identifying human TRPML3 modulators, preferably TRPML3 enhancers. Compounds that modulate TRPML3 function in the assay are expected to affect salty taste in humans. The inventive electrophysiological assays, such as the two-electrode voltage-clamp technique, facilitate the identification of compounds which specifically modulate human TRPML3. The assays of the invention provide a robust screen useful to detect compounds that facilitate (enhance) or inhibit TRPML3 function. Compounds that enhance or block TRPML3 channel activity should thereby modulate salty taste. In addition, these compounds may be used to regulate sodium excretion, urinary output and other biological functions relating to sodium levels and TRPML3 related functions.

### RELATED PROVISIONAL AND UTILITY APPLICATIONS

This application is a continuation-in-part of U.S. Ser. No. 12/134,390 filed on Jun. 6, 2008 which further relates to relates to, and claims priority to U.S. Ser. No. 11/808,356, filed on Jun. 8, 2007. In addition, this application based on its priority claim to U.S. Ser. No. 12/134,390 filed on Jun. 6, 2008 further relates to and claims priority to earlier filed provisional applications by the present Assignee Senomyx Inc relating to a novel rationale for identifying primate taste specific genes and in particular for identification of the primate salt receptor gene or genes. These provisional applications are U.S. Application Ser. No. 60/929,017, filed Jun. 8, 2007; U.S. Application Ser. No. 60/929,007, filed Jun. 8, 2007; U.S. Application Ser. No. 60/947,052, filed Jun. 29, 2007; U.S. Application Ser. No. 60/935,297, filed Aug. 3, 2007; U.S. Application Ser. No. 60/987,611, filed Nov. 13, 2007; U.S. Application Ser. No. 60/988,938, filed Nov. 19, 2007; U.S. Application Ser. No. 60/991,274, filed Nov. 30, 2007; U.S. Application Ser. No. 60/991,289, filed Nov. 30, 2007; U.S. Application Ser. No. 60/992,502, filed Dec. 5, 2007; U.S. Application Ser. No. 60/992,517, filed Dec. 5, 2007; U.S. Application Ser. No. 61/017,244, filed Dec. 28, 2007; U.S. Application Ser. No. 61/021,437, filed Jan. 16, 2008; U.S. application Ser. No. 61/043,257, filed Apr. 8, 2008; and U.S. Application Ser. No. 61/053,310, filed May 15, 2008. All of the afore-mentioned provisional and non-provisional applications are incorporated by reference in their entireties herein.

These applications include disclosure relating to various taste specific genes including TRPML3 or MCOLN3 as this gene is alternatively known. This gene was highlighted as a gene to be functionalized by the inventors in electrophysiological assays. This gene was theorized by the inventors to be a candidate gene encoding a salty taste receptor because it was predicted to encode a taste specific sodium ion channel regulating salty taste in primates (e.g., humans), rodents and other animals. As described herein, in vitro and in vivo (animal) assays using cells (mammalian and amphibian cells) and rodents expressing wild type or mutated TRPML3 polypeptides have confirmed that TRPML3 or MCOLN3 is involved in salty taste perception as well as affecting other biological functions unrelated to taste such as hearing and balance (as evidenced by mutations in rodent TRPML3, that kill hairy cells in the inner ear, having adverse effects on hearing (deafness) and causing impaired balance, as well as resulting in a disruption in normal pigmentation apparently attributable to the loss of melanocytes as a result of this same TRPML3 mutation). Also, results obtained by the inventors disclosed herein, and previously unknown, suggest that this gene, because of its probable role in sodium transport, excretion and absorption, its role in sodium sensing, and based upon the tissues where it is known to be specifically expressed including the kidney, adrenal gland and the pituitary gland, indicate that it modulates or participates with aldosterone and/or vasopressin in the regulation of sodium transport, metabolism, excretion and other sodium and possibly other ion related functions involving aldosterone and/or vasopressin as these hormones are play an important role in sodium transport, metabolism, and excretion in different mammals including humans and rodents. More specifically, results disclosed herein indicate that TRPML3 plays a role in sodium homeostasis, including blood pressure regulation in hypertension (high blood pressure) and hypotension (low blood pressure); modulating renal sodium balance to control blood pressure; modulation of plasma osmolarity, including decreases in osmolarity (hypoosmotic) and increases in osmolarity (hyperosmotic) via reabsorption of water in the kidney in normal and diseased states such as diabetis insipidus; modulation of ENaC function and/or trafficking; modulation of aquaporin-2 function and/or trafficking; modulation of vasopressin (antidiuretic hormone) secretion from the pituitary gland and modulation of aldosterone secretion from the adrenal glands.

### SEQUENCE LISTING

The sequence listing in the file named “67824o703305.txt” having a size of 157168 bytes that was created Dec. 4, 2008 is hereby incorporated by reference in its entirety.

### FIELD OF THE INVENTION

This invention relates to the discovery that a specific ion channel polypeptide (TRPML3 or MCOLN3) which is member of the TRPML subfamily of the transient receptor potential cation channel superfamily is involved in taste (salty) perception, e.g., sodium taste sensing and sodium homeostasis in general.

More specifically, this invention relates to the discovery that a specific ion channel polypeptide (TRPML3 or MCOLN3) is involved in taste (salty) perception in primates (human and non-human primates), rodents, and other mammals, and likely other vertebrates (avians, reptiles, amphibians), given the importance of maintaining sodium and potassium ion levels within different concentration thresholds, and given the important effect of these ions on physiological processes important to the well being of different organisms.

In addition, the invention relates to the discovery that TRPML3 or MCOLN3 polypeptides or functional variants thereof when expressed separately or in combination with other taste specific polypeptides (e.g., other accessory molecules such as GPCRs or ion channels including e.g., TRPML1, TRPML2, NKAIN3 or NALCN) functions as a taste receptor that responds to salty taste stimuli and potentially other taste eliciting molecules.

In addition the invention relates to the discovery that this gene, because of its role in sodium sensing, and the tissues where it is known to be specifically expressed, which include the adrenal and the pituitary gland, encodes a polypeptide that alone or in association with other accessory molecules plays a role in modulating the levels and release of aldosterone and vasopressin and thereby sodium related physiological activities regulated by aldosterone and/or vasopressin.

In addition the invention relates to the discovery that TRPML3 modulators, because TRPML3 is involved in sodium sensing, and further based on its expression in the parathyroid organ can be used to treat diseases involving the parathyroid including but not limited to calcium homeostasis. hyperparathyroidism, hypoparathyroidism, hypercalcemia, osteitis cystica, pseudoparathyroidism, Jansen's metaphyseal chondroplasia, Blomstrand's chondroplasia, and osteoporosis of different causes including e.g., age related, menopausal, or drug, chemotherapy or radiotherapy induced. In addition the invention relates to the discovery that TRPML3 modulators, because TRPML3 is involved in sodium homeostasis, including blood pressure regulation in hypertension (high blood pressure) and hypotension (low blood pressure) that TRPML3 enhancers and blockers can be used to modulate renal sodium balance to control blood pressure; that TRPML3 enhancers and blockers can be used to modulate plasma osmolarity, including decreases in osmolarity (hypoosmotic) and increases in osmolarity (hyperosmotic) via reabsorption of water in the kidney in normal and diseased states such as diabetis insipidus; that TRPML3 enhancers and blockers can be used to modulate ENaC function and/or trafficking; that TRPML3 enhancers and blockers can be used to modulate aquaporin-2 function and/or trafficking; that TRPML3 enhancers and blockers can be used to modulate vasopressin (antidiuretic hormone) secretion from the pituitary gland; and that TRPML3 enhancers and blockers can be used to modulate aldosterone secretion from the adrenal glands.

The invention also relates to the use of an animal model (Varitint waddler mouse) containing a mutated form of TRPML3 or MCOLN3 gene, where TRPML3 salty taste cells are specifically ablated from taste buds and where salty taste is greatly diminished in order to study the effect of TRPML3 on salty taste and other effects of this ion channel on sodium transport, metabolism, and excretion, including plasma osmolarity, blood pressure, fluid retention, renal function, etc. as the gene likely has the same effects in other animals including humans.

The invention also relates to the use of a mutant TRPML3 gene (A419P TRPML3) to specifically ablate cell types including taste cells, kidney cells, adrenal cells, pituitary cells, or melanocytes and create mouse model systems lacking these different cell populations.

The invention also relates to the use of a mutant TRPML3 ion channel polypeptide (A419P TRPML3) as a toxin to kill specific cell types such as salty taste cells or melanocytes or adrenal cells or pituitary cells or kidney cells.

The invention also relates to applications of this gene and the corresponding polypeptide or variants thereof including allelic variants, chimeras, fragments and engineered mutants (e.g., mutants designed to modulate (increase or decrease) activity or to maintain ion channel in open or closed position) in assays for identifying TRPML3 modulatory compounds (TRPML3 agonists, antagonists, enhancers, blockers). These compounds potentially may be used as taste modulators and therapeutics that modulate or treat physiological functions and conditions involving aberrant vasopressin release, aldosterone production, melanocyte function or sodium transport, absorption and/or excretion. For example these compounds may be used as salty taste blockers, enhancers, or inhibitors, or for treating hypertension, urinary function, cardiovascular diseases, for treating melanocyte related conditions such as pigmentation disorders, melanomas, and mucous related conditions such as mucolipidosis type IV. More specifically these compounds may be used to treat sodium homeostasis, including blood pressure regulation in hypertension (high blood pressure) and hypotension (low blood pressure); TRPML3 enhancers and blockers can be used to modulate renal sodium balance to control blood pressure; TRPML3 enhancers and blockers can be used to modulate plasma osmolarity, including decreases in osmolarity (hypoosmotic) and increases in osmolarity (hyperosmotic) via reabsorption of water in the kidney in normal and diseased states such as diabetis insipidus; TRPML3 enhancers and blockers can be used to modulate ENaC function and/or trafficking; TRPML3 enhancers and blockers can be used to modulate aquaporin-2 function and/or trafficking; TRPML3 enhancers and blockers can be used to modulate vasopressin (antidiuretic hormone) secretion from the pituitary gland; and TRPML3 enhancers and blockers can be used to modulate aldosterone secretion from the adrenal glands.

The invention therefore also relates to therapeutic and prophylactic methods of modulating sodium homeostasis in a subject in need thereof comprising administering a modulatory effective amount of a TRPML3 modulatory compound which may be an agonist, antagonist, enhancer or blocker of TRPML3. These methods may be used in particular to regulate blood pressure, e.g., hypertension or hypotension.

The invention also specifically relates to therapeutic and prophylactic methods of modulating plasma osmolarity in a subject in need thereof comprising administering a modulatory effective amount of a TRPML3 modulatory compound. These methods may be used in particular to treat decreases in osmolarity (hypoosmotic) or increases in osmolarity (hyperosmotic) and also to promote the reabsorption of water in the kidney. The treated subjects may be normal (e.g., dehydrated as a result of exertion or overheating or may have a disease that affects osmolarity such as diabetis insipidus.

The invention also specifically relates to therapeutic and prophylactic methods of modulating ENaC function and/or trafficking in a subject in need thereof comprising administering an effective amount of a TRPML3 modulatory compound, e.g., a TRPML3 enhancer, blocker, agonist or antagonist.

The invention also specifically relates to therapeutic and prophylactic methods of modulating aquaporin-2 function and/or trafficking in a subject in need thereof comprising administering an effective amount of a TRPML3 modulatory compound, e.g., a TRPML3 enhancer, blocker, agonist or antagonist.

The invention also specifically relates to therapeutic and prophylactic methods of modulating vasopressin (antidiuretic hormone) secretion from the pituitary gland trafficking in a subject in need thereof comprising administering an effective amount of a TRPML3 modulatory compound, e.g., a TRPML3 enhancer, blocker, agonist or antagonist.

The invention also specifically relates to therapeutic and prophylactic methods of modulating aldosterone secretion from the adrenal glands in a subject in need thereof comprising administering an effective amount of a TRPML3 modulatory compound, e.g., a TRPML3 enhancer, blocker, agonist or antagonist.

Also, the invention specifically relates to genetic screening assays for detecting abnormalities that affect one or more of sodium homeostasis, plasma osmolarity, ENaC function and/or trafficking, aquaporin-2 function and/or trafficking, vasopressin (antidiuretic hormone) secretion from the pituitary gland, and/or aldosterone secretion from the adrenal glands comprising using a wild type TRPML3 sequence as a probe and detecting for mutations including substitution, deletion, addition mutations in the TRPML3 coding sequence or mutations in the regulatory regions that impair normal TRPML3 transcription and/or translation.

Based on the foregoing, the present invention specifically relates to a mammalian cell-based high-throughput screening assays (HTS assays) for the identification of TRPML3 modulators. In an exemplary embodiment, the invention teaches the use of cells expressing an active variant of TRPML3 (A419P-TRPML3) in cell-based assays for the identification of enhancers or blockers of TRPML3 function. Compounds that modulate TRPML3 function in a cell-based assay are anticipated to affect salty taste in humans and other mammals. The assays described in the present invention can be run in standard 96 or 384 well culture plates in high-throughput mode.

In an more specific embodiment this invention identifies and provides functional (electrophysiological), in vivo and immunohistochemistry data which provide evidence that TRPML3 (MCOLN3) encodes a polypeptide that functions as a primate (e.g., human) salty taste receptor

In a related embodiment the present invention provides the use of TRPML3 polypeptides and nucleic acid sequences and probes specific thereto as markers which can be used to enrich, identify or isolate salt receptor and other TRPML3 expressing cells.

In a related embodiment the present invention provides the use of TRPML3 polypeptides and nucleic acid sequences and probes specific thereto as markers which can be used to identify mutations in TRPML3 that may correlate to specific diseases and conditions relating to aberrant TRPML3 function or expression such as diseases involving abnormal sodium sensing, transport, excretion and absorption, melanocyte function (cancer, pigmentation disorders, et al.) and diseases involving aberrant aldosterone or vasopressin production or release such as cardiovascular and urinary diseases.

In another specific aspect this invention provides in vitro and in vivo assays which use TRPML3 (MCOLN3) and TRPML3 expressing cells or TRPML3 transgenic animal models to identify agonist, antagonist or enhancer compounds which elicit or modulate (block or enhance) salty taste in primates including humans. These assays use cells or animals which express TRPML3 (wild type or a variant thereof) or use cells which express the TRPML3 ion channel or a variant (e.g., functional variant having enhanced activity or genetically engineered to be fixed in the “open” or “closed” orientation) in association with other accessory polypeptides such as other taste specific polypeptides such as NALCN or NKAIN3, GPCRs or related ion channels such as TRPML1 and/or TRPML2.

In another aspect this invention provides transgenic animals, preferably rodents, and the use thereof to confirm the role of TRPML3 in salty taste in mammals and in other physiological functions involving sodium and other ions such as potassium, calcium, lithium and on ion (sodium) metabolism, blood pressure, fluid retention and excretion, urinary function and cardiac function.

In another aspect this invention provides in vitro and in vivo assays which use TRPML3 and TRPML3 expressing cells or transgenic animals in assays, e.g., neurophysiological behavioral or electrophysiological assays, in order to identify therapeutic compounds which alleviate diseases and conditions involving or caused by deficiencies in the expression of this ion channel polypeptide and/or its effects on specific cells including ablation. These conditions include by way of example conditions involving TRPML3 hyperexpression, hypoexpression, and mutations in the TRPML3 polypeptide that affect its ability to function as a taste specific sodium channel in a mammal including e.g., human and non-human primates. Other conditions include by way of example Addison's disease and diseases involving aberrant aldosterone or vasopressin production or release such as hypertension, hypotension, fluid retention, and impaired urinary or cardiac function such as arrhythmia, heart attack and stroke.

In another embodiment the invention relates to assays that identify compounds that modulate the function of the use of TRPML3 alone or expressed in association with another taste specific gene such as NALCN or NKAIN3 or TRPML1 or TRPML2 and the use of the identified compounds to modulate salty taste perception in humans and other mammals or vertebrates. In another embodiment the invention relates to assays that identify compounds that modulate the function of the use of TRPML3 alone or expressed in association with another taste specific gene such as NALCN or NKAIN3 or TRPML1 or TRPML2 and the use of the identified compounds to modulate salty taste perception in humans and other mammals or vertebrates.

The present invention further provides specific methods of isolating, purifying and marking salty and other TRPML3 expressing cell types and taste cell lineages including as well as taste stem cells and other immature and mature taste cell lineages including cells that differentiate into taste bud cells, taste cell neurons, taste immune cells et al. based on the expression or absence of expression of TRPML3 identified using the methods provided herein. These isolation and purification methods include both positive and negative cell separation methods. For example desired taste cell lineages or types may be isolated by positive cell selection methods e.g., by the use of fluorescence activated cell sorting (FACS), magnetic bead cell selection e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of one or several undesired cell lineages e.g., by contacting a mixed cell suspension containing the desired taste cells and undesired cells e.g., derived from the tongue, oral cavity or gastrointestinal tract and associated organs with cytotoxic antibodies specific to a target gene or genes expressed on the undesired taste cell type(s) which are to be removed.

Also, the invention relates to the use of the Varitint waddler mice to detect the effect of TRPML3 function on melanocytes, pituitary, adrenal, taste, urinary (e.g., kidney) or taste cells.

Also, the invention relates to the use of the Varitint waddler mice to detect the effect of TRPML3 function on melanocytes, pituitary, adrenal, taste, urinary (kidney et al.) and taste cells.

In addition the invention relates to the discovery that TRPML3 modulators, because TRPML3 is involved in sodium sensing, and further based on its expression in the parathyroid organ can be used to treat diseases involving the parathyroid including but not limited to calcium homeostasis. hyperthyroidism, hypothyroidism, hypercalcemia, osteitis cystica, pseudothyroidism, Jansen's metaphyseal chondroplasia, Blomstrand's chondroplasia, and osteoporosis of different causes including e.g., age related, menopausal, or drug, chemotherapy or radiotherapy induced.

Also, the invention relates to the use of the Varitint waddler mice in assays to detect genes specifically expressed in salty taste cells and not in the Varitint waddler mice (as salty taste cells are ablated therein) which genes may modulate TRPML3 function, or function as a salty taste receptor or modulate transmission of salty taste signaling from TRPML3 to the nerve fibers and/or control the development differentiation or apoptosis of salty taste cells. These gene detection assays may comprise the use of gene chips or microarray technology to compare the genes expressed in salty taste cells versus genes expressed in Varitint waddler mice.

The present invention further provides specific methods of isolating, purifying and marking salty and other TRPML3 expressing cell types and taste cell lineages including as well as taste stem cells and other immature and mature taste cell lineages including cells that differentiate into taste bud cells, taste cell neurons, taste immune cells et al. based on the expression or absence of expression of TRPML3 identified using the methods provided herein. These isolation and purification methods include both positive and negative cell separation methods. For example desired taste cell lineages or types may be isolated by positive cell selection methods e.g., by the use of fluorescence activated cell sorting (FAGS), magnetic bead cell selection e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of one or several undesired cell lineages e.g., by contacting a mixed cell suspension containing the desired taste cells and undesired cells e.g., derived from the tongue, oral cavity or gastrointestinal tract and associated organs with cytotoxic antibodies specific to a target gene or genes expressed on the undesired taste cell type(s) which are to be removed. Also, the invention relates to the use of the Varitint waddler mice to detect the effect of TRPML3 function on melanocytes, pituitary, adrenal, taste, urinary or taste cells.

Also, the invention relates to the use of the Varitint waddler mice to detect the effect of TRPML3 function on melanocytes, pituitary, adrenal, taste, urinary (kidney) or taste cells.

Also, the invention relates to the use of the Varitint waddler mice in assays to detect genes specifically expressed in salty taste cells and not in the Varitint waddler mice (as salty taste cells are ablated therein) which genes may modulate TRPML3 function, or function as a salty taste receptor or modulate transmission of salty taste signaling from TRPML3 to the nerve fibers o and/or control the development differentiation or apoptosis of salty taste cells. These gene detection assays may comprise the use of gene chips or microarray technology to compare the genes expressed in salty taste cells versus genes expressed in Varitint waddler mice.

Also the invention relates to the use of markers e.g., antibodies or oligonucleotides, that are specific to TRPML3 and/or related accessory polypeptides in mapping regions of the tongue and oral cavity which are involved in specific taste (salty) and non-taste specific functions, mapping of cell comprised on specific (salty) taste sensing cells in the gastrointestinal tract and associated organs such as the intestinal epithelium or urinary tract that express specific taste specific genes and which therefore are involved in one or more of the taste cell specific functions disclosed herein, and/or the use of the subject genes and markers specific thereto in taste cell differentiation studies, e.g. for identifying compounds that induce the differentiation or dedifferentiation of taste cells e.g., adult or embryonic stem cells and other pluripotent or immature cell types into desired taste cell lineages and taste cell types.

### BACKGROUND OF THE INVENTION

Various ion channels have been studied in order to elucidate their potential involvement in salty taste and regulation of sodium transport, metabolism and excretion. In particular, epithelial sodium channels (ENaC) which are members of the ENaC/degenerin family of ion channels that includes acid-sensing ion channels (ASIC) in mammals, mechanosensitive degenerin channels in worms, and FMRF-amide peptide-gated channels in mollusks (Kellenger, S. and Schild, L. (2002) Physiol. Rev. 82:735-767) have been extensively studied. ENaC mediates amiloride-sensitive apical membrane Na⁺ transport across high resistance epithelia in numerous tissues including kidney, colon, and lung.

ENaC is known to be a heterotrimeric channel comprised of alpha, beta, and gamma subunits or delta, beta, and gamma subunits. This heterotrimeric channel has been hypothesized to be involved in human salty taste perception. Previously, assays have been developed by the present assignee using ENaC sequences to identify compounds that modulate the delta beta gamma and alpha beta gamma human ENaC to examine if these compounds will potentially modulate human salty taste perception. Also, these compounds potentially may be used to treat human pathologies involving aberrant ENaC function.

Unlike other mammals, amiloride only slightly reduces the intensity of sodium chloride taste, i.e., by about 15-20% when used at concentrations that specifically modulate ENaC function (Halpem, B. P. (1998) Neuroscience and Behavioral Reviews. 23: 5-47). Experiments conducted by the inventors have shown that amiloride, or the more potent amiloride derivative phenamil did not elicit a significant effect on perceived human salt intensity when tested at levels 300-fold (for amiloride) and 3000-fold (for benzamil) above IC50 values for alpha beta gamma ENaC (equivalent to 10-fold for amiloride and 100-fold for benzamil over IC50 values for delta beta gamma ENaC). Based thereon, it was theorized that other genes (non-ENaC genes) were involved in human salty taste perception.

In addition, it has been recently reported that taste receptors may be expressed in non-oral tissues, e.g., in the digestive system and potentially other organs such as the kidney, suggesting that they have non-taste related activities, such as in food sensing and regulation of digestion et al. Particularly it has been reported that sweet, umami and bitter taste receptors are expressed in cells other than in the oral cavity such as gastrointestinal cells. (See, e.g., Stermini et al., Amerj Physiol. Gastrointestinal and Liver Physiology, 292:G457-G461, 2007; Mace, O. J. et al, J. Physiology. 10.1113/jphysiol.2007.130906. Published online May 10, 2007). Also, it has been reported by various groups (Margolskee et al., Bezencon et al., Rozengurt et al, and Stermini et al. (2007) (Id)) that bitter and umami taste receptors and other taste signaling molecules such as TRPM5 and gustducin are expressed in specialized cells in the gastrointestinal tract. (See e.g., Margolskee et al., Genes Brain Behavior 2007 (epub March 21); Rozengurt et al., Amer. J. Physiol. Gastroent. Liver Physiol. 291(2):G171-7 (2006); Bezencon et al., Chem Senses 32(1):41-47 (2007)). Margolskee et al. (Id) further reports that the loss of T1R3 or gustducin in rodents resulted in changes in insulin metabolism and the release of satiety peptides such as GLP-1 (glucagon-like peptide 1).

Based on these observations with other taste receptors, it is likely that salty receptors are expressed in tissues that play important roles in controlling sodium ion homeostasis such as the adrenal gland, pituitary gland, and kidney. Because taste receptors are expressed on non-taste cells such as digestive organs and likely organs in the urinary system they are involved in functions not directly related to taste such as digestive functions such as gastric motility, absorption, food detection, metabolism, and immune regulation of the oral or digestive tract and may also affect functions relating to sodium absorption, excretion and transport such as blood pressure and fluid retention. Therefore, the identification of taste cell specific genes and identifying what specific cells these genes are specifically expressed should facilitate a better understanding of other non-taste functions of these taste receptors and also facilitate the use of these genes, gene products and cells which express same in assays for identifying novel therapeutics, e.g., for treating digestive diseases such as autoimmune, inflammatory and cancers, metabolism, diabetes, eating disorders, obesity, taste cell turnover, hypertension, fluid retention, and immune regulation of the digestive system. In the specific case of sodium (salty) taste receptors, elucidating the specific identity of the gene or genes which are significant for salty taste sensing should facilitate an understanding of the role of these genes on other sodium related functions and polypeptides such as vasopressin or aldosterone which are involved in sodium transport, metabolism and excretion, critical to urinary and cardiovascular function.

As mentioned above, this invention relates specifically to the discovery that TRPML3 is an ion channel polypeptide that is involved in sodium (salty) taste sensing in mammals and potentially other vertebrates given the importance of sodium and other ions (such as potassium, calcium, lithium) to many physiological functions which would indicate that this gene may be conserved in different vertebrates. Prior to the specific discovery of the inventors herein, i.e. that TRPML3 (MCOLN3) encodes an ion channel that is involved in salty taste perception in primates and other mammals, (and further likely plays a role in related physiological functions involving sodium transport and excretion), this gene had been reported previously to be responsible for the phenotype of a mouse mutant called varitint-waddler that exhibits early-onset hearing loss, vestibular defects, pigment abnormalities and perinatal lethality (DiPalma et al., Mutations in Mcoln3 associated with deafness and pigmentation defects in varitint-waddler (Va) mice. Proc. Natl. Acad. Sci. USA 99: 14994-14999; 2002). It was further reported that MCOLN3 or TRPML3 is expressed in the hair cells and plasma membrane of stereocilia (in the ears). Particularly, a mutation in this polypeptide that resulted in an ala 419 to pro substitution in the fifth transmembrane domain has been reported to result is a hyperactive MCOLN3 that results in the death of cells expressing this molecule, i.e., the hair cells of the ear (hence the deafness of the Va mouse) (Grimm C et al., Proc Natl. Acad. Sci. USA 104: 19583-8; 2007).

While in mice, the A419P-TRPML3 mutation results in a severe form of the varitint-waddler phenotype. (Kim et al., J. Biol. Chem., 2007 Dec. 14; 282(50):36138-42. Epub 2007 Oct. 25; Nagata et al., Proc. Natl. Acad. Sci, USA, 2008 Jan. 8; 105(1):353-8. Epub 2007 Dec. 27; Grimm et al., Proc. Natl. Acad. Sci, USA, 2007 Dec. 4, 104(49):19583-8. Epub 2007 Nov. 28), characterized by pigmentation defects, hearing loss and embryonic lethality, another TRPML3 mutation (TRPML3(1362T/A419P) results in a milder form of the varitint-waddler phenotype.

How the mutations cause each phenotype are not known. It has been reported that the first channel properties of TRPML3 are as a strongly inward rectifying cation channel with a novel regulation by extracytosolic Na+. (Kim et al. 2007 (Id.)) They further report that preincubating the extracytosolic face of TRPML3 in Na+-free medium is required for channel activation, but then the channel slowly inactivates. Therefore, the A419P mutation locks the channel in an open unregulated state. The Kim et al. researchers further observed similar gain of function with the A419G mutation, which, like A419P, is expected to destabilize the alpha-helical fifth transmembrane domain of TRPML3. By contrast, Kim et al., observed that the 1362T mutation results in an inactive channel, but the channel properties of TRPML3(I362T/A419P) were similar to those of TRPML3(A419P). However, they reported that the surface expression and current density of TRPML3(I362T/A419P) are lower than those of TRPML3(A419P) and that the A419P mutation reportedly affects channel glycosylation and causes massive cell death. Their findings reportedly further confirmed that the varitint-waddler phenotype is due to a gain of function of TRPML3(A419P) that is reduced by the TRPML3(1362T/A419P) mutant, resulting in a milder phenotype.

In addition, it had been reported for a related member of the TRPML3 gene family, TRPML1 (mucolipin 1/MCOLN1), that some mutations result in mucolipidosis type IV, a severe inherited neurodegenerative disease. Xu et al., 2007, Nov. 13 Proc Acad Sci USA, 104(46):18321-6 Epub 2007 Nov. 7). This disease is a specific form of mucolipidosis, which is an autosomal recessive lysosomal storage disorder characterized by severe neurodegeneration, achlorhydria, and visual impairments such as corneal opacity and strabismus. The disease arises due to mutations in a group 2 transient receptor potential (TRP)-related cation channel, TRPML1. (Venkatanulum et al., J. Biol. Chem. 2006 Jun. 23 281(25):17517-27 epub 2006 Apr. 10).

It has also been reported that the members of the TRPML3 gene family associate with one another. For example the same reference Venkatanulum et al. (id.) suggests the propensity of these (TRPML1, 2 and 3) proteins to multimerize, and teaches that the subcellular distribution and mechanisms that regulate their trafficking are limited. They also allege that TRPMLs interact to form homo- and heteromultimers. Moreover, Venkatanulum et al. also purport that the presence of either TRPML1 or TRPML2 specifically influences the spatial distribution of TRPML3. They allege that while TRPML1 and TRPML2 homomultimers are lysosomal proteins, that TRPML3 homomultimers are in the endoplasmic reticulum. In addition, they allege that TRPML3 localizes to lysosomes when co-expressed with either TRPML1 or TRPML2 and is comparably mislocalized when lysosomal targeting of TRPML1 and TRPML2 is disrupted. Conversely, they state that TRPML3 does not cause retention of TRPML1 or TRPML2 in the endoplasmic reticulum. Also, Venkatanulum et al. suggest that there is a hierarchy controlling the subcellular distributions of the TRPMLs such that TRPML1 and TRPML2 which dictates the localization of TRPML3 and not vice versa.

Also it had been reported in public gene databases that MCOLN3 or TRPML3 is strongly expressed in the adrenal glands which glands are known to play an important role in the regulation of sodium metabolism in the body. Further, it had been reported that a human autoimmune disease (Addison's) is characterized by the destruction of the adrenal glands and that this disease has as one of its telltale symptoms strong salt cravings.

Still further, it had been reported that TRPML3 is strongly expressed in the pituitary glands and is expressed in melanocytes. As noted above, the varitint mutation, as well as resulting in the death of hearing cells, results in the death of melanocytes. However, to the best of the inventors' knowledge no one had previously suggested that TRPML3 or the related genes TRPML1 or TRPML2 as being involved in salty taste or to encode a taste receptor polypeptide that senses and responds to salty taste stimuli in different mammals or other vertebrates.

### BRIEF DESCRIPTION AND OBJECTS OF THE INVENTION

Therefore, it is an object of the invention to provide the discovery and supporting data which establishes for the first time that TRPML3 plays an active role in taste, specifically salty taste in different mammals and potentially other vertebrates and that based thereon this ion channel, alone or in association with other ion channel genes such as TRPML1, TRPML2, or NALCN, NKAIN3 or other accessory proteins and also based on its specific level of expression in kidney cells involved in sodium homeostasis and further based on expression of this gene in the pituitary and adrenal gland cells also plays a significant role in sodium related cellular and physiological activities such as sodium absorption, transport, and excretion and related ancillary effects and activities such as urine output, blood pressure regulation, and the like. More specifically, it is an object of the invention to teach that this gene plays a role in and/or modulators of TRPML3 may be used to modulate or treat conditions involving sodium homeostasis, including blood pressure regulation in hypertension (high blood pressure) and hypotension (low blood pressure); TRPML3 enhancers and blockers can be used to modulate renal sodium balance to control blood pressure; TRPML3 enhancers and blockers can be used to modulate plasma osmolarity, including decreases in osmolarity (hypoosmotic) and increases in osmolarity (hyperosmotic) via reabsorption of water in the kidney in normal and diseased states such as diabetis insipidus; TRPML3 enhancers and blockers can be used to modulate ENaC function and/or trafficking; TRPML3 enhancers and blockers can be used to modulate aquaporin-2 function and/or trafficking; TRPML3 enhancers and blockers can be used to modulate vasopressin (antidiuretic hormone) secretion from the pituitary gland; and TRPML3 enhancers and blockers can be used to modulate aldosterone secretion from the adrenal glands.

It is also an object of the invention to provide transformed or transfected cells or transgenic animals that express TRPML3 or variants thereof and optionally other ion channels or accessory proteins such as taste specific GPCRs suitable for use in assays in identifying TRPML3 modulators or for study of the effect of TRPML3 on salty taste and other physiologic processes involving sodium transport, metabolism, and excretion.

Also, it is an object of the invention to provide assay systems that comprise test cells, preferably recombinant mammalian cells or amphibian oocytes, or endogenous TRPML3 expressing salty taste cells or other TRPML3 expressing cells (e.g., pituitary or adrenal) that express a functional TRPML3 or a variant, fragment or functional equivalent as well as mammalian cell-based and amphibian oocyte cell-based assays, preferably high-throughput, for putative modulators of TRPML3.

More specifically, it is an object of the invention to provide human cell lines, e.g., HEK293T cells, CHO cells and amphibian oocytes, that express a functional TRPML3 or a variant, fragment or functional equivalent that can be used in cell-based assays to screen for TRPML3 modulators.

Also, it is an object of the invention to provide mammalian cells and amphibian oocytes that express functional TRPML3 or a variant, fragment or functional equivalent for use in functionally characterizing TRPML3 activity, and to identify compounds that either enhance or block salty taste perception (herein referred to as salty taste modulators). These compounds can be used as ingredients in foods, medicinals and beverages to enhance, modulate, inhibit or block salty taste.

As disclosed in the provisional applications and in an earlier utility patent application to which this application claims benefit of priority, the inventors identified this gene initially as encoding a taste specific gene using a novel rationale for identifying primate taste specific genes (disclosed in detail in the provisional applications incorporated by reference herein). These applications showed that this gene which encodes a multitransmembrane protein is expressed in the top of the taste buds, in the taste sensory cells, that conducts sodium. It was conjectured based thereon that TRPML3 may be involved in salty taste perception and other sodium related functions.

Data disclosed herein including functional (electrophysiological) data both in vitro and in vivo (using varitint-waddler mouse which expresses a mutant form of TRPML3 gene that causes deafness, pigment loss and impaired balance), immunohistochemical data, and other information provided infra substantiate the inventors' original supposition and provide convincing experimental validation that this gene encodes a salt receptor that allows sensory taste cells in the tongue's taste buds to detect sodium chloride (salt) and is involved in sodium homeostasis (maintaining sodium levels within certain levels of concentration) in cells throughout the body.

Based on the foregoing it is also an object of the invention to provide therapeutic and prophylactic methods of modulating sodium homeostasis in a subject in need thereof comprising administering a modulatory effective amount of a TRPML3 modulatory compound which may be an agonist, antagonist, enhancer or blocker of TRPML3. These methods may be used in particular to regulate blood pressure, e.g., hypertension or hypotension and other physiological functions involving sodium homeostasis.

Based on the foregoing it is also an object of the invention to provide therapeutic and prophylactic methods of modulating plasma osmolarity in a subject in need thereof comprising administering a modulatory effective amount of a TRPML3 modulatory compound. These methods may be used in particular to treat decreases in osmolarity (hypoosmotic) or increases in osmolarity (hyperosmotic) and also to promote the reabsorption of water in the kidney. The treated subjects may be normal (e.g., dehydrated as a result of exertion or overheating or may have a disease that affects osmolarity such as diabetis insipidus.

Based on the foregoing it is also an object of the invention to provide therapeutic and prophylactic methods of modulating ENaC function and/or trafficking in a subject in need thereof comprising administering an effective amount of a TRPML3 modulatory compound, e.g., a TRPML3 enhancer, blocker, agonist or antagonist.

Based on the foregoing it is also an object of the invention to provide therapeutic and prophylactic methods of modulating aquaporin-2 function and/or trafficking in a subject in need thereof comprising administering an effective amount of a TRPML3 modulatory compound, e.g., a TRPML3 enhancer, blocker, agonist or antagonist.

Based on the foregoing it is also an object of the invention to provide therapeutic and prophylactic methods of modulating vasopressin (antidiuretic hormone) secretion from the pituitary gland trafficking in a subject in need thereof comprising administering an effective amount of a TRPML3 modulatory compound, e.g., a TRPML3 enhancer, blocker, agonist or antagonist.

Based on the foregoing it is also an object of the invention to provide therapeutic and prophylactic methods of modulating aldosterone secretion from the adrenal glands in a subject in need thereof comprising administering an effective amount of a TRPML3 modulatory compound, e.g., a TRPML3 enhancer, blocker, agonist or antagonist.

Also, it is an object of the invention to provide genetic screening assays for detecting abnormalities that affect one or more of sodium homeostasis, plasma osmolarity, ENaC function and/or trafficking, aquaporin-2 function and/or trafficking, vasopressin (antidiuretic hormone) secretion from the pituitary gland, and/or aldosterone secretion from the adrenal glands comprising using a wild type TRPML3 sequence as a probe and detecting for mutations including substitution, deletion, addition mutations in the TRPML3 coding sequence or mutations in the regulatory regions that impair normal TRPML3 transcription and/or translation.

Based on the fact that this gene is specifically expressed in the kidney, adrenal and pituitary glands, it is believed to participate or regulate in association with other polypeptides such as vasopressin and aldosterone the general regulation of sodium transport, metabolism, and excretion in different cells, tissues and organs in the body.

The evidence contained herein which in combination provide convincing evidence that the TRPML3 gene encodes an ion channel that functions as a salty taste receptor in different animals include the following:
(1) MCOLN3 or TRPML3 is specifically expressed at least in primate and human taste and not lingual epithelial cells and is specifically expressed in the top of the taste buds, in a subset of taste sensory cells that do not express TRPM5 (that is, they are not sweet, bitter or umami), do not express PKD2L1 (that is, they are not sour) and are found towards the taste pore. Therefore MCOLN3 positive cells comprise a separate subset of taste cells distinct from known taste cells involved in detecting other (non-salt) taste modalities.
(2) MCOLN3 or TRPML3 is also expressed in sensory cells of other organs, like the ear. It is therefore a ‘professional’ sensory gene.
(3) MCOLN3 or TRPML3 is strongly expressed in the adrenal glands. These glands play a very important role in the regulation of sodium metabolism in the body. MCOLN3 is therefore likely (based on this and other data obtained by the inventors) to be a key molecule in the regulation of sodium metabolism and may regulate or participate in the production of aldosterone by the adrenal glands.
(4) A human autoimmune disease (Addison's) is characterized by the destruction of the adrenal glands. One of the telltale symptoms of this disease is salt craving. The latter is likely the result of the presence of autoantibodies against MCOLN3, or a mutation in this gene that disrupt the function of MCOLN3 or TRPML3 in taste buds.
(5) MCOLN3 or TRPML3 is also highly expressed in the pituitary glands which are involved in vasopressin release, which in turn affects urine production and kidney function. Also, MCOLN3 or TRPML3 is expressed in specific cells of the kidney which are actively involved in sodium transport and excretion and sodium homeostasis in general. Also data obtained in rodents suggests that animals with a deficiency in this gene that impairs function exhibit evidence of aberrant sodium transport and excretion. Based thereon, and other data obtained by the inventors relating to TRPML3, this polypeptide likely regulates or is actively involved in sodium homeostasis and vasopressin release and thereby sodium excretion in the urine.
(6) MCOLN3 or TRPML3 conducts sodium in electrophysiology studies and exhibits the right biochemical characteristics predicted for a primate salty taste receptor (i.e., the detection of K+, Li+ and amiloride insensitivity).
(7) Neurophysiological experiments (nerve recordings) using sodium in the varitint mouse (having TRPML3 mutation) indicate that the Varitint mouse is impaired in its response to sodium (does not exhibit a robust salty taste response). These mice are ablated of the TRPML3 expressing taste cells (salty taste cells) in the taste bud confirming that these specific cells are a prerequisite for detection of salty taste.
(8) Cell based assays using mammalian cells and amphibian oocytes which express mutated TRPML3 polypeptides (mutation results in the ion channel being fixed in the “open” orientation) have identified TRPML3 enhancers and blockers which should enhance or block salty taste in taste tests.

Therefore, based on the foregoing it is an object of the invention to establish the identity of MCOLN3 or TRPML3 as a human salty taste receptor and as an ion channel that regulates sodium homeostasis in cells throughout the body and based thereon to design screening assays using cells or animals transfected with this gene or a variant (e.g., functional chimera, mutant possessing enhanced activity, fixed in open orientation or other desired change in TRPML3 protein facilitating its use in assays) for the purpose of identifying agonists, antagonists or enhancers (modulators) of the function of this molecule which will modulate salty taste and other TRPML3 functions.

Also, it is an object of the invention to provide an isolated taste, adrenal, pituitary or urinary (e.g., kidney or specific kidney cell subsets) organ cell or enriched cell sample comprising a taste, adrenal, pituitary or urinary organ cell that expresses TRPML3 that is involved in salty taste perception, sodium metabolism, aldosterone production, and/or vasopressin release wherein said isolated taste, adrenal, pituitary, or urinary organ cell or enriched taste cell sample expresses TRPML3 gene or a variant thereof that encodes a sodium channel that modulates at least one of salty taste, sodium transport, metabolism, or excretion and/or aldosterone or vasopressin release or production. Preferably the cell will be of human, non-human primate or rodent origin.

Also, it is a specific object of the invention to identify TRPML3 modulators which will be useful in treating diseases involving aldosterone release or production. Diseases and conditions treatable using TRPML3 modulators include diseases treatable by compounds which agonize or antagonize aldosterone and thereby sodium transport and excretion and include by way of example edema, blood pressure (hyper or hypotension), liver cirrhosis, primary hyperaldosteronemia, renal dysfunction, diabetes (Type I or II) and the pathological symptoms associated therewith including circulatory problems, edema, ocular disorders relating to poor circulation, hypercortisolaemia, atherosclerosis or obesity, e.g., abdominal obesity, as well as liver disease, sexual dysfunction (male or female), cerebrovascular disease, vascular disease, retinopathy, neuropathy, insulinopathy, endothelial dysfunction, baroreceptor dysfunction, migraine headaches, hot flashes, and premenstrual tension and other cardiovascular conditions such as atherosclerosis, heart failure, congestive heart failure, vascular disease, stroke, myocardial infarction, endothelial dysfunction, ventricular hypertrophy, renal dysfunction, target-organ damage, thrombosis, cardiac arrhythmia, plaque rupture and aneurysm.

Also, it is a specific object of the invention to identify TRPML3 modulators which will be useful in treating diseases involving vasopressin release or production. Diseases and conditions treatable using TRPML3 modulators include diseases or conditions treatable by compounds that agonize or antagonize vasopressin and similarly include by way of example diabetes, obesity, kidney diseases such as cystic kidney disease, acquired renal cystic disease, ocular circulation related disorders such as myopia; nausea, emesis, sexual dysfunction (male or female), edema, hypertension, congestive heart failure (ranging from class II of the New York Heart Association to florid pulmonary edema), periodic idiopathic edema, nephritic syndrome, ascites due to cirrhosis or other causes, cerebral edema of various causes, as well as dilutional hyponatremia and metabolic alterations collectively known as the syndrome of inappropriate ADH secretion and other diseases or conditions wherein vasodilation and/or antioxytocic activity is therapeutically desirable.

Also it is an object of the invention to provide therapeutic and prophylactic methods of modulating sodium homeostasis in a subject in need thereof comprising administering a modulatory effective amount of a TRPML3 modulatory compound which may be an agonist, antagonist, enhancer or blocker of TRPML3. These methods may be used in particular to regulate blood pressure, e.g., hypertension or hypotension.

Also it is an object of the invention to provide therapeutic and prophylactic methods of modulating plasma osmolarity in a subject in need thereof comprising administering a modulatory effective amount of a TRPML3 modulatory compound. These methods may be used in particular to treat decreases in osmolarity (hypoosmotic) or increases in osmolarity (hyperosmotic) and also to promote the reabsorption of water in the kidney. The treated subjects may be normal (e.g., dehydrated as a result of exertion or overheating or may have a disease that affects osmolarity such as diabetis insipidus.

Also it is an object of the invention to provide therapeutic and prophylactic methods of modulating ENaC function and/or trafficking in a subject in need thereof comprising administering an effective amount of a TRPML3 modulatory compound, e.g., a TRPML3 enhancer, blocker, agonist or antagonist.

Also it is an object of the invention to provide therapeutic and prophylactic methods of modulating aquaporin-2 function and/or trafficking in a subject in need thereof comprising administering an effective amount of a TRPML3 modulatory compound, e.g., a TRPML3 enhancer, blocker, agonist or antagonist.

Also it is an object of the invention to provide therapeutic and prophylactic methods of modulating vasopressin (antidiuretic hormone) secretion from the pituitary gland trafficking in a subject in need thereof comprising administering an effective amount of a TRPML3 modulatory compound, e.g., a TRPML3 enhancer, blocker, agonist or antagonist.

Also it is an object of the invention to provide therapeutic and prophylactic methods of modulating aldosterone secretion from the adrenal glands in a subject in need thereof comprising administering an effective amount of a TRPML3 modulatory compound, e.g., a TRPML3 enhancer, blocker, agonist or antagonist.

Also, it is an object to provide genetic screening assays for detecting abnormalities that affect one or more of sodium homeostasis, plasma osmolarity, ENaC function and/or trafficking, aquaporin-2 function and/or trafficking, vasopressin (antidiuretic hormone) secretion from the pituitary gland, and/or aldosterone secretion from the adrenal glands comprising using a wild type TRPML3 sequence as a probe and detecting for mutations including substitution, deletion, addition mutations in the TRPML3 coding sequence or mutations in the regulatory regions that impair normal TRPML3 transcription and/or translation.

In addition, it is an object of the invention to provide gene therapies in individuals identified as having mutations in TRPML3 that impair sodium homeostasis or other TRPML3 functions.

Also more specifically, it is an object of the invention to provide an isolated taste receptor that modulates salty taste perception comprising a TRPML3 polypeptide or variant thereof such as one engineered to possess enhanced ion channel activity or to remain fixed in the open orientation or a chimera or fragment of TRPML3 that modulates salty taste in mammals including humans. This taste receptor may be monomeric or polymeric (heteropolymeric or homopolymeric) and may comprise other taste specific polypeptides, e.g., other ion channel polypeptides such as TRPML2, TRPML1, NKAIN3 or NALCN.

Also more specifically, it is an object of the invention to provide a transgenic non-human animal which has been genetically engineered to knock out the expression of endogenous TRPML3 and/or to further genetically engineer the knocking in of an ortholog or variant thereof, e.g. one engineered to enhance ion channel activity or to fix the channel in the “open” position. These animals, including those expressing human or other primate TRPML3 genes or variants may be used to identify compounds that modulate (enhance or block) salty taste in humans and other mammals.

More specifically, it is an object of the invention to provide a transgenic non-human animal which has been genetically engineered to express a heterologous TRPML3 polypeptide, e.g., a human TPML3 or variant.

Also more specifically, it is an object of the invention to provide assay methods of using a transgenic animal that expresses TRPML3 or a mutant form such as the mutation causing the Varitint-waddler phenotype in screens to identify antagonists, agonists or enhancers of TRPML3 and to study the effects of TRPML3 on different physiological activities including salty taste and sodium transport, metabolism and excretion.

Also more specifically, it is an object of the invention to provide methods of using a transgenic animal according wherein the TRPML3 gene has been “knocked out” in order to elucidate the effect of TRPML3 on taste, and on cardiac or urinary function and in particular on aldosterone production, sodium metabolism, salty taste perception or vasopressin release. It is anticipated that these animals may comprise conditions relating to sodium transport and metabolism such as hypertension, hypotension, fluid retention, heart attack and stroke. Therefore, the invention further includes the use of these animals as disease models and for evaluation of potential therapeutics for treating or preventing such conditions.

Also, more specifically it is an object of the invention to use mutant forms of TRPML3 polypeptide, including the mutated form that gives rise to the Varitint-Waddler phenotype, in order to kill or ablate specific cells including salty taste cells, melanocytes, pituitary or adrenal cells.

Also, more specifically it is an object of the invention to use mutant forms of the gene encoding the TRPML3 polypeptide, including the mutated form that gives rise to the Varitint-Waddler phenotype in order to create transgenic animals wherein specific cells are ablated and to use these transgenic animals in order to test potential therapeutics and as disease models, especially for conditions involving aberrant plasma osmolarity, sodium homeostasis, blood pressure and the like.

Also, more specifically it is an object of the invention to use mutant forms of TRPML3 polypeptide, including the mutated form that gives rise to the Varitint-Waddler phenotype as toxins to kill specific cells.

Also it is an object of the invention to provide the use of molecules that modulate or bind TRPML3, e.g., which agonize or antagonize or specifically bind to this polypeptide for the treatment of melanoma, adrenal cancer, pituitary cancer, et al. and other conditions involving melanocytes such as pigmentation disorders or pituitary or adrenal related disorders.

Also more specifically, it is an object of the invention to provide a recombinant cell which expresses a salty taste receptor comprising TRPML3 or a variant thereof that encodes a functional sodium ion channel polypeptide.

Also more specifically, it is an object of the invention to provide an assay for identifying compounds that agonize, antagonize or enhance an activity of TRPML3 comprising contacting a recombinant or endogenous taste or other cell that expresses TRPML3 with a putative TRPML3 enhancer, agonist or antagonist and determining the effect thereof on TRPML3 activity. Preferably these assays will be electrophysiological assays e.g., patch clamp or two electrode voltage clamping assays, which may be automated and typically will use mammalian or amphibian cells.

Also more specifically, it is an object of the invention to provide methods for identifying TRPML3 modulators by an ion flux assay.

Also more specifically, it is an object of the invention to provide methods for identifying TRPML3 modulators (enhancers, blockers) by an automated electrophysiological (patch clamp) assay, i.e., IonWorks assay system

Also more specifically, it is an object of the invention to provide methods for identifying TRPML3 modulators (enhancers, blockers) by electrophysiological assays using frog oocytes.

Also more specifically, it is an object of the invention to provide methods for identifying TRPML3 modulators (enhancers, blockers) by electrophysiological assays using mammalian cells.

Also more specifically, it is an object of the invention to provide such TRPML3 assays wherein the identified agonist, antagonist, or enhancer compounds are evaluated in a taste test. Also more specifically, it is an object of the invention to provide such TRPML3 assays wherein the effect of the identified agonist, antagonist, or enhancer compounds on sodium homeostasis, blood pressure, plasma osmolarity, sodium homeostasis, including blood pressure regulation in hypertension (high blood pressure) and hypotension (low blood pressure); plasma osmolarity, including decreases in osmolarity (hypoosmotic) and increases in osmolarity (hyperosmotic) via reabsorption of water in the kidney in normal and diseased states such as diabetis insipidus; ENaC function and/or trafficking; aquaporin-2 function and/or trafficking; vasopressin (antidiuretic hormone) secretion from the pituitary gland; or aldosterone secretion from the adrenal glands is tested in an animal.

Also more specifically, it is an object of the invention to provide such TRPML3 assays wherein the effect of the identified agonist, antagonist, or enhancer compounds on aldosterone production is tested in an animal.

Also more specifically, it is an object of the invention to provide such TRPML3 assays wherein the effect of the identified agonist, antagonist, or enhancer compounds on vasopressin release is tested in an animal.

Also more specifically, it is an object of the invention to provide such TRPML3 assays wherein the effect of the identified agonist, antagonist, or enhancer compounds on sodium homeostasis is tested in an animal.

Also more specifically, it is an object of the invention to provide such TRPML3 assays wherein the effect of the identified agonist, antagonist, or enhancer compounds on blood pressure is tested in an animal.

Also more specifically, it is an object of the invention to provide such TRPML3 assays wherein the effect of the identified agonist, antagonist, or enhancer compounds on ENac function and/or trafficking or aquaporin-2 function and/or trafficking is tested in an animal.

Also more specifically, it is an object of the invention to provide such TRPML3 assays wherein the effect of the identified agonist, antagonist, or enhancer compounds on at least one of cardiac or urinary function and more specifically on blood pressure, fluid retention, sodium metabolism or urine production, wherein this is tested in an animal.

Also more specifically, it is an object of the invention to provide the use of the identified agonist, antagonist, or enhancer compounds for treating a disease or condition involving aldosterone production comprising administering an effective amount of a compound that modulates TRPML3.

Also more specifically, it is an object of the invention to provide the use of the identified agonist, antagonist, or enhancer compounds for treating a disease or condition involving vasopressin release comprising administering an effective amount of a compound that modulates TRPML3.

Also more specifically, it is an object of the invention to provide the use of the identified agonist, antagonist, or enhancer compounds for modulating cardiac function, e.g., blood pressure, arrhythmia, or stroke or fluid retention in a subject in need thereof comprising administering an effective amount of a compound that modulates TRPML3.

Also, more specifically it is an object of the invention to provide isolated taste, adrenal, pituitary, melanocyte, or urinary organ cells or an enriched taste cell sample wherein said isolated or enriched cell sample comprises cells that express a TRPML3 ion channel polypeptide.

Also, more specifically it is an object of the invention to provide an isolated taste receptor that modulates salty taste perception comprising a TRPML3 polypeptide or variant thereof that modulates salty taste in mammals.

Also, more specifically it is an object of the invention to provide a transgenic non-human animal which has been genetically engineered to knock out or to impair the expression of endogenous TRPML3 with the proviso that said transgenic animal is not a Varitint mouse.

Also, more specifically it is an object of the invention to provide a transgenic non-human animal which has been genetically engineered to express a heterologous TRPML3 polypeptide with the proviso that said transgenic animal is not a Varitint mouse.

Also, more specifically it is an object of the invention to provide a method of using the transgenic animal in screens to identify salty taste modulating compounds.

Also, more specifically it is an object of the invention to provide a method of using the transgenic animal to identify antagonists, agonists or enhancers of TRPML3 and wherein the wherein the identified compounds are further optionally evaluated in human taste tests.

Also, more specifically it is an object of the invention to provide a method of using a transgenic animal in order to elucidate the effect of TRPML3 on aldosterone production, sodium metabolism, salty taste perception or vasopressin release.

Also, more specifically it is an object of the invention to provide a method of using a transgenic animal (non-human) that expresses a TRPML3 gene that encodes an ion channel that is toxic to cells which express the ion channel in order to assess potential therapeutic regimens for diseases or conditions involving aberrant aldosterone production, vasopressin release, sodium metabolism or homeostasis, plasma osmolarity, and/or melanocyte loss.

Also, the invention relates to the use of the Varitint waddler mice to detect the effect of TRPML3 function on melanocytes, pituitary, adrenal, taste, urinary or taste cells.

Also, the invention relates to the use of the Varitint waddler mice in assays to detect genes specifically expressed in salty taste cells and not in the Varitint waddler mice (as salty taste cells are ablated therein) which genes may modulate TRPML3 function, or function as a salty taste receptor or modulate transmission of salty taste signaling from TRPML3 to the nerve fibers and/or control the development differentiation or apoptosis of salty taste cells. These gene detection assays may comprise the use of gene chips or microarray technology to compare the genes expressed in salty taste cells versus genes expressed in Varitint waddler mice.

Also, the invention provides methods of treating parathyroid related diseases such as calcium homeostasis, hypercalcemia, osteitis, hypoparathyroidism, hyperparathyroidism, osteitis fibrosis cystica, pseudoparahypothyroidism, Jansen's metaphyseal chondroplasia, Blomstrand's chondroplasia, and osteoporosis of different causes such as diseases, age, menopause, chemotherapy, radiation therapy, drugs and the like.

Also, more specifically it is an object of the invention to provide a recombinant cell which expresses a salty taste receptor comprising TRPML3 or a variant thereof, e.g., a yeast, amphibian, insect, bacterial, reptile, avian, or mammalian cell., preferably a mammalian cell or frog oocyte, such as a CHO-K1, HEK-293, COS, CHO, BHK cell which may transiently expresses said TRPML3 polypeptide or stably express said TRPML3 polypeptide.

Also, more specifically it is an object of the invention to provide a method of identifying putative salty taste modulators in a binding assay comprising providing a TRPML3 polypeptide or cell which expresses TRPML3 and contacting said polypeptide or cell with putative TRPML3 modulatory compounds and identifying potential TRPML3 modulators based on their specific binding to TRPML3 polypeptide.

Also, more specifically it is an object of the invention to provide a method of modulating blood pressure or fluid retention in a subject in need thereof comprising administering an effective amount of a compound that modulates TRPML3.

Also, more specifically it is an object of the invention to provide a method of modulating urine production and/or excretion in a subject in need thereof comprising administering an effective amount of a compound that modulates TRPML3.

Also, more specifically it is an object of the invention to provide a method of treating Addison's disease or type IV mocolipidosis in a subject in need thereof comprising administering an effective amount of a compound that modulates TRPML3.

Also, more specifically it is an object of the invention to provide specific codon optimized and TRPML3 mutated sequences and assays using these sequences.

Also more specifically, it is an object of the invention to provide the use of such identified agonist, antagonist, or enhancer compounds for modulating urine production and/or excretion or edema in a subject in need thereof comprising administering an effective amount of a compound that modulates TRPML3.

Also more specifically, it is an object of the invention to provide such identified agonist, antagonist, or enhancer compounds which may include polypeptides, antibodies, small molecules, siRNAs, antisense RNAs, ribozymes et al.

More specifically, it is an object of the present invention to provide mammalian and oocyte cell-based assays, preferably high or medium throughput, for the profiling and screening of a salty taste receptor (TRPML3) which assays optionally may include the addition of a compound that modulates TRPML3 function. Such methods can be used to functionally characterize TRPML3 activity and to identify the specific motifs or residues required for salt sensing in different TRPML3 ion channels as well as to identify compounds that either enhance or block salty taste perception (herein referred to as salty taste modulators).

It is also an object of the invention to provide novel methods for treatment or prevention of conditions relating to sodium transport and metabolism such as hypertension, hypotension, fluid retention, heart attack and stroke and conditions mentioned above by administration of TRPML3 modulators.

In a specific aspect, the invention provides a method for identifying a modulator of TRPML3 utilizing a mammalian cell or oocyte that expresses a functional TRPML3 sodium channel with a putative TRPML3 modulatory compound, comprising: (i) assaying the effect of said compound on sodium transport through the TRPML3 channel; and (ii) identifying whether said compound is an TRPML3 modulator based on its enhancing or inhibitory effect on sodium transport. The invention further comprises (iii) confirming that the compound identified modulates salty taste in human or mammalian taste tests. In one embodiment, the TRMPL3 is mammalian TRMPL3. In yet another embodiment, TRMPL3 is human, non-human primate, rodent (mouse or rat), cow, pig, horse or sheep TRMPL3.

In a further embodiment, the in vivo effect of the identified compound on sodium extraction or urinary function or cardiovascular or other functions relating to TRPML3 is tested in humans or mammals. In one embodiment, the TRMPL3 is mammalian TRMPL3. In yet another embodiment, TRMPL3 is human, non-human primate, rodent, cow, pig, horse or sheep TRMPL3.

In one aspect of the present invention, the mammalian cell is selected from the group consisting of a HEK293, HEK293T, Swiss3T3, CHO, BHiK, NIH3T3, and COS cells. In a second aspect, the oocyte is a mammalian, amphibian, avian or reptilian oocyte. In a further aspect, the amphibian oocyte is a Xenopus oocyte. In another aspect of the invention, the cell expresses an additional taste gene, preferably an ion channel.

In a related embodiment of the invention, these assays are used to identify a human TRPML3 enhancer or inhibitor wherein an oocyte is contacted with an inhibitor or activator of human TRPML3 prior to contacting with a putative human TRPML3 enhancer. In an additional embodiment, the assay further comprises a negative control using an oocyte that has not been microinjected with human TRPML3 cRNAs. In an additional aspect of the invention, the putative modulator is applied at a concentration ranging from around 1 nM to about 1 mM. In another aspect of the invention, the human TRPML3 enhancer exhibits an enhancement factor of at least 20%. In a further aspect, the human TRPML3 enhancer exhibits an enhancement factor of at least 50%. In yet a further aspect, the human TRPML3 enhancer exhibits an enhancement factor of at least 100%.

Also more generally, it is an object of the invention to provide a method or rationale for identifying a gene encoding a polypeptide involved in salty taste perception in a primate (human or non-human) comprising:

(i) identifying a set of genes including genes which are expressed in fungiform and optionally circumvallate, foliate, or palate taste cells but which are not expressed in lingual cells and/or genes which are expressed in taste cells at substantially higher levels than in lingual cells;

(ii) of the genes identified in (i) optionally identifying a set of genes which are not expressed in taste cells which express umami, sweet, bitter, or sour taste receptors or markers of these cells (T1Rs or T2Rs, TRPM5, and PKD2L1/PKD1L3);

(iii) optionally identifying a subset of the taste specific genes contained in the genus of genes identified after step (i) or step (ii) which are specifically expressed in the top half of taste buds and not the bottom half of taste buds or which are enriched (expressed at least 1.2-1.5 fold greater) in the top half than in the bottom half of taste buds; and

(iv) functionally expressing one or more genes identified according to (ii) or (iii) and determining which of said genes functions as a sodium responsive ion channel or sodium responsive receptor or transporter and thereby identifying this gene or genes as a putative gene(s) that modulates salty taste.

(Preferably, the identified gene which is functionalized is one which is enriched by at least 1.2-1.5 fold in the top half of the taste buds relative to the bottom half of the taste buds).

In these methods step (i) preferably comprises the use of laser capture microdissection (LCD) to dissect and purify taste tissues from non-taste tissues and/or step (i) comprises RNA amplification of genes from taste cells and lingual cells and the amplified genes are screened against a gene chip containing a sample of genes specific to the particular mammal from which the taste and lingual tissues are obtained.

Further in these methods step (i) preferably comprises high throughput PCR using primers for each ion channel in the human or non-human primate genome and step (ii) is preferably effected by in situ hybridization using antisense RNA probes specific for the genes identified in step (i) to determine level of expression in taste versus lingual cells or by use of immunocytochemical detection using a labeled antibody specific to the protein encoded by gene or genes identified in step (i).

Also more generally, it is an object of the invention to provide a method for identifying a gene encoding a polypeptide involved in salty taste perception in a primate (human or non-human) comprising:
(i) identifying a set of genes including genes which are expressed in fungiform, circumvallate, foliate, or palate taste cells but which are not expressed in lingual cells and/or genes which are expressed in said taste cells at substantially higher levels than in lingual cells;
(ii) of the genes identified in (i) identifying a set of genes which are not expressed in taste cells which express umami, sweet, bitter, or sour taste receptors or markers of these cells (T1Rs or T2Rs or TRPM5 or PKD2L1/PKD1L3);
(iii) of the genes identified in (i) or (ii) optionally identifying whether the gene is specifically expressed in the top half of taste buds and not the bottom half or is enriched (expressed at least 1.2-1.5 fold higher) in the top half of taste buds relative to expression in the bottom half of taste buds; and
(iv) determining, in a primary neuron which expresses one or more genes identified according to (ii), which of said genes functions as a sodium responsive ion channel or sodium responsive receptor or transporter and thereby identifying this gene or genes as a putative gene that modulates salty taste.
   (Again the selected taste specific gene which is functionalized will preferably be enriched i.e., expressed 1.2-1.5 fold higher in the top half of taste buds versus the bottom half of taste buds.)

Also more specifically, it is an object of the invention to provide an assay for identifying a compound having potential in vivo application for modulating human salty taste comprising the following:
(i) contacting a cell that expresses a gene encoding a TRPML3 ion channel alone or with NALCN, NKAIN3, TRPML1, or TRPML2 or an ortholog or a gene encoding a polypeptide possessing at least 90% sequence identity to the polypeptide encoded thereby with at least one putative antagonist, agonist or enhancer compound;
(ii) assaying sodium conductance, receptor activity or sodium transport in the presence and absence of said putative agonist, antagonist or enhancer; and
(iii) identifying the compound as a potential salty taste enhancer based on whether it modulates sodium conductance and other conductance properties consistent with a human salt receptor.

Also more specifically, it is an object of the invention to provide a method of using a probe specific to a TRPML3 gene or gene product to identify and/or isolate and or enrich salty taste specific cell, preferably primate alt receptor expressing cells, in a taste cell sample.

Also more specifically, it is an object of the invention to provide the use of TRPML3 to purify or enrich a desired taste cell subtype or taste cell lineage that includes the use of a fluorescence activated cell sorter (FACS) or the use of labeled magnetic beads.

Also more specifically, it is an object of the invention to provide the use of TRPML3 to purify or enrich a desired taste cell subtype or taste cell lineage wherein the desired taste cell subtype or taste cell lineage is isolated, purified, enriched or marked by a method that includes a negative cell selection technique that eliminates at least one non-target taste cell subtype or lineage based on the expression or absence of expression of at least one other taste cell specific gene., e.g., by the use of cytotoxic antibodies that specifically kill at least one non-target cell type or lineage.

Also more specifically, it is an object of the invention to provide methods of identifying, isolating or enriching salty taste receptor cells using TRPML3 alone or in association with other taste specific genes such as TRPML1, TRPML2, NALCN and/or NKAIN3 as a marker.

Based on the foregoing, it can be appreciated that this invention was in part the result of a novel protocol for identifying taste specific genes. These genes were identified using two different techniques, gene chips and a polymerase chain reaction (PCR) screen, to identify novel salt receptor target genes. First, Affymetrix gene chips containing most all known macaque genes are used to determine which genes are specifically expressed in primate circumvallate at the back of the tongue and fungiform papilla taste cells at the front of the tongue and not lingual epithelial cells isolated by laser capture microdissection. Second, PCR is used to determine which ion channels, from channels we have cataloged in the human/macaque genomes, are specifically expressed in macaque fungiform and/or circumvallate (CV) papilla taste cells but not lingual epithelial cells isolated by laser capture microdissection. In addition, of these genes a subset which is expressed specifically in the top half of taste buds, or which is enriched (expressed at least 1.2-1.5 fold higher) is identified as being especially preferred candidates for functionalizaton. Taste-specific expression of genes identified by either approach, are confirmed using an independent histological method such as in situ hybridization or immunohistochemistry, to determine which genes are expressed in taste cells. Using double labeling histological methods, it is determined what novel taste-specific genes are expressed in sweet, bitter, and umami cells that express the taste-specific ion channel TRPM5, sour cells that express the taste-specific ion channel PKD2L1/PKD1L3, or a unique cell type that does not express TRPM5 or PKD2L1/PKD1L3. A taste-specific gene, preferably an ion channel, that is conductive or activated by sodium and is expressed in a TRPM5- and PKD2L1/PID1L3-negative cell population is a probable candidate for screening efforts to identify the gene(s) that encode mammalian salty taste receptors, as well as specific cell types wherein these salty taste receptor genes are expressed such as in the oral cavity and urinary tract, and also for use in high throughput assays designed to identify enhancers of saltiness in humans. Using these general methods TRPML3 was identified as a potential salty taste receptor.

Novel taste-specific genes identified using these rationales as well as affecting salt perception (and other biological activities likely affected thereby such as sodium absorption, transport and excretion and the effects thereof such as fluid retention and blood pressure regulation) may alternatively affect other taste modalities and flavor perception in general. While, the inventors have identified TRPML3 as being a salty taste receptor and have convincing functional data in support thereof it is anticipated that TRPML3 is involved in non-taste biological functions such as discussed above. Therefore, this gene is a useful target in therapeutic screening assays, e.g., for identifying therapeutics for the treatment of diseases related to TRPML3 such as Addison's Disease, mocoid disorders such as mucolipidosis type IV, urinary disorders, and cardiovascular disorders and pathologies associated with sodium transport, metabolism, and excretion and vasopressin or aldosterone release or production and plasma osmolarity or blood pressure irregularities.

Also, the invention generally relates to use of the inventive taste specific genes and probes specific thereto in isolation and purification methods that include both positive and negative cell separation methods. For example, desired taste cell lineages or types may be isolated by positive cell selection methods e.g., by the use of fluorescence activated cell sorting (FACS), magnetic bead cell selection e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of one or several undesired cell lineages e.g., by contacting a mixed cell suspension containing the desired taste (salty) cells and undesired cells e.g., derived from the tongue, oral cavity or gastrointestinal tract and associated organs with cytotoxic antibodies specific to a target gene or genes expressed on the undesired taste cell type(s) which are to be removed.

Also, the invention generally relates to use of the inventive taste specific gene which is involved in specific taste and non-taste specific functions, mapping of cell comprised on specific regions of the gastrointestinal tract and associated organs such as the intestinal epithelium or urinary tract that express specific taste specific genes and which therefore are involved in one or more of the taste cell specific functions disclosed herein, and/or the use of the subject genes and markers specific thereto in taste cell differentiation studies, e.g. for identifying compounds that induce the differentiation or dedifferentiation of taste cells e.g., adult or embryonic stem cells and other pluripotent or immature cell types into desired taste cell lineages and taste cell types.

Also more specifically, as described in detail infra, the invention more broadly provides a rationale and criteria for a candidate salty taste gene, preferably an ion channel which are:
a) Specific expression in primate (macaque) taste cells, particularly fungiform and/or circumvallate papilla derived taste cells, but also foliate and palate taste cells, and not lingual epithelial cells or expression at higher levels in taste cells than lingual cells
b) Expression in a taste cell by histological methods. Specifically, expression in a unique taste cell type that does not express the sweet, bitter, and umami cell marker TRPM5 or the sour cell marker PKD2L1/PKD1L3. This unique cell type will likely correspond to unique taste cell lineage, e.g., a dedicated salt sensing or fat sensing cell.
c) Functional expression as a sodium channel or a sodium-activated receptor with basal, constitutive function (i.e. a fraction of the channel population is open and passing sodium at rest) in heterologous expression systems (such as Xenopus oocytes and mammalian cells) or primary neurons (such as dorsal root ganglia neurons).
d) Optionally, specific expression or enrichment in the top fraction of taste bud cells, preferably at least 1.2-1.5 fold higher expression in the top half versus bottom half of taste buds.

Genes fulfilling these criteria are advanced into high-throughput screening efforts to identify compounds that enhance human salt perception. These methods coupled with in vitro functional assays and neurophysiological data in mice expressing a mutant TRPML3 gene that gives rise to the Varitint-waddler phenotype have revealed that TRPML3 is a salty taste receptor in primates (humans) and non-human primates and most likely other animals including e.g., other mammals such as dogs, cats, horses, bovines, pigs, sheep, and other vertebrates. More specifically, as described in detail infra, the invention provides a rationale and criteria for a candidate salty taste gene, preferably an ion channel which are:
a) Specific expression in primate (macaque) taste cells, particularly fungiform and/or circumvallate papilla derived taste cells, but also foliate and palate taste cells, and not lingual epithelial cells or expression at higher levels in taste cells than lingual cells
b) Expression in a taste cell by histological methods. Specifically, expression in a unique taste cell type that does not express the sweet, bitter, and umami cell marker TRPM5 or the sour cell marker PKD2L1/PKD1L3. This unique cell type could be a dedicated salt sensing cell.
c) Functional expression as a sodium channel or a sodium-activated receptor with basal, constitutive function (i.e. a fraction of the channel population is open and passing sodium at rest) in heterologous expression systems (such as Xenopus oocytes and mammalian cells) or primary neurons (such as dorsal root ganglia neurons).

### SUMMARY OF THE INVENTION

Using a novel rationale for identifying taste specific genes disclosed in earlier provisional patent applications incorporated by reference herein and which are claimed in a related application filed on even date as this application the present inventors have identified a taste specific polypeptide that functions as a primate (human) salty taste receptor polypeptide and which in all likelihood in involved in other physiological functions involving sodium transport, absorption and excretion such as urinary and cardiac functions.

Particularly, the inventors have identified a gene, Mucolipin 3 (MCOLN3) or TRPML3 as it is alternatively named that encodes a multitransmembrane protein expressed in the top of the taste buds, in the taste sensory cells, that conducts sodium. Various lines of evidence convincingly demonstrate that this polypeptide as a primate salty taste receptor polypeptide.

Specifically, this gene represents a salt receptor that by itself and/or in association with other taste specific polypeptides or ion channels (related family members) such as TRPML1, TRPML2, NALCN or NKAIN3 which allows sensory taste cells in the tongue's taste buds to detect sodium chloride (salt). In addition, because this gene is highly expressed in the adrenal and pituitary glands, it is reasonably anticipated to play an active role in the regulation of sodium metabolism in the body. The evidence that points to this gene being the human salt receptor includes at least the following:
(1) Using the novel rationale for identifying putative taste receptor genes it was determined by the inventors that MCOLN3 is specifically expressed in the top of the taste buds, in a subset of taste sensory cells that do not express TRPM5 (that is, they are not sweet, bitter or umami), do not express PKD2L1 (that is, they are not sour) and towards the taste pore.
(2) It is known that MCOLN3 is also expressed in sensory cells of other organs, like the ear. It is therefore a ‘professional’ sensory gene.
(3) It is further known that MCOLN3 is strongly expressed in the adrenal glands. These glands play a very important role in the regulation of sodium metabolism in the body. MCOLN3 is therefore likely to be a key molecule in the regulation of sodium metabolism and may regulate the production of aldosterone by the adrenal glands.
(4) Related to the foregoing it is also known that a human autoimmune disease

(Addison's) is characterized by the destruction of the adrenal glands. One of the telltale symptoms of this disease is salt craving. The latter is likely to result from the presence of autoantibodies against MCOLN3, or a mutation in this gene that disrupts the function of MCOLN3 in taste buds.
(5) It is also known that MCOLN3 or TRPML3 is highly expressed in pituitary glands which produce vasopressin that is involved in urine production, further substantiating the probable role of this gene in sodium excretion and urinary function.
(6) As substantiated by the data contained in the experimental examples infra, MCOLN3 conducts sodium in electrophysiology studies and exhibits biochemical characteristics predicted and consistent for a human salt receptor (detection of K+, Li+ and amiloride insensitivity).
(7) Neurophysiological experiments (nerve recordings) using sodium in the varitint-waddler mouse (having TRPML3 mutation) indicate that the Varitint mouse is impaired in its response to sodium (does not exhibit a robust salty taste response). In addition, it has been confirmed that these same mice are ablated of TRPML3 or MCOLN3 expressing taste cells (salty taste cells) establishing further that the unique taste cell subset of TRPML3 expressing taste bud cells is functional, i.e., they are a prerequisite for salty taste perception.
(8) Cell based assays using mammalian cells and amphibian oocytes which express mutated TRPML3 polypeptides (mutation results in the ion channel being fixed in the “open” orientation) have identified TRPML3 enhancers and blockers which should enhance or block salty taste in taste tests.

Further evidence that TRPML3 is the salty taste receptor and is involved in sodium homeostasis throughout the body is the further discovery that TRPML3 is significantly expressed in kidney cells responsible for sodium reabsorption and the disruption of renal sodium homeostasis in heterozygous varitint-waddler mice (Va/+), a mouse model where TRPML3 salty taste cells are specifically ablated from taste buds and where salty taste is greatly diminished. These data provide direct evidence for the physiological function of TRPML3 in modulating sodium homeostasis in the kidney and further support the identification of TRPML3 as the human salty taste receptor.

As described above, in addition to being expressed in taste buds, it has been observed that genes which are known or predicted to encode functional taste receptors are also expressed in non-gustatory tissues throughput the body. For example, T1Rs (sweet and umami receptors) and T2Rs (bitter receptors) are expressed in the gut where they may detect sweet, bitter, and umami ligands in digestive juices and affect processes including the cephalic phase insulin response, whereas PKD2L1 (candidate sour receptor) expressed in neurons lining the central canal of the spinal column may detect cerebrospinal fluid pH. (See Chen M C, Wu S V, Reeve J R, Jr., & Rozengurt E (2006) Bitter stimuli induce Ca2+ signaling and CCK release in enteroendocrine STC-1 cells: role of L-type voltage-sensitive Ca2+ channels. American journal of physiology 291:C726-739; Huang A L, Chen X, Hoon M A, Chandrashekar J, Guo W, et al. (2006) The cells and logic for mammalian sour taste detection. Nature 442:934-938; Jang H J, Kokrashvili Z, Theodorakis M J, Carlson O D, Kim B J, et al. (2007) Gut-expressed gustducin and taste receptors regulate secretion of glucagon-like peptide-1. Proceedings of the National Academy of Sciences of the United States of America 104:15069-15074; and Stermini C (2007) Taste receptors in the gastrointestinal tract. IV. Functional implications of bitter taste receptors in gastrointestinal chemosensing. Am J Physiol Gastrointest Liver Physiol 292:G457-461).

Based on this information, and further based on our prediction that TRPML3 plays an active role in sodium homeostasis, we reasoned that TRPML3 would be expressed in tissues important for sodium homeostasis such as the kidney. Using in situ hybridization, TRPML3 was identified in renal tubules. (See example and figures referenced therein infra). Double label in situ hybridization studies localized TRPML3 to principal cells in cortical and outer medullary collecting ducts. TRPML3 kidney cells express both aquaporin-2 water channels that reabsorb water to maintain plasma osmolarity, as well as ENaC sodium channels that reabsorb sodium to maintain salt balance. Unlike taste cells, TRPML3 cells in the kidney were not ablated in Va/+ mice, suggesting that the A419P TRPML3 mutation did not compromise ionic homeostasis to an extent that compromised cell viability in kidney cells. Since principal cells respond to the sodium-conserving hormone aldosterone, we measured plasma sodium and aldosterone levels in Va/+ mice. Va/+ mice exhibit a pronounced hyponatremia (low blood sodium concentration) and hyperaldosteronemia (high blood aldosterone concentration), consistent with a deficiency in principal cell function, while other electrolytes including plasma chloride and potassium as well as glucose and protein levels were not significantly different from wild-type controls. TRPML3 is, therefore, expressed in kidney epithelial cells responsible for sodium balance, and Va/+ mice exhibit deficiencies in sodium homeostasis, similar, in part, to pseudohypoaldosteronism type I, a salt wasting human genetic disease due to loss of function mutations in ENaC subunits. (Chang S S, Grunder S, Hanukoglu A, Rosier A, Mathew P M, et al. (1996) Mutations in subunits of the epithelial sodium channel cause salt wasting with hyperkalaemic acidosis, pseudohypoaldosteronism type 1, Nature genetics 12:248-253; and Zennaro M C & Lombes M (2004) Mineralocorticoid resistance. Trends in endocrinology and metabolism: TEM 15:264-270). A419P TRPML3 disrupts ion homeostasis in principal cells of Va/+ mice such that cells are unable to efficiently respond to aldosterone and reabsorb sodium.

These discoveries are significant as the identification of MCOLN3 or TRPML3 as a human and other primates and rodent salty taste receptor (and presumably a salty taste receptor in other mammals or vertebrates) and also its role in sodium homeostasis in general allows for the design of screening assays using cells transfected with this gene for the purpose of identifying agonists, antagonists or enhancers (modulators) of the function of this molecule. These compounds may be used as taste modulators and also may be useful as therapeutic agents for treating and modulating cardiac and urinary related functions and conditions such as high or low blood pressure, stroke, heart attack, arrhythmia, fluid retention, aberrant plasma osmolarity, aberrant sodium and metabolism and excretion of other ions and other diseases associated therewith such as diabetes.

As noted above, this gene was originally identified as a putative salty taste receptor using a whole genome screening strategy aimed at identifying genes specifically expressed in the top of the human taste buds. (See provisional applications incorporated by reference herein which identify TRPML3 as being a taste specific gene in rodents and primates.) The inventors had also earlier determined from previous experiments that the top of the taste buds contain cells that over-express known taste receptor genes and other taste specific genes including the sodium ion channel TRPML3 which is similarly enriched in the top half of taste buds. In contrast, the bottom of the taste buds contains precursor cells of the sensory taste bud cells that reside in the top portion of the taste bud. This database allowed the inventors to identify many genes specifically expressed by the top (sensory) cells of the taste buds including TRPML3.

It was further noted by the inventors in reviewing the taste specific genes identified that one of these genes, MCOLN3 or TRPML3 had been previously reported to be responsible for the phenotype of a mouse mutant called varitint-waddler that exhibits early-onset hearing loss, vestibular defects, pigment abnormalities and perinatal lethality (DiPalma et al., Mutations in Mcoln3 associated with deafness and pigmentation defects in variant-waddler (Va) mice. Proc. Natl. Acad. Sci. USA 99: 14994-14999; 2002). As noted in the background of the invention, MCOLN3 or TRPML3 is expressed in the hair cells and plasma membrane of stereocilia (in the ears) and a mutation resulting in an ala 419 to pro substitution in the fifth transmembrane domain had specifically been reported to result is a hyperactive MCOLN3 that causes in the death of cells expressing this molecule, such as the hair cells of the ear (hence the deafness of the Va mouse) (Grimm C et al., Proc Natl. Acad. Sci. USA 104: 19583-8; 2007).

Based on the inventors' elucidation of TRPML3 as a salt receptor in primates and likely other mammals it was further predicted that this mouse would also exhibit salty taste abnormalities (due to the abnormal MCOLN3 molecule and its effect in the taste bud cells of the tongue which will likely impair salty taste perception). In fact this has been conformed by the inventors. The inventors have conducted neurophysiology studies (described infra in the experimental examples) using mice which express this mutant TRPML3 gene (varitint mouse) and have confirmed, as hoped and anticipated that that these mice exhibit impaired responses to salty taste stimuli (as evidenced by nerve recording results in these mice stimulated with salty taste stimuli at concentrations where a positive nerve recording response would normally be observed). Using CT nerve recordings, Varitint waddler mice were shown to exhibit a deficiency in the response to sodium chloride. Specifically, Varitint waddler mice have a greatly reduced benzamil-insensitive CT nerve response to sodium chloride.

Also, the inventors have molecular and immunohistochemical data which revealed that these same mice have taste buds which are ablated of the TRPML3 expressing taste bud cells. This confirms the inventors' supposition that this unique taste cell subset was involved in detecting salty taste and a prerequisite for salty taste perception.

Therefore, the inventors have in vivo evidence substantiating a conclusion that the presence of a functional TRPML3 ion channel in specific taste cells (“professional” salty taste cell) is a necessary prerequisite for salty taste perception in rodents and likely other mammals including most especially humans and other primates.

In addition, because, the inventors have determined using public databases that MCOLN3 is expressed strongly in the adrenal and pituitary glands this is further supportive of the inventors' discovery as well as suggesting other applications of the gene and compounds that specifically detect or target this gene and/or modulate its function. The fact that this gene is expressed in adrenal and pituitary glands is a key observation because the adrenal glands represent one of the main sodium metabolism regulators of the body. These glands monitor salt levels of the blood, and secrete aldosterone (a mineralocorticoid) that regulates blood pressure and water and salt balance in the body by helping the kidney retain sodium and excrete potassium. When aldosterone production falls too low, the kidneys are not able to regulate salt and water balance, causing blood volume and blood pressure to drop.

Also, the pituitary glands produce vasopressin (ASP) a hormone that involves sodium levels in the urine and plays a role in sodium excretion through the urine. Particularly, one of the most important roles of AVP is to regulate the body's retention of water; it is released when the body is dehydrated and causes the kidneys to conserve water, thus concentrating the urine, and reducing urine volume. It also raises blood pressure by inducing moderate vasoconstriction.

In addition AVP increases the permeability to water of the collecting ducts in the nephrons of kidneys and thus allows water reabsorption and excretion of a smaller volume of concentrated urine-antidiuresis. This occurs through insertion of additional water channels (Aquaporin-2) into the apical membrane of the collecting duct epithelial cells. The aquaporin allows water to pass out of the nephron at the collecting ducts and into the cell, increasing the amount of water re-absorbed from the filtrate.

AVP also increases permeability of the medullary portion of the collecting duct to urea, allowing increased reabsorption of urea into the medullary interstitium, down the concentration gradient created from the removal of water in the cortical collecting duct. Moreover, another renal role for AVP is that it stimulates sodium reabsorption in the thick-ascending loop of henle. Therefore, based on the inventors' discovery that TRPML3 is involved in salty taste detection, it is not surprising that TRPML3 is expressed in 2 glands which produce polypeptides very significantly involved in sodium transport and excretion and that this gene, aside from being involved in salty taste perception plays an active role in regulating other processes involving sodium transport, absorption and excretion and in particular processes regulated by vasopressin or aldosterone.

Therefore, based on these observations and the elucidation of this gene as a salty taste receptor, MCOLN3 was further identified by the inventors as being a key salt/sodium monitoring molecule in the adrenal glands that controls the production of aldosterone and/or in regulating vasopressin release by the pituitary glands.

In the tongue, as is anticipated for a molecule identified as being a taste receptor that detects salty taste stimuli, MCOLN3 or TRPML3 is expressed by a subset of taste sensory cells located in the top of the taste buds which are responsible for detecting salty taste. Therefore, this invention provides compelling proof of the pivotal role of MCOLN3 or TRPML3 in detecting and regulating salt in various tissues.

While MCOLN3 had previously been reported to be a sodium conducting channel, there are numerous sodium ion channel polypeptides and this channel had not been previously recognized as being involved in salty taste perception or in regulating sodium metabolism, excretion, transport or sodium related processes involving vasopressin and aldosterone. Therefore, this invention constitutes a new and unexpected discovery as it provides a new use (salty taste receptor) for a known gene (MCOLN3). In the pituitary, the inventors further anticipate based on their discoveries and data contained herein substantiating the role of TRPML3 as a salty taste receptor that MCOLN3 is further likely involved in the regulation of vasopressin release. As mentioned, vasopressin is a key regulator of urine production through its effects on the kidneys. Importantly, vasopressin release from the posterior pituitary is known to be regulated by NaCl concentration. This protein is highly expressed in the pituitary glands. Therefore, based on its expression in the pituitary, TRPML3 through its probable effect on vasopressin release, likely regulates NaCl metabolism in the body, through its effects on fluid retention, NaCl sensing and concentration, and blood pressure.

Therefore, in one embodiment the invention identifies MCOLN3 or TRPML3 as a human salty taste receptor and based thereon provides screening assays using cells transfected with this gene for the purpose of identifying agonists, antagonists or enhancers (modulators) of the function of this molecule which will modulate salty taste and other taste related TRPML3 functions and non-taste related functions such as those involving sodium excretion, metabolism, and transport in different tissues and pathological conditions relating to aberrant TRPML3 expression such as are identified herein. In another embodiment the invention identifies MCOLN3 or TRPML3 as a receptor involved in sodium homeostasis, including blood pressure regulation in hypertension (high blood pressure) and hypotension (low blood pressure); modulation of renal sodium balance to control blood pressure; modulation of plasma osmolarity, including decreases in osmolarity (hypoosmotic) and increases in osmolarity (hyperosmotic) via reabsorption of water in the kidney in normal and diseased states such as diabetis insipidus; modulation of ENaC function and/or trafficking; modulation of aquaporin-2 function and/or trafficking; modulation of vasopressin (antidiuretic hormone) secretion from the pituitary gland and modulation of aldosterone secretion from the adrenal glands and based thereon provides screening assays using cells transfected with this gene for the purpose of identifying agonists, antagonists or enhancers (modulators) of the function of this molecule which will modulate these TRPML3 functions such as those involving sodium excretion, metabolism, and transport in different tissues and pathological conditions relating to aberrant TRPML3 expression such as are identified herein.

More specifically, in another embodiment the invention provides an isolated and purified taste, adrenal, pituitary or urinary organ cell or enriched taste cell sample comprising a taste, adrenal, pituitary or urinary organ cell that expresses TRPML3 that is involved in salty taste perception, sodium metabolism, aldosterone production, and/or vasopressin release wherein said isolated taste, adrenal, pituitary, or urinary organ cell or enriched taste cell sample expresses TRPML3 gene or a variant thereof that encodes a sodium channel that modulates at least one of salty taste, sodium metabolism, aldosterone production and vasopressin release. Preferably the cell will be of human, non-human primate or rodent origin.

Also in another embodiment the invention provides an isolated taste receptor that modulates salty taste perception comprising a TRPML3 polypeptide or variant thereof that is useful in assays for identifying TRPML3 modulators and/or which taste receptor polypeptide modulates salty taste in mammals including humans. This taste receptor may be monomeric or polymeric (homopolymeric or heteropolymeric) and may comprise other taste specific polypeptides, e.g., other ion channel polypeptides such as NKAIN3 or NALCN or related ion channels such as TRPML1 or TRPML2. This TRPML3 polypeptide or nucleic acid sequence may be of mammalian or other species origin. As mentioned, given the importance of sodium metabolism and excretion to organism's homeostasis and well being, it is likely that this gene and its various species orthologs play a role in salty taste perception and salt (sodium) metabolism, and excretion in different mammals and likely other vertebrates such as reptiles, amphibians and avians.

The TRPML3 genes according to the invention may be wild-type or may be genetically engineered to introduce desired mutations that affect (enhance or inhibit) ion channel function and/or which fix the ion channel in an open or closed orientation. Also, it may be modified by the substitution of host preferred codons. Such mutations are exemplified herein and one skilled in the art will be able to design others using methods known in the art. Therefore, it should be understood that the TRPML3 polypeptides herein may be modifies relative to the native TRPML3 polypeptide, and may possess 80, 85, 90, 95, 96, 97, 98, 99, or greater sequence identity to native TRPML3 polypeptide or a functional fragment. In addition, the subject TRPML3 polypeptides may comprise chimeric ion channels, i.e., wherein one or more domains or regions of the endogenous TRPML3 ion channel are substituted by the corresponding domain or region of a related (e.g. an ortholog) ion channel, an ion channel in the same TRPML family (TRPML1 or TRPML2) or another ion channel, e.g., another sodium channel such as NALCN or NKAIN3.

These chimeras may be constructed based on the known TRPML3 protein topology. This topology is depicted schematically below.

In this schematic the transmembrane domains are listed 1 through 6. The amino (N) and carboxy (C) are inside the cell. In addition, there is a large extracellular loop between TM1 and TM2 that resides outside the cells. Chimeras that are functional (still respond to sodium) can e.g., potentially be constructed by substituting the extracellular loop region spanning TM1 and TM2 with that of another TRPML3 or another ion channel polypeptide or by substituting a TM with the corresponding TM of another ion channel. Also, chimeras can be made between human and mouse TRPML3 in which the large extracellular loop between TM1 and TM2 is swapped.

Also, residues around the pore region and TM5 potentially may be modified, e.g., by corresponding residues in other ion channels.

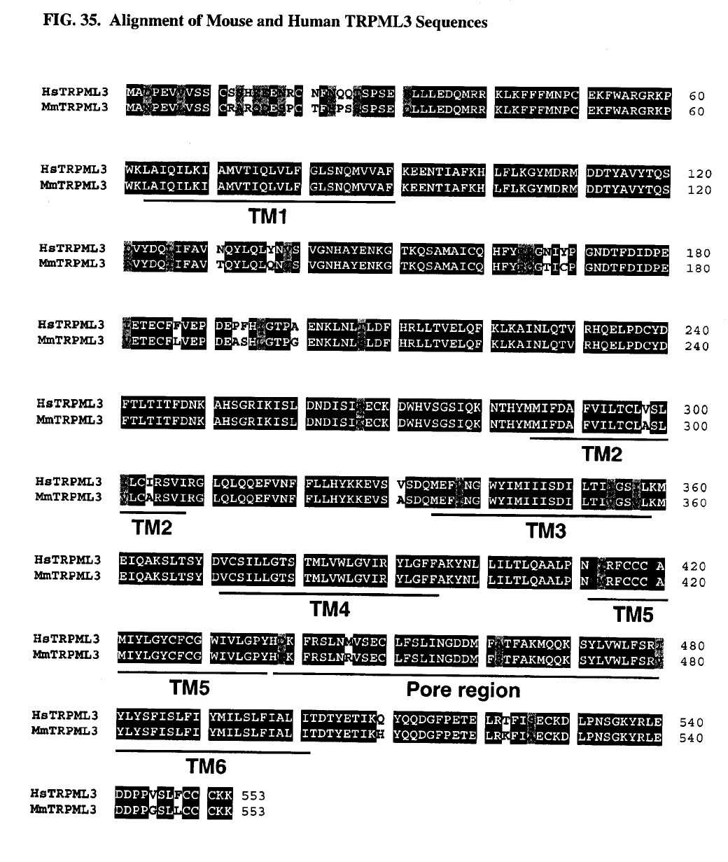

Based on a comparison and alignment of the protein sequences derived from human (NM_{—}018298) and mouse (NM_{—}134160) TRPML3 sequences (FIG. 35) wherein human is denoted Hs and mouse is denoted Mm it can be seen that these proteins are 91% identical and 96% similar. This substantiates the inventors' supposition (reasonable) that TRPML3 likely is well conserved in different mammals and potentially other vertebrates given the important physiological functions it regulates and that chimeras and mutants which are functional may be constructed. In fact, this application contains sequences for avian and fish TRPML3 genes in the Sequence Listing preceding the claims. Other orthologs may be identified using these and other TRPML3 genes as probes.

The alignment in the figure (FIG. 35) similarly shows the six transmembrane domains in the TRPML3 polypeptides for both human and mouse TRPML3 are underlined TM1 through TM6. The pore region between TM5 and TM6 is denoted ‘pore region’. As in the schematic above, the amino and carboxy termini are predicted to be located inside the cells. In constructing mutants it may be desirable to retain the residues in the pore region intact or to modify very few residues with these modifications if present corresponding to the residues present in the pore region of other TRPML3 polypeptides or to the corresponding residues in the pore region of TRPML1 or TRPML2.

The A419P mutation discussed herein and found in the varitint-waddler mouse locks TRPML3 in the open conformation and is in TM5 and is highlighted in FIG. 35.

As discussed and shown in the examples infra, this mutation is useful in assays for TRPML3 modulators (blockers) and in particular may be used in FLIPR assays. Another mutation, V412P, partially activates TRPML3 and is denoted in magenta. This mutation increases TRPML3 activity and can be used to screen for enhancers in a FLIPR assay. Also, other mutations potentially can be made around TM5 and the pore region to alter TRPML3 ion channel activity and generate active channels that could be used in high-throughput screens.

In general, this invention uses assays that include the use of a wild-type or mutated TRPML3 polypeptide or one wherein the codons are optimized for the host cell wherein expression takes place, e.g., a human cell. However, in some instances it is desired to determine all of the effects of TRPML3 in vivo by eliminating the expression of the TRPML3 polypeptide. Therefore, in another embodiment the invention provides a transgenic non-human animal which has been genetically engineered to knock out the expression of endogenous TRPML3 or to express a non-functional TRPML3 polypeptide.

Also in another embodiment the invention provides a transgenic non-human animal which has been genetically engineered to express a heterologous TRPML3 polypeptide or a mutant or chimeric TRPML3 polypeptide so that the animal may be screened to identify modulators of this heterologous TRPML3 polypeptide, e.g., human or other primate TRPML3 or a TRPML3 of a domesticated animal (dog, cat, etc.)

Also in another embodiment the invention provides assay methods of using a transgenic animal that expresses a mutant or heterologous TRPML3 in screens to identify antagonists, agonists or enhancers of TRPML3 (so that the animal may be screened to identify modulators of the mutant or heterologous TRPML3 polypeptide).

Also, in a related embodiment the invention produces transgenic animals containing the TRPML3 mutation found in the Varitint-mouse or other functionally equivalent mutations in order to create transgenic animals depleted of specific types of TRPML3 expressing cells, such as salty taste cells, pituitary cells, adrenal cells, melanocytes, or urinary organ system cells and to study the effects of these cell ablations in these animals or to use these animals as disease models for conditions involving these ablated cells.

Also in another embodiment the invention provides methods of using a transgenic animal according wherein the TRPML3 gene has been “knocked out” in order to elucidate the effect of TRPML3 on taste, and on cardiac or urinary function and in particular on aldosterone production, sodium metabolism, salty taste perception or vasopressin release.

Also, as the TRPML3 knock-out should give rise to conditions or abnormalities involving aberrant sodium transport, absorption and excretion and related urinary or cardiovascular effects these transgenic animals potentially may be used as models for these conditions and for testing potential therapeutics and therapeutic regimens, especially regimens for modulating blood pressure, plasma osmolarity, sodium transport, excretion and homeostasis and other conditions involving sodium levels.

Also, in a more specific embodiment the invention provides the use of use of Varitint waddler mice to study salty taste behavior in the absence of TRPML3 taste cells.

Also, in a more specific embodiment the invention relates to the use of the Varitint waddler mice to detect the effect of TRPML3 function on melanocytes, pituitary, adrenal, taste, urinary or taste cells.

Also, in a more specific embodiment the invention relates to the use of the Varitint waddler mice in assays to detect genes specifically expressed in salty taste cells and not in the Varitint waddler mice (as salty taste cells are ablated therein) which genes may modulate TRPML3 function, or function as a salty taste receptor or modulate transmission of salty taste signaling from TRPML3 to the nerve fibers and/or control the development differentiation or apoptosis of salty taste cells. These gene detection assays may comprise the use of gene chips or microarray technology to compare the genes expressed in salty taste cells versus genes expressed in Varitint waddler mice.

Also, in a more specific embodiment the invention provides methods of treating parathyroid related diseases such as calcium homeostasis, hypercalcemia, osteitis, hypoparathyroidism, hyperparathyroidism, osteitis fibrosis cystica, pseudohypoparathyroidism, Jansen's metaphyseal chondroplasia, Blomstrand's chondroplasia, and osteoporosis of different causes such as diseases, age, menopause, chemotherapy, radiation therapy, drugs and the like.

Also in another embodiment the invention provides a recombinant cell which expresses a salty taste receptor comprising TRPML3 or a variant thereof that encodes a sodium ion channel polypeptide.

Also, in another embodiment the invention provides the use of A419P TRPML3 polypeptide as a toxin to kill specific cell types, e.g. salty taste cells, pituitary cells, adrenal cells, melanocytes, and/or urinary organ system cells which express TRPML3.

Also, in another embodiment the invention provides the use of labeled molecules that specifically bind TRPML3 to study sodium transport, metabolism, or excretion by the body.

Also, in another embodiment the invention provides the use of molecules that specifically bind TRPML3 to direct therapeutics or diagnostic agents to specific sites, e.g., salty taste cells, adrenal cells, melanocytes, pituitary cells, kidney cells involved in sodium homeostasis, and other TRPML3 expressing cells involved in regulation of sodium levels and thereby blood pressure, plasma osmolarity and the like.

Also more specifically, in another embodiment the invention provides an assay for identifying compounds that agonize, antagonize or enhance an activity of TRPML3 comprising contacting a recombinant or endogenous taste or other cell that expresses TRPML3 with a putative TRPML3 enhancer, agonist or antagonist and determining the effect thereof on TRPML3 activity. Preferably these assays will be electrophysiological assays e.g., patch clamp or two electrode voltage clamping assays.

Also more specifically, in another embodiment the invention provides methods for identifying TRPML3 modulators by an ion flux assay.

Also more specifically, in another embodiment the invention provides such TRPML3 assays wherein the identified agonist, antagonist, or enhancer compounds are evaluated in a taste test.

Also more specifically, in another embodiment the invention provides such TRPML3 assays wherein the effect of the identified agonist, antagonist, or enhancer compounds on aldosterone production is tested in an animal.

Also more specifically, in another embodiment the invention provides such TRPML3 assays wherein the effect of the identified agonist, antagonist, or enhancer compounds on vasopressin release is tested in an animal.

Also more specifically, in another embodiment the invention provides such TRPML3 assays wherein the effect of the identified agonist, antagonist, or enhancer compounds on at least one of cardiac or urinary function and more specifically on blood pressure, fluid retention, sodium metabolism or urine production, wherein this is tested in an animal. Also more specifically, in another embodiment the invention provides such TRPML3 assays wherein the effect of the identified agonist, antagonist, or enhancer compounds on at least one of sodium homeostasis, including blood pressure regulation in hypertension (high blood pressure) and hypotension (low blood pressure); blood pressure., plasma osmolarity, including decreases in osmolarity (hypoosmotic) and increases in osmolarity (hyperosmotic) via reabsorption of water in the kidney in normal and diseased states such as diabetis insipidus; ENaC function and/or trafficking; aquaporin-2 function and/or trafficking; vasopressin (antidiuretic hormone) secretion from the pituitary gland; or aldosterone secretion from the adrenal glands is evaluated in an animal.

Also more specifically, in another embodiment the invention provides the use of the identified agonist, antagonist, or enhancer compounds for treating a disease or condition involving aldosterone production comprising administering an effective amount of a compound that modulates TRPML3.

Also more specifically, in another embodiment the invention provides the use of the identified agonist, antagonist, or enhancer compounds for treating a disease or condition involving vasopressin release comprising administering an effective amount of a compound that modulates TRPML3. As mentioned, these conditions include by way of example diseases and conditions treatable using TRPML3 modulators which agonize or antagonize vasopressin such as diabetes, obesity, kidney diseases such as cystic kidney disease, acquired renal cystic disease, ocular circulation related disorders such as myopia; nausea, emesis, sexual dysfunction (male or female), edema, hypertension, congestive heart failure (ranging from class II of the New York Heart Association to florid pulmonary edema), periodic idiopathic edema, nephritic syndrome, ascites due to cirrhosis or other causes, cerebral edema of various causes, as well as dilutional hyponatremia and metabolic alterations collectively known as the syndrome of inappropriate ADH secretion and other diseases or conditions wherein vasodilation and/or antioxytocic activity is therapeutically desirable.

Also more specifically, in another embodiment the invention provides the use of the identified agonist, antagonist, or enhancer compounds for treating a disease or condition involving aldosterone production comprising administering an effective amount of a compound that modulates TRPML3 and thereby aldosterone. Diseases and conditions treatable using TRPML3 modulators which agonize or antagonize aldosterone and thereby sodium transport and excretion include by way of example edema, blood pressure (hyper or hypotension), liver cirrhosis, primary hyperaldosteronemia, renal dysfunction, diabetes (Type I or II) and the pathological symptoms associated therewith including circulatory problems, edema, ocular disorders relating to poor circulation, hypercortisolaemia, atherosclerosis or obesity, e.g., abdominal obesity, as well as liver disease, sexual dysfunction (male or female), cerebrovascular disease, vascular disease, retinopathy, neuropathy, insulinopathy, endothelial dysfunction, baroreceptor dysfunction, migraine headaches, hot flashes, and premenstrual tension and other cardiovascular conditions such as atherosclerosis, heart failure, congestive heart failure, vascular disease, stroke, myocardial infarction, endothelial dysfunction, ventricular hypertrophy, renal dysfunction, target-organ damage, thrombosis, cardiac arrhythmia, plaque rupture and aneurysm.

Also more specifically, in another embodiment the invention provides the use of the identified agonist, antagonist, or enhancer compounds for treating a disease or condition involving TRPML3 such as Addison's disease, or type IV mucolipidosis.

Also more specifically, in another embodiment the invention provides the use of the identified agonist, antagonist, or enhancer compounds for modulating cardiac function, e.g., blood pressure, arrhythmia, or stroke or fluid retention in a subject in need thereof comprising administering an effective amount of a compound that modulates TRPML3.

Also more specifically, in another embodiment the invention provides the use of such identified agonist, antagonist, or enhancer compounds for modulating urine production and/or excretion in a subject in need thereof comprising administering an effective amount of a compound that modulates TRPML3.

Also more specifically it is another embodiment to provide the use of TRPML3 modulators, e.g., enhancers, agonists or antagonists, for treating conditions involving melanocytes such as melanoma and pigmentation disorders and to promote the growth and coloration of hair or skin which has lost coloration, e.g. because of disease, aging, UV radiation, chemotherapy, or hormone imbalance.

Also more specifically it is another embodiment to provide the use of TRPML3 modulators, e.g., enhancers, agonists or antagonists, for treating conditions involving pituitary cells such as pituitary cancer or diabetes or pituitary diseases.

Also more specifically it is another embodiment to provide the use of TRPML3 modulators, e.g., enhancers, agonists or antagonists, for treating conditions involving adrenal cells such as adrenal cancer or other adrenal conditions.

Also more specifically it is another embodiment to provide the use of TRPML3 modulators, e.g., enhancers, agonists or antagonists, for treating conditions involving taste buds such as taste bud related malignancies other taste bud related conditions.

Also more specifically, in another embodiment the invention provides such identified agonist, antagonist, or enhancer compounds useful in taste and therapeutic applications which may include polypeptides, antibodies, small molecules, siRNAs, antisense RNAs, ribozymes et al.

The discovery of TRPML3 as a salty taste receptor was based in part on the hypothesis that human salty taste may be mediated, in part, by a sodium or other ion channels as well as transporters and GPCRs expressed specifically in taste cells. The compounds identified using these gene products and their derivatives that modulate the activity of these target genes potentially can be used as modulators of human salty taste in foods, beverages and medicinals for human consumption. Also, such compounds and their derivatives potentially may be used to treat diseases involving aberrant ion channel function. Further the compounds identified using genes identified according to the invention and cells which express same are useful in therapeutic screening assays as discussed herein for identifying potential therapeutics that modulate other taste-cell related functions and phenotypes.

This gene was deemed significant by the inventors based on its selective expression in primate fungiform papilla taste cells found at the front of the tongue and circumvallate papilla taste cells found at the back of the tongue using gene-chips microarrays from taste receptor cells as compared to non-taste lingual epithelial cells isolated by laser capture microdissection (LCM). This protocol also identified 2 other taste specific ion channels NKAIN3 and NALCN which are enriched in the top half of the taste buds. Since salt perception is most prevalent at the front of the tongue, a salt receptor gene was predicted to be contained within this set of identified genes. (It is stated throughout the application that the inventors have identified “a human or mammalian salty taste receptor” rather than “the human or mammalian salty taste receptor” since it is conceivable that humans or other mammals may have some redundancy in the genes that regulate salty taste and sodium metabolism.

The subject gene was initially identified as being a taste specific ion channel polypeptide putatively involved in salty taste in mammals. This protocol involved the steps of (i) identifying a set of genes including genes which are expressed in macaque taste (fungiform and circumvallate papilla taste cells) but which are not expressed in lingual epithelial cells and/or genes which are expressed in taste cells at substantially higher levels than in lingual cells; (ii) identifying a subset of genes within the set of genes identified in (i) which are selected based on criteria which suggest that they are likely salt receptor candidates, i.e., putative ion channels and/or encode multidomain transmembrane proteins. These genes were then examined to determine whether these genes are expressed or not expressed in taste cells which express umami, sweet or bitter taste receptors (T1Rs or T2Rs) or sour taste receptors (PKD2L1/PKD1L3); and (iii) functionally expressing one or more genes in the subset identified according to (ii) and determining which of these genes function as a sodium responsive ion channel or sodium responsive receptor or transporter and thereby identifying this gene or genes as a putative gene that modulates salty taste. Typically, the taste tissues for this method are derived from human, primate, or rodent sources. In one preferred embodiment of the method, the genes in step (iii) function as sodium responsive ion channels, and more preferably, when the genes are expressed, a fraction of the channel population is open and passing sodium at rest.

In a preferred embodiment, step (i) comprises the use of laser capture microdissection (LCM) to dissect and purify taste tissues from non-taste tissues. In one mode of this embodiment, step (i) comprises RNA amplification of genes from taste cells and lingual cells and the amplified genes are screened against a gene chip containing a sample of genes specific to the particular mammal from which the taste and lingual tissues are obtained, and preferably, the gene chips include a set of annotated human genes. In an alternative mode of this embodiment, step (i) comprises high throughput PCR using primers for each ion channel in a mammalian genome.

In another preferred embodiment, step (ii) is affected by in situ hybridization using antisense RNA probes specific for the set of genes identified in step (i) to determine level of expression in taste versus lingual cells. In an alternative preferred embodiment, step (ii) is affected by use of immunochemical detection using a labeled antibody specific to the protein encoded by gene or genes identified in step (i).

In another embodiment of the method for identifying a gene encoding a polypeptide involved in salty taste perception in a mammal, the method of this invention comprises the steps of (i) identifying a set of macaque genes including genes which are expressed in taste cells but which are not expressed in lingual cells and/or genes which are expressed in taste cells at substantially higher levels than in macaque lingual cells; (ii) identifying a subset of genes within the set of genes identified in (i) which are not expressed in taste cells which express umami, sweet or bitter taste receptors (T1Rs or T2Rs) or sour taste receptors (PID2L1/PID1L3); and (iii) determining, in a primary neuron which expresses one or more genes in the subset identified according to (ii), which of said genes functions as a sodium responsive ion channel or sodium responsive receptor or transporter and thereby identifying this gene or genes as a putative gene that modulates salty taste. In one mode of this embodiment, step (iii) comprises contacting the neuron with an antibody which specifically binds the gene and inhibits its function.

In another generic mode, this invention provides an assay for identifying a compound having potential in vivo application for modulating human salty taste. This method comprises the steps of (i) contacting a cell that expresses a gene encoding an ion channel, receptor or transporter identified as a putative salty taste affecting gene according to any one of the methods above, or a gene encoding a polypeptide possessing at least 90% sequence identity to the polypeptide encoded thereby, with at least one putative enhancer compound; (ii) assaying sodium conductance, receptor activity or sodium transport in the presence and absence of said putative enhancer; and (iii) identifying the compound as a potential salty taste enhancer based on whether it increases sodium conductance, the activity of said receptor or sodium transport. In various embodiments, the gene encodes an ion channel or the gene encodes a GPCR. Preferably, the gene is a human gene. More preferably, the method further includes testing the effect of the compound or a derivative thereof in a human or animal taste test. Preferably, the selected compound promotes sodium ion transport into taste bud cells. The putative salty taste affecting gene may be expressed in an amphibian oocyte, or in a mammalian cell, preferably a Xenopus oocyte or a mammalian cell selected from the group consisting of a HEK293, HEK293T, Swiss3T3, CHO, BHiK, NIH3T3, monkey L cell, African green monkey kidney cell, Ltk-cell and COS cell. Preferably, the putative salty taste affecting gene is expressed under the control of a regulatable promoter. A putative salty taste affecting gene may be expressed stably or transiently. In a preferred mode, the salty taste affecting gene is TRPML3.

In a preferred mode, the assay of step (ii) is an electrophysiological assay which uses a sodium sensitive dye, and preferred dyes include membrane potential dyes selected from the group consisting of Molecular Devices Membrane Potential Kit (Cat#R8034), Di-4-ANEPPS (pyridinium, 4-(2-(6-(dibutylamino)-2-naphthalen-yl)ethenyl)-1-(3-sulfopropyl)hydroxide, inner salt, DiSBACC4(2)(bis-(1,2-dibabituric acid)-triethine oxanol), Cc-2-DMPE (Pacific Blue 1,2-dietradecanoyl-sn-glycerol-3phosphoethanolamine, triethylammonium salt) and SBFI-AM (1,3-benzenedicrboxylic acid, 4,4-[1,4,10-trioxa-7,13-diazacylopentadecane-7,13-diylbis (5-methoxy-6,1,2-benzofurandiyl)}bis-tetrakis {(acetyloxy)methyl}ester (Molecular Probes), more preferably, the sodium sensitive dye is sodium green tetraacetate (Molecular Probes) or Na-sensitive Dye Kit (Molecular Devices). In another preferred mode, the assay of step (ii) is a two electrode voltage clamping assay in Xenopus oocytes, or the assay is a patch clamp assay in mammalian cells. Preferably, the assay measures activity by an ion flux assay, including using atomic absorption spectroscopy to detect ion flux.

Alternatively, the assay may use a fluorescence plate reader (FLIPR), or a voltage imaging plate reader (VIPR), which is used to increase ion channel-dependent sodium or fluid absorption. In a preferred embodiment of this method, the activity of the putative salty taste affecting gene is assayed in a frog oocyte electrophysiologically by two electrode voltage clamping, or in a mammalian cell, preferably using an automatic imaging instrument, which may be a fluorescence plate reader (FLIPR) or a voltage imaging plate reader (VIPR) or patch-clamping.

In yet another mode, this invention provides an assay for identifying a compound having potential in vivo application for modulating human sweet, bitter, umami, or sour taste. This method comprises the steps of (i) contacting a cell that expresses a gene identified according to the invention with at least one putative enhancer or blocker compound; (ii) assaying sodium conductance, receptor activity or taste gene product function in the presence and absence of said putative enhancer or blocker; and (iii) identifying the compound as a potential enhancer or blocker for sweet, bitter or umami taste based on whether it modulates sodium conductance, the activity of said receptor or taste gene product function.

In yet another mode, this invention provides an assay for identifying a compound having potential in vivo application for as a potential therapeutic. This method comprises the steps of (i) contacting a cell that expresses a gene identified according to the invention with at least one putative enhancer or blocker compound; (ii) assaying sodium conductance, receptor activity or taste gene product function in the presence and absence of said putative enhancer or blocker; and (iii) identifying the compound as a potential therapeutic that may be used to modulate a taste cell related function or phenotype that does not directly involve taste such a digestive disorder or disease, taste cell or taste bud turnover or regeneration, immune regulation of the oral or digestive system, or treatment of a metabolic disorder such as diabetes, obesity, eating disorder et al., based on whether it modulates sodium conductance, the activity of said receptor or taste gene product function.

### DETAILED DESCRIPTION OF THE FIGURES

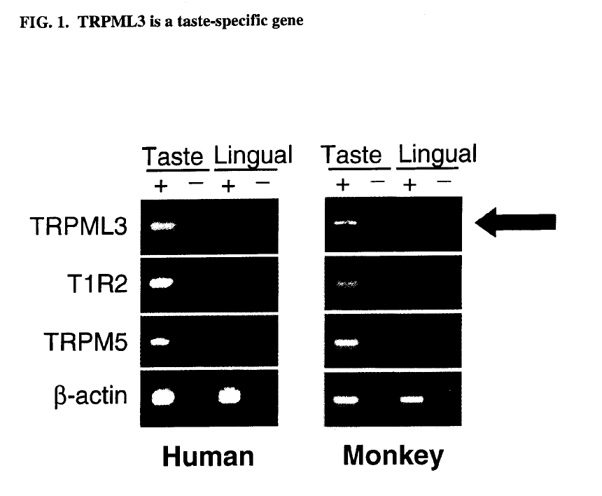

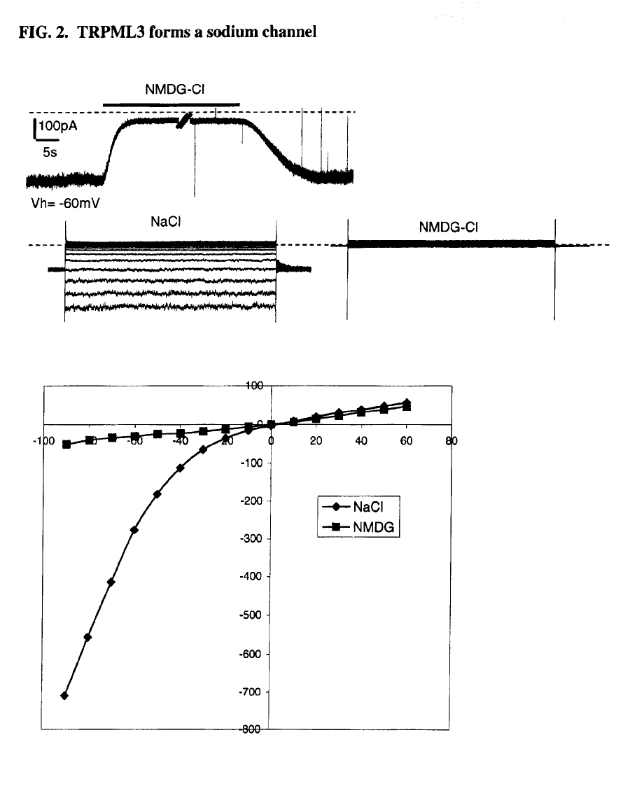

FIG. 1 contains RT-PCR data that reveal that TRPML3 is a taste-specific gene. In this experiment RT-PCR was affected using human (left) and monkey (right) taste buds (taste) and lingual epithelial cells (lingual) samples collected by laser capture microdissection. It can be seen that TRPML3 is only expressed in taste cells, similar to the known taste-specific genes T1R2 and TRPM5. The housekeeping gene beta-actin is expressed in both taste and lingual cells demonstrating that RNA from both samples is of high quality. ‘+’ indicates reverse transcription was performed and ‘−’ indicates that no reverse transcription was performed (negative control). Bands are only observed with reverse transcription. All bands were cloned and sequenced to confirm gene identities.

FIG. 2 contain electrophysiological experiments which show that TRPML3 forms a sodium channel. Whole cell patch clamp electrophysiology of cells expressing human TRPML3 was affected. The results of these experiments reveal that TRPML3 generates a sodium leak current that is blocked upon removal of sodium and replacement with the large impermeant cation NMDG. The top trace in FIG. 2 shows current at a holding potential of −60 mV. The middle traces in the Figure show current-voltage traces from −100 mV to +60 mV in the presence (NaCl) and absence (NMDG-Cl) of sodium. The bottom graph in FIG. 2 shows current voltage curves in the presence (dark blue line; diamonds) and absence (magenta line; squares) of sodium. It can be seen that TRPML3 exhibits inward rectification (more current at negative voltages compared to positive voltages)

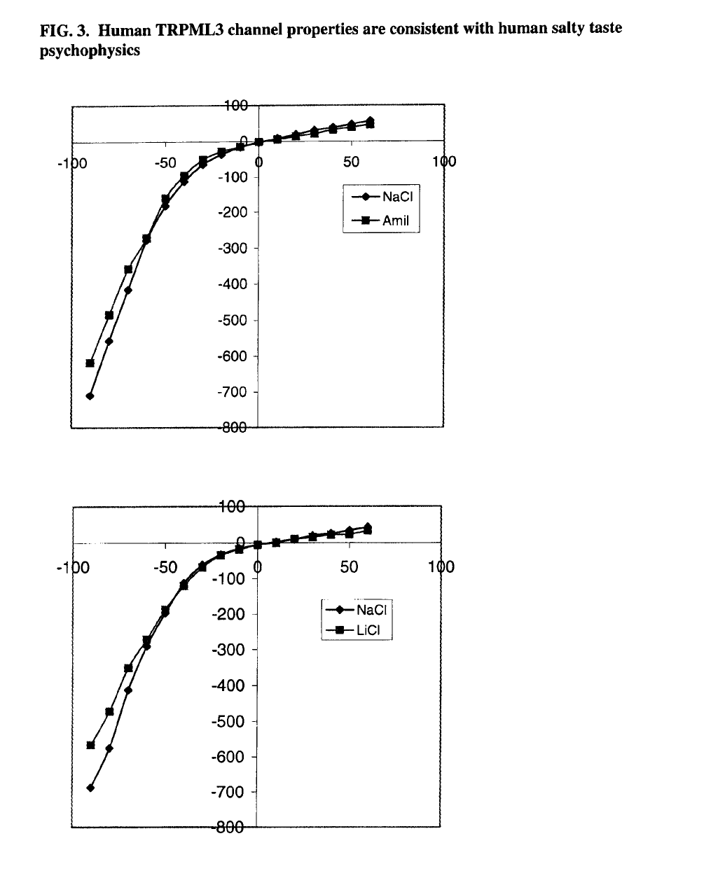

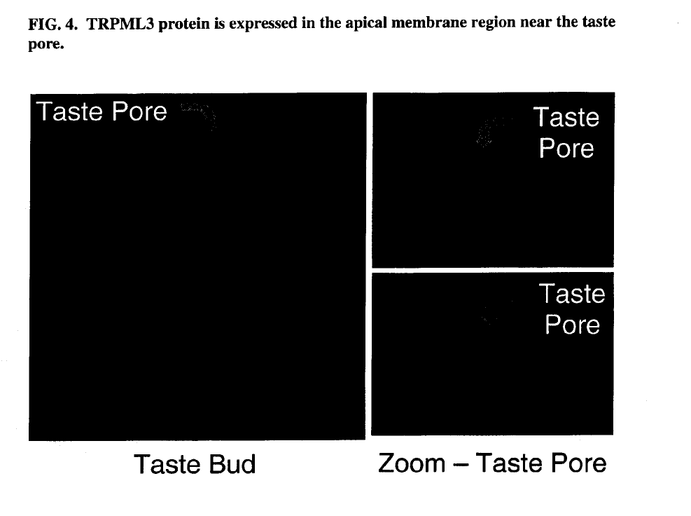

FIG. 3 contains the results of other electrophysiology experiments which indicate that human TRPML3 channel properties are consistent with human salty taste psychophysics. The top graph in FIG. 3 contains current-voltage curves showing TRPML3 sodium conductance (dark blue line; diamonds) is not blocked by 30 uM amiloride (magenta line; squares). Both human salty taste and TRPML3 are not blocked by amiloride. The bottom graph in the same Figure contains current-voltage curves showing TRPML3 is equally permeable to the salty cations sodium (dark blue line; diamonds) and lithium (magenta line; squares). This result is consistent with TRPL3 being a human salty taste receptor given the fact that sodium and lithium are known to be equally salty to humans since both cations equally permeate the human TRPML3 channel.

FIG. 4 contains an experiment which shows that the TRPML3 protein is expressed in the apical membrane region near the taste pore. It can be seen that the TRPML3 antibody labels taste cell processes extending to the taste pore (left image). Magnification of the apical taste bud domain facing the saliva clearly demonstrates that TRPML3 protein is expressed at the taste pore region (3 right images; taste pore denoted with blue arrows). This observation is also consistent with TRPML3 being the human salty taste receptor since this location is ideally suited for TRPML3 to sense sodium in the saliva. Similar to TRPML3, other known taste receptors (sweet, bitter, umami, and sour) are also polarized to the taste pore where they sample saliva for their requisite tastants.

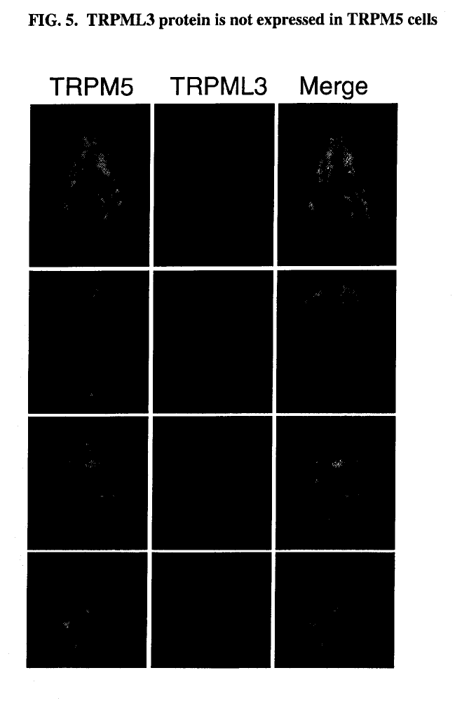

FIG. 5 contains the data of an immunochemistry double labeling experiment which indicates that the TRPML3 protein is not expressed in TRPM5 cells. The figure shows the results of double label immunohistochemistry with TRPM5 (green; left images) and TRPML3 (red; middle images) in monkey CV papilla. It can be seen in the Figure that cells expressing TRPM5 and TRPML3 are distinct (merged images on the right). These data indicate that TRPML3 is not expressed in TRPM5 cells (encompassing sweet, bitter, and umami cells) but exclusively in professional salty taste cells.

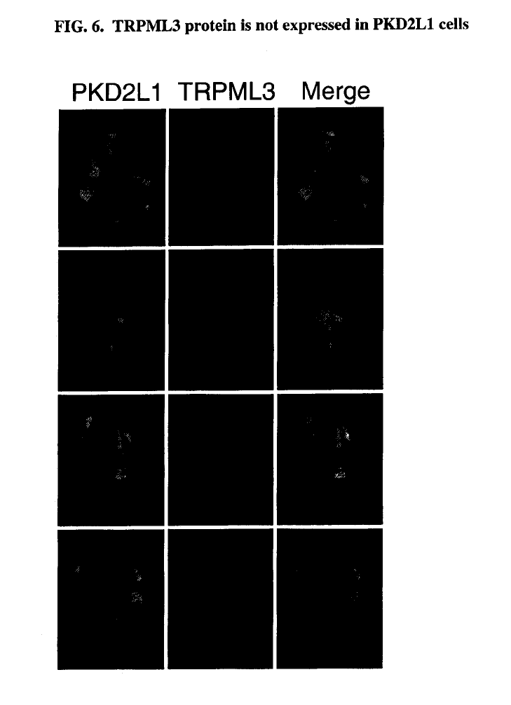

FIG. 6 contains the data of another immunochemistry double labeling experiment. The data contained in FIG. 6 reveal that the TRPML3 protein is not expressed in PID2L1 cells. The Figure contains the results of a double label immunohistochemistry with PKD2L1 (green; left images) and TRPML3 (red; middle images) in monkey CV papilla. It can be seen from the Figure that cells expressing PID2L1 and TRPML3 are distinct (merged images on the right). These data indicate that TRPML3 is not expressed in PKD2L1 cells (encompassing sour cells) but in professional salty taste cells.

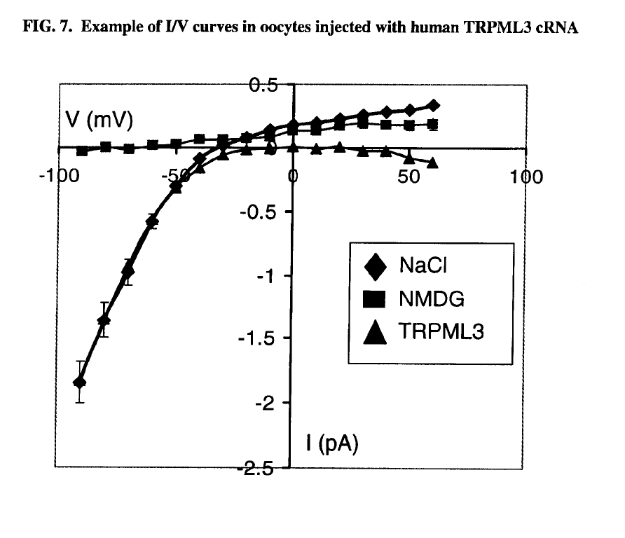

FIG. 7 illustrates an example of I/V curves in oocytes injected with human TRPML3 cRNA.

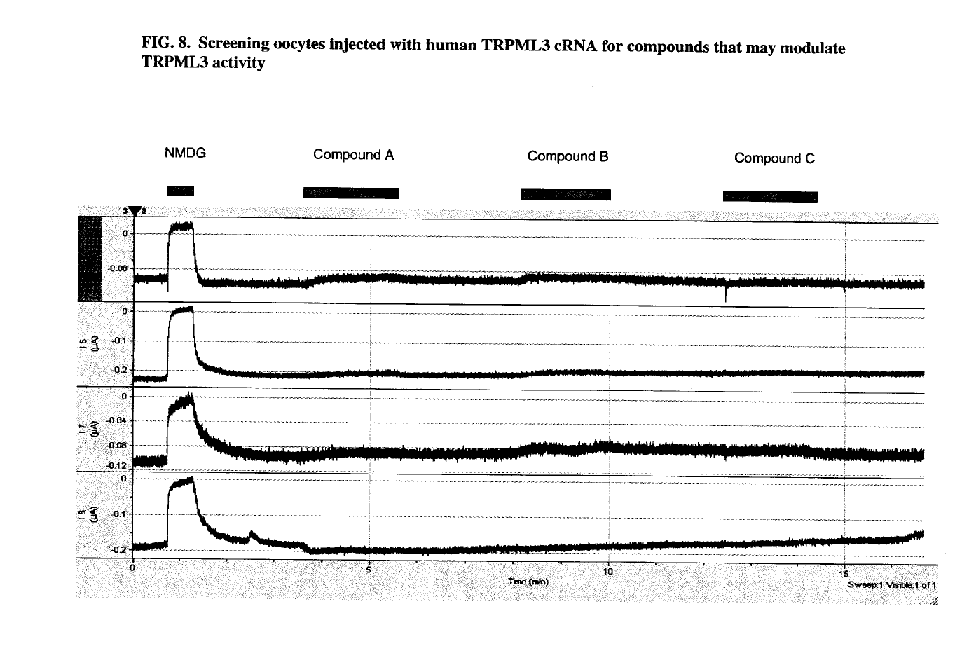

FIG. 8 illustrates an example of screening oocytes with human TRPML3 cRNA for compounds that may modulate TRPML3 activity.

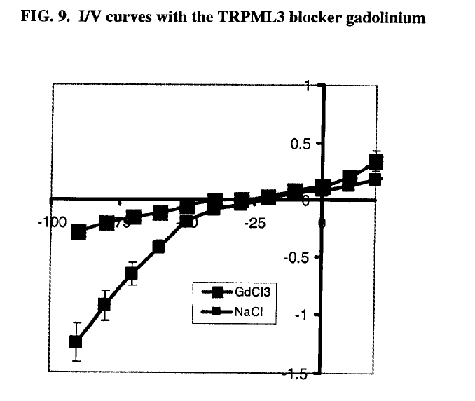

FIG. 9 illustrates an example of I/V curves with the TRPML3 blocker gadolinium.

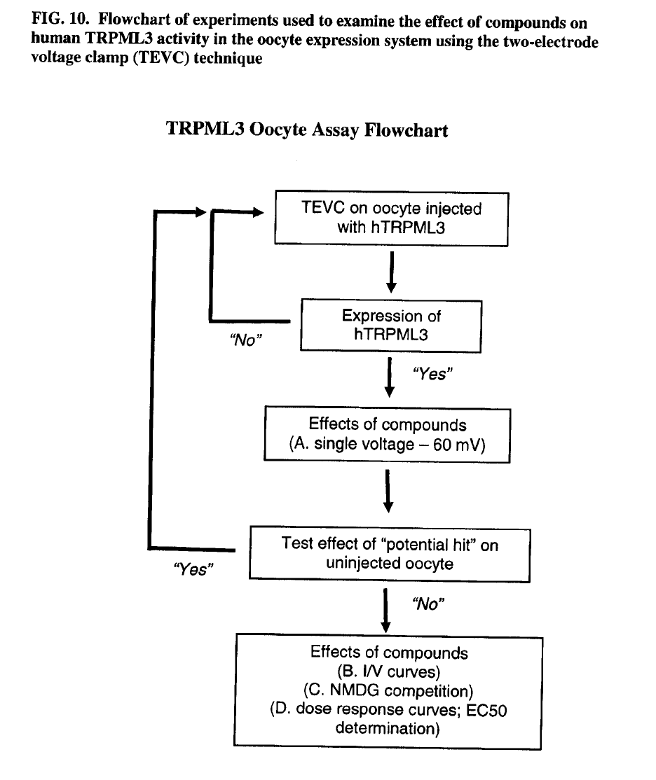

FIG. 10 is a flowchart of experiments used to examine the effect of compounds in human TRPML3 activity in the oocyte expression system using the two-electrode voltage clamp.

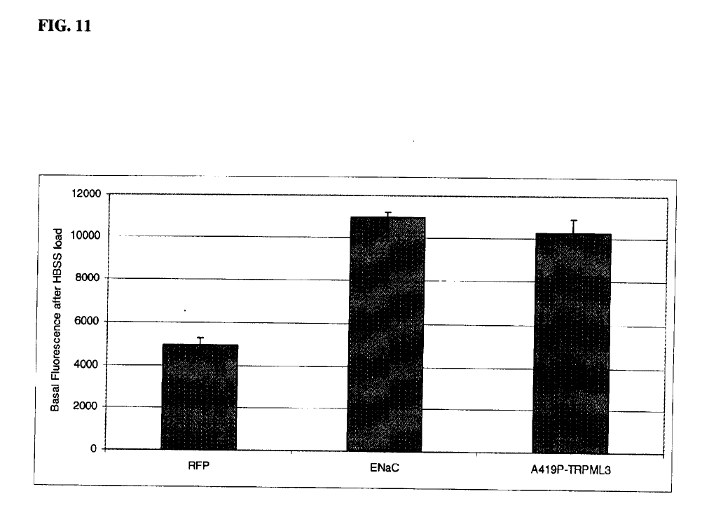

FIG. 11 illustrates the expression of constitutively active sodium channels increase basal fluorescence in cells loaded with specific membrane potential dyes.

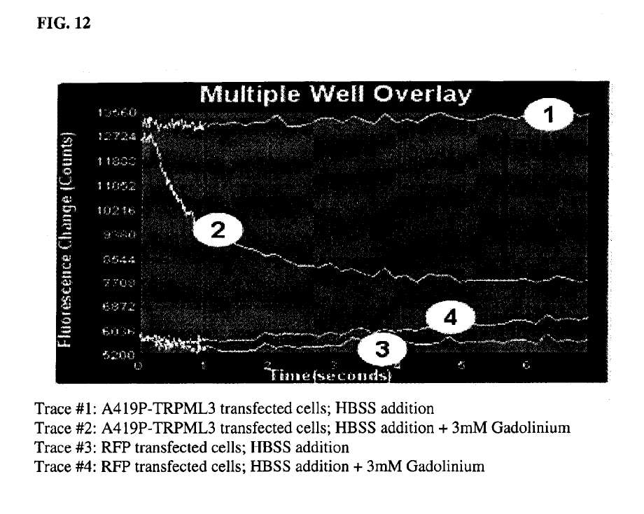

FIG. 12 illustrates the application of gadolinium reduces the increase in basal fluorescence in cells expressing A419P-TRPML3.

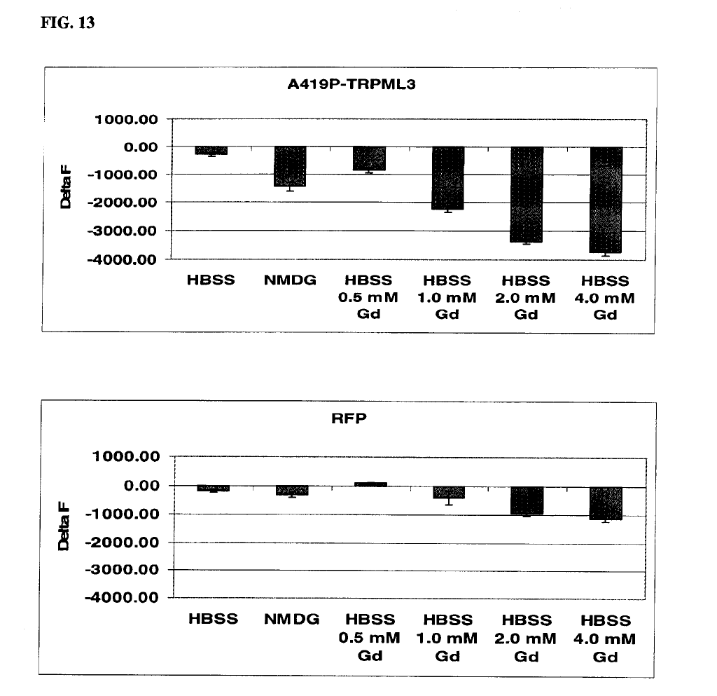

FIG. 13 illustrates the application of gadolinium reduces the increase in basal fluorescence in cells expressing A419P-TRPML3 in a dose-dependent fashion.

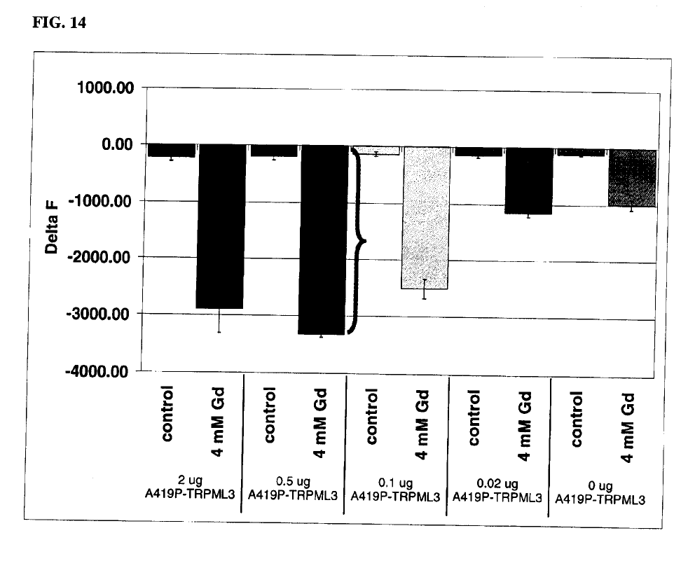

FIG. 14 illustrates the titration of TRPML3 plasmid.

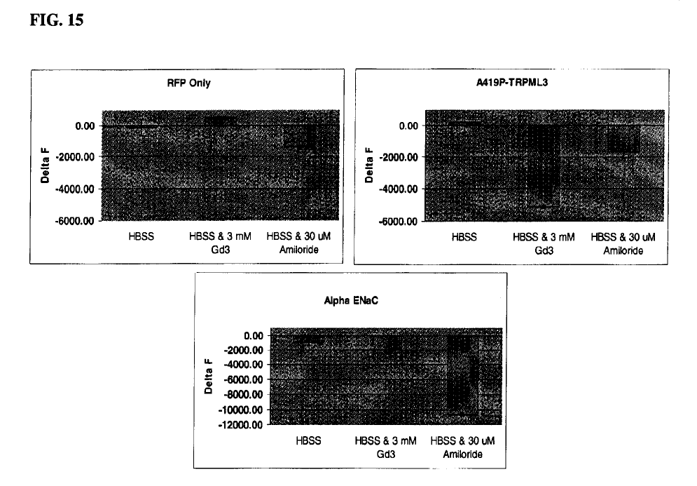

FIG. 15 illustrates the effect of gadolinium is specific for TRPML3.

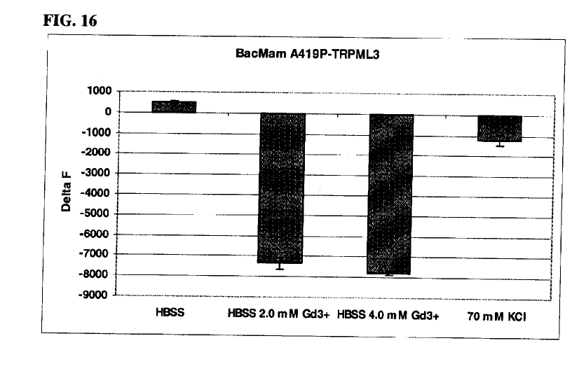

FIG. 16 illustrates transducing HEK<293 cells with baculovirus encoding A419P-TRPML3 doubles the assay window.

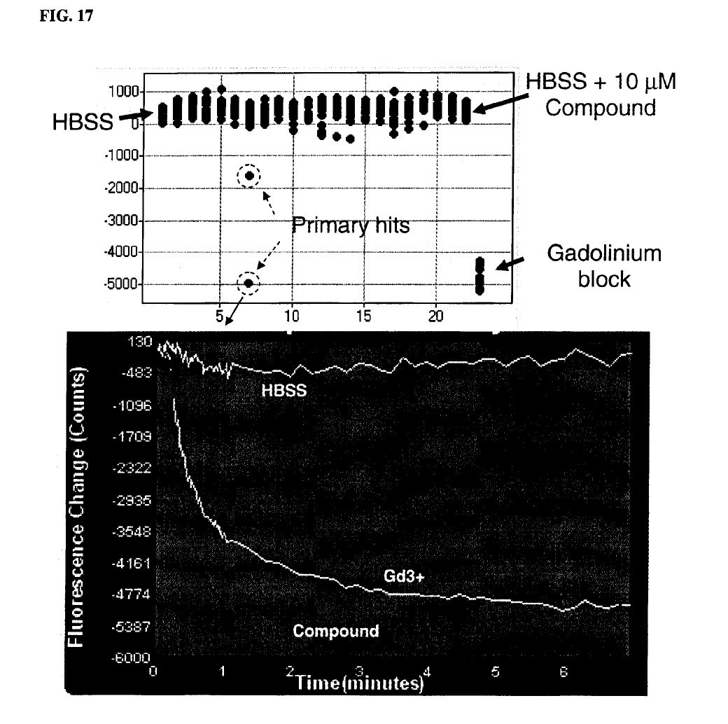

FIG. 17 is an example of screening data obtained with A419P-TRPML3 expressing cells.

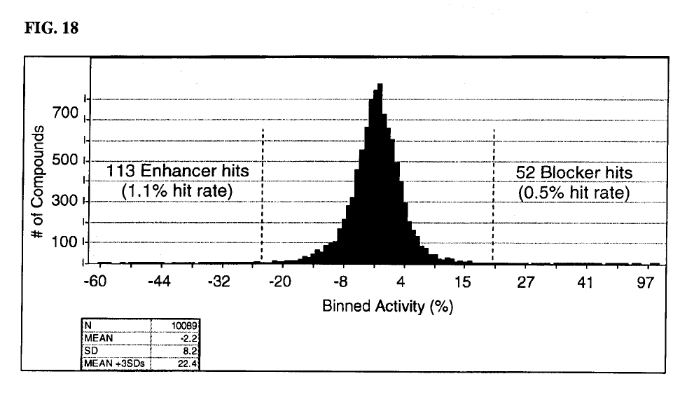

FIG. 18 is a summary of a 10,000 compound miniscreen with A419P-TRPML3 expressing cells.

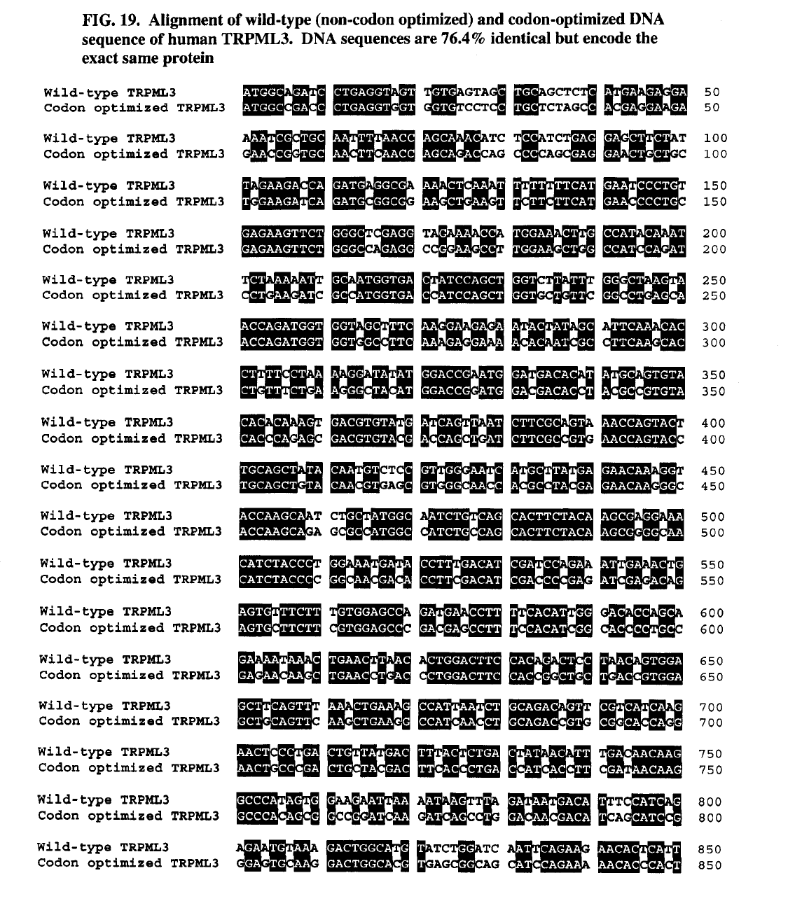

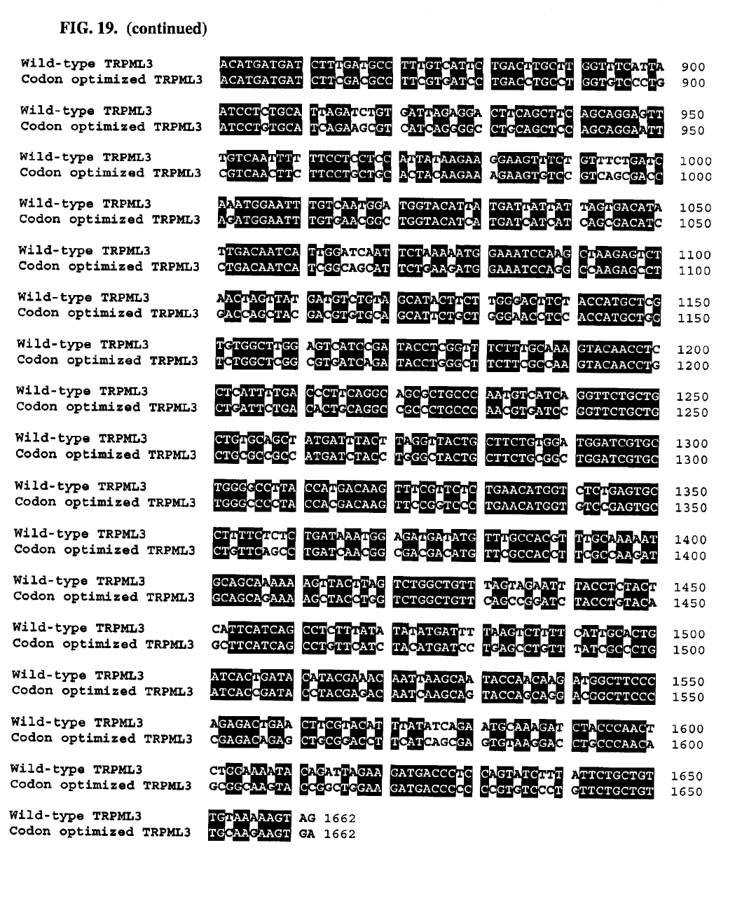

FIG. 19 shows the alignment of wild-type (non-codon optimized) and codon-optimized DNA sequence of human TRPML3 and that these DNA sequences are 76.4% identical. Wild-type (non-codon optimized), codon-optimized, and A419P TRPML3 were expressed in oocytes and sodium currents were measured.

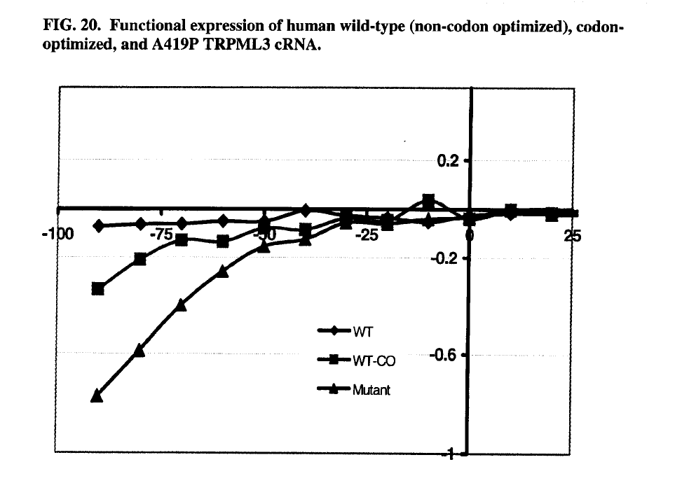

FIG. 20: Functional expression of human wild-type (non-codon optimized; labeled WT with blue diamonds), codon-optimized (labeled WT-CO with pink squares), and A419P (labeled mutant with yellow triangles) TRPML3 cRNA. The inwardly rectifying I/V curves, denoted by more current at hyperpolarized potentials (more negative potentials) and less current at depolarized potentials (more positive potentials), indicate functional expression of TRPML3 ion channels. Note the augmented currents observed with codon-optimized TRPML3 and A419P TRPML3 compared to wild-type TRPML3 with no codon optimization

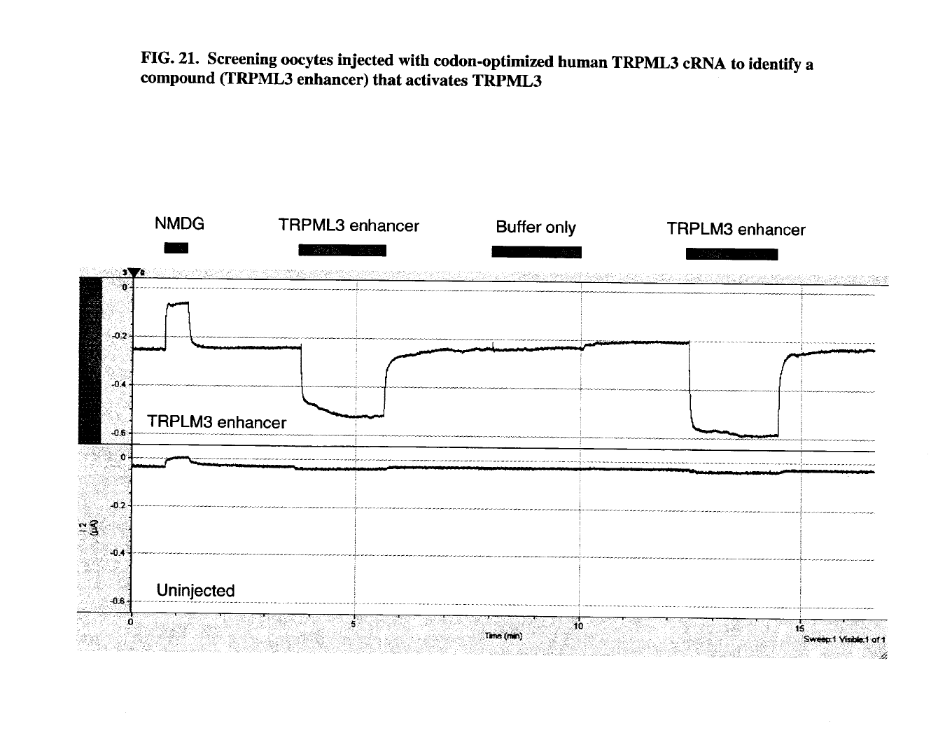

FIG. 21: Example of screening oocytes injected with codon-optimized human TRPML3 cRNA to identify a compound (TRPML3 enhancer) that activates TRPML3. In multiple oocytes, the TRPML3 enhancer increased TRPML3 activity by 169+/−26% from (representative trace on top) and had no effect on uninjected oocytes with no TRPML3 expression (representative trace on bottom). Addition of buffer only had no effect on TRPML3 currents and the effects of the TRPML3 enhancer were reproducible upon a second application.

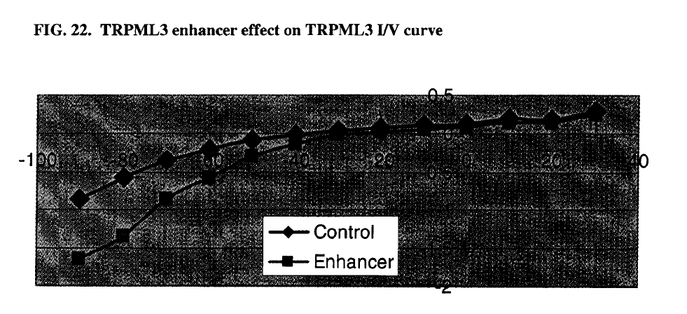

FIG. 22: Example of TRPML3 enhancer effect on TRPML3 I/V curve. Oocytes injected with codon-optimized human TRPML3 cRNA were untreated (blue triangles labeled control) or stimulated with TRPML3 enhancer (magenta squares labeled enhancer) and currents were measured at voltages from −90 to +30 mV. Note that the TRPML3 enhancer activates TRPML3 current at negative voltages (inward currents are larger with enhancer compared to with control), resulting in an increase in the slope of the I/V curve. Note also that the zero current shifts to the right, indicating an increased sodium conductance in the presence of the enhancer

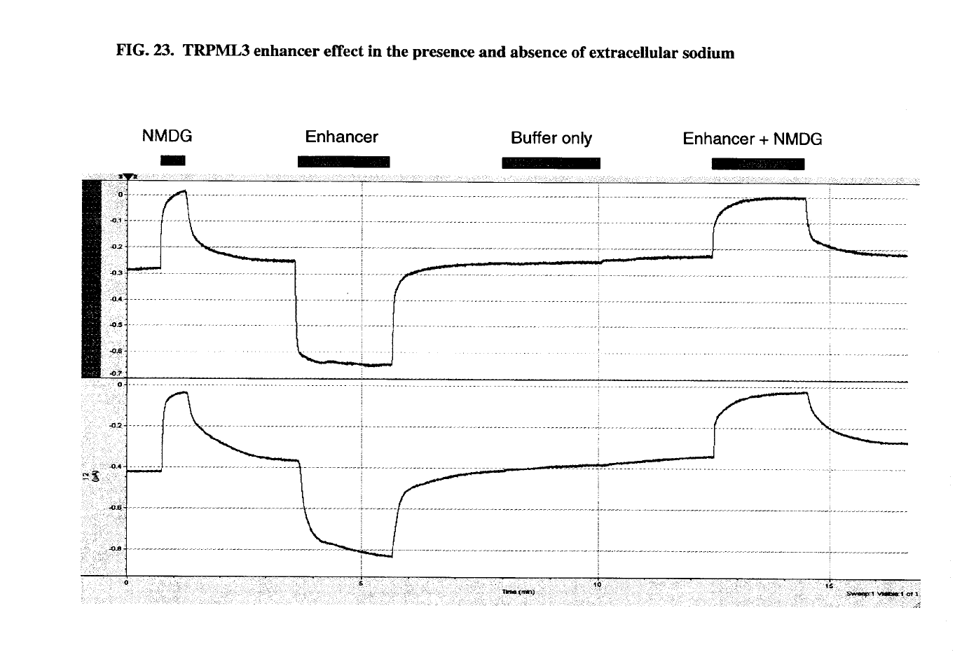

FIG. 23: Example of TRPML3 enhancer effect in the presence and absence of extracellular sodium. Oocytes expressing codon-optimized human TRPML3 cRNA were stimulated with NMDG (no sodium), TRPML3 enhancer plus sodium, buffer only, or TRPML3 enhancer plus NMDG (no sodium). Note that TRPML3 enhancer increased TRPML3 activity in the presence of sodium but had no effect in the absence of sodium. These data demonstrate that the TRPML3 enhancer opens TRPML3 channels and increases the flow of sodium ions into the oocyte.

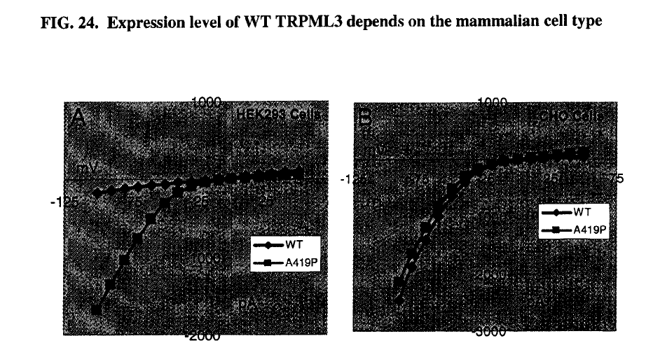

FIG. 24: Expression level of WT TRPML3 depends on the mammalian cell type. A. Current voltage analysis (I/V plot) of cells expressing WT and the A419P mutant TRPML3 channel in HEK1293 cells. A419P mutant TRPML3 channels express large inward rectifying currents (pink), whereas only small WT TRPML3 currents are observed (blue). B. WT and A419P mutant TRPML3 channels have similar functional characteristics in CHO cells.

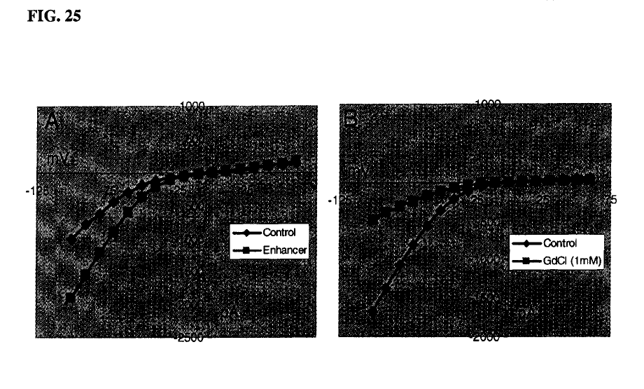

FIG. 25: Use of TRPML3 for enhancer and blocker screening in CHO cells. A. WT human TRPML3 channels transiently expressed in CHO cells are used to identify channel enhancers. I/V plot shows that compared to buffer alone (blue; control), use of the enhancer results in an increase in inward current at negative potentials (pink). B. Mutant A419P TRPML3 channel stably expressed in CHO cells are used to identify channel blockers. Compared to buffer alone (blue; control) addition of 1 mM GdCl₃ (gadolinium chloride) results in a decrease in inward current (pink).

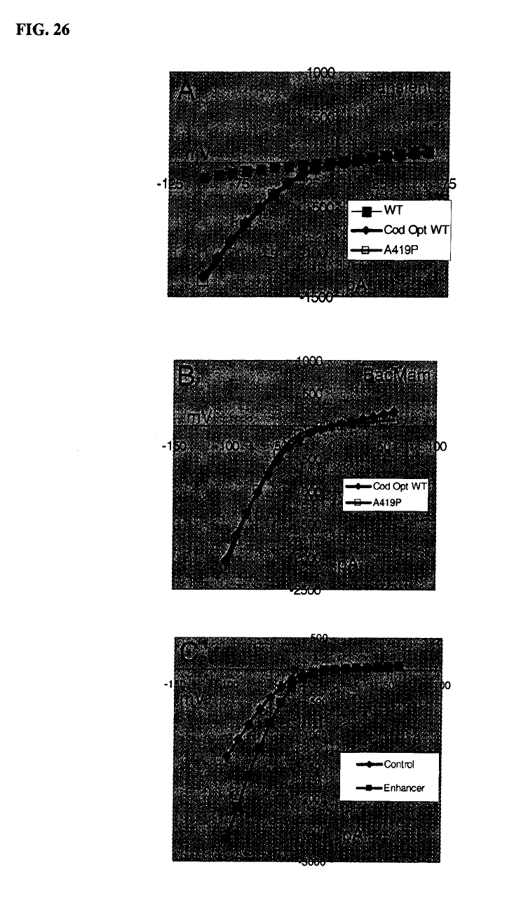

FIG. 26: Use of codon optimized WT TRPML3 for the screening of compounds which enhance TRPML3 function. A. Transient expression of non codon optimized WT TRPML3 (light blue) results in little current in HEK293 cells. In contrast, use of codon optimized WT TRPML3 (Dark Blue; Cod Opt WT) results in currents with similar average amplitude as A419P mutant channel (pink). B. Use of codon optimized WT TRPML3 (blue) delivered with Baculovirus transduction results in similar average currents as A419P TRPML3 (pink). C. Cells transduced with codon optimized WT TRPML3 baculovirus is used to identify enhancers of TRPML3 function. Compared to buffer alone (blue; control) addition of enhancer compound results in an increase in inward current (pink).

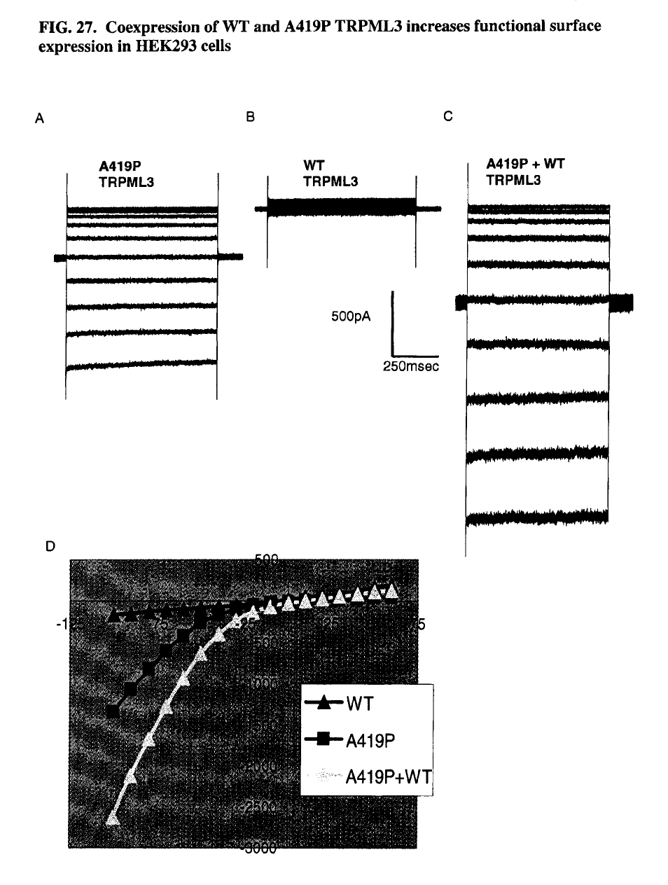

FIG. 27: Coexpression of WT and A419P TRPML3 increases functional surface expression in HEK1293 cells. A. Currents elicited from A419P TRPML3 cDNA (0.5 ug) transfected into HEK1293 cells, yielding currents with characteristic inward rectification. B. WT non codon optimized TRPML3 (1.5 ug) is expressed in HE<1293 cells and yields no currents. C. Coexpression of A419P (0.5 ug) with WT (1.5 ug) TRPML3 cDNAs in HEK1293 cells result in large inward currents which are twice as large as those when expressing A419P cDNA alone. D. I/V plot of the average currents elicited from WT (blue), A419P (pink) and coexpression of WT and A419P (yellow) TRPML3 cDNAs in HEK293 cells.

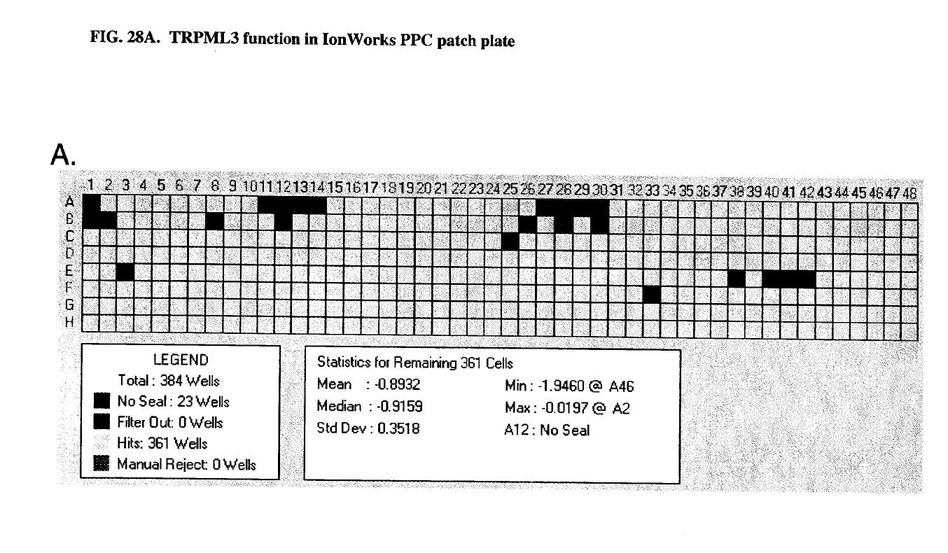

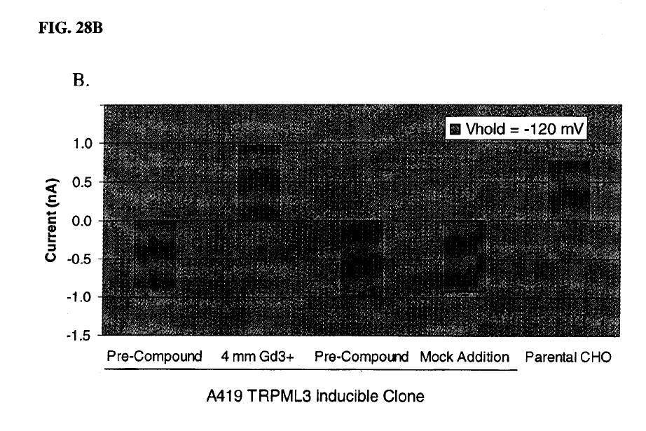

FIG. 28 contains an example of TRPML3 function in IonWorks PPC patch plate. A, View of all 384 wells from a PPC patch plate with an A419 TRPML3 inducible clone showing the results of the pre-compound scan. Yellow indicates wells where the current at −120 mV was <0 nA (in control experiments with parental CHO-1K1 cells none of the wells were labeled yellow). Blue indicates wells were the average seal was too low (<10 mOhm) to measure the current reliably. A419P TRPML3 currents could be measured in 94% of the wells. B, Average currents±SEM before and after addition of 4 mM GdCl3 or extracellular buffer (mock addition) from the patch plate shown in A. GdCl3 was added to column 1-38 while extracellular buffer was added to columns 39-48. For comparison, data is included from a separate experiment with parental CHO-K1 cells. The stability of the TRPML3 current after mock addition indicates that the assay should detect compounds that either enhance or block TRPML3 currents.

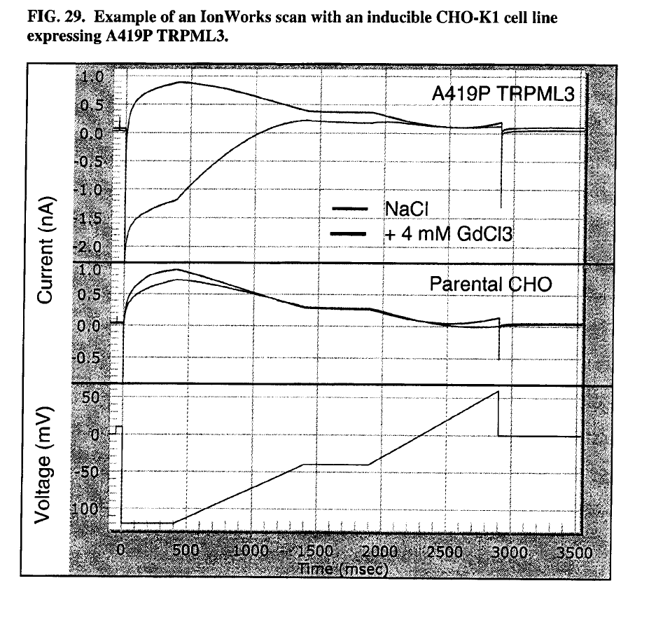

FIG. 29 contains an example of an IonWorks scan with an inducible CHO-K1 cell line expressing A419P TRPML3 (top panel). TRPML3 inwardly rectifies, denoted by more current at hyperpolarized potentials (more negative potentials) and less current at depolarized potentials (more positive potentials). Addition of GdCl3 blocks TRPML3 current. Red line denotes scan in sodium (NaCl) solution. Blue line denotes scan in 4 mM GdCl3 solution. The middle panel is from parental CHO-1K1 cells used as a negative control. The positive currents at negative potentials are due to leak subtraction overcorrecting the current at negative potentials. The bottom panel show the voltage command protocol used to record currents. The step from 0 mV to 10 mV is used to calculate the leak current (current flowing through leaks in the seal) which is subtracted from the total current to obtain the current flowing through the membrane. Results are from single wells in a PPC patch plate and represent the average current of up to 64 cells.

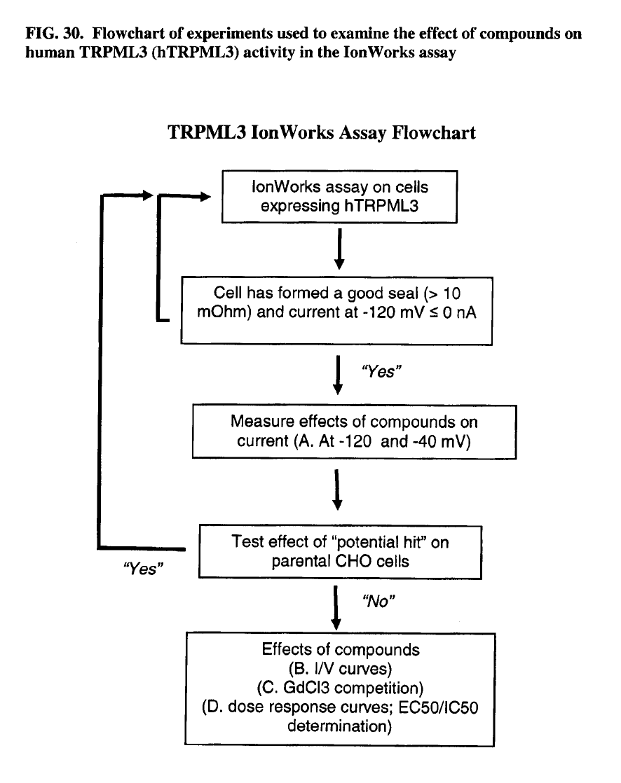

FIG. 30 contains a flowchart of experiments used to examine the effect of compounds on human TRPML3 (hTRPML3) activity in the IonWorks assay.

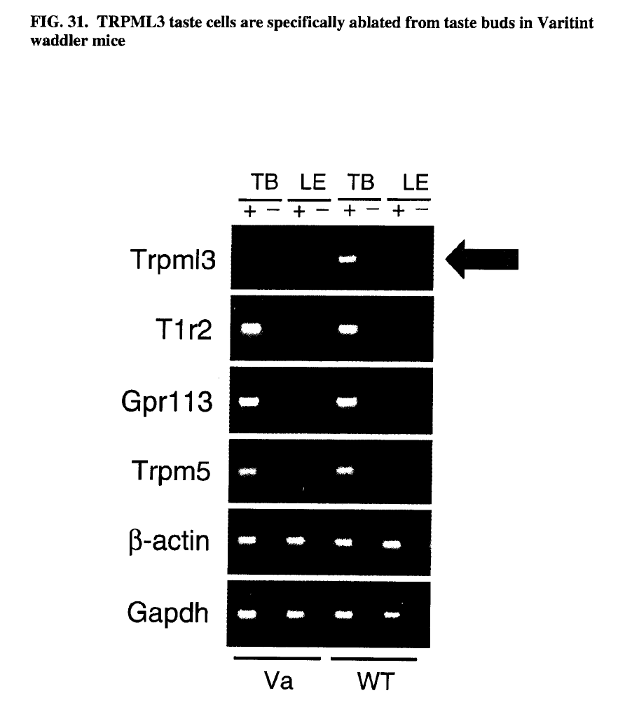

FIG. 31 shows that TRPML3 cells are specifically ablated from taste buds in Varitint waddler mice. End-point RT-PCR experiments on taste buds (TB) and lingual epithelial cells (LE) of Varitint waddler (Va) or wild-type (mice isolated by laser-capture microdissection. TRPML3 is only expressed in taste buds of WT mice and absent in taste buds of Va mice, whereas all other taste genes (T1R2, GPR113, TRPM5) as well as housekeeping genes (beta-actin, GAPDH) are equally expressed in TB and LE. ‘+’ indicates that reverse transcription was performed and ‘−’ indicates that no reverse transcription was performed. PCR bands were only observed with reverse transcriptase indicating that PCR products are derived from mRNA and not genomic DNA.

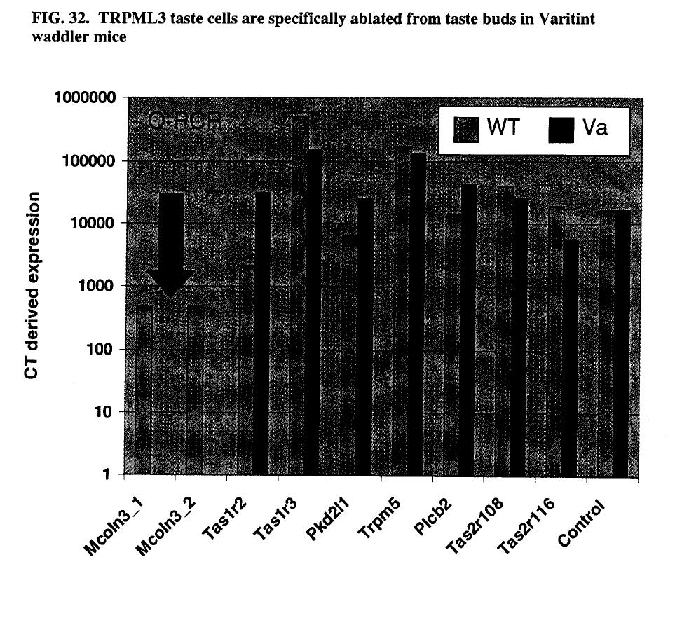

FIG. 32 also shows by use of real-time PCR that TRPML3 cells are specifically ablated from taste buds in Varitint waddler mice. Real-time quantitative RT-PCR experiments on taste buds of Varitint waddler (Va) or wild-type (mice isolated by laser-capture microdissection. TRPML3 is only expressed in taste buds of WT mice and absent in taste buds of Va mice (similar results were obtained using two different primer sets labeled Mcoln3_{—}1 and Mcoln3_{—}2), whereas all other taste genes (Tas1r2, Tas1r3, PID2l1, TRPM5, Plcb2, Tas2r108, and Tas2r116) as well as a housekeeping gene (control) are expressed in taste buds from Va and WT mice.

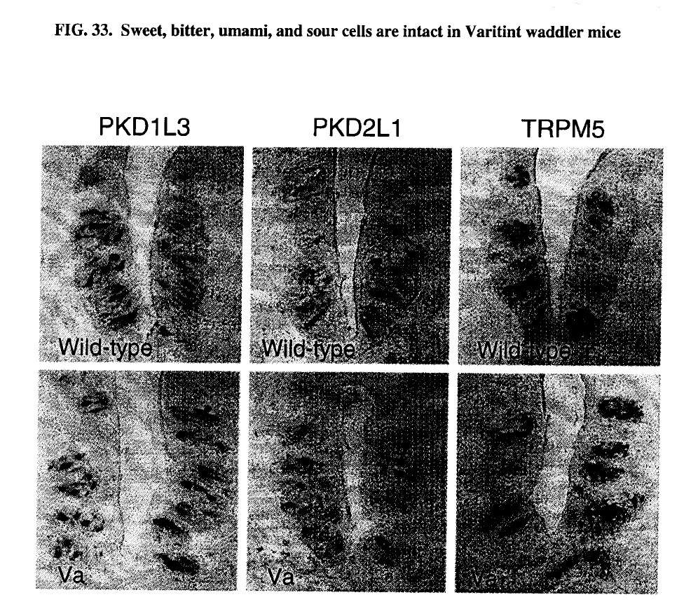

FIG. 33 contains an experiment showing that sweet, umami, bitter and sour cells are intact in the taste buds of the Varitint-waddler mouse. In situ hybridization of circumvallate papilla from the back of the tongue of wild-type (top row of images) and Varitint waddler (Va; bottom row of images) mice. PID1L3 (left; sour), PID2L1 (middle; sour), and TRPM5 (right; sweet, bitter, umami, and GPR113) taste cells were present at similar levels in wild-type and Va mice.

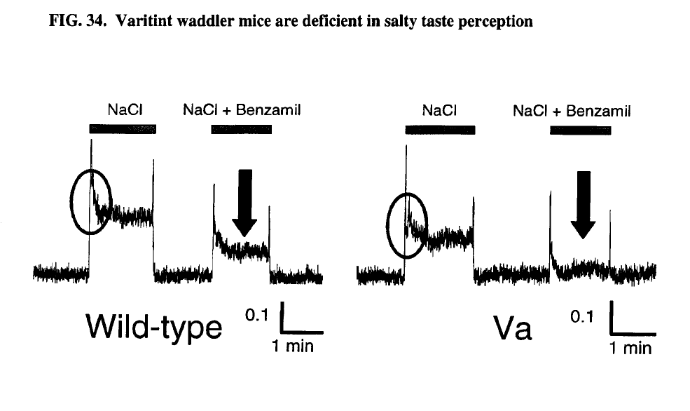

FIG. 34 contains a CT nerve recording experiment demonstrating that the Varitint waddler mice are deficient in salty taste. CT nerve recordings from wild-type (left) or Varitint waddler (Va; right) mice. Anterior tongues were stimulated with 0.1 M NaCl or 0.1 M NaCl plus 5 uM benzamil to inhibit the amiloride-sensitive component of the CT nerve response. Tongues were rinsed with a low salt solution containing 10 mM KCl in between NaCl stimulations. Note that the benzamil-insensitive component of the CT nerve response is largely eliminated in the Va mouse (red arrows), indicating that ablation of TRPML3 taste cells significantly impairs salty taste perception. In addition, the immediate phasic response to NaCl is greatly reduced in the Va mouse (red circles). Scale bars indicate time frames of salt application (x-axis) and the magnitude of the CT response (y-axis; arbitrary units).

FIG. 35 contains an alignment of the sequences of human and mouse TRPML3 genes and polypeptides. The transmembrane domains, extracellular loop and pore regions are identified as well as the residue (419) that gives rise to the Varitint waddler mouse

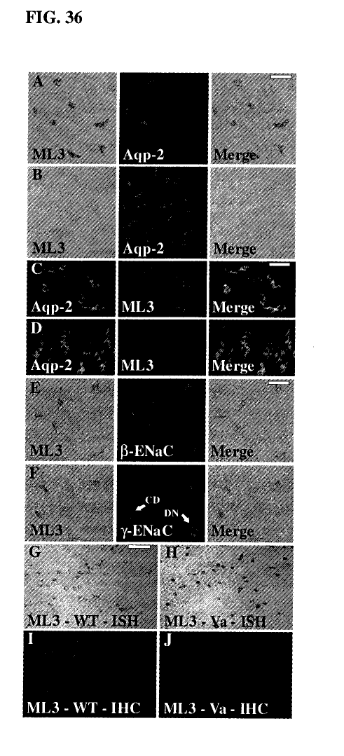

FIG. 36 contains experiments showing TRPML3 expression in kidney collecting duct principal cells. (A) TRPML3 (ML3; blue; left) and aquaporin-2 (Aqp-2; red; center) are coexpressed in collecting duct cells (merge; right). (B) TRPML3 is not expressed in inner medullary collecting duct cells that express aquaporin-2. For images A and B, TRPML3 was visualized by In situ hybridization while aquaporin-2 was visualized by immunohistochemistry. (C) Aquaporin-2 (green; left) and TRPML3 (red; middle) are coexpressed in collecting duct cells (merge; right). (D) TRPML3 is not expressed in inner medullary collecting duct cells that express aquaporin-2. For images in C and D, TRPML3 and aquaporin-2 were visualized by immunohistochemistry. (E) TRPML3 (blue; left) and βENaC (red; center) are coexpressed in collecting duct cells (merge; right). (F) TRPML3 (blue; left) and γENaC (red; center) are coexpressed in collecting duct (CD) cells (merge; right). γENaC is also expressed in more distal nephron (DN) segments such as connecting tubules. For images in E and F, TRPML3 was visualized by in situ hybridization while ENaCs were visualized by immunohistochemistry. (G) In situ hybridization of TRPML3 in wild-type (WT) kidney. (H) In situ hybridization of TRPML3 in Va/+ kidney showing no loss of TRPML3 cells. (I) Immunohistochemistry of TRPML3 in wild-type (WT) kidney. (J) Immunohistochemistry of TRPML3 in Va/+ kidney showing no loss of TRPML3 cells. Scale bar, 60 um (A-B), 50 um (C-D), 60 uM (E-F), and 80 um (G-J).

### DETAILED DESCRIPTION OF THE INVENTION

The present invention relates to the identification of a gene that regulates salty taste perception in mammals and potentially other vertebrates, e.g., avians, reptiles and amphibians. This gene, TRPML3 or MCOLN3 is specifically expressed in taste cells that respond to salty taste stimuli and is also expressed in pituitary, adrenal, kidney cells involved in sodium homeostasis, and melanocytes. This gene encodes an ion channel polypeptide that alone or potentially in association with other accessory molecules or ion channels such as TRPML1 TRPML2, NALCN or NKAIN3 detects salty taste stimuli and regulates sodium transport, metabolism and excretion and/or further may affect sodium related processes involving aldosterone and/or vasopressin based on the fact that this ion channel is substantially expressed in kidney cells that regulate sodium homeostasis, plasma osmolarity, aquaporin-2 function and trafficking, ENaC function and trafficking, blood pressure and the like, is highly expressed in the adrenal gland which produces aldosterone, a hormone significantly involved in sodium related processes that affect the urinary and cardiovascular system as well as other organs as well as being substantially expressed by the pituitary, which secretes vasopressin, another hormone which plays a very important role in sodium transport, metabolism and excretion and which affects among other things blood pressure, urine output and fluid retention. Therefore, the subject ion channel polypeptide plays a significant role in sodium transport, metabolism and excretion by different cells and organs, as well as being involved in salty taste perception.

This gene, Mucolipin 3 (MCOLN3), or TRPML3 (identified using a novel rationale disclosed herein and in other provisional applications incorporated by reference herein) encodes a multitransmembrane protein expressed in the top of the taste buds, in the taste sensory cells, that conducts sodium. This gene is believed to encode a salt receptor that allows sensory taste cells in the tongue's taste buds to detect sodium chloride (salt). In addition, because this gene is expressed in the adrenal and pituitary glands and in specialized cells in the kidney involved in sodium homeostasis, plasma osmolarity, blood pressure, fluid retention et al. it also is believed to participate in the regulation of sodium metabolism in the body. The evidence obtained by the inventors and earlier reports relating to this gene that suggest this gene being the human salt receptor is as follows:
(1) MCOLN3 is expressed in the top of the taste buds, in a subset of taste sensory cells that do not express TRPM5 (that is, they are not sweet, bitter or umami) and towards the taste pore.
(2) MCOLN3 is also expressed in sensory cells of other organs, like the ear. It is therefore a ‘professional’ sensory gene.
(3) MCOLN3 is strongly expressed in the adrenal glands. These glands play a very important role in the regulation of sodium metabolism in the body. MCOLN3 is therefore likely to be a key molecule in the regulation of sodium metabolism and may regulate the production of aldosterone by the adrenal glands.
(4) A human autoimmune disease (Addison's) is characterized by the destruction of the adrenal glands. One of the telltale symptoms of this disease is salt craving. The latter is likely to result from the presence of autoantibodies against MCOLN3, or a mutation in this gene that disrupts the function of MCOLN3 in taste buds.
(5) MCOLN3 is highly expressed by the pituitary glands that are involved in vasopressin release that regulates urine production. This further supports the importance of this ion channel in sodium metabolism and excretion by the body.
(6) MCOLN3 is highly expressed by kidney collecting duct principal cells in the kidney that are involved in fluid retention, sodium homeostasis, particularly in cortical and outer medullary collecting ducts. TRPML3 kidney cells express both aquaporin-2 water channels that reabsorb water to maintain plasma osmolarity, as well as ENaC sodium channels that reabsorb sodium to maintain salt balance. Unlike taste cells, TRPML3 cells in the kidney were not ablated in Va/+ mice, suggesting that the A419P TRPML3 mutation did not compromise ionic homeostasis to an extent that compromised cell viability in kidney cells. Since principal cells respond to the sodium-conserving hormone aldosterone, plasma sodium and aldosterone levels in Va/+ mice were measured and it was found that Va/+ mice exhibit a pronounced hyponatremia (low blood sodium concentration) and hyperaldosteronemia (high blood aldosterone concentration), consistent with a deficiency in principal cell function (results infra), while other electrolytes including plasma chloride and potassium as well as glucose and protein levels were not significantly different from wild-type controls. TRPML3 is, therefore, expressed in kidney epithelial cells responsible for sodium balance, and Va/+ mice exhibit deficiencies in sodium homeostasis as a consequence of a mutation in TRPML3 that affects function. Expression of TRPML3 in both taste cells on the tongue and principal cells in kidney collecting ducts highlights an important role of TRPML3 cells in global sodium balance.
(7) MCOLN3 conducts sodium in electrophysiology studies and exhibits the right biochemical characteristics predicted for a salt receptor (detection of K+, Li+ and amiloride sensitivity).
(8) Neurophysiological experiments (nerve recordings) using sodium in the varitint mouse (having TRPML3 mutation) indicate that the Varitint mouse is deficient in its response to sodium (does not exhibit a robust salty taste response).
(9) Cell based assays using mammalian cells and amphibian oocytes which express mutated TRPML3 polypeptides (mutation results in the ion channel being fixed in the “open” orientation) have identified TRPML3 enhancers and blockers which should enhance or block salty taste in taste tests.

The discovery that MCOLN3 is a human salty taste receptor and as a receptor that regulates sodium homeostasis and other physiological functions associated therewith such as blood pressure, fluid retention, plasma osmolarity, et al. also has enabled the design of screening assays using cells transfected with this gene for the purpose of identifying agonists, antagonists or enhancers (modulators) of the function of this molecule.

This gene was originally identified by the inventors using a whole genome screening strategy aimed at identifying genes specifically expressed in taste cells and screening of a subset thereof enriched in the top of the human taste buds. The inventors had deduced from their previous experiments that the top of the taste buds contain cells that over-express the known taste receptor genes. In contrast, the bottom of the taste buds contains precursor cells of the sensory taste bud cells that reside in the top portion of the taste bud. This database allowed the inventors to identify many genes specifically expressed by the top (sensory) cells of the taste buds. One of these genes, MCOLN3, was previously described to be responsible for the phenotype of a mouse mutant called varitint-waddler that exhibits early-onset hearing loss, vestibular defects, pigment abnormalities and perinatal lethality (DiPalma et al., Mutations in MCOLN3 associated with deafness and pigmentation defects in varitint-waddler (Va) mice. Proc. Natl. Acad. Sci. USA 99: 14994-14999; 2002). MCOLN3 is expressed in the hair cells and plasma membrane of stereocilia (in the ears)). This mutation results in an ala 419 to pro substitution in the fifth transmembrane domain. The result is a hyperactive MCOLN3 that results in the death of cells expressing this molecule, like the hair cells of the ear (hence the deafness of the Va mouse) (Grimm C et al., Proc Natl. Acad. Sci. USA 104: 19583-8; 2007). Therefore the inventors anticipated that this mouse would also exhibit salty taste abnormalities (due to the abnormal MCOLN3 molecule and its probable effect in the salty taste bud cells of the tongue). Indeed, as shown in neurophysiology experiments and data contained herein, these mice when stimulated with salty taste stimuli at concentrations that should normally elicit salty taste perception do not respond robustly to salty taste stimuli (nerve recoding results in Varitint mice discussed infra substantiate that the TRPML3 mutation, which disrupts the activity of TRPML3 and causes deafness and balance problems because of the loss of hairy cells in the inner ear also disrupts salty taste, further substantiating that a normally functioning TRPML3 or MCOLN3 ion channel is a prerequisite to salty taste perception).

Also, since public databases, indicate that MCOLN3 is expressed strongly in the adrenal and pituitary glands, and since the adrenal glands represent one of the main sodium metabolism regulators of the body, TRPML3 likely is significantly involved in sodium metabolism. (The adrenal glands monitor salt levels of the blood, and secrete aldosterone (a mineralocorticoid) that regulates blood pressure and water and salt balance in the body by helping the kidney retain sodium and excrete potassium. Moreover, when aldosterone production falls too low, the kidneys are not able to regulate salt and water balance, causing blood volume and blood pressure to drop.)

Therefore, it is also predicted that MCOLN3 is a key salt/sodium monitoring molecule in the adrenal glands that controls the production of aldosterone. By contrast, in the tongue, MCOLN3 is expressed by s subset of taste sensory cells located in the top of the taste buds and is responsible for detecting salty taste. Either way, MCOLN3 has a pivotal role in detecting salt in various tissues. This invention therefore constitutes a significant discovery with significant applications.

In addition, since TRPML3 or MCOLN3 is also substantially expressed in the pituitary, the inventors also predict that MCOLN3 is involved in the regulation of vasopressin release. Vasopressin is a key regulator of urine production through its effects on the kidneys. Importantly, vasopressin release from the posterior pituitary is known to be regulated by NaCl concentration. In yet an additional manner, MCOLN3 appears to be a key regulator of NaCl metabolism in the body, through its effects on fluid retention, NaCl sensing and concentration, and blood pressure.

Further, since TRPML3 is substantially expressed in cortical and outer medullary collecting ducts this ion channel plays a significant role in sodium homeostasis. With respect thereto, these same TRPML3 kidney cells express both aquaporin-2 water channels that reabsorb water to maintain plasma osmolarity, as well as ENaC sodium channels that reabsorb sodium to maintain salt balance. Unlike taste cells, TRPML3 cells in the kidney were not ablated in Va/+ mice, suggesting that the A419P TRPML3 mutation did not compromise ionic homeostasis to an extent that compromised cell viability in kidney cells. However, since principal cells respond to the sodium-conserving hormone aldosterone, plasma sodium and aldosterone levels in Va/+ mice were measured to discern if in fact this mutation in TRPML3 expressed in Va/+ mice had an effect on sodium homeostasis. It was found that Va/+ mice exhibit a pronounced hyponatremia (low blood sodium concentration) and hyperaldosteronemia (high blood aldosterone concentration), consistent with a deficiency in principal cell function (results infra), while other electrolytes including plasma chloride and potassium as well as glucose and protein levels were not significantly different from wild-type controls. TRPML3 is, therefore, expressed in kidney epithelial cells responsible for sodium balance, and Va/+ mice exhibit deficiencies in sodium homeostasis consistent with the fact that TRPML3 plays a significant role in sodium homeostasis. Essentially, the expression of TRPML3 in both taste cells on the tongue and principal cells in kidney collecting ducts highlights an important role of TRPML3 cells in global sodium balance.

Therefore this invention identifies and provides functional (electrophysiological) and immunohistochemistry data and animal data (neurophysiological studies) which indicate that TRPML3 (MCOLN3) encodes a polypeptide that functions as a primate (e.g., human) salty taste receptor and plays a significant role in sodium sensing and metabolism systemically.

Based thereon, the present invention has as a significant focus the development of reliable and efficient assays for identifying compounds that modulate TRPML3 polypeptides (block, enhance, activate) as these compounds should modulate salty taste as well as modulating biological functions relating to TRPML3 such as sodium transport, absorption and excretion by cells, tissues and organs as well as having an effect (agonistic or antagonistic) on aldosterone or vasopressin related activities and conditions wherein modulation of aldosterone or vasopressin release or production is therapeutically warranted. As is well known in the art compounds which agonize or antagonize vasopressin and aldosterone find well known application in therapy, especially urinary and cardiovascular conditions as well as conditions involving edema or aberrant circulation. For example, these compounds are used in treating hypertension, edema, congestive heart failure, diabetes and symptoms thereof, among numerous other conditions.

In addition, as TRPML3 has an effect on melanocytes and hairy cells compounds which modulate TRPML3 may be useful in promoting the proliferation and differentiation of melanocytes, may be useful in treating pigmentation disorders, may prevent or restore grey hair or skin to its normal coloration (lost e.g., because of disease, age, hormonal dysfunction, UV radiation, or chemotherapy) and may promote hair follicle growth and proliferation. Also, TRPML3 modulators may be useful in treating melanoma as they potentially may selectively kill melanoma cells expressing TRPML3.

Based on the foregoing the present invention provides assay systems that comprise test cells, preferably mammalian cell-based and oocyte cells, that express a functional TRPML3 which may comprise a wild-type TRPML3 of any desired species, a mutated TRPML3 wherein the mutations naturally occur or are introduced by design, e.g., in order to modify TRPML3 function (enhance or inhibit) or to maintain it in a fixed open pore orientation to facilitate its use in modulator screening assays, or it may comprise a chimeric TRPML3 ion channel or a functional fragment wherein a domain or extracellular look of TRPML3 is exchanged with that of another TRPML3 ion channel or another ion channel such as TRPML1, TRPML2, NALCN or NKAIN3 or another TRP ion channel. Exemplary mutations to TRPML3 are disclosed herein and may be designed by a skilled artisan using the information disclosed herein and methods well known to those skilled in the art. Also, it may be advantageous, as described herein, to provide TRPML3 encoding nucleic acid sequences which are comprised of host preferred codons, i.e., codons preferred in the cell wherein the assays are to be conducted. For example, the inventors provide infra a TRPML3 gene constituted of human preferred codons in order to enhance TRPML3 ion channel activity in human (e.g., HEK-293 cells) used in preferred assays according to the invention.

Preferably, the invention provides mammalian cell-based and oocyte cell-based assay, preferably high or medium throughput, for the profiling and screening of the salty taste receptor (TRPML3). More specifically, the invention provides amphibian oocytes, that express TRPML3 that can be used in cell-based assays for the screening of TRPML3 modulators. Also the invention provides amphibian oocytes that express a functional TRPML3 for use in functionally characterizing TRPML3 activity, and that may be used to identify compounds that either enhance or block salty taste perception (herein referred to as salty taste modulators). These compounds can be used as ingredients in foods, medicinals and beverages to enhance, modulate, inhibit or block salty taste. Also, these compounds have potential therapeutic application, e.g., in regulating blood pressure, cardiac function, renal function especially urine production and excretion, sodium homeostasis, plasma osmolarity, ENaC function, in treating Addison's disease, type IV mucolipidosis, and physiological effects of aldosterone and/or vasopressin and diseases wherein the administration of an aldosterone or vasopressin agonist or antagonist is therapeutically warranted such as diabetis insipidus et al.

Therefore this invention identifies and provides functional (electrophysiological), molecular, and immunohistochemistry data which indicate that TRPML3 (MCOLN3) encodes a polypeptide that functions as a primate (e.g., human) salty taste receptor and which further is involved in sodium homeostasis in general and in maintaining or modulating blood pressure, ENaC function, plasma osmolarity, fluid retention, and the like.

Further the present invention provides the use of these taste specific genes as markers which can be used to enrich, identify or isolate salt receptor expressing cells or sodium homeostasis regulating cells.

Also this invention provides in vitro and in vivo assays which use TRPML3 (MCOLN3) and TRPML3 expressing cells or TRPML3 transgenic animal models to identify agonist, antagonist or enhancer compounds which elicit or modulate (block or enhance) salty taste in primates including humans or which may be used as therapeutics to treat conditions and symptoms associated with aberrant sodium homeostasis, plasma osmolarity, fluid retention, blood pressure and the like. These taste modulator assays may use cells which express TRPML3 alone or cells which express the TRPML3 ion channel in association with other taste specific polypeptides such as NALCN or NKAIN3.

In addition this invention provides transgenic animals, preferably rodents, and the use thereof to confirm the role of TRPML3 in salty taste in mammals and in other physiological functions involving sodium and other ions such as sodium metabolism, blood pressure, fluid retention and excretion, plasma osmolarity, ENaC function, aquaporin-2 function and/or trafficking, urinary function and cardiac function.

Also this invention provides in vitro and in vivo assays which use TRPML3 and TRPML3 expressing cells or transgenic animals in assays, preferably electrophysiological assays in order to identify therapeutic compounds which alleviate diseases and conditions involving deficiencies in the expression of this polypeptide including hyper expression, hypo expression, and mutations in the TRPML3 polypeptide that affect its ability to function as a taste specific sodium channel in mammals including e.g., human and non-human primates. These conditions include by way of example Addison's disease, diabetes, conditions involving aberrant plasma osmolarity, and sodium homeostasis such as diabetis insipidus, ENaC function, aquaporin-2 function and/or trafficking and other diseases involving or affected by aberrant renal function, aldosterone production or vasopressin release such as hypertension, hypotension, fluid retention, and impaired urinary or cardiac function such as arrhythmia, heart attack and stroke.

The subject gene was initially identified by the use of the following methodologies, to identify novel taste-specific genes:
1) Laser capture microdissection (LCM) and RNA amplification: In laser capture microdissection, a fine laser beam is used to dissect and purify taste cells from histological sections. This method isolates taste cells, devoid of contaminating lingual epithelial cells and connective tissue, and allows one to perform molecular biology experiments on a highly enriched taste cell population. In parallel, lingual epithelial cells are isolated by LCM and used as a negative control devoid of taste cells. LCM is advantageous to manual or enzymatic dissection of taste papilla because these crude techniques yield a heterogeneous mixture of taste and lingual cells in which taste cells comprise 1-20% of collected material. RNA amplification amplifies total RNAs from taste cells and lingual cells isolated by LCM up to 1 million-fold in a non-biased fashion to generate sufficient genetic material to perform molecular biology studies (gene chips or PCR). We have found that 5,000 taste cells are sufficient for gene chip experiments with macaque taste tissue and greater than 10,000 taste cells are sufficient for PCR experiments with macaque taste tissue.
2) Gene Chips: Gene chips contain most all annotated genes on a small chip. Hybridizing RNA, isolated and amplified from taste and lingual cells, to gene chips can be used to determine which specific genes are expressed in taste cells and not lingual cells and which specific genes are expressed at higher levels in taste cells compared to lingual cells. Gene chips experiments were conducted using paired macaque fungiform (FG) and circumvallate (CV) taste and lingual samples using Affymetrix rhesus macaque genome arrays and analyzed using GeneSpring GX v7.3 software (Agilent Technologies). 5000 fungiform and CV taste and lingual cells were separately isolated by LCM and total RNA was purified for each sample. RNA was then amplified and hybridized to gene chips. Data analyses are performed using two separate algorithms: Affymetrix Microarray Suite 5 (MAS5) which takes into account both perfect match and mismatch probes on gene chips, and robust multi-chip algorithm (RMA) which only takes into account perfect match probes on gene chips. Taste-specific genes encoding transmembrane proteins are identified in this analysis.
3) PCR: High-throughput PCR is performed in 96 well plates using primers specific for ion channels in the human/macaque genome and amplified RNA from human/macaque taste and lingual cells isolated by LCM. Detection of products of the appropriate size in taste cells but not lingual cells and DNA sequencing of PCR products (to confirm gene identity) indicates the ion channel of interest is a taste-specific gene. Prior to high-throughput PCR using primers against ion channels identified in the macaque genome, quality-control PCR reactions are first performed on up to 4 known taste-specific genes and 2 housekeeping genes to ensure that taste and lingual RNAs are of high quality. Four taste-specific genes which may be examined are the G alpha protein gustducin (GNAT3), the sweet receptor component TiR2, the ion channel TRPM5, and the enzyme phospholipase C isoform beta2 (PLCβ2); the two housekeeping genes examined are beta-actin and GAPDH. Specific expression of the taste genes by taste cells but not lingual cells plus expression of the ubiquitous housekeeping genes by both taste and lingual cells indicates high quality RNA material.

PCR products are analyzed on agarose gels to determine if bands of the appropriate size are present in taste cells but not lingual cells. Genes with this expression pattern are putative taste-specific genes. All taste-specific genes were cloned and sequenced to confirm the gene identities.
4) In Situ Hybridization: Antisense RNA probes specific for an individual gene(s) (identified by gene chips or PCR) are hybridized to tissue sections containing taste cells to determine if the mRNA transcript for the gene of interest is expressed in taste cells, specifically in sour, sweet, bitter, and/or umami cells or in a unique cell type that may be involved in salty taste detection. In double labeling in situ hybridization, two different RNA probes are generated to label two different genes, specifically two different taste-specific genes identified by gene chip and/or PCR approaches. Alternatively, one probe can be generated to label a single gene to determine if the gene is expressed in taste cells. For double labeling studies, the first gene is labeled with a FITC probe that generates one color in a fluorescent microscope while the second gene is labeled with a digoxygenin (DIG) probe that generates a different color in a fluorescent microscope. Superimposition of probe 1 and probe 2 reveals if genes are expressed in the same or in different cell types. For example, if a unique ion channel identified by gene chip or PCR approaches colocalizes to cells expressing TRPM5, that unique ion channel is expressed in cells responsible for sweet, bitter, and/or umami taste. By contrast, if a unique ion channel identified by gene chip or PCR approaches does not colocalize to cells expressing TRPM5, that unique ion channel is expressed in a different cell type that may be responsible for salty taste (or another taste modality) and that unique ion channel may be directly involved in sodium detection.
5) Immunohistochemistry: Antibodies specific for an individual protein (whose gene was identified by gene chips or PCR) are applied to tissue sections containing taste cells to determine if the protein of interest is expressed in taste cells, specifically in sour, sweet, bitter, and/or umami cells or in a unique cell type that may be involved in salty taste detection. In double labeling immunohistochemistry, two different antibody probes are used to label two different proteins, specifically two different taste-specific proteins whose genes were identified by gene chip and/or PCR approaches. Alternatively, one antibody probe can be used to label a single protein to determine if the protein is expressed in taste cells. For double labeling studies, the first protein is labeled with an antibody at a very dilute concentration that can only be detected with a sensitive detection method termed tyramide signal amplification (TSA). The second protein is then labeled with another antibody and detected using a non-TSA method. The dilute first antibody cannot be detected by the standard non-TSA method; therefore, two different antibodies from the same species (e.g. rabbit polyclonal antibodies) will not cross-react and, therefore, can be used in double labeling experiments. Superimposition of protein label 1 and protein label 2 reveals if proteins are expressed in the same or in different cell types. For example, if a unique ion channel identified by gene chip or PCR approaches colocalizes to cells expressing TRPM5, that unique ion channel is expressed in cells responsible for sweet, bitter, and/or umami taste. By contrast, if a unique ion channel identified by gene chip or PCR approaches does not colocalize to cells expressing TRPM5, that unique ion channel is expressed in a different cell type that may be responsible for salty taste (or another taste modality) and that unique ion channel may be directly involved in sodium detection.

Further, the identification of the subject ion channel gene as an ion channel potentially involved in salty taste perception further included the following rationale to select potential salty taste receptor or ion channel candidates.

First taste buds are isolated using LCM as described above from macaque (Macaca fascicularis). Macaque genes are on average 90-95% identical to human genes and the macaque is an excellent experimental model for study of human biology including taste. Thus taste genes identified in the macaque will be highly similar to their human orthologs and carry out similar functions to those seen in humans. Using LCM a fine laser beam is used to dissect and purify taste cells from histological sections. This method isolates taste cells devoid of contaminating lingual epithelial cells and connective tissue and allows molecular biology experiments to be effected on a highly enriched taste cell population. In parallel, lingual epithelial cells are isolated by LCM and used as a negative control devoid of taste cells. LCM is advantageous to manual or enzymatic dissection of taste papilla because these crude techniques tend to yield a heterogeneous mixture of taste and lingual cells in which taste cells only comprise about 1-20% of the collected material.

Secondly, RNA isolated from taste and non-taste cells is analyzed using gene chips/microarrays. Gene chips contain most all annotated genes on a small chip. Hybridizing RNA, isolated from taste and lingual cells, to gene chips can be used to determine which specific genes are expressed in taste cells and not lingual cells as well as which specific genes are expressed at higher levels in taste cells compared to lingual cells. In order to identify genes for which probe sets are not functional on gene chips, gene chips were performed on 21 macaque non-taste tissues. Probe sets for genes not yielding data above background levels include both probe sets that do not hybridize efficiently to gene targets as well as probe sets not represented within the panel of 21 macaque tissues. These genes, representing genes not covered by the gene chip approach, are analyzed separately by PCR and/or histology to identify genes, specifically genes encoding transmembrane proteins, which are expressed in taste cells and not lingual cells as well as genes expressed at higher levels in taste cells compared to lingual cells isolated by LCM.

Third, taste-specific genes identified by gene chips and/or PCR are examined by histology using double labeling approaches. With in situ hybridization antisense probes specific for individual genes are hybridized to tissue sections containing taste cells to determine if the mRNA transcript for the gene of interest is expressed in taste cells, specifically in sweet bitter, sour and/or umami taste cells or in a unique cell type that may be involved in salt or other taste modality, e.g., fat taste detection. Using immunohistochemistry antibodies specific for an individual protein (which gene was identified by gene chips) these antibodies are applied to tissue sections containing taste cells to determine if the protein of interest is expressed in taste cells, specifically in sweet, bitter, sour and/or umami cells or in a unique cell type that may be involved in salt or fat taste detection. Genes expressed in taste cells expressing TRPM5, a marker for sweet, bitter, and umami cells, would encode proteins that may modulate sweet, bitter and/or umami taste. Genes expressed in taste cells expressing PKD2L1 or PKD1L3, markers for sour cells, would encode proteins that may modulate sour taste. Genes expressed in taste cells expressing neither TRPM5 nor PKD2L1 or PKD1L3 would encode proteins expressed in a unique cell type that may correspond to a salt or fat cell. Therefore, genes expressed in a unique taste cell type may correspond to a salty taste receptor or a fat taste receptor and may modulate salty or fat taste detection.

Fourth, using similar LCM procedures and gene chip or PCR expression methods experiments are conducted to identify which set of genes are specifically expressed in the top half of taste buds and not in the bottom half or which are enriched in the top half, i.e., expressed at least 1.2-1.5 fold higher in the cells comprising the top half of the taste bud relative to cells in the bottom half. (These genes are preferred candidates for human taste receptors given their orientation on the taste bud).

Fifth, taste-specific genes expressed in a unique cell type are analyzed by use of functional assays including electrophysiology to determine of gene products expressed in heterologous systems such as HEK1293 cells, CHO cells, or Xenopus oocytes generate sodium-responsive receptors or sodium-conducting ion channels. A salt receptor target should respond to sodium ions at concentrations relevant for human taste (between 20-140 mM sodium).

Sixth, to ultimately validate the role of a gene as a salt receptor, genes meeting the criteria set forth above are advanced into high-throughput screens to identify enhancers and blockers and these compounds are tested in salty taste tests to determine if they augment or repress salty taste perception. In parallel, mouse knockouts are generated lacking the gene of interest (or expressing a variant form as herein with the Varitint mouse) and physiological (nerve recordings) and behavioral (2-bottle preference tests and gustometer tests) experiments are performed to determine if the animals are deficient in or lack salty taste perception.

Therefore, the subject TRPML3 gene was identified as encoding a polypeptide ion channel that is involved in sodium taste sensing and likely sodium sensing and metabolism more broadly based on the following criteria: 1) Genes expressed specifically in taste cells or at higher levels in taste cells than lingual cells in gene chip and/or PCR experiments (these are defined as taste-specific genes); 2) Genes expressed in a unique cell type, that does not correspond to sweet, bitter, sour, and/or umami cells by histology; 3) Gene products that generate sodium responsive receptors or sodium channels in electrophysiology or functional experiments; and 4) Enhancers or blockers of gene products modulate salty taste perception and/or mouse knockouts or expressing inactive or variant forms of the ion channel gene of interest are deficient in or lack salty taste responsiveness.

Using such rationale, methods and protocols a large number of primate genes were initially identified as taste cell specific. These genes including TRPML3 are contained in earlier provisional applications incorporated by reference herein. This large set of genes given the comprehensive and accurate methodologies used to identify these genes is predicted by the inventors to be comprehensive of the genes which are specifically expressed in primate taste cells.

From this large group of genes a small subset of taste specific genes which are taste specific, and which are specifically expressed or are enriched in the top half of taste buds and which encode sodium ion channels was identified. In particular three ion channel taste specific genes were identified, i.e. TRPML3, NKAIN3 and NALCN. Of these 3 genes it was confirmed that TRPML3 encodes an ion channel that is involved and required for salty taste perception.

Specifically, the functional (electrophysiological) and immunohistochemical data contained in the examples infra and the data obtained in the Varitint mouse indicate that MCOLN3 (TRPML3) functions as a salty taste receptor in rodents, humans as well as other primates and most likely other mammals and also likely plays a role in other physiological functions involving sodium metabolism, absorption and excretion such as those relating to aldosterone production and vasopressin release. The criteria further that supported the testing of the selected ion channel genes is summarized in Table 1 below.

TABLE 1

| | TB vs LE | Top vs Bottom | Reference reporting that this gene encodes |
|---|---|---|---|
| Gene Name | Ratio | TB Ratio | a sodium channel |
| NALCN (aka | 11.2 | 7.2 | Cell. 2007 Apr 20; 129(2): 371-83 |
| VGCNL1) | | | The neuronal channel NALCN contributes |
| | | | resting sodium permeability and is required for |
| | | | normal respiratory rhythm. |
| | | | Lu B, Su Y, Das S, Liu J, Xia J, Ren D. |
| | | | Describes NALCN as a sodium leak channel, |
| | | | consistent with a predicted salt receptor. |
| TRPML3 | 10.2 | 1.6 | J Biol Chem. 2007 Oct 25; [Epub ahead of |
| (aka | | | print] |
| MCOLN3) | | | Gain-of-function mutation in TRPML3 causes |
| | | | the mouse varitint-waddler phenotype. |
| | | | Kim HJ, Li Q, Tjon-Kon-Sang S, So I, Kiselyov K, |
| | | | Muallem S. |
| | | | First description of TRPML3 as a channel |
| | | | permeable to sodium, consistent with a salt |
| | | | receptor. |
| NKAIN3 | 3.3 | 1.5 | Hum Mol Genet. 2007 Oct 15; 16(20): 3394-410. |
| (aka | | | Epub 2007 Jul 2 |
| FAM77D) | | | A novel family of transmembrane proteins |
| | | | interacting with {beta} subunits of the Na, K- |
| | | | ATPase. |
| | | | Gorokhova S, Bibert S, Geering K, Heintz N. |
| | | | Describes a Drosophila homologue of |
| | | | NKAIN3 as an amiloride-insensitive sodium |
| | | | channel, consistent with a salt receptor. |

Therefore, based on the foregoing, the subject invention generally relates to methods for identifying taste genes, including genes involved in salty taste perception or other taste perception such as fat taste perception and the use in screening assays for identifying human salty taste enhancers and other taste modulatory compounds and for identifying potential therapeutics that modulate other taste cell related functions and phenotypes including diseases and conditions not directly related to taste transduction, i.e., those relating to aberrant sodium transport, metabolism and excretion and sensing by different tissues.

The compounds which modulate TRPML3 have potential application in modulating human salty taste perception and the afore-mentioned therapeutic and prophylactic applications. Compounds identified for example in electrophysiological assays and their biologically acceptable derivatives are to be tested in human taste tests using human volunteers to confirm their effect on human salty taste perception. In addition compounds identified as potential therapeutics will be evaluated in appropriate in vitro and in vivo models depending on the nature of the intended application. For example compounds identified as potential therapeutics for regulating renal function, sodium homeostasis, plasma osmolarity, blood pressure, diabetes may be evaluated in well known animal models. Foe example in the case of diabetes diabetic animal models such the NOD mouse model or BB rat model.

The cell-based assays used to identify taste, e.g., salty taste modulatory or therapeutic compounds will preferably comprise high throughput screening platforms to identify compounds that modulate (enhance) the activity of genes involved in salty taste perception using cells that express the genes disclosed herein or combinations thereof. Additionally, these sequences may be modified to introduce silent mutations or mutations having a functional effect such as defined mutations that affect ion (sodium) influx. As noted above, the assays will preferably comprise electrophysiological assays effected in amphibian oocytes or assays using mammalian cells that express a an ion channel according to the invention using fluorescent ion sensitive dyes or membrane potential dyes, e.g., sodium-sensitive dyes. Preferably, compounds that modulate such ion channels are identified by screening using electrophysiological assays effected with oocytes that express an ion channel identified herein (e.g., patch clamping or two electrode voltage clamping).

Still alternatively, compounds that modulate the subject ion channels putatively involved in salty taste may be detected by ion flux assays, e.g., radiolabeled-ion flux assays or atomic absorption spectroscopic coupled ion flux assays. As disclosed supra, these compounds have potential application in modulating human salty taste perception or for modulating other biological processes involving aberrant or normal ion channel function.

The subject cell-based assays use mutant nucleic acid sequences which are expressed in desired cells, preferably oocytes or human cells such as HEI-293 cells, or other human or mammalian cells conventionally used in screens for identifying ion channel or GPCR modulatory compounds. These cells may further be engineered to express other sequences, e.g., other taste GPCRs, i.e., T1Rs or T2Rs such as is described in other patent applications by the present Assignee Senomyx as well as appropriate G proteins. The oocyte system is advantageous as it allows for direct injection of multiple mRNA species, provides for high protein expression and can accommodate the deleterious effects inherent in the overexpression of ion channels. The drawbacks are however that electrophysiological screening using amphibian oocytes is not as amenable to high throughput screening of large numbers of compounds and is not a mammalian system. As noted, the present invention embraces assays using mammalian cells, preferably high throughput assays. These high throughput screening assays typically will use mammalian cells transfected or seeded into wells or culture plates wherein functional expression in the presence of test compounds is allowed to proceed and activity is detected using membrane-potential fluorescent or ion (sodium) fluorescent dyes.

These methods of screening are used to identify TRPML3 modulators, e.g., activators, inhibitors, stimulators, enhancers, etc., of human salty taste or other taste modalities and potential therapeutics that target other taste cell functions or phenotypes using the nucleic acids and proteins, sequences provided herein. Such modulators can affect salty taste or other taste modalities or taste cell related functions and phenotypes, e.g., by modulating transcription, translation, mRNA or protein stability; by altering the interaction of the ion channel with the plasma membrane, or other molecules; or by affecting ion channel protein activity.

Compounds are screened, e.g., using high throughput screening (HTS), to identify those compounds that can bind to and/or modulate the activity of a taste receptor or taste ion channel polypeptide or transporter or fragment thereof. In the present invention, proteins are recombinantly expressed in cells, e.g., human cells, or frog oocytes and the modulation of activity is assayed by using any measure of ion channel, receptor or transporter function, such as measurement of the membrane potential, or measures of changes in intracellular sodium or lithium levels. Methods of assaying ion, e.g., cation, channel function include, for example, patch clamp techniques, two electrode voltage clamping, measurement of whole cell currents, and fluorescent imaging techniques that use ion sensitive fluorescent dyes and ion flux assays, e.g., radiolabeled-ion flux assays or ion flux assays.

An enhancer of a gene identified according to the invention can be used for a number of different purposes. For example, it can be included as a flavoring agent to modulate the salty taste of foods, beverages, soups, medicines, and other products for human consumption. Additionally, the invention provides kits for carrying out the herein-disclosed assays. Also, it can be used in treating conditions involving TRPML3 function as afore-mentioned.

In fact, as described in the examples such TRPML3 cell-based assays have already identified TRPML3 blockers and enhancers which were tested in taste tests to establish their effect on salty taste perception. As noted therein these results are preliminary as these compounds may be optimized, other molecules may be identified possessing greater antagonist or agonist function, these compounds may be screened in additional assays in order to identify those which are selective in their effect (blocking or enhancing on salty taste), i.e., which do not elicit other (non-salty) taste modalities such as bitterness or off-taste.

Also the present invention provides the use of TRPML3 as a marker which can be used to enrich, identify or isolate salt receptor expressing cells.

Also this invention provides in vitro and in vivo assays which use TRPML3 (MCOLN3) and TRPML3 expressing cells or TRPML3 transgenic animal models to identify agonist, antagonist or enhancer compounds which elicit or modulate (block or enhance) salty taste in primates including humans. These assays use cells which express TRPML3 alone or cells which express the TRPML3 ion channel in association with other taste specific polypeptides such as NALCN or NKAIN3 or related TRPML members such as TRPML1 or TRPML2.

Further this invention provides transgenic animals, preferably rodents, and the use thereof to confirm the role of TRPML3 in salty taste in mammals and in other physiological functions involving sodium and other ions such as sodium metabolism, blood pressure, fluid retention and excretion, plasma osmolarity, aquaporin-2 function and/or trafficking, ENaC function and/or trafficking, urinary function and cardiac function. In particular, TRPML3 is TRPML3 is a target for modulation of sodium homeostasis, including blood pressure regulation in hypertension (high blood pressure) and hypotension (low blood pressure), and based thereon TRPML3 enhancers and blockers can be used to modulate renal sodium balance to control blood pressure; TRPML3 enhancers and blockers can be used to modulate plasma osmolarity, including decreases in osmolarity (hypoosmotic) and increases in osmolarity (hyperosmotic) via reabsorption of water in the kidney in normal and diseased states such as diabetis insipidus; TRPML3 enhancers and blockers can be used to modulate ENaC function and/or trafficking; TRPML3 enhancers and blockers can be used to modulate aquaporin-2 function and/or trafficking; TRPML3 enhancers and blockers can be used to modulate vasopressin (antidiuretic hormone) secretion from the pituitary gland; and TRPML3 enhancers and blockers can be used to modulate aldosterone secretion from the adrenal glands.

In addition, this invention provides in vitro and in vivo assays which use TRPML3 and TRPML3 expressing cells or transgenic animals in assays, preferably electrophysiological assays in order to identify therapeutic compounds which alleviate diseases and conditions involving deficiencies in the expression of this polypeptide including hyperexpression, hyporexpression, and mutations in the TRPML3 polypeptide that affect its ability to function as a taste specific sodium channel in mammal including e.g., human and non-human primates. As mentioned, these conditions include conditions wherein modulation of plasma osmolarity is therapeutically warranted, including decreases in osmolarity (hypoosmotic) and increases in osmolarity (hyperosmotic) via reabsorption of water in the kidney in normal and diseased states such as diabetis insipidus; conditions wherein modulation of ENaC function and/or trafficking; aquaporin-2 function and/or trafficking; vasopressin (antidiuretic hormone) secretion from the pituitary gland; and aldosterone secretion from the adrenal glands is therapeutically or prophylactically warranted. Other conditions involving TRPML3 function include by way of example Addison's disease and diseases involving aberrant aldosterone production or vasopressin release such as hypertension, hypotension, fluid retention, and impaired urinary or cardiac function such as arrhythmia, heart attach and stroke and diabetis insipidus or pseudohypoaldosteronism type I, a salt wasting human genetic disease.

### DEFINITIONS

“Putative salty taste receptor or salty taste ion channel gene” refers to a gene expressed in taste cells that is not expressed in lingual cells or is expressed substantially less in lingual cells that moreover preferably is not expressed in taste cells that express a T1R, T2R, TRPM5, or PKD2L1/PKD1L3 gene. Preferably these genes are specifically expressed or are enriched (expressed at least 1.2-1.5 fold higher) in the top versus the bottom half of the cells which comprise the taste buds. This includes chemosensory or taste cells, particularly those of macaque and likely other mammals. As noted and in the preferred aspect of the invention TRPML3 has been identified as one such salty taste receptor (sodium ion channel) polypeptide.

“TRPML3” or “MCOLN3” refers to a gene or a variant thereof that is involved in salty taste perception in rodents, humans and non-human primates and likely other mammals and vertebrates including birds, reptiles and amphibians. This application contains immediately preceding the claims exemplary sequences for human, mouse, bovine, murine, zebra fish, chicken, and other mammalian species TRPML3 genes and polypeptides. Comparison of these sequences reveals that the polypeptide sequence of TRPML3 polypeptides are very similar in different species, i.e., mouse and human TRPML3 are 96% sequence identity and 91% sequence similarity. Therefore, it should be relatively straightforward for one skilled in the art to identify TRPML3 genes in other mammalian gene or genomic and polypeptide libraries. Also, it is likely that other vertebrates express TRPML3 given the essential role of sodium metabolism to cell vitality and the general well being of organisms. According this invention is intended to broadly encompass salty receptors containing TRPML3 genes and functional variants such as chimeras of different species, but most preferably humans and other mammals. Also, the TRPML3 genes and polypeptides herein specifically embrace TRPML3 mutated genes and fragments which have been mutated at one or more sites, e.g., in order to modify (enhance or decrease TRPML3 activity), render the ion channel more suitable for use in assays such as by modifying the polypeptide so that the ion channel is fixed in the “open” or “closed” position or by creating fragments or chimeras wherein a domain or extracellular loop or a portion thereof of one TRPML3 polypeptide is swapped with that of another ion channel. e.g., a TRPML3 of a different species or a non-TRPML3 ion channel such as e.g., TRPML1, TRPML2, NKAIN3, or NALCN. Also, the term TRPML3 polypeptides and nucleic acid sequences specifically encompasses TRPML3 ion channel polypeptides which posses at least 80% sequence identity to those disclosed herein, more preferably at least 90% sequence identity or still more preferably at least 95, 96, 97, 98 or 99% sequence identity to a native TRPML3 polypeptide, e.g., a native human, non-human primate, rodent (rat, mouse, et al.), dog, cat, horse, bovine, sheep, etc., TRPML3 polypeptide. TRPML3 nucleic acid sequences further include all nucleic acid sequences encoding therefore such as cDNAs, genomic sequences, cRNAs, mRNAs, as well as single stranded, double stranded and triple stranded nucleic sequences and their complements. In humans there are 3 major forms of TRPML3 mRNA in taste buds, pituitary and the adrenal the sequences of which are contained in the List of sequences before the claims.

Also, TRPML3 sequences include sequences that specifically hybridize to the subject TRPML3 encoding nucleic acid sequences or their complements which encode a sodium ion channel involved in salty taste perception and/or sodium transport, metabolism or excretion. Exemplary hybridization conditions suitable for identifying other TRPML3 orthologs and related genes are known in the art and are defined in this application below.

“Taste Cell” refers to a cell that when mature expresses at least one receptor, transporter, or ion channel that directly or indirectly regulates or modulates a specific taste modality such as sweet, sour, umami, salty, bitter, fatty, metallic or other taste perception or general taste perception such as taste intensity or the duration of a taste response. This includes in particular genes that are expressed specifically in chemosensory or taste cells, particularly macaque and likely other mammalian taste cells. Taste cells express mRNA and/or a protein for the gene C6orf15 (chromosome reading frame 15)—also known as STG. This gene has been described as a taste-specific gene (M. Neira et al. Mammalian Genome 12: 60-66, 2001) and is among the macaque taste specific genes reported herein. In addition a mature taste receptor cell typically will express mRNA and/or protein for alpha ENaC. We have data (not shown herein) that reveals that alpha ENaC is expressed in at least sweet, bitter, umami, sour and most likely salty taste cells. Further, a mature taste receptor cell will typically express mRNA and/or protein for cytokeratin 19. This protein is only expressed in mature taste cells and is not found in basal or stem cells. (L. Wong et al. Chemical Senses 19(3): 251-264, 1994). Furthermore, taste cells can be identified by those skilled in the art base on their characteristic morphology. In particular mature taste receptor taste cells are elongated and spindle-shaped. Also, a mature taste receptor cell has the apex of the cell (apical membrane) penetrating into the taste pore thereby gaining access or exposure to saliva. By contrast, an immature taste cell, e.g., a basal cell or stem cell is rounded and is not exposed to the taste pore and saliva. Also, unlike mature taste cells, basal and stem cells tend to be localized towards the base of taste buds.

“Chemosensory cells” are cells that are involved in sensing of chemical stimulants such as tastants and other chemical sensory stimuli such as odorants. Chemosensory cells herein include in particular taste receptor cells and cells comprised in the digestive or urinary tract or other organs that when mature express one or more taste receptors. For example, gastrointestinal chemosensory cells are known which express T1Rs or T2Rs and which cells are likely involved in food sensing, metabolism, digestion, diabetes, food absorption, gastric motility, et al. In addition, cells found in the urinary tract likely express salty taste receptors and are involved in sodium transport, excretion and functions associated therewith such as blood pressure and fluid retention. Further, in the digestive system chemosensory cells that express taste receptors may also express chromogranin A, which is a marker of secretory granules. (C. Stermini, “Taste Receptors in the Gastrointestinal Tract. IV. Functional Implications of Bitter Taste Receptors in Gastrointestinal Chemosensing”. American Journal of Physiology, Gastrointestinal and Liver Physiology.”, 292:G457-G461, 2007).

“Taste-cell associated gene” herein refers to a gene expressed by a taste cell that is not expressed by lingual cell that is involved in a taste or non-taste related taste cell function or phenotype. This includes in particular genes reported herein and in earlier provisional applications cited herein that are expressed specifically in chemosensory or taste cells, particularly those from macaque. Taste cells include cells in the oral cavity that express taste receptors such as the tongue and taste cells in other areas of the body that express taste receptors such as the digestive system and urinary tract. Such genes include those contained in the tables in the applications incorporated by reference herein. These genes include genes involved in taste and non-taste related functions such a taste cell turnover, diseases affecting the digestive system or oral cavity, immunoregulation of the oral cavity and/or digestive system, digestive and metabolic functions involving taste cells such a diabetes, obesity, blood pressure, fluid retention et al. In referring to the particular taste specific genes identified therein these genes include the nucleic acid sequences corresponding the genes contained therein as well as orthologs thereof and chimeras and variants including allelic variants thereof. In particular such variants include sequences encoding polypeptides that are at least 80% identical, more preferably at least 90% or 95% identical to the polypeptides encoded by the genes corresponding to the recited genes or to orthologs thereof, especially human and non-human primate orthologs. In addition, the genes include nucleic acid sequences that hybridize under stringent hybridization conditions to a nucleic acid sequence corresponding to one of the gene sequences corresponding to the genes in the earlier provisional patent applications.

“Cation channels” are a diverse group of proteins that regulate the flow of cations across cellular membranes. The ability of a specific cation channel to transport particular cations typically varies with the valency of the cations, as well as the specificity of the given channel for a particular cation.

“Homomeric channel” refers to a cation channel composed of identical alpha subunits, whereas “heteromeric channel” refers to a cation channel composed of two or more different types of alpha subunits. Both homomeric and heteromeric channels can include auxiliary beta subunits.

A “beta subunit” is a polypeptide monomer that is an auxiliary subunit of a cation channel composed of alpha subunits; however, beta subunits alone cannot form a channel (see, e.g., U.S. Pat. No. 5,776,734). Beta subunits are known, for example, to increase the number of channels by helping the alpha subunits reach the cell surface, change activation kinetics, and change the sensitivity of natural ligands binding to the channels. Beta subunits can be outside of the pore region and associated with alpha subunits comprising the pore region. They can also contribute to the external mouth of the pore region.

The term “authentic” or wild-type” or “native” nucleic acid sequences refer to the wild-type nucleic acid sequences contained in the Sequence Listing herein as well as splice, allelic and other variants and other nucleic acid sequences generally known in the art.

The term “authentic” or “wild-type” or “native” polypeptides refers to the polypeptide encoded by the genes and nucleic acid sequence disclosed in this and earlier provisional patent applications which are incorporated by reference.

The term “modified enhance receptor nuclear acid sequence” or “optimized nucleic acid sequence” refers to a nucleic acid sequence which contains one or more mutations, particularly those that affect (inhibit or enhance) gene activity in recombinant host cells, and most especially oocytes or human cells such as HEK-293 cells. Particularly, these mutations include those that affect gating by the resultant ion channel containing the mutated subunit sequence. The ion channel may comprise such mutations in one or several of the three subunits that constitute the particular ion channel. The modified nucleic acid sequence for example may contain substitution mutations in one subunit that affect (impair) gating function or defective surface expression. The invention embraces the use of other mutated gene sequences, i.e., splice variants, those containing deletions or additions, chimeras of the subject sequences and the like. Further, the invention may use sequences which may be modified to introduce host cell preferred codons, particularly amphibian or human host cell preferred codons.

The term receptor or ion channel protein or fragment thereof, or a nucleic acid encoding a particular taste receptor or ion channel or transporter or a fragment thereof according to the invention refers to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by the wild-type nucleic acid or amino acid sequence of the taste protein, e.g., proteins encoded by the gene nucleic acid sequences contained in the Table 1 herein as well as fragments thereof, and conservatively modified variants thereof; (3) polypeptides encoded by nucleic acid sequences which specifically hybridize under stringent hybridization conditions to an anti-sense strand corresponding to a nucleic acid sequence encoding a gene encoded by one of said genes, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a nucleic acid, e.g., those disclosed herein.

A putative salty or other taste specific gene or polynucleotide or polypeptide sequence is typically from a mammal including, but not limited to, primate, e.g., human; rodent, e.g., rat, mouse, hamster; cow, pig, horse, sheep, or any other mammal. However, as noted the TRPML3 ion channel is expressed in other (non-mammal) vertebrates where it likely has a similar function. The nucleic acids and proteins of the invention include either naturally occurring or recombinant molecules. Typically these genes will encode proteins that have ion channel activity, i.e., they are permeable to sodium or lithium. In particular this includes primate TRPML3 genes and their human and other mammalian orthologs as well as fragments and variants that retain TRPML3 functionality, i.e., behave analogously in electrophysiological assays that monitor sodium, lithium conductance and responsiveness (lack) to amiloride as well as in other suitable functional assays.

By “determining the functional effect” or “determining the effect on the cell” is meant assaying the effect of a compound that increases or decreases a parameter that is indirectly or directly under the influence of a taste gene, preferably salty taste gene identified herein e.g., functional, physical, phenotypic, and chemical effects. Such functional effects include, but are not limited to, changes in ion flux, membrane potential, current amplitude, and voltage gating, a as well as other biological effects such as changes in gene expression of any marker genes, and the like. The ion flux can include any ion that passes through the channel, e.g., sodium or lithium, and analogs thereof such as radioisotopes. Such functional effects can be measured by any means known to those skilled in the art, e.g., patch clamping, using voltage-sensitive dyes, or by measuring changes in parameters such as spectroscopic characteristics (e.g., fluorescence, absorbance, refractive index), hydrodynamic (e.g., shape), chromatographic, or solubility properties. Suitable electrophysiological assays using TRPML3 expressing cells are exemplified in the experimental examples infra.

“Inhibitors,” “activators,” and “modulators” of the subject taste cell expressed polynucleotide and polypeptide sequences are used to refer to activating, inhibitory, or modulating molecules identified using in vitro and in vivo assays of these polynucleotide and polypeptide sequences. Inhibitors are compounds that, e.g., bind to, partially or totally block activity, decrease, prevent, delay activation, inactivate, desensitize, or down regulate the activity or expression of these taste specific proteins, e.g., antagonists. “Activators” are compounds that increase, open, activate, facilitate, enhance activation, sensitize, agonize, or up regulate protein activity. Inhibitors, activators, or modulators also include genetically modified versions of the subject taste cell specific proteins, e.g., versions with altered activity, as well as naturally occurring and synthetic ligands, antagonists, agonists, peptides, cyclic peptides, nucleic acids, antibodies, antisense molecules, siRNA, ribozymes, small organic molecules and the like. Such assays for inhibitors and activators include, e.g., expressing the subject taste cell specific protein in vitro, in cells, cell extracts, or cell membranes, applying putative modulator compounds, and then determining the functional effects on activity, as described above.

Samples or assays comprising the proteins encoded by genes identified herein that are treated with a potential activator, inhibitor, or modulator are compared to control samples without the inhibitor, activator, or modulator to examine the extent of activation or migration modulation. Control samples (untreated with inhibitors) are assigned a relative protein activity value of 100%. Inhibition of an ion channel is achieved when the activity value relative to the control is about 80%, preferably 50%, more preferably 25-0%. Activation of an ion channel is achieved when the activity value relative to the control (untreated with activators) is 110%, more preferably 150%, more preferably 200-500% (i.e., two to five fold higher relative to the control), more preferably 1000-3000% or higher.

The term “test compound” or “drug candidate” or “modulator” or grammatical equivalents as used herein describes any molecule, either naturally occurring or synthetic compound, preferably a small molecule, or a protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, siRNA, oligonucleotide, ribozyme, etc., to be tested for the capacity to modulate cold sensation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., a gene or sequence contained in the Table 1 herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nucl. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci., USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, or complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine %), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., transmembrane domains, pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of .beta.-sheet and .alpha.-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include³²¹, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The term antibody, as used herein, also includes antibody fragments either produced by the modification of whole antibodies, or those synthesized de novo using recombinant DNA methodologies (e.g., single chain Fv), chimeric, humanized or those identified using phage display libraries (see, e.g., McCafferty et al., Nature 348:552-554 (1990)) For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988) and Harlow & Lane, Using Antibodies, A Laboratory Manual (1999); and Goding, Monoclonal Antibodies: Principles and Practice (2d ed. 1986)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies rose to a protein, polymorphic variants, alleles, orthologs, and conservatively modified variants, or splice variants, or portions thereof, can be selected to obtain only those polyclonal antibodies that are specifically immunoreactive with proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

Recombinant Expression of Taste (Salty) Gene Identified Herein

To obtain high level expression of a cloned gene, such as those cDNAs encoding the subject genes, one typically subclones the gene into an expression vector that contains a strong promoter to direct transcription, a transcription/translation terminator, and if for a nucleic acid encoding a protein, a ribosome binding site for translational initiation. Suitable eukaryotic and prokaryotic promoters are well known in the art and described, e.g., in Sambrook et al., and Ausubel et al., supra. For example, bacterial expression systems for expressing the taste specific protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. For example, retroviral expression systems may be used in the present invention. As described infra, the subject putative salty taste affecting genes are preferably expressed in human cells such as HEK-293 cells which are widely used for high throughput screening.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the identified gene and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells. A particularly preferred expression system is the BacMam expression system which uses a baculovirus based vector to express a polypeptide in mammalian cells, herein a TRPML3 polypeptide expressed in HEK-293 cells.

Expression of proteins from eukaryotic vectors can also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

The vectors used in the invention may include a regulatable promoter, e.g., tet-regulated systems and the RU-486 system (see, e.g., Gossen & Bujard, Proc. Nat'l Acad. Sci. USA 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a gene sequence under the direction of the polyhedrin promoter or other strong baculovirus promoters.

The elements that are typically included in expression vectors also include a replicon that functions in the particular host cell. In the case of E. coli, the vector may contain a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods may be used to produce bacterial, mammalian, yeast or insect cell lines that express large quantities of the desired taste specific protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the gene.

After the expression vector is introduced into the cells, the transfected cells are cultured under conditions favoring expression of the gene. In some instances, such polypeptides may be recovered from the culture using standard techniques identified below.

### Assays for Modulators of Putative TRPML3 Specific Gene Products

Modulation of a putative taste cell specific protein, can be assessed using a variety of in vitro and in vivo assays, including cell-based models as described above. Such assays can be used to test for inhibitors and activators of the protein or fragments thereof, and, consequently, inhibitors and activators thereof. Such modulators are potentially useful in medications or as flavorings to modulate salty or other taste modalities or taste in general or for usage as potential therapeutics for modulating a taste cell related function or phenotype involving one or several of the identified taste cell specific genes reported herein.

Assays using cells expressing the subject taste specific proteins, either recombinant or naturally occurring, can be performed using a variety of assays, in vitro, in vivo, and ex vivo, as described herein. To identify molecules capable of modulating activity thereof, assays are performed to detect the effect of various candidate modulators on activity preferably expressed in a cell.

The channel activity of ion channel proteins in particular can be assayed using a variety of assays to measure changes in ion fluxes including patch clamp techniques, measurement of whole cell currents, radiolabeled ion flux assays or a flux assay coupled to atomic absorption spectroscopy, and fluorescence assays using voltage-sensitive dyes or lithium or sodium sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)). For example, a nucleic acid encoding a protein or homolog thereof can be injected into Xenopus oocytes or transfected into mammalian cells, preferably human cells such as HEK-293 cells or CHO cells. Channel activity can then be assessed by measuring changes in membrane polarization, i.e., changes in membrane potential.

A preferred means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., Pflugers. Archiv. 391:185 (1981).

Channel activity is also conveniently assessed by measuring changes in intracellular ion levels, i.e., sodium or lithium. Such methods are exemplified herein. For example, sodium flux can be measured by assessment of the uptake of radiolabeled sodium or by using suitable fluorescent dyes. In a typical microfluorimetry assay, a dye which undergoes a change in fluorescence upon binding a single sodium ion, is loaded into the cytosol of taste cell specific ion channel-expressing cells. Upon exposure to an agonist, an increase in cytosolic sodium is reflected by a change in fluorescence that occurs when sodium is bound.

The activity of the subject taste cell specific polypeptides can in addition to these preferred methods also be assessed using a variety of other in vitro and in vivo assays to determine functional, chemical, and physical effects, e.g., measuring the binding thereof to other molecules, including peptides, small organic molecules, and lipids; measuring protein and/or RNA levels, or measuring other aspects of the subject polypeptides, e.g., transcription levels, or physiological changes that affects the taste cell specific protein's activity. When the functional consequences are determined using intact cells or animals, one can also measure a variety of effects such as changes in cell growth or pH changes or changes in intracellular second messengers such as IP3, cGMP, or cAMP, or components or regulators of the phospholipase C signaling pathway. Such assays can be used to test for both activators and inhibitors of KCNB proteins. Modulators thus identified are useful for, e.g., many diagnostic and therapeutic applications.

### In Vitro Assays

Assays to identify compounds with modulating activity on the subject genes are preferably performed in vitro. The assays herein preferably use full length protein according to the invention or a variant thereof. This protein can optionally be fused to a heterologous protein to form a chimera. In the assays exemplified herein, cells which express the full-length polypeptide are preferably used in high throughput assays are used to identify compounds that modulate gene function. Alternatively, purified recombinant or naturally occurring protein can be used in the in vitro methods of the invention. In addition to purified protein or fragment thereof, the recombinant or naturally occurring taste cell protein can be part of a cellular lysate or a cell membrane. As described below, the binding assay can be either solid state or soluble. Preferably, the protein, fragment thereof or membrane is bound to a solid support, either covalently or non-covalently. Often, the in vitro assays of the invention are ligand binding or ligand affinity assays, either non-competitive or competitive (with known extracellular ligands such as menthol). These in vitro assays include measuring changes in spectroscopic (e.g., fluorescence, absorbance, refractive index), hydrodynamic (e.g., shape), chromatographic, or solubility properties for the protein.

Preferably, a high throughput binding assay is performed in which the protein is contacted with a potential modulator and incubated for a suitable amount of time. A wide variety of modulators can be used, as described below, including small organic molecules, peptides, antibodies, and ligand analogs. A wide variety of assays can be used to identify modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. In such assays the known ligand is bound first, and then the desired compound i.e., putative enhancer is added. After the particular protein is washed, interference with binding, either of the potential modulator or of the known ligand, is determined. Often, either the potential modulator or the known ligand is labeled.

In addition, high throughput functional genomics assays can also be used to identify modulators of cold sensation by identifying compounds that disrupt protein interactions between the taste specific polypeptide and other proteins to which it binds. Such assays can, e.g., monitor changes in cell surface marker expression, changes in intracellular calcium, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the protein encoded by a cDNA according to the invention can be isolated using a yeast two-hybrid system, mammalian two hybrid system, or phage display screen, etc. Targets so identified can be further used as bait in these assays to identify additional components that may interact with the particular ion channel, receptor or transporter protein which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

### Cell-Based In Vivo Assays

In preferred embodiments, wild-type and mutant taste cell specific proteins are expressed in a cell, and functional, e.g., physical and chemical or phenotypic, changes are assayed to identify modulators that modulate function or which restore the function of mutant genes, e.g., those having impaired gating function. Cells expressing proteins can also be used in binding assays. Any suitable functional effect can be measured, as described herein. For example, changes in membrane potential, changes in intracellular lithium or sodium levels, and ligand binding are all suitable assays to identify potential modulators using a cell based system. Suitable cells for such cell based assays include both primary cells and recombinant cell lines engineered to express a protein. The subject taste cell specific proteins therefore can be naturally occurring or recombinant. Also, as described above, fragments of these proteins or chimeras with ion channel activity can be used in cell based assays. For example, a transmembrane domain of an ion channel according to the invention can be fused to a cytoplasmic domain of a heterologous protein, preferably a heterologous ion channel protein. Such a chimeric protein would have ion channel activity and could be used in cell based assays of the invention. In another embodiment, a domain of the taste cell specific protein, such as the extracellular or cytoplasmic domain, is used in the cell-based assays of the invention.

In another embodiment, cellular polypeptide levels of the particular target taste polypeptide can be determined by measuring the level of protein or mRNA. The level of protein or proteins related to ion channel activation are measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, protein expression can be measured using a reporter gene system. Such a system can be devised using a promoter of the target gene operably linked to a reporter gene such as chloramphenicol acetyltransferase, firefly luciferase, bacterial luciferase, □-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited ion channel will potentially alter the properties of target enzymes, second messengers, channels, and other effector proteins. The examples include the activation of phospholipase C and other signaling systems. Downstream consequences can also be examined such as generation of diacyl glycerol and IP3 by phospholipase C.

Assays for ion channel activity include cells that are loaded with ion or voltage sensitive dyes to report activity, e.g., by observing sodium influx or intracellular sodium release. Assays for determining activity of such receptors can also use known agonists and antagonists for these receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. Radiolabeled ion flux assays or a flux assay coupled to atomic absorption spectroscopy can also be used.

### Isolation of Nucleic Acids Encoding TRPML3 Proteins

This invention relies, in part, on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

Nucleic acids that encode TRPML3 proteins, polymorphic variants, orthologs, and alleles can be isolated using TRPML3 nucleic acid probes and oligonucleotides under stringent hybridization conditions, by screening libraries. Alternatively, expression libraries can be used to clone TRPML3 protein, polymorphic variants, orthologs, and alleles by detecting expressed homologous immunologically with antisera or purified antibodies made against TRPML3 or portions thereof.

To make a cDNA library, one should choose a source that is rich in TRPML3 RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

Alternatively, TRPML3 cRNA encoding TRPML3 may be generated from TRPML3 DNA plasmids using T7 RNA polymers to transcribe cRNA in vitro from DNA linearized with appropriate restriction enzymes and the resultant cRNA microinjected into suitable cells, e.g., oocytes, preferably frog oocytes.

An alternative method of isolating TRPML3 nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of TRPML3 directly from mRNA, from cDNA, from genomic libraries or cDNA libraries. Degenerate oligonucleotides can be designed to amplify TRPML3 homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of TRPML3 encoding mRNA in physiological samples, for nucleic acid sequencing, or for other purposes. Genes amplified by the PCR reaction can be purified from agarose gels and cloned into an appropriate vector.

Gene expression of TRPML3 can also be analyzed by techniques known in the art, e.g., reverse transcription and amplification mRNA, isolation of total RNA or poly A⁺ RNA, northern blotting, dot blotting, in situ hybridization, RNase protection, high density polynucleotide array technology, e.g. and the like.

Nucleic acids encoding TRPML3 proteins can be used with high-density oligonucleotide array technology (e.g., GeneChip™) to identify TRPML3 protein, orthologs, alleles, conservatively modified variants, and polymorphic variants in this invention. In the case where the homologs being identified are linked to modulation of T cell activation and migration, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); and Hacia et al., Nucleic Acids Res. 26:3865-3866 (1998).

As noted, a preferred assay for identification of compounds that modulate, i.e., enhance, inhibit or block TRPML3 comprises an electrophysiological assay that monitors changes in electrical current in cells that express TRPML3 that are contacted with at least one putative TRPML3 modulator (enhancer or inhibitor). These assays may use any cell that expresses a functional TRPML3. In the preferred embodiment, the cells will comprise oocytes, preferably frog oocytes, mammalian cells, yeast cells or insect cells, or another expression system that is suitable for expressing a functional TRPML3 ion channel. Preferably, the expression system will exhibit robust and rapid TRPML3 sodium channel expression and desirably will not express any or very few endogenous ion channels, thereby facilitating the identification of compounds that specifically modulate TRPML3 sodium channel function. Thereby, an undesirable background response is minimized or eliminated. Moreover, robust cells, such as oocytes, are desirable as this enables the cells to be reused in assays according to the invention. Oocytes have been reported previously to rapidly and robustly express other functional ion channels (Pascal, CRC Crit. Rev. Biotech. 22(4):317-87 (1987); Wagner et al., Cell Physiol. Biochem. 10:1-12 (2000); Canessa et al., Nature 367:463-467 (1994)).

A particularly preferred electrophysiological assay is a moderate throughput assay that measures TRPML3 channel function in frog oocytes by the two-electrode voltage clamp technique. This robust, fast expression system provides for the expression of up to millions of ion channels in an oocyte membrane after only about 18-24 hours. Moreover, because oocytes are relatively large (1 mm in diameter, relatively large compared to most mammalian cells), they are easy to handle and work with.

Based on these advantages, a single oocyte can be used to obtain multiple and repetitive electrophysiological recording. Also, an oocyte typically expresses few endogenous channels. Thereby, oocytes allow for repeated direct measurement of the effect of target compounds on TRPML3 sodium channel function.

In a preferred two-electrode voltage clamp assay according to the invention, frog oocytes that have been microinjected with TRPML3 cRNAs are transferred to glass scintillation vials and incubated under appropriate conditions to facilitate TRPML3 protein expression.

After TRPML3 sodium ion channel expression is obtained, typically around 24 hours post-cRNA microinjection, TRPML3 function is measured according to the two-electrode voltage clamp technique using an appropriate two-electrode voltage measuring device, e.g., OpusXpress 6000A parallel oocyte voltage clamp system (MDS Analytical Technologies). The two-electrode voltage clamp technique measures the macroscopic electrical current flowing across the entire oocyte membrane through the TRPML3 sodium ion channels. Oocytes are punctured with a voltage-sensing electrode and a current sensing electrode; the voltage, or potential difference across the oocyte membrane, is clamped to a particular value using the voltage-sensing electrode and the current, or the flow of ions across the oocyte membrane, required to maintain the voltage is measured using the current-sensing electrode. The OpusXpress system is one example of a commercially available two-electrode voltage measuring device which is semi-automated and which comprises a workstation that permits electrophysiological recordings to be made from eight oocytes simultaneously. This system also provides for automated oocyte impalement and delivery of target compounds by a computer-controlled fluid handler that delivers compound into 96-well compound plates. This system can best be described as a medium or moderate-throughput system as it allows for the evaluation of up to 100 compounds per week. Of course more compounds can be screened by the addition of other voltage measuring devices, as described.

In this assay system, TRPML3 enhancers will result in an increase in current passing through the TRPML3 channels in the oocyte membrane. This value is calculated by a standard formula. Such assays also may include appropriate negative controls, e.g., known TRPML3 inhibitors. Therefore, this compound functions both as an internal control to verify that oocytes express functional TRPML3, and allows for the screening of putative TRPML3 enhancers after compounds are applied (if the target compound is a TRPML3 enhancer it will result in an increase in current passing through TRPML3 channels in the oocyte membrane).

Desirably, a % enhancement factor is calculated for each enhancer. For example, a 100% enhancer increases TRPML3 activity 100% relative to the basal control value (no compound).

Negative controls are also desirably performed to confirm that oocytes which are not injected with TRPML3 cRNAs do not exhibit the same effects.

More complex analyses are also desirably performed on compounds that exhibit robust % enhancement valves e.g., current/voltage (I/V) curves, competitive experiments and dose-response curves to determine the concentration at which the compound exhibits half-maximal activity (EC50 value). These experiments will further confirm that the effect of the compound is TRPML3-specific.

These assays will provide for the identification of TRPML3 modulators, preferably TRPML3 enhancers, which may be used as additives for foods, beverages, pharmaceuticals and the like in order to modulate the salty taste associated therewith. Desirably, a TRPML3 enhancer will exhibit at least 20% enhancement factor, more preferably at least 50% and even more preferably at least an 100% enhancement factor.

The compounds tested as modulators of TRPML3 protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of a TRPML3 protein. Typically, test compounds will be small organic molecules, peptides, lipids, and lipid analogs. Preferably, the tested compounds are safe for human consumption.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including ChemDiv (San Diego, Calif.), Sigma-Aldrich (St. Louis, Mo.), Fluka Chemika-Biochemica-Analytika (Buchs Switzerland) and the like.

In the preferred embodiment, moderate or high throughput screening methods involve providing a small organic molecule or peptide library containing a significant number of potential TRPML3 modulators (potential activator or inhibitor compounds). Such “chemical libraries” are then screened in assays, as described herein, to identify those library members (particular chemical species or subclasses) that display a desired characteristic activity. The compounds thus identified can serve as conventional “lead compounds” or can themselves be used as potential or actual products. As noted, the preferred oocyte two-voltage clamp electrode system (a single device) permits about 60 compounds to be tested per week.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Intj. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Compounds are screened, e.g., using high throughput screening (HTS), to identify those compounds that can bind to and/or modulate the activity of a taste receptor or taste ion channel polypeptide or transporter or fragment thereof. In the present invention, proteins are recombinantly expressed in cells, e.g., mammalian cells, or frog oocytes and the modulation of activity is assayed by using any measure of ion channel, receptor or transporter function, such as measurement of the membrane potential, or measures of changes in intracellular sodium or lithium levels. Methods of assaying ion, e.g., cation, channel function include, for example, patch clamp techniques, two electrode voltage clamping, measurement of whole cell currents, and fluorescent imaging techniques that use ion sensitive fluorescent dyes and ion flux assays, e.g., radiolabeled-ion flux assays or ion flux assays.

### Food and Beverage Compositions Containing TRPML3 Modulatory Compound Identified Using Disclosed Assays

The compounds identified using assays which identify TRPML3 modulatory compounds are potentially useful as ingredients or flavorants in ingestible compositions, i.e., foods and beverages as wells as orally administered medicinals. Compounds that modulate or enhance salty taste perception can be used alone or in combination as flavorants in foods or beverages. In the preferred application, the modulator will be incorporated into a food or beverage with a reduced level of sodium and the salty taste of the resulting product will be similar to that of the high sodium product. Examples of such foods and beverages include snack foods such as, potato chips, crackers, soups, dips, soft drinks, packaged meat products, pretzels among others.

The salty taste flavor enhancers or blockers identified according to the present invention can be blended in various foods and beverages. Other examples of the foods and beverages include a wide range of foods and beverages, for example, beverages such as fruit juice beverage, sports drink, vegetable juice, fermented lactic drink, carbonated beverage, coffee, cocoa, black tea, oolong tea, green tea, sake, alcohol, and powdered drink; confectionery products such as candy, chewing gum, tabletted candy, gummy candy, soda-pop candy, and chocolate; bakery products such as cookie, biscuit, and bread; deserts such as yoghurt and ice cream; snacks such as potato chips and cracker; stew, curry, soup, dressing, dip, noodle soup, bouillon stock, miso, instant bouillon, sauce, bouillon, jam, sprinkling topping, Japanese pancake, miso soup, pickles, rice-ball topping, topping for tea and rice, semi-cooked or cooked foods such as wheat, buckwheat, and Chinese noodles, or the chilled and frozen foods thereof; instant foods such as instant noodle; and seasoning such as mixed powdery seasonings and mayonnaise.

Among the foods and beverages to which a salt flavor enhancer is compounded, particularly favorable for improvement in body are nutritious and nourishing drinks, functional drinks including nutrition supplement drinks, snack products such as potato chips and flavored cracker, savory processed foods such as curries, stews and soups, and the like. The forms of the savory processed food above include cooked and semi-cooked foods and the retort-pouch, chilled or frozen foods thereof.

The amount of the salt flavor enhancer or blocker of the present invention blended may vary according to the form of the salt flavor enhancer or blocker and the food or beverage to be blended with, but is preferred in the range of 0.000001 to 1.0 wt %, more preferably 0.0001 to 0.1 wt %, and still more preferably 0.00001 to 0.01 wt % with respect to the food or beverage. The salt flavor enhancer or blocker of the present invention can be blended by any one of known methods.

Alternatively, compounds that block or inhibit salty taste perception can be used as ingredients or flavorants in foods that naturally contain high salt concentrations in order to block or camouflage the salty taste thereof. These materials include sports beverages and other compositions wherein a high amount of electrolytes including sodium are present, e.g., for medicinal or replacement purposes after sickness or vigorous exercise which depletes the electrolyte balance.

The compositions for ingestion which may include a TRPML3 modulatory compounds according to the invention will include compositions for ingestion by humans, animals (domesticated, zoo animals, pets) and will include foods, beverages, medicaments, nutriceuticals and cosmetics. The amount of such TRPML3 modulatory compound(s) will be an amount that yields the desired degree of salty taste perception. Of course compounds used in such applications will be determined to be safe for human consumption and to be acceptable in human taste tests.

### Medicaments Containing TRPML3 Modulatory Compound Identified Using Disclosed Assays

The compounds identified using assays which identify TRPML3 modulatory compounds are potentially useful as therapeutics e.g. for regulating sodium homeostasis, fluid retention, plasma osmolarity, blood pressure, ENaC function, sodium homeostasis, including blood pressure regulation in hypertension (high blood pressure) and hypotension (low blood pressure), can be used to modulate plasma osmolarity, including decreases in osmolarity (hypoosmotic) and increases in osmolarity (hyperosmotic) via reabsorption of water in the kidney in normal and diseased states such as diabetis insipidus; can be used to modulate ENaC function and/or trafficking; can be used to modulate aquaporin-2 function and/or trafficking; can be used to modulate vasopressin (antidiuretic hormone) secretion from the pituitary gland; can be used to modulate aldosterone secretion from the adrenal glands and other functions and diseases involving TRPML3 function. These compounds will be administered by any suitable means including orally, via injection, transdermal, inhalation, rectal, et al. These compounds will be formulated using suitable carriers, excipients, etc. well known in the art. These formulations may be formulated for immediate release, extended release, or an intermediate thereof. The amount of the TRPML3 compound therein will comprise a dosage sufficient to elicit a desired therapeutic or prophylactic response on TRPML3 function, such as modulating blood pressure within desired ranges. In addition these formulations may contain other actives such as diuretics, blood pressure regulators, cardiac drugs, et al. depending on the particular condition involving TRPML3 function being treated.

### Preferred Assay Embodiment

### Measurement of TRPML3 Currents in Oocytes Using Two-Electrode Voltage Clamp Electrophysiological Recordings

Electrophysiological Assay for Identifying TRPML3 Modulators Using Amphibian Oocytes that Express Functional Human TRPML3

The oocyte expression system has intrinsic advantages (expression levels, robust, low endogenous ion channel expression) that render it useful to examine the effects of compounds on sodium transport through TRPML3 channels. These compounds are candidates for enhancing salty taste perception. The oocyte expression system has been used earlier for the rapid and robust expression of ion channels in functional studies (Dascal, CRC Crit. Rev. Biochem. (1987) 22(4): 317-387; Wagner, et al, Cellular Physiology and Biochemistry (2000) 10:1-12; Canessa, et al, Nature (1994) 367: 463-467). Therefore, this system was selected for use in a two-electrode voltage clamp assay using methods and materials as described below.

The oocyte expression system is comprised of the following steps and methodologies, which collectively comprise the screen for TRPML3 enhancers: frog surgery and oocyte isolation, cRNA preparation, oocyte microinjection, and measurement of TRPML3 currents in oocytes using two-electrode voltage clamp electrophysiological recordings. The following references describe general practices for frog surgery and oocyte isolation (Marcus-Sekura, et al, Methods in Enzymology (1987) 152: 284-288; Goldin, Methods in Enzymology (1992) 207: 266-279), cRNA preparation (Swanson, et al, Methods in Enzymology (1992) 207: 310-319; Goldin, et al, Methods in Enzymology (1992) 207: 279-297), oocyte microinjection (Matten, et al, Methods in Enzymology (1995) 254: 458-466; Hitchcock, et al, Methods in Enzymology (1987) 152: 276-284), and two-electrode voltage clamp electrophysiological recording (Stuhmer, Methods in Enzymology (1992) 207: 319-339; Wagner, e t a 1, Cellular Physiology and Biochemistry (2000) 10:1-12). Each of these methodologies, as they pertain to the screen for TRPML3 enhancers, is described in further detail below.

### Frog Surgery and Oocyte Isolation

Female Xenopus laevis South African clawed frogs greater than or equal to 9 cm in length are obtained from NASCO (Fort Atkinson, Wis.). Frogs are anesthetized in 0.15% MS-222 (tricaine or ethyl-3-aminobenzoate methanesulfonate; Sigma) in distilled water and placed on ice. Using sterile surgical tools, sequential 1-2 cm incisions are made in the abdomen through both the outer skin layer and the inner peritoneal layer. Excised ovarian lobes (containing 1000-2000 oocytes) are placed in OR-2 calcium-free media (82.5 mM NaCl, 2 mM KCl, 1 mM MgCl₂, 5 mM HEPES pH 7.5 with NaOH) and sequentially digested with 2 mg/ml collagenase type IA (Sigma), prepared immediately before use, for 45 min followed by 1 mg/ml collagenase type IA for 15 min on a rocking platform at room temperature. After enzymatic digestion, at which point the majority of oocytes are released from the ovarian lobes, oocytes are rinsed in OR-2 without collagenase and transferred to a Petri dish containing Barth's saline (88 mM NaCl, 2 mM KCl, 0.82 mM MgSO₄, 0.33 mM Ca(NO₃)₂, 0.41 mM CaCl₂, 2.4 mM NaHCO₃, and 5 mM HEPES pH 7.5; Specialty Media) supplemented with 2.5 mM sodium pyruvate. Mature stage V or VI oocytes (˜1 mm diameter) containing distinct animal poles, corresponding to the dark side of the egg containing melanin pigment granules, and vegetal poles, corresponding to the light side of the egg containing yolk proteins, are selected for microinjection Frogs are sutured using a C6 needle with a 3-0 black braid suture (Harvard Apparatus) and reused for oocyte isolation following a 2-3 month recovery period.

cRNA Preparation

TRPML3 cRNA is generated using the mMESSAGE mMACHINE kit according to the manufacturer's instructions (Ambion) from human TRPML3 DNA plasmids described in WO 02/087306 A2 using T7 RNA polymerase to transcribe cRNA in vitro from DNA linearized with restriction enzymes. cRNA quality is checked by denaturing agarose gel electrophoresis and spectrophometric absorbance readings at 260 and 280 nm to ensure that full-length, non-degraded cRNA is generated.

### Microinjection

Microinjection needles are pulled on a Model P-97 Flaming/Brown Micropipette Puller (Sutter Instrument Co.) using borosilicate glass capillaries (World Precision Instruments Inc.), back-filled with mineral oil (Sigma), and then front-filled with TRPML3 cRNA using a Nanoliter 2000 injector with a Micro4 MicroSyringe Pump Controller (World Precision Instruments). Oocytes are microinjected in the animal pole with 10-25 nl containing 10-25 ng of human TRPML3 cRNA. Following microinjection, oocytes are transferred to glass scintillation vials containing Barth's solution supplemented with 2.5 mM sodium pyruvate and incubated at 18-19° C. overnight under normal atmospheric conditions. During this time, the oocytes translate injected TRPML3 cRNA into protein.

### Measurement of TRPML3 Currents in Oocytes Using Two-Electrode Voltage Clamp

Twenty-four to forty-eight hours following microinjection of 10-25 ng human TRPML3 cRNA, TRPML3 function is measured in oocytes using the two-electrode voltage clamp technique on an OpusXpress 6000A parallel oocyte voltage clamp system (MDS Analytical Technologies). The two-electrode voltage clamp technique is an electrophysiology method that measures the macroscopic electrical current flowing across the entire oocyte membrane though protein channels (Stuhmer, Methods in Enzymology (1992) 207: 319-339). Oocytes are impaled with a voltage-sensing electrode and a current-sensing electrode; the voltage, or potential difference across the oocyte membrane, is clamped to a particular value using the voltage-sensing electrode and the current, or the flow of ions across the oocyte membrane, required to maintain that voltage is measured using the current-sensing electrode. The OpusXpress system is a semi-automated two-electrode voltage clamp workstation that allows recordings to be made from 8 oocytes simultaneously. Oocyte impalement is automated and compound delivery is performed by computer-controlled fluid handlers from 96-well compound plates.

Oocytes are placed in the OpusXpress system and bathed in ND-96 solution (96 mM NaCl, 2.5 mM KCl, 1 mM CaCl₂, 1 mM MgCl₂, and 5 mM HEPES pH 7.5 with NaOH). Oocytes are impaled with voltage-sensing and current-sensing electrodes, pulled on a Model P-97 Flaming/Brown Micropipette Puller (Sutter Instrument Co.) using borosilicate glass capillaries (World Precision Instruments Inc.) and back-filled with 3M KCl, containing silver chloride wires. Electrodes exhibit resistances between 2-10 Mohm for voltage-sensing electrodes and between 0.5-2 Mohm for current-sensing electrodes. Following impalement, oocytes are voltage clamped to −60 mV and experimental recordings are initiated. Data are acquired at 50 Hz and low-pass filtered at 5 Hz using a 4-pole Bessel filter.

A flowchart illustrating the sequence of experiments performed to examine the effect of a compound on TRPML3 function is depicted in FIG. 10, including screening at a holding potential of −60 mV, I/V curves, NDMG competition tests, dose-response curves, and testing uninjected oocytes.

### Preferred Assay Embodiment

### Measurement of TRPML3 Currents in Oocytes Using Two-Electrode Voltage Clamp Electrophysiological Recordings and Codon Optimized TRPML3 Sequence

Codons comprise three nucleotides that encode a specific amino acid in a protein sequence. Since there are 61 different codon nucleotide triplets that encode 20 amino acids, most amino acids can be encoded by more than one codon. Codon optimization, the use of the favored codon for each amino acid in a particular species, can improve the functional expression of proteins by increasing the speed and accuracy of translation without changing the protein sequence.

A codon-optimized version of the human TRPML3 gene was generated that is 76.4% homologous to non-codon optimized TRPML3 at the DNA level (FIG. 19). Codons were optimized for optimal translation of human sequences. We also generated an active form of TRPML3 (A419P TRPML3) by mutating alanine 419 to proline in the 5^{th} transmembrane domain. This mutation results in TRPML3 channels that are in an open confirmation (Xu et al. PNAS 104(46): 18321-18326, 2007; Grimm et al. PNAS 104(49): 19583-19588, 2007; Nagata et al. PNAS 105(1): 353-358, 2008; Kim et al. J. Biol. Chem. 282(50): 36138-36142, 2007); therefore, A419P TRPML3 is particularly useful for identification of TRPML3 blockers.

Wild-type (non-codon optimized), codon-optimized, and A419P TRPML3 are expressed in oocytes and sodium currents were measured. FIG. 20 illustrates that wild-type TRPML3 yields low sodium current levels, codon-optimized wild-type TRPML3 yields intermediate sodium current levels, and A419P TRPML3 yields high sodium current levels. Thus, codon-optimized wild-type TRPML3 and A419P TRPML3 facilitate screening for compounds that modulate TRPML3 function.

Codon-optimized wild-type TRPML3 can be used to screen for compounds that open (enhance) TRPML3 function as described previously. The data in the examples infra demonstrates how the electrophysiology oocyte assay using these sequences can be used to identify TRPML3 enhancers that are candidate human salty taste enhancers, and how wild-type codon-optimized TRPML3 facilitates identification of TRPML3 enhancers in the oocyte electrophysiology assay.

### Preferred Assay Embodiment

### TRPML3 Mammalian Cell Electrophysiological Assays

The assays may be effected using different mammalian cells. In a preferred exemplary embodiment immortalized mammalian cells or tissue culture cells such as human embryonic kidney cells (HEK293 cells) or Chinese hamster ovary cells (CHO cells) are used to examine the effects of compounds on sodium transport through exogenously expressed human TRPML3 cation channels. These compounds are candidates for modulating salty taste perception. The expression of ion channels in mammalian tissue culture cell lines is widely used for the rapid and robust expression of ion channels for functional studies. Advantages of using cultured mammalian cells as an expression system include: multiple and well established methods for introducing cDNA of interest into cells including the ability to generate stable cell lines, relative ease to perform patch clamp experiments, high level of currents in comparison to the current stemming from an endogenously expressed ion channel; the ability to directly measure ion channel function.

Electrophysiological recording from mammalian tissue culture cell lines is comprised of the following steps and methodologies, familiar to those skilled in the art of electrophysiology, tissue culture and molecular biology: cell maintenance in culture, cDNA preparation and purification, introduction of cDNA into cells by transfection and/or viral transduction, stable clone selection, and patch-clamp electrophysiology. The following references describe general practices for patch clamp electrophysiology of mammalian cells (Sackman B. and E. Neher (eds.). 1995. Single-channel recording, 2^{nd} Ed.; Hille B. 2001. Ion channels of excitable membranes, 3^{rd} Ed.).

### Measurement of TRPML3 Currents in Mammalian Cells Using Whole Cell Voltage Clamp Electrophysiological Recordings

The whole cell voltage clamp technique is an electrophysiology method that measures the macroscopic electrical current flowing across the entire plasma membrane though protein channels. Live mammalian cells are placed in a special microscope chamber containing extracellular solution. Guided by a micromanipulator and under visual control, a small diameter glass pipette filled with an electrically conductive salt solution is first attached to the cell membrane using gentle negative pressure resulting in a high resistance gigaOhm seal. The membrane patch within the pipette is disrupted using further suction resulting in the whole cell patch clamp configuration. The whole cell patch clamp configuration allows the combined measurement of all ion channels proteins within the membrane or macroscopic current. Using the patch clamp amplifier in combination with the whole-cell patch clamp configuration allows the operator to control the voltage, or potential difference across the entire cell membrane, as well as the both the internal and external ionic composition of the cell. Thus the technique provides a highly sensitive and flexible platform for the biophysical study of ion channels properties including (but not limited to) voltage dependence, activation and deactivation kinetics and permeability to different ions as well as a screening platform for ion channel blockers, enhancers and modulators. Utilization of the computer controlled patch clamp amplifier in concert with a valve controller allows for voltage protocols to be automatically executed and for the extracellular solution to be rapidly exchanged. Thus a single cell may be subject to multiple voltage protocols and compound additions.

Prior to any electrophysiological assay the efficient delivery of TRPML3 cDNA into mammalian tissue culture cells must be obtained. This may be achieved in at least three ways: 1) transient transfection of TRPML3 cDNA using lipid based methods 2) transduction using viral infection such as baculovirus, adenovirus, and lentivirus 3) stable expression of cDNA through the stable incorporation of TRPML3 into a chromosome and selection of clones expressing TRPML3. The electrophysiological protocols utilized to screen mammalian cells for enhancers and blockers of TRPML3 currents are analogous to those previously described for Xenopus oocytes. In brief these include current voltage analysis, NMDG competition and dose responses for candidate TRPML3 blockers and enhancers. In addition, the whole-cell patch clamp electrophysiological technique can overcome some limitations imposed by two electrode voltage clamping of oocytes. For example, the smaller size of mammalian cells allows for more detailed biophysical analysis of fast processes such as the effects of compound on activation and deactivation kinetics. Also, the ability to control the intracellular solution of the cell allows measurement of any changes in channel permeability due to compound addition. Finally, the ability for cell attached, inside-out and outside-out patch configurations allows for the ability to measure single channel currents allowing detailed characterization of the mechanism of action of any enhancer or blocker.

Use of CHO Cells for Functional Expression of Wild Type TRPML3 and Screening of TRPML3 Modulators.

Previous reports show little or no function for wild type TRPML3 in HEK293 cells using patch clamp assay (Xu et al. PNAS 104(46): 18321-18326, 2007; Grimm et al. PNAS 104(49): 19583-19588, 2007; Nagata et al. PNAS 105(1): 353-358, 2008; Kim et al. J. Biol. Chem. 282(50): 36138-36142, 2007). In contrast the A419P mutant TRPML3 is believed to be unregulated in HEK1293 cells resulting in robust currents when transiently expressed. In FIG. 24A, HEK1293 cells are transiently transfected with WT and A419P mutant TRPML3 and their currents assayed by a series of voltage steps from −100 to +60 mV in order to generate a current-voltage relation plot (I/V plot). Expression of A419P mutant TRPML3 channel results in large, inward rectifying currents compared to WT. We describe in the examples infra the use of an alternative cell line such as CHO cells that allow for increased function of WT TRPML3 (FIG. 24 B). In addition, we show the average macroscopic currents and inward rectification are the same for WT and A419P TRPML3 channels. Thus, the use of specific cell lines such as CHO cells allows for more efficient functional expression of WT TRPML3 in a mammalian system providing a platform for salty taste modulator screening. In FIG. 25, we demonstrate the practice of using WT and A419P mutant TRPML3 channels expressed in CHO cells to test and study potential salty taste enhancers and blockers. In whole cell electrophysiological assays, an enhancer of TRPML3 would be observed as an increase in inward current when the cell is voltage clamped to negative potentials. The I/V analysis in FIG. 25 A shows WT TRPML3 being enhanced by compound. In the same assay, a blocker of TRPML3 results in a reduction of inward current at negative potentials. In FIG. 25 B, the A419P mutant TRPML3 channel expressed constitutively in CHO cells is blocked by the compound Gadolinium chloride, an inhibitor of many ion channels.

Use of Codon Optimized cDNA for Efficient Expression of TRPML3 in Mammalian Cells and Screening of Salty Taste Modulators.

Codons comprise three nucleotides that encode a specific amino acid in a protein sequence. Since there are 61 different codon nucleotide triplets that encode 20 amino acids, most amino acids can be encoded by more than one codon. Codon optimization, the use of the favored codon for each amino acid in a particular species, can improve the functional expression of proteins by increasing the speed and accuracy of translation without changing the protein sequence. As stated previously a codon-optimized version of the human TRPML3 gene was generated that is 76.4% homologous to non-codon optimized TRPML3 at the DNA level (FIG. 19). Codons were optimized for optimal translation of human sequences. Previously, we showed the WT TRPML3 channel did not express functional channels efficiently in HEK293 cells (FIG. 24 A). We demonstrate in FIG. 26 that use of the codon optimized TRPML3 largely overcomes the expression problems observed in HEK1293 cells. When codon-optimized WT TRPML3 is expressed in HEK1293 cells by either transient transfection or using baculovirus transduction (FIG. 26 B), robust currents are observed with similar properties as the A419P TRPML3 mutant channel. Thus the use of codon optimized TRPML3 allows for improved functional expression of WT TRPML3 ion channels in HEK293 cells providing a platform for the screening of salty taste modulators. In FIG. 26C, we demonstrate the practice of using codon optimized WT TRPML3 channels to test and study potential salty taste modulators. In this experiment codon optimized WT TRPML3 cDNA is delivered to mammalian cells via transduction by baculovirus. Codon optimized WT TRPML3 mediated inward currents are enhanced by the TRPML3 activating compound (FIG. 26C)

### Use of TRPML3 Heteromultimers for Expression and Screening of TRPML3 Modulators.

The TRPML3 ion channel subunit is a member of the larger 6TMD ion channel family of ion channel subunits. Similar to other 6TMD ion channels, it is believed that up to 4 TRPML subunits are necessary to generate a single ion channel (Hille B. 2001. Ion channels of excitable membranes, 3^{rd} Ed.; Venkatachalam et al. J Biol. Chem. 2006 Jun. 23; 281(25):17517-27). Functional channels may be composed entirely of the same subunit (homomeric) or associate with closely related subunits (heteromeric). A common feature of heteromeric channels is that they often posses intermediate biophysical functions as compared with their homomeric counterparts, thus increasing the potential functional diversity of the channel. Activity may also be modulated by different composition of subunits through changes in plasma membrane trafficking, and post-translational modification, such as phosphorylation, ubiquination, and glycosylation. The study of heteromeric channels in mammalian cells can be achieved by the delivery of multiple channel subunit cDNAs via co-transfection in mammalian cells or co-injection of cRNA in Xenopus oocytes. In addition, multimerization can also be achieved by covalently linking channel subunit cDNAs together, generating stable cell lines expressing multiple cDNAs, or viral transduction with multiple viruses which deliver cDNA for multiple channel subunits.

In practice, multimerization of WT and A419P TRPML3 channel subunits can be used to increase the level of surface activity (FIG. 27). As already shown, channels consisting exclusively of A419P TRPML3 subunits express functional channels in HEK293 cells (FIG. 27 A). In the same cells, when non codon optimized WT TRPML3 subunits are expressed, no currents are observed even when using 3-fold the amount of cDNA (FIG. 27 B). In contrast, coexpression of A419P TRPML3 cDNA with WT TRPML3 cDNA in HEK1293 cells results in a cooperative effect resulting in larger currents than would be predicted by simple addition of two separate channel populations (FIG. 27 C-D). This data suggests that functional channels consisting of WT and A419P mutant subunits exist at the membrane and may be utilized for TRPML3 enhancer and blocker assays. As shown in this example, using limited amounts of A419P cDNA versus WT should increase the proportion of WT TRPML3 subunits within the channel tetramer, possibly conferring intermediate biophysical function to the channel. Therefore since it has been suggested that homomeric A419P TRPML3 channels may already have a high probability of opening (Po) (Xu et al. PNAS 104(46): 18321-18326, 2007), use of heteromeric A419P/WT TRPML3 channels may be more suitable for the screening of TRPML3 enhancers.

### Preferred Assay Embodiment

### Monitoring of Variant TRPML3 Function Using Membrane Potential Dyes

Specific cell-based assays for the discovery of TRPML3 modulators were developed which that could ultimately be used in food and beverages to modulate saltiness perception. A419P-TRPML3 function was monitored in HEK1293 cells transiently transfected or transduced with the gene encoding A419P-TRPML3 and using specific membrane potential dyes (FMPs; Molecular Devices).

In one embodiment of the invention, the mammalian or frog oocyte cell expressing the TRPML3 or a variant, fragment or functional equivalent is preloaded with a membrane potential fluorescent dye or a sodium fluorescent dye. The cell is then contacted with a TRPML3 putative modulator compound in the presence of sodium or lithium. Cation-mediated changes in fluorescence of the cell in the presence of the putative modulator are compared to changes in the absence of the modulator to determine the extent of TRPML3 modulation.

Alternatively, the mammalian cell may be transfected with a functional TRPML3 splice variant and fragments. The cells are then seeded in the well of a multi-well plate and incubating for a time sufficient to reach at least about 70% confluence. The cells are then dye-loaded with a membrane potential dye and contacted with at least one putative modulating compound and sodium. Any changes in fluorescence of the membrane potential dye due to modulator/TRPML3 interactions are monitored using a fluorescence plate reader or voltage intensity plate reader. A putative modulator of salty taste may then be identified by the changes in fluorescence.

### Preferred Assay Embodiment

### IonWorks TRPML3 Patch Clamp Assay

The IonWorks automated patch clamp system is used to examine the effects of compounds on sodium transport through human TRPML3 cation channels. These compounds are candidates for modulating salty taste perception. The IonWorks system is widely used for high-throughput electrophysiology. With its 384-well format the IonWorks system can examine thousands of compounds per day, the highest throughput of any automated electrophysiology system. The IonWorks instrument has the advantage that it can be run in the standard mode where each well corresponds to a single cell or in the population patch clamp (PPC) mode where each well gives the average current of 64 cells thus increasing the overall success rate and reducing well to well variability. Other advantages of the IonWorks system are that it uses mammalian cells and it provides a direct measurement of ion channel function by recording ion currents.

### Measurement of TRPML3 Currents in CHO-1K1 Cells Using the IonWorks Patch Clamp Assay

Different versions of human TRPML3 can be assayed, included but not limited to a wild-type version and a gain of function mutant version encoding an A419P substitution. TRPML3 can be expressed in CHO-K1 cells by one of three methods: (i) transient transfection (ii) BacMam transduction or (iii) a stable transfection and TRPML3 function measured using the perforated patch clamp technique on an IonWorks Quattro instrument (MDS Analytical Technologies). Cells are dissociated with Detachin cell detachment solution (Genlantis), centrifuged, and resuspended in external recording buffer (150 mM NaCl, 2 mM KCl, 1.5 mM CaCl2, 1 mM MgCl2, 10 mM HEPES pH 7.4 with NaOH). Dissociated cells are added to the 384-well PPC plate where each well has 64 holes in the substrate with each hole having a diameter of 1-2 μm. One cell lands on each hole and negative pressure is used to form a high mega-Ohm seal between the cell and the hole. After the seal has formed the perforated patch clamp technique is used to gain electrical access to the inside of the cell. In this technique, amphotericin, a pore-forming antibiotic, is applied below the hole and forms small pores on an isolated, exposed section of the plasma membrane resulting in electrical access to the cell from below the PPC plate. Once access to the cell is obtained, experimental recordings are initiated. Two types of electrodes, a common ground electrode below the plate and separate recording electrodes dipped into each well, allow for control of the voltage (the potential across the cell membrane) and for recording the flow of ionic current across the membrane of the entire cell. The IonWorks Quattro system is a semi-automated patch clamp workstation that allows recordings to be made from 384 wells. Dispensing of cells to the patch plate, seal formation and electrical access are automated while compound delivery is performed by computer-controlled fluid handlers from 384-well compound plates.

The following description illustrates the IonWorks screening assay to identify compounds that modulate (activate or block) TRPML3 function. For a well in a plate to provide data two criteria must be met: (i) initially, the well must have most of its holes open (the average resistance across the 64 holes must be >1 mOhm and <10 mOhm) and (ii) after cell addition, most holes must have a cell forming a high mOhm seal with the patch plate (the average resistance across the 64 holes must be >10 mOhm). If these two criteria are met, the instrument collects data from pre-compound and post-compound scans. In the example shown (FIG. 28), 94% of wells have a resistance greater than 10 mOhm. In each scan currents are measured as the voltage is modified (see example voltage command trace in FIG. 29).

Since TRPML3 channels exhibit inward rectification, the recordings will show large inward currents at hyperpolarized potentials and small outward currents at depolarized potentials (FIG. 29). Compounds will be applied at concentrations between ˜1 uM and ˜100 uM. If the compound functions as a TRPML3 enhancer, the current passing through TRPML3 channels in the cell membrane increases. If the compound functions as a TRPML3 blocker, the current passing through TRPML3 channels in the membrane decreases. TRPML3 currents can be examined at two different voltages: for example, −120 mV and −40 mV. Compounds that enhance TRPML3 by affecting the voltage dependent rectification will be preferentially detected at −40 mV where the rectification is strong while compounds that block TRPML3 will be preferentially detected at −120 mV where the TRPML3 currents are larger. To quantitate the effect of a compound on TRPML3 function, we use the following formula: [(A−Ao)/(B−Bo)]×100. B and Bo are the currents measured before compound addition while A and Ao are the currents measured after compound addition. A and B are the currents at the test voltage (either −120 mV or −40 mV) while Ao or Bo is the current at 0 mV. This value leads to a % modulation factor that is used to gauge the activity of compounds in our assay. For example, if the % modulation factor is equal to 200%, then the compound doubles TRPML3 activity compared to control values in the absence of compound. If the % modulation factor is equal to 50%, then the compound decreases TRPML3 activity by one-half over basal control values (in the absence of compound).

Negative control experiments are performed in parental cells to demonstrate that effects observed with compounds in TRPML3 expressing cells are due to currents flowing through TRPML3 channels and not due to currents flowing through channels endogenously expressed in the cell membrane. Compounds specifically modulating TRPML3 should not affect currents in control CHO-K1 cells and should exhibit % modulation factors near 100%.

More complex analyses are performed on compounds displaying large % modulation factors and having no effect on control CHO-K1 cells. The assays include current/voltage (I/V) curves, GdCl3 competition experiments (GdCl3 is a blocker of TRPML3), and dose-response curves. For I/V curves, currents are measured in voltage steps from −120 to +60 mV, in 10 mV increments in the presence and absence of compound, to investigate the magnitude of compound modulation. The slope of the I/V curve is indicative of the magnitude of current modulation by the compound of interest. Strong enhancers increase the slope of I/V curves, indicative of increased opening of TRPML3 ion channels. Strong blockers decrease the slope of I/V curves, indicative of increased closing of TRPML3 ion channels. Control I/V curves performed in the presence of compound should be identical and superimposable with I/V curves performed in the absence of compound.

GdCl3 competition experiments are performed to demonstrate that compound effects are TRPML3 dependent. First, compound is applied to determine the % modulation factor then a saturating dose of GdCl3 (or some other TRPML3 blocker) is applied. For an enhancer to work directly on the TRPML3 channel, currents from cells treated with enhancer plus GdCl3 should resemble currents seen for cells treated only with GdCl3. This experiment shows that when the channel is blocked the compound does not have an enhancing effect; therefore, the compound must directly modulate TRPML3 channel function.

Dose-response curves are performed to determine the concentration at which the compound exhibits half-maximal activity (EC50 for enhancers and IC50 for blockers). The lower the EC50 or IC50 value, the more active the compound is as a TRPML3 modulator. Dose-response curves are performed by sequentially applying increasing concentrations of compound starting from low doses (˜1 nM) and progressing to high doses (˜1 mM). % modulation factors are calculated as described above and plotted as a function of compound concentration on a logarithmic scale to determine an EC50 or IC50 value for the compound.

A flowchart illustrating the sequence of experiments performed to examine the effect of a compound on TRPML3 function is depicted in FIG. 30, including screening at a holding potential of −120 mV and −40 mV, I/V curves, GdCl3 competition tests, dose-response curves, and negative control experiments.

### Animal Models

Animal models also find potential use in screening for modulators of gene activity. Transgenic animal technology results in gene overexpression, whereas siRNA and gene knockout technology results in absent or reduced gene expression following homologous recombination with an appropriate gene targeting vector. The same technology can also be applied to make knock-out cells. When desired, tissue-specific expression or knockout of the target gene may be necessary. Transgenic animals generated by such methods find use as animal models of responses related to the gene target. For example such animals expressing a gene or genes according to the invention may be used to derive supertaster phenotypes such as for use in screening of chemical and biological toxins, rancid/spoiled/contaminated foods, and beverages or for screening for therapeutic compounds that modulate taste stem cell differentiation.

Knock-out cells and transgenic mice can be made by insertion of a marker gene or other heterologous gene into an endogenous gene site in the mouse genome via homologous recombination. Such mice can also be made by substituting an endogenous gene with a mutated version of the target gene, or by mutating an endogenous gene, e.g., by exposure to known mutagens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual (1988) and Teratocarcinomas and Embryonic Stem Cells: A Practical Approach (Robertson, ed., 1987).

### Preferred Animal Model Assay Embodiment

### Varitint-Waddler Mice Studies

This invention also contemplates the use of a mouse model, termed the Varitint waddler mouse, or similar transgenic animals where TRPML3 salty taste cells are specifically ablated from taste buds and where salty taste is greatly diminished to study the effect of TRPML3 in vivo on salty taste and other functions relating to sodium metabolism as well as the use of this gene mutation in creating animals depleted in specific cell types such as salty taste cells, melanocytes, pituitary cells, and adrenal cells.

The Varitint waddler mouse has a gain of function A419P mutation in the TRPML3 ion channel (Di Palma et al PNAS 99(23): 14994-14999, 2002) that arose from a spontaneous mutation in 1942 (Cloudman et al J. Heredity 36: 258-263, 1945). These mice are termed Varitint waddler due to their two most obvious phenotypes: a variegated coat color (variable tint of fur) and deficiency in the vestibular system (circling behavior and waddling that resembles a duck). The increased activity of A419P TRPML3 alters the ionic equilibrium of cells expressing TRPML3, including melanocytes in the skin as well as hair cells in the inner ear and vestibular system, and results in death of these cell populations (Xu et al. PNAS 104(46): 18321-18326, 2007; Grimm et al. PNAS 104(49): 19583-19588, 2007; Nagata et al. PNAS 105(1): 353-358, 2008; kim et al. J. Biol. Chem. 282(50): 36138-36142, 2007). Cell death is likely attributable to uncontrolled entry of sodium and/or calcium ions into the cytoplasm. Thus, the Varitint waddler mouse is a model for cell ablation where cells expressing A419P TRPML3 die off.

Since TRPML3 is specifically expressed in taste cells on the tongue, experiments were performed to determine if taste cells expressing TRPML3 were ablated in the Varitint waddler mouse. TRPML3 is not detectable in purified taste cells from Varitint waddler mice using end-point PCR (FIG. 31) or real-time quantitative PCR (FIG. 32). By contrast, expression of genes involved in sweet, bitter, umami, and sour taste are similar in Varitint waddler and wild-type control mice (FIGS. 31-32). FIG. 33 illustrates that TRPM5 (sweet, bitter, umami, GPR113) and PKD2L1/PKD1L3 (sour) taste cells are intact in Varitint waddler mice using the histological in situ hybridization technique. Therefore, taste cell populations that do not express TRPML3 (including sweet, bitter, umami, GPR113, and sour) are unaffected in the Varitint waddler mouse model. Therefore, Varitint waddler mice contain taste buds lacking TRPML3 taste cells and Varitint waddler mice can be used to study salty taste in the absence of this cell population.

In addition, electrophysiological CT nerve recordings are an established method to study taste biology in rodent systems and have been used to elucidate the effect of genetic mutations on physiological responses to diverse taste stimuli (Damak et al, Science. 2003 301(5634):850-3; Lyall et al, J. Physiol. 2004 558(Pt 1):147-59; Zhao et al, Cell. 2003 115(3):255-66; Mueller et al, Nature. 2005 434(7030):225-9). The CT nerve innervates the anterior tongue encompassing taste buds in fungiform and some foliate taste papilla; thus, CT nerve activity represents a measure of taste receptor cell function in response to tastant application to the front of the tongue. CT nerve recording methodology was carried out as previously described (Lyall et al, J. Physiol. 2004 558(Pt 1):147-59; Treesukosol et al, Am J Physiol Regul Integr Comp Physiol. 2007 May; 292(5):R1799-809; Dahl et al, Brain Research. 1997 756:22-34), using procedures familiar to those skilled in the art.

Using CT nerve recordings, Varitint waddler mice were shown to exhibit a deficiency in the response to sodium chloride. Specifically, Varitint waddler mice have a greatly reduced benzamil-insensitive CT nerve response to sodium chloride (FIG. 34). Since TRPML3 is not blocked by amiloride or the amiloride analog benzamil, the benzamil-insensitive CT response is largely attributable to TRPML3. Both the initial (phasic) and sustained (tonic) components of the CT nerve response were attenuated in Varitint waddler mice. These data indicate that elimination of TRPML3 taste cells substantially reduces the ability of mice to taste salt, and point to a central role of TRPML3 taste cells as professional salty taste cells.

These results further show that the Varitint waddler mice have taste buds in which TRPML3 taste cells are specifically ablated and that these mice can be used in taste studies wherein salty taste is specifically affected.

Also, these results show that Varitint waddler mice exhibit a deficiency in the benzamil-insensitive CT nerve response to sodium chloride and Varitint waddler mice exhibit a deficiency in the initial (phasic) and sustained (tonic) components of the CT nerve response to sodium chloride.

Importantly, these results show that the expression of A419P TRPML3 can be used in order to specifically ablate cell types and create mouse model systems lacking different cell populations.

Also, these animals can be used to study the effect of A419P TRPML3 as a toxin to kill specific cell types.

### Candidate Modulators

The compounds tested as modulators of the putative taste related proteins or other non-taste related functions and phenotypes involving taste cells can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of a protein. Typically, test compounds will be small organic molecules, peptides, lipids, and lipid analogs. In one embodiment, the compound is a menthol analog, either naturally occurring or synthetic.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md.). C. Solid State and Soluble High Throughput Assays

Additionally soluble assays can be affected using a target taste specific protein, or a cell or tissue expressing a target taste protein disclosed herein, either naturally occurring or recombinant. Still alternatively, solid phase based in vitro assays in a high throughput format can be effected, where the protein or fragment thereof, such as the cytoplasmic domain, is attached to a solid phase substrate. Any one of the assays described herein can be adapted for high throughput screening, e.g., ligand binding, calcium flux, change in membrane potential, etc.

In the high throughput assays of the invention, either soluble or solid state, it is possible to screen several thousand different modulators or ligands in a single day. This methodology can be used for assaying proteins in vitro, or for cell-based or membrane-based assays comprising a protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferring, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immunol. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

Having described the invention supra, the examples provided infra further illustrate some preferred embodiments of the invention. These examples are provided only for purposes of illustration and should not be construed as limiting the subject invention.

The following examples relating to TRPML3 provide confirmatory evidence suggesting that TRPML3 encodes a salty taste receptor polypeptide and is involved in sodium homeostasis and related physiological functions and were affected using the materials and methods described supra. These examples are put forth so as to provide those of ordinary skill in the art with a complete disclosure and description of how to make and use the subject invention, and are not intended to limit the scope of what is regarded as the invention.

### EXAMPLES

### Example 1

This example relates to the experiments and molecular biology data which are contained in FIG. 1 that show that TRPML3 is a taste-specific gene. RT-PCR of human (left) and monkey (right) taste buds (taste) and lingual epithelial cells (lingual) collected by laser capture microdissection was affected. FIG. 1 shows that TRPML3 is only expressed in taste cells, similar to the known taste-specific genes T1R2 and TRPM5. The Figure also shows that the housekeeping gene beta-actin is expressed in both taste and lingual cells demonstrating that RNA from both samples is of high quality. ‘+’ indicates reverse transcription was performed and ‘−’ indicates that no reverse transcription was performed (negative control). Bands are only observed with reverse transcription. All bands were cloned and sequenced to confirm gene identities.

### Example 2

This example contains the electrophysiological assays contained in FIG. 2 which reveal that TRPML3 forms a sodium channel. Whole cell patch clamp electrophysiology of cells expressing human TRPML3 was affected as depicted therein. It can be seen that TRPML3 generates a sodium leak current that is blocked upon removal of sodium and replacement with the large impermeant cation NMDG. The top trace in the same Figure shows current at a holding potential of −60 mV. The middle traces in FIG. 2 show current-voltage traces from −100 mV to +60 mV in the presence (NaCl) and absence (NMDG-Cl) of sodium. The bottom graph in the Figure shows current voltage curves in the presence (dark blue line; diamonds) and absence (magenta line; squares) of sodium. It can be seen that TRPML3 exhibits inward rectification (more current at negative voltages compared to positive voltages).

### Example 3

This example relates to the electrophysiological assays the results of which are contained in FIG. 3. These results obtained using the human TRPML3 channel properties are consistent with human salty taste psychophysics. The top graph in the Figure contains current-voltage curves showing TRPML3 sodium conductance (dark blue line; diamonds) is not blocked by 30 uM amiloride (magenta line; squares). Both human salty taste and TRPML3 are not blocked by amiloride. The bottom graph in the same figure contains current-voltage curves showing TRPML3 is equally permeable to the salty cations sodium (dark blue line; diamonds) and lithium (magenta line; squares). This is consistent with TRPML3 encoding a human salty taste receptor since sodium and lithium are equally salty to humans and both cations permeate the human TRPML3 channel.

### Example 4

This example relates to the immunohistochemical labeling experiments contained in FIG. 4. It can be seen therein that the TRPML3 protein is expressed in the apical membrane region near the taste pore. Particularly, it is seen that the TRPML3 antibody labels taste cell processes extending to the taste pore (left image). In addition magnification of the apical taste bud domain facing the saliva clearly demonstrates that the TRPML3 protein is expressed at the taste pore region (3 right images; taste pore denoted with blue arrows). This location is ideally suited for TRPML3 to sense sodium in the saliva. Similar to TRPML3, other taste receptors (sweet, bitter, umami, and sour) are also polarized to the taste pore where they sample saliva for their requisite tastants

### Example 5

This example relates to the immunochemical labeling experiments contained in FIG. 5. These results show that the TRPML3 protein is not expressed in TRPM5 cells. Specifically, double label immunohistochemistry with TRPM5 (green; left images) and TRPML3 (red; middle images) in monkey CV papilla was effected. It can be seen from the figure that cells expressing TRPM5 and TRPML3 are distinct (merged images on the right). These data indicate that TRPML3 is not expressed in TRPM5 cells (encompassing sweet, bitter, and umami cells) but only in professional salty taste cells.

### Example 6

This example relates to the immunohistochemical labeling experiments contained in FIG. 6. These results show that the TRPML3 protein is not expressed in PID2L1 cells. Double label immunohistochemistry was effected with PKD2L1 (green; left images) and TRPML3 (red; middle images) in monkey CV papilla. It can be seen from the figure that the cells expressing PKD2L1 and TRPML3 are distinct (merged images on the right). These data indicate that TRPML3 is not expressed in PID2L1 cells (encompassing sour cells) but in professional salty taste cells.

### Example 7

I/V curves in oocytes injected with human TRPML3 cRNA.

The following example illustrates the oocyte screening assay to identify compounds that modulate (activate or block) TRPML3 function. Oocytes expressing TRPML3 are identified by I/V curve analysis and sodium-replacement with NMDG. See FIG. 7. Currents are measured in voltage steps from −100 to +60 mV, in 10 mV increments. Since TRPML3 channels exhibit inward rectification, oocytes with large inward currents at hyperpolarized potentials and small outward currents at depolarized potentials express TRPML3 channels. NMDG is a large cation that cannot permeate TRPML3. Thus, current inhibited by NMDG represents the TRPML3-dependent sodium current. Inhibition of current with NMDG is used as another internal control to verify that the oocytes express functional TRPML3 protein.

The inwardly rectifying I/V curves, denoted by more current at hyperpolarized potentials (more negative potentials) and less current at depolarized potentials (more positive potentials), indicate that oocytes express TRPML3 ion channels. Replacement of sodium with NMDG blocks TRPML3 current. Subtraction of I/V curves in sodium and NMDG yields the TRPML3 specific sodium current. Diamonds (♦) denote I/V curve in sodium (NaCl) solution; squares (▪) denote I/V curve in sodium-free NMDG solution; triangles (▴) denote subtraction of NaCl and NMDG I/V curves and represents the TRPML3-specific sodium current.

### Example 8

Screening oocytes injected with human TRPML3 cRNA for compounds that may modulate TRPML3 activity.

A compound at a concentration between ˜1 μM and 100 μM is applied to oocytes exhibiting TRPML3 function. See FIG. 2. If the compound functions as a TRPML3 enhancer, the current passing through TRPML3 channels in the oocyte membrane increases (becomes more negative). If the compound functions as a TRPML3 blocker, the current passing through TRPML3 channels in the oocyte membrane decreases (becomes less negative). To quantitate the effect of a compound on TRPML3 function, the following formula is used:

[(A−Ao)/(B−Bo)]×−100

A is the current following compound treatment, Ao is the current preceding compound treatment, B is the current following NMDG treatment, and Bo is the current preceding NMDG treatment. This value leads to a % modulation factor that is used to gauge the activity of compounds in the assay. For example, if the % modulation factor is equal to 100%, then the compound increases TRPML3 activity 100% over basal control values (in the absence of compound). If the % modulation factor is equal to −100%, then the compound decreases TRPML3 activity 100% over basal control values (in the absence of compound). % modulation factors are calculated individually for each of the oocytes in the OpusXpress system and then an average and standard deviation are determined for each compound.

Negative control experiments are performed in oocytes not injected with TRPML3 cRNA to demonstrate that effects observed with compounds in TRPML3 expressing oocytes are due to currents flowing through TRPML3 channels and not due currents flowing through channels endogenously expressed in the oocyte membrane. Compounds specifically modulating TRPML3 should not affect currents in uninjected oocytes and should exhibit % modulation factors near zero.

As illustrated in FIG. 8, for each compound screened, a % modulation factor is calculated. This value corresponds to the magnitude of the current change due to compound divided by the magnitude of the current change due to NMDG multiplied by −100%. In this example, three compounds (100 uM) are screened in succession in four, out of a possible maximum eight, oocytes voltage clamped to −60 mV in the OpusXpress system. All four oocytes expressed TRPML3, as evidenced by the inhibitory effect of NMDG on measured oocyte currents. All three compounds did not significantly modulate TRPML3 function as currents were similar before and after compound addition.

### Example 9

### I/V Curves with the TRPML3 Blocker Gadolinium

More complex analyses are performed on compounds displaying large % modulation factors and having no effect on oocytes not injected with TRPML3 cRNA. The assays include current/voltage (I/V) curves, NMDG competition experiments, and dose-response curves. For I/V curves, currents are measured in voltage steps from −100 to +60 mV, in 10 mV increments in the presence and absence of compound, to investigate the magnitude of compound modulation. The slope of the I/V curve is indicative of the magnitude of current modulation by the compound of interest. Strong enhancers increase the slope of I/V curves, indicative of increased opening of TRPML3 ion channels. Strong blockers decrease the slope of I/V curves, indicative of increased closing of TRPML3 ion channels. FIG. 9 illustrates how gadolinium blocks TRPML3 and decreases the slope of the I/V curve. In oocytes not injected with TRPML3 cRNA, I/V curves performed in the presence of compound should be identical and superimposable with I/V curves performed in the absence of compound. Small squares illustrate an I/V curve in sodium solution and large squares illustrate an I/V curve in 300 uM gadolinium (GdCl₃) solution. Note that gadolinium blocks TRPML3 current and decreases the slope of the I/V curve.

### Example 10

### NMDG Competition Experiments

NMDG competition experiments are performed to demonstrate that compound effects are TRPML3 dependent. First, NMDG is applied to demonstrate TRPML3 expression in the oocytes. Then, compound is applied to determine the % modulation factor. Finally, NMDG and compound are co-applied. For an enhancer to work directly on the TRPML3 channel, co-application of NMDG plus compound should exhibit a NMDG-type response, meaning that currents are inhibited and not enhanced. This experiment shows that when sodium is absent, and replaced with the non-permeant cation NMDG, the compound cannot have an enhancing effect; therefore, the compound must directly modulate TRPML3 sodium channel function.

### Example 11

### Dose-Response Curves

Dose-response curves are performed to determine the concentration at which the compound exhibits half-maximal activity (EC50 for enhancers and IC50 for blockers). The lower the EC50 or IC50 value, the more active the compound is as a TRPML3 modulator. Dose-response curves are performed by sequentially applying increasing concentrations of compound starting from low doses (˜1 nM) and progressing to high doses (˜1 mM). % modulation factors are calculated as described above and plotted as a function of compound concentration on a logarithmic scale to determine an EC50 or IC50 value for the compound.

### Example 12

### Expression of Constitutively Active Sodium Channels Increase Basal Fluorescence in Cells Loaded with Specific Membrane Potential Dyes

HEK<293 cells were transiently transfected with RFP, αENaC or A419P-TRPML3 and were loaded with a membrane potential dye (R-8034; Molecular Devices) in HBSS at room temperature for 30 minutes. Membrane potential (fluorescent signal) was monitored on a FLIPR system (Molecular Devices). See FIG. 11. Results show that cells expressing A419P-TRPML3 and αENaC show an elevated basal fluorescence when compared to RFP transfected cells (RFP is a control vector). These results mean that the constitutive activity of A419P-TRPML3 and αENaC causes the cell membrane to be more depolarized and that we can measure activity of A419P-TRPML3 in this FLIPR assay.

### Example 13

### Application of Gadolinium Reduces the Increase in Basal Fluorescence in Cells Expressing A419P-TRPML3

HEK293 cells were transiently transfected with RFP or A419P-TRPML3 and were loaded with a membrane potential dye (R-8034; Molecular Devices) in HBSS at room temperature for 30 minutes. See FIG. 12. Membrane potential (fluorescent signal) was monitored on a FLIPR system (Molecular Devices). Results show that addition of gadolinium (large, short arrowhead) significantly reduces the basal fluorescence close to values obtained in RFP-transfected cells (long arrow and trace 2). These results indicate that both the constitutive activity of A419P-TRPML3 and the activity of a TRPML3 modulator can be detected in this assay.

### Example 14

### Application of Gadolinium Reduces the Increase in Basal Fluorescence in Cells Expressing A419P-TRPML3 in a Dose-Dependent Fashion

HEK93 cells were transiently transfected with RFP or A419P-TRPML3 and were loaded with a membrane potential dye (R-8034; Molecular Devices) in HBSS at room temperature for 30 minutes. HBSS, NMDG and increasing concentrations of gadolinium were added to the cells and resulting changes in membrane potential (fluorescent signal) was monitored on a FLIPR system (Molecular Devices). See FIG. 13. Results show that increasing concentration of gadolinium significantly reduces the basal fluorescence to a much greater extent to the effect observed in RFP-transfected cells.

### Example 15

### Titration of TRPML3 Plasmid

HEK93 cells were transiently transfected with RFP (0 ug) or increasing amounts of A419P-TRPML3 plasmid (from 0.02 ug to 2 ug) and were loaded with a membrane potential dye (R-8034; Molecular Devices) in HBSS at room temperature for 30 minutes. See FIG. 4. HBSS (control) and 4 mM gadolinium were added to the cells and resulting changes in membrane potential (fluorescent signal) was monitored on a FLIPR system (Molecular Devices). Results show that increasing the amount of TRPML3 plasmid up to 0.5 g increases the size of the gadolinium effect.

### Example 16

### Effect of Gadolinium is Specific for TRPML3

HEK93 cells were transiently transfected with RFP, A419P-TRPML3 or αENaC plasmid and were loaded with a membrane potential dye (R-8034; Molecular Devices) in HBSS at room temperature for 30 minutes. HBSS (control), 3 mM gadolinium and 30 uM Amiloride were added to the cells and resulting changes in membrane potential (fluorescent signal) was monitored on a FLIPR system (Molecular Devices). See FIG. 15. Results show that gadolinium preferentially reduces basal fluorescence counts in A419P-TRPML3 transfected cells while amiloride preferentially reduces basal fluorescence counts in αENaC-transfected cells.

### Example 17

### Transducing HEK293 Cells with Baculovirus Encoding A419P-TRPML3 Doubles the Assay Window

HEK293 cells were transduced with a modified baculovirus allowing expression of A419P-TRPML3 in mammalian cells (BacMaM). After 24 hours infected cells were loaded with a membrane potential dye (R-8034; Molecular Devices) in HBSS at room temperature for 30 minutes. HBSS (control), 2 mM and 3 mM gadolinium were added to the cells and resulting changes in membrane potential (fluorescent signal) was monitored on a FLIPR system (Molecular Devices). See FIG. 16.

### Example 17

### Example of Screening Data Obtained with A419P-TRPML3 Expressing Cells

HEK293 cells were transduced with a modified baculovirus allowing expression of A419P-TRPML3 in mammalian cells (BacMaM). After 24 hours infected cells were loaded with a membrane potential dye (R-8034; Molecular Devices) in HBSS at room temperature for 30 minutes. See FIG. 17. 320 different compounds (red dots), HBSS (black dots) and gadolinium (blue dots) were added to the cells, from a 384 well compound plate, and resulting changes in membrane potential (fluorescent signal) was monitored on a FLIPR system (Molecular Devices). In this experiment two primary hits that apparently block A419P-TRMPL3 were identified.

### Example 18

### Summary of a 10,000 Compound Miniscreen with A419P-TRPML3 Expressing Cells

HEK293 cells were transduced with a modified baculovirus allowing expression of A419P-TRPML3 in mammalian cells (BacMaM) After 24 hours infected cells were loaded with a membrane potential dye (R-8034; Molecular Devices) in HBSS at room temperature for 30 minutes and treated as described in FIG. 17. See FIG. 18. 10,000 compounds were screened and several primary hits were identified, including 52 blocker hits and 113 enhancer hits.

Examples 11-18 show that under these experimental conditions, cells expressing A419P-TRPML3 showed a significant increase in basal fluorescence relative to cells transfected with a control vector (RFP). Gadolinium, a blocker of TRP channels, reversed the increase in fluorescence in a dose-dependent and specific fashion. Amiloride, a blocker of αENaC, had no effect on the increased basal fluorescence elicited by expression of A419P-TRPML3. Several thousand compounds have now been screened in this assay and several hits have been identified. These hits will be further evaluated by electrophysiology and taste tests.

### Example 19

### Construction of Codon Optimized TRPML3 Gene and Mutant

As shown in FIG. 19 a codon optimized TRPML3 gene was constructed. Codons comprise three nucleotides that encode a specific amino acid in a protein sequence. Since there are 61 different codon nucleotide triplets that encode 20 amino acids, most amino acids can be encoded by more than one codon. Codon optimization, the use of the favored codon for each amino acid in a particular species, can improve the functional expression of proteins by increasing the speed and accuracy of translation without changing the protein sequence.

The inventors used a codon-optimized version of the human TRPML3 gene that is 76.4% homologous to non-codon optimized TRPML3 at the DNA level (FIG. 19). Codons were optimized for optimal translation of human sequences. An active form of TRPML3 (A419P TRPML3) was also generated by mutating alanine 419 to proline in the 5^{th} transmembrane domain. This mutation results in TRPML3 channels that are in an open confirmation (Xu et al. PNAS 104(46): 18321-18326, 2007; Grimm et al. PNAS 104(49): 19583-19588, 2007; Nagata et al. PNAS 105(1): 353-358, 2008; Kim et al. J. Biol. Chem. 282(50): 36138-36142, 2007); therefore, A419P TRPML3 is particularly useful for identification of TRPML3 blockers.

### Example 20

As shown in the experiment contained in FIG. 20, expression of wild-type TRPML3 yields low sodium current levels, codon-optimized wild-type TRPML3 yields intermediate sodium current levels, and A419P TRPML3 yields high sodium current levels. Thus, codon-optimized wild-type TRPML3 and A419P TRPML3 facilitate screening for compounds that modulate TRPML3 function.

### Example 21

### Screening Oocytes Injected with Codon-Optimized Human TRPML3 cRNA

FIG. 21 contains an experiment involving screening oocytes injected with codon-optimized human TRPML3 cRNA to identify a compound (TRPML3 enhancer) that activates TRPML3. The results contained therein illustrate the identification of an enhancer that activates TRPML3 when oocytes are voltage clamped to −60 mV

In multiple oocytes, the TRPML3 enhancer increased TRPML3 activity by 169+/−26% from (representative trace on top) and had no effect on uninjected oocytes with no TRPML3 expression (representative trace on bottom). Addition of buffer only had no effect on TRPML3 currents and the effects of the TRPML3 enhancer were reproducible upon a second application.

### Example 22

### TRPML3 Enhancer Effect on TRPML3 I/V Curve

FIG. 22 contains an experiment illustrating an example of TRPML3 enhancer effect on TRPML3 I/V curve. This figure shows that the same enhancer as in prior example activates TRPML3 at negative voltages in an I/V curve analysis. Oocytes injected with codon-optimized human TRPML3 cRNA were untreated (blue triangles labeled control) or stimulated with TRPML3 enhancer (magenta squares labeled enhancer) and currents were measured at voltages from −90 to +30 mV. Note that the TRPML3 enhancer activates TRPML3 current at negative voltages (inward currents are larger with enhancer compared to with control), resulting in an increase in the slope of the I/V curve. Note also that the zero current shifts to the right, indicating an increased sodium conductance in the presence of the enhancer.

### Example 23

FIG. 23 contains an experiment which exemplifies TRPML3 enhancer effect in the presence and absence of extracellular sodium. Oocytes expressing codon-optimized human TRPML3 cRNA were stimulated with NMDG (no sodium), TRPML3 enhancer plus sodium, buffer only, or TRPML3 enhancer plus NMDG (no sodium). Note that TRPML3 enhancer increased TRPML3 activity in the presence of sodium but had no effect in the absence of sodium. These data demonstrate that the TRPML3 enhancer opens TRPML3 channels and increases the flow of sodium ions into the oocyte. FIG. 23 illustrates that this same enhancer does not activate TRPML3 in the absence of extracellular sodium. Since this compound opens TRPML3 and increases sodium flux into the cell, it is a candidate salty taste enhancer. Collectively, these data demonstrate how the electrophysiology oocyte assay can be used to identify TRPML3 enhancers that are candidate human salty taste enhancers, and how wild-type codon-optimized TRPML3 facilitates identification of TRPML3 enhancers in the oocyte electrophysiology assay.

### Example 24

### Expression Level of WT TRPML3 in Different Mammalian Cell Types

As shown in the experiment contained in FIG. 24, the expression level of WT TRPML3 depends on the mammalian cell type. In panel A of the Figure is a current voltage analysis (I/V plot) of HEK293 cells expressing WT and the A419P mutant TRPML3 channel. It shows that the A419P mutant TRPML3 channels express large inward rectifying currents (pink), whereas only small WT TRPML3 currents are observed (blue). B. WT and A419P mutant TRPML3 channels have similar functional characteristics in CHO cells.

### Example 25

### Use of TRPML3 for Enhancer and Blocker Screening in CHO Cells

The experiment in FIG. 25 relates to the use of TRPML3 for enhancer and blocker screening in CHO cells. Panel A shows WT human TRPML3 channels transiently expressed in CHO cells which were used to identify channel enhancers. I/V plot shows that compared to buffer alone (blue; control), use of the enhancer results in an increase in inward current at negative potentials (pink). Panel B. shows mutant A419P TRPML3 channel stably expressed in CHO cells are used to identify channel blockers. Compared to buffer alone (blue; control) addition of 1 mM GdCl₃ results in a decrease in inward current (pink).

### Example 26

### Screening Assays Using Codon-Optimized TRPML3 in Mammalian Cells

The experiment in FIG. 26 contains an experiment in mammalian cells using the same codon optimized WT TRPML3 contained in FIG. 19 for the screening of compounds which enhance TRPML3 function. Panel A shows the transient expression of non codon optimized WT TRPML3 (light blue) results in little current in HEK293 cells. In contrast, use of codon optimized WT TRPML3 (Dark Blue; Cod Opt WT) results in currents with similar average amplitude as A419P mutant channel (pink). Panel B shows the use of codon optimized WT TRPML3 (blue) delivered with Baculovirus transduction results in similar average currents as A419P TRPML3 (pink). Panel C. shows cells transduced with codon optimized WT TRPML3 baculovirus is used to identify enhancers of TRPML3 function. Compared to buffer alone (blue; control) addition of enhancer compound results in an increase in inward current (pink).

### Example 27

### Coexpression of WT and A419P TRPML3

The experiment in FIG. 27 relates to an experiment involving the coexpression of WT and A419P TRPML3. The results in the figure indicate that this increases functional surface expression in HEK1293 cells. Panel A shows currents elicited from A419P TRPML3 cDNA (0.5 ug) transfected into HEK293 cells, yielding currents with characteristic inward rectification. Panel B shows WT non codon optimized TRPML3 (1.5 ug) is expressed in HEK1293 cells and yields no currents. Panel C shows the coexpression of A419P (0.5 ug) with WT (1.5 ug) TRPML3 cDNAs in HEK293 cells result in large inward currents which are twice as large as those when expressing A419P cDNA alone. Panel D contains I/V plot of the average currents elicited from WT (blue), A419P (pink) and coexpression of WT and A419P (yellow) TRPML3 cDNAs in HEK1293 cells.

### Example 28

The experiment in FIG. 28 is an example of TRPML3 function measured in an IonWorks PPC patch plate experiment. Panel A contains a view of all 384 wells from a PPC patch plate with an A419 TRPML3 inducible clone showing the results of the pre-compound scan. Yellow indicates wells where the current at −120 mV was <0 nA (in control experiments with parental CHO-K1 cells none of the wells were labeled yellow). Blue indicates wells were the average seal resistance was too low (<10 mOhm) to measure the current reliably. A419P TRPML3 currents could be measured in 94% of the wells.

Panel B contains the average currents±SEM before and after addition of 4 mM GdCl3 or extracellular buffer (mock addition) from the patch plate shown in A. GdCl3 was added to column 1-38 while extracellular buffer was added to columns 39-48. For comparison, data is included from a separate experiment with parental CHO-K1 cells. The stability of the TRPML3 current after mock addition indicates that the assay should detect compounds that either enhance or block TRPML3 currents.

### Example 29

FIG. 29 contains an example of an IonWorks scan with an inducible CHO-1K1 cell line expressing A419P TRPML3 (top panel). TRPML3 inwardly rectifies, denoted by more current at hyperpolarized potentials (more negative potentials) and less current at depolarized potentials (more positive potentials). Addition of GdCl3 blocks TRPML3 current. Red line denotes scan in sodium (NaCl) solution. Blue line denotes scan in 4 mM GdCl3 solution. The middle panel is from parental CHO-K1 cells used as a negative control. The positive currents at negative potentials are due to leak subtraction overcorrecting the current at negative potentials. The bottom panel show the voltage command protocol used to record currents. The step from 0 mV to 10 mV is used to calculate the leak current (current flowing through leaks in the seal) which is subtracted from the total current to obtain the current flowing through the membrane.

Results are from single wells in a PPC patch plate and represent the average current of up to 64 cells. FIG. 30 contains a flowchart of experiments used to examine the effect of compounds on human TRPML3 (hTRPML3) activity in the IonWorks assay.

### Example 30

As shown by the results of the real-time PCR experiment contained in FIG. 31, TRPML3 taste cells are specifically ablated from taste buds in the Varitint waddler mice. End-point RT-PCR experiments on taste buds (TB) and lingual epithelial cells (LE) of Varitint waddler (Va) or wild-type (WT) mice isolated by laser-capture microdissection are shown.

The results indicate that TRPML3 is only expressed in taste buds of WT mice and absent in taste buds of Va mice, whereas all other taste genes (T1R2, Gpr113, TRPM5) as well as housekeeping genes (beta-actin, GAPDH) are equally expressed in TB and LE. ‘+’ indicates that reverse transcription was performed and ‘−’ indicates that no reverse transcription was performed. PCR bands were only observed with reverse transcriptase indicating that PCR products are derived from mRNA and not genomic DNA.

### Example 31

This example relates to experiments summarized in FIG. 32 containing the results of a real-time PCR experiment showing that TRPML3 cells are specifically ablated from taste buds of Varitint waddler mice. Real-time quantitative RT-PCR experiments on taste buds of Varitint waddler (Va) or wild-type (WT) mice isolated by laser-capture microdissection. TRPML3 is only expressed in taste buds of WT mice and absent in taste buds of Va mice (similar results were obtained using two different primer sets labeled Mcoln3_{—}1 and Mcoln3_{—}2), whereas all other taste genes (Tas1r2, Tas1r3, PID2l1, TRPM5, Plcb2, Tas2r108, and Tas2r116) as well as a housekeeping gene (control) are expressed in taste buds from Va and WT mice.

### Example 32

This example relates to an experiment in FIG. 33 showing that sweet, bitter, umami and sour taste cells remain intact in the Varitint waddler mice. In situ hybridization of circumvallate papilla from the back of the tongue of wild-type (top row of images) and Varitint waddler (Va; bottom row of images) mice. PID1L3 (left; sour), PID2L1 (middle; sour), and TRPM5 (right; sweet, bitter, and umami) taste cells were present at similar levels in wild-type and Va mice.

### Example 33

This example relates to an experiment using CT nerve recordings in wild-type and Varitint waddler mice stimulated with salty taste stimuli contained in FIG. 34 showing that the Varitint waddler mice are deficient in salty taste perception and that the wild-type mice detected salty taste under the same conditions. CT nerve recordings from wild-type (left) or Varitint waddler (Va; right) mice. Anterior tongues were stimulated with 0.1 M NaCl or 0.1 M NaCl plus 5 uM benzamil to inhibit the amiloride-sensitive component of the CT nerve response. Tongues were rinsed with a low salt solution containing 10 mM KCl in between NaCl stimulations. Note that the benzamil-insensitive component of the CT nerve response is largely eliminated in the Va mouse (red arrows), indicating that ablation of TRPML3 taste cells significantly impairs salty taste perception. In addition, the immediate phasic response to NaCl is greatly reduced in the Va mouse (red circles). Scale bars indicate time frames of salt application (x-axis) and the magnitude of the CT response (y-axis; arbitrary units).

FIG. 35 contains an alignment of the protein sequences derived from human (NM_{—}018298) and mouse (NM_{—}134160) TRPML3 sequences wherein human is denoted Hs and mouse is denoted Mm. Human and mouse protein sequences are 91% identical and 96% similar. The six transmembrane domains in the TRPML3 polypeptides for both human and mouse TRPML3 are underlined TM1 through TM6. The pore region between TM5 and TM6 is denoted ‘pore region’. The amino and carboxy termini are predicted to be located inside the cell. The A419P mutation discussed herein and found in the varitint-waddler mouse locks TRPML3 in the open conformation is in TM5 and is highlighted in red. Another mutation, V412P, partially activates TRPML3 and is denoted in magenta.

### Example 34

### TRPML3 Expression in Kidney Collecting Duct Principal Cells in Wild-Type and Va/+ Mice

As shown by the results contained in FIG. 36 double label in situ hybridization studies localized TRPML3 to principal cells in cortical and outer medullary collecting ducts. Double labelling studies localized TRPML3 to principal cells in cortical and outer medullary collecting ducts. TRPML3 kidney cells express both aquaporin-2 water channels (FIG. 36A-D) that reabsorb water to maintain plasma osmolarity, as well as ENaC sodium channels that reabsorb sodium to maintain salt balance (FIG. 36E-F). TRPML3 kidney transcripts were not reduced by qPCR (WT Ct of 28.4; Va/+Ct of 27.5) and TRPML3 kidney cells were not ablated in Va/+ mice, pointing to variable penetrance of the A419P TRPML3 mutation (FIG. 36G-J). Since principal cells respond to the sodium-conserving hormone aldosterone, we measured plasma sodium and aldosterone levels in Va/+ mice. Va/+ mice exhibit a pronounced hyponatremia and hyperaldosteronemia, consistent with a deficiency in principal cell function (Table 2), while plasma chloride, potassium, glucose, and protein levels were not significantly different from wild-type controls. Comparable levels of aldosterone synthase (CYP11B2) transcripts in wild-type and Va/+ adrenal glands by qPCR argues that elevated plasma aldosterone was not primarily due to perturbation of aldosterone secretion from adrenal glomerulosa cells (WT Ct of 23.4; Va/+Ct of 23.7). Thus, TRPML3 is expressed in kidney epithelial cells responsible for sodium balance, and Va/+ mice exhibit deficiencies in sodium homeostasis.

More specifically, in FIG. 36 (A) TRPML3 (ML3; blue; left) and aquaporin-2 (Aqp-2; red; center) are coexpressed in collecting duct cells (merge; right). (B) TRPML3 is not expressed in inner medullary collecting duct cells that express aquaporin-2. For images A and B, TRPML3 was visualized by in situ hybridization while aquaporin-2 was visualized by immunohistochemistry. (C) Aquaporin-2 (green; left) and TRPML3 (red; middle) are coexpressed in collecting duct cells (merge; right). (D) TRPML3 is not expressed in inner medullary collecting duct cells that express aquaporin-2. For images in C and D, TRPML3 and aquaporin-2 were visualized by immunohistochemistry. (E) TRPML3 (blue; left) and βENaC (red; center) are coexpressed in collecting duct cells (merge; right). (F) TRPML3 (blue; left) and γENaC (red; center) are coexpressed in collecting duct (CD) cells (merge; right). γENaC is also expressed in more distal nephron (DN) segments such as connecting tubules. For images in E and F, TRPML3 was visualized by in situ hybridization while ENaCs were visualized by immunohistochemistry. (G) in situ hybridization of TRPML3 in wild-type (WT) kidney. (H) in situ hybridization of TRPML3 in Va/+ kidney showing no loss of TRPML3 cells. (I) immunohistochemistry of TRPML3 in wild-type (kidney. (J) Immunohistochemistry of TRPML3 in Va/+ kidney showing no loss of TRPML3 cells. Scale bar, 60 um (A-B), 50 um (C-D), 60 uM (E-F), and 80 um (G-J).

### Example 35

### Blood Chemistry in Va/+ Mice

As mentioned previously, unlike in taste cells, TRPML3 cells in the kidney were not ablated in Va/+ mice, suggesting that the A419P TRPML3 mutation did not compromise ionic homeostasis to an extent that compromised cell viability in kidney cells (FIG. 36 G-J). Since principal cells respond to the sodium-conserving hormone aldosterone, we measured plasma sodium and aldosterone levels in Va/+ mice. Va/+ mice exhibit a pronounced hyponatremia (low blood sodium concentration) and hyperaldosteronemia (high blood aldosterone concentration), consistent with a deficiency in principal cell function (Table 2 below), while other electrolytes including plasma chloride and potassium as well as glucose and protein levels were not significantly different from wild-type controls. TRPML3 is, therefore, expressed in kidney epithelial cells responsible for sodium balance, and Va/+ mice exhibit deficiencies in sodium homeostasis.

TABLE 2
Blood chemistry reveals alterations in sodium homeostasis
in Va/+ mice.

| Measurement | WT | Va/+ |
|---|---|---|
| Aldosterone (pg/ml) | 161.3 +/− 34.4 | 896.3 +/− 110.5* |
| Sodium (mEq/ml) | 151.0 +/− 1.0 | 141.3 +/− 0.3* |
| Chloride (mEq/ml) | 100.3 +/− 0.3 | 100.0 +/− 0.6 |
| Potassium (mEq/ml) | 5.3 +/− 0.2 | 5.0 +/− 0.1 |
| Glucose (mg/dl) | 204.7 +/− 6.8 | 192.3 +/− 3.8 |
| Protein (g/dl) | 6.7 +/− 0.2 | 6.7 +/− 0.1 |
| Albumin (g/dl) | 4.3 +/− 0.3 | 4.6 +/− 0.3 |

In Table 2 the values are means±SEM for three mice. Similar data were obtained in separate litters of mice. WT, wild-type mice; Va/+, heterozygous varitint-waddler mice. * p<0.05 by 2-tailed, unpaired Student's t-test.

### Example 36

### Taste Test Studies

### Preliminary

Taste Studies Using Putative Salty Taste Enhancer and Blocker Compounds

Using TRPML3 electrophysiological functional assays disclosed herein several candidate salty taste enhancers and blocker compounds were identified. These results are preliminary as it is anticipated that these compounds may be “optimized” or that compounds which function as more potent enhancers or blockers may be identified using the same or similar TRPML3 screening assays. In addition further screening may be required to identify TRPML3 modulatory compounds which do not elicit undesired effects on taste (such as bitterness or “off-taste”) which potentially may be the result of the identified compounds interacting with other taste receptors such as T2Rs.

These results are described herein.

### Salty Enhancer Taste Studies

TABLE 3

| | 50 uM | 20 uM | 40 uM | 40 uM | 15 uM | 50 uM |
|---|---|---|---|---|---|---|
| | S7437 | S6927 | S6927 | S6927 | S5203 | S2480 |
| 100 mM NaCl + | 15 | 23 | 19 | 17 | 23 | 16 |
| compound | | | | | | |
| selected as | | | | | | |
| saltier | | | | | | |
| 100 mM NaCl | 7 | 19 | 11 | 15 | 19 | 14 |
| selected as | | | | | | |
| saltier | | | | | | |
| # panelists | 22 pan × | 14 pan × | 15 pan × | 16 pan × | 14 pan × | 15 pan × |
| | 1 rep | 3 rep | 2 rep | 2 rep | 3 rep | 2 rep |
| p-value | p = 0.134 | P = 0.644 | p = 0.200 | p = 0.720 | p = 0.644 | p = 0.856 |

This Table shows the results of 2 alternative forced choice (paired comparison) test results measuring salt enhancement using putative TRPML3 enhancers. In these studies panelists compared 100 mM NaCl+compound vs. 100 mM NaCl. P-values are calculated for 100 mM NaCl+compound as being saltier.

TABLE 4

| | 40uM S6927 |
|---|---|
| 50 mM NaCl + compound | 9 |
| selected as saltier | |
| 50 mM NaCl selected as | 15 |
| saltier | |
| # panelists | 12 pan × 2 rep |
| p-value | p = 0.307 |

Table 4 contains the results of another 2-alternative forced choice (paired comparison) test results measuring salt enhancement. Panelists compared 50 mM NaCl+compound vs. 50 mM NaCl. P-value is calculated for 50 mM NaCl as being saltier due to the trend being opposite from what was expected.

TABLE 5

| | 40 uM S6927 |
|---|---|
| 100 mM KCl + compound | 15 |
| selected as saltier | |
| 100 mM KCl selected as | 15 |
| saltier | |
| # panelists | 15 pan × 2 rep |
| p-value | p > 0.856 |

This table contains the results of a third 2-alternative forced choice (paired comparison) measuring salt enhancement. Panelists compared 100 mM KCl+ compound vs. 100 mM KCl. P-value is calculated for 100 mM KCl+ compound as being saltier.

Conclusions: These results (preliminary) indicated in some tests that the putative salt enhancer compounds showed trends toward increased saltiness. While these preliminary results were not statistically significant it is theorized that this may be because these compounds do not possess adequate binding affinity to the receptor (and therefore may need to be optimized) or may not enhance TRPML3 function sufficiently to cause a perceptible change in salty taste perception. Alternatively the tested compounds may be eliciting another taste modality (e.g., bitterness) that may preclude the taste panelists from detecting any change in saltiness.

Of the identified putative TRPML3 enhancers the compound referred to internally as S6927 seemed to work better in 100 mM NaCl compared to 50 mM NaCl. This compound did not seem to have any effect on saltiness of KCl.

TABLE 6

| | | 45uM S7923 |
|---|---|---|
| | | (panelists who |
| | | did not |
| | 45uM S7923 | perceive |
| | (all panelists) | bitterness) |
| 100 mM NaCl selected as | 18 | 13 |
| saltier | | |
| 100 mM NaCl + compound | 30 | 23 |
| selected as saltier | | |
| # panelists | 16 pan × 3 rep | 12 pan × 3 rep |
| p-value | 0.111 | 0.132 |

This Table contains another 2-alternative forced choice (paired comparison) with test results measuring salt enhancement. Panelists compared 100 mM NaCl+compound vs. 100 mM NaCl. P-value is calculated for 100 mM NaCl+compound as being saltier.

The screened compound S7923 contains a functional group which exhibits similar properties to those compounds for which humans are known to have differing sensitivities, i.e. some subjects find the sample to be bitter while others do not. Results are shown across all panelists as well as with removing those who perceive bitterness from S7923.

TABLE 7

| | | 45uM S7923 |
|---|---|---|
| | | (panelists who |
| | | did not |
| | 45uM S7923 | perceive |
| | (all panelists) | bitterness) |
| 100 mM NaCl selected as | 26 | 16 |
| saltier | | |
| 100 mM NaCl + compound | 22 | 17 |
| selected as saltier | | |
| # panelists | 16 pan × 3 reps | 11 pan × 3 reps |
| p-value | 0.665 | >0.860 |

This Table repeats taste tests using the same putative enhancer compound S7923. Again the Table shows the results of a 2-alternative forced choice (paired comparison) measuring salt enhancement. Panelists compared 100 mM NaCl+compound vs. 100 mM NaCl. P-value is calculated for 100 mM NaCl+compound as being saltier.
Conclusions: Strong trends toward increased saltiness were seen with S7923 in the first test, but not in the repeated test. Results were not statistically significant at p<0.1 in either set of tests.

TABLE 8

| | | | | | | 50 uM |
|---|---|---|---|---|---|---|
| | | | | | | S6927 + |
| | 20 uM | 15 uM | 30 uM | 10 uM | 40 uM | 50 uM |
| | S5559 | S9047 | S1435 | S0582 | S1617 | S969 |
| 100 mM NaCl + | 15 | 19 | 21 | 18 | 18 | 8 |
| compound | | | | | | |
| selected as | | | | | | |
| saltier | | | | | | |
| 100 mM NaCl | 18 | 32 | 27 | 27 | 27 | 11 |
| selected as | | | | | | |
| saltier | | | | | | |
| # panelists | 11 pan × | 17 pan × | 16 pan × | 15 pan × | 15 pan × | 19 pan × |
| | 3 rep | 3 rep | 3 rep | 3 rep | 3 rep | 1 rep |
| p-value | 0.728 | 0.092 | 0.471 | 0.233 | 0.233 | 0.648 |

This Table shows the results of another 2-alternative forced choice (paired comparison) test results measuring salt enhancement. Panelists compared 100 mM NaCl+compound vs. 100 mM NaCl. P-values are calculated for 100 mM NaCl as being saltier.

Conclusions: The data in Table 8 shows a trend toward NaCl being saltier than NaCl plus putative salty taste enhancer. These tests showed trends toward decreased saltiness when compound was added (as opposed to enhancement), however results were not statistically significant at p<0.05.

### Salt Blocker Taste Tests (Preliminary)

TABLE 9

| | 50 uM S9946 | | 100 uM S9946 | | |
|---|---|---|---|---|---|
| | Test 1 | Test 2 | Test 1 | Test 2 | Test 3 |
| 100 mM | 28 | 24 | 10 | 7 | 4 |
| NaCl | | | | | |
| selected | | | | | |
| as saltier | | | | | |
| 100 mM | 14 | 21 | 4 | 7 | 9 |
| NaCl + | | | | | |
| compound | | | | | |
| selected | | | | | |
| as saltier | | | | | |
| # panelists | 14 pan × | 15 pan × | 14 pan × | 14 pan × | 13 pan × |
| | 3 rep | 3 rep | 1 rep | 1 rep | 1 rep |
| p-value | p = 0.044 | p = 0.766 | p = 0.180 | p > 0.791 | p = 0.267* |

*p-value is calculated for 100 mM NaCl + compound as being saltier.

This Table contains the results of 2-alternative forced choice (paired comparison) test results measuring salt blocking using putative salty taste blockers identified in TRPML3 electrophysiological functional assays which selected for compounds that blocked the functionality of TRPML3. In these tests, panelists compared 100 mM NaCl vs. 100 mM NaCl+compound. P-values are calculated for 100 mM NaCl as being saltier.

Conclusions: In some tests, the proprietary putative salty taste blocker compound S9946 showed trends toward decreased saltiness, but these results are still preliminary and were not consistently statistically significant.

TABLE 10

| | Trial 1: 15 uM S9853 | | | | Trial 2: 15 uM S9853 | | | |
|---|---|---|---|---|---|---|---|---|
| | with 1 minute rest periods | | | | with 15 minute rest periods | | | |
| | between tests | | | | between tests | | | |
| | | | | Combined | | | | Combined |
| | Test 1 | Test 2 | Test 3 | Results | Test 1 | Test 2 | Test 3 | Results |
| 100 mM NaCl | 10 | 6 | 7 | 23 | 10 | 8 | 6 | 24 |
| selected as saltier | | | | | | | | |
| 100 mM NaCl + | 4 | 8 | 7 | 19 | 4 | 6 | 8 | 18 |
| compound | | | | | | | | |
| selected as saltier | | | | | | | | |
| # panelists | 14 | 14 | 14 | 42 | 14 | 14 | 14 | 42 |
| p-value | 0.180 | 0.791 | 0.791 | 0.644 | 0.180 | 0.791 | 0.791 | 0.441 |

This Table contains the results of a further 2-alternative forced choice (paired comparison) test results measuring salt enhancement using putative salty taste blockers identified using electrophysiological assays that screened for compounds that blocked TRPML3 function. Panelists compared 100 mM KCl+compound vs. 100 mM KCl. P-value is calculated for 100 mM KCl+compound as being saltier.

Conclusions: 15 uM of salt blocker compound S9853 did not significantly reduce saltiness of 100 mM NaCl (p>0.1) across all tests. However, the compound did consistently trend toward blocking within the first test of each of the two trials (with and without 15 minute wait between tests). This may be indicative of the irreversible effect on the salt channel, which prevents any subsequent tests from demonstrating appreciable differences between the two samples.

TABLE 11

| | | | | | | | | | Trial 3: |
|---|---|---|---|---|---|---|---|---|---|
| | | | | | | | | | 15 uM |
| | | | | | | | | | S9853 |
| | Trial 1: 15 uM S9853 | | | | Trial 2: 15 uM S9853 | | | | tested by |
| | with 1 minute rest periods | | | | with 15 minute rest periods | | | | 45 |
| | between tests | | | | between tests | | | | different |
| | | | | Combined | | | | Combined | panelists |
| | Test 1 | Test 2 | Test 3 | Results | Test 1 | Test 2 | Test 3 | Results | Results |
| 100 mM NaCl | 10 | 6 | 7 | 23 | 10 | 8 | 6 | 24 | 27 |
| selected as | | | | | | | | | |
| saltier | | | | | | | | | |
| 100 mM NaCl + | 4 | 8 | 7 | 19 | 4 | 6 | 8 | 18 | 18 |
| compound | | | | | | | | | |
| selected as | | | | | | | | | |
| saltier | | | | | | | | | |
| # panelists | 14 | 14 | 14 | 42 | 14 | 14 | 14 | 42 | 45 |
| p-value | 0.180 | 0.791 | 0.791 | 0.644 | 0.180 | 0.791 | 0.791 | 0.441 | 0.233 |

This Table combines the results of 3 taste trials with the same putative salty taste blocker compound S9583 in Table 10 supra. This test similarly involved a paired comparison taste test for salt blocking using 100 mM NaCl vs. 100 mM NaCl+15 uM S9853.

Conclusions: Panelists found 100 nM NaCl+15 uM S9583 was not significantly different in saltiness than 100 mM NaCl (p=0.233). By contrast panelists found 120 mM NaCl was significantly saltier than 100 mM NaCl (p<0.001) and that 110 mM NaCl was significantly saltier than 100 mM NaCl (p<0.001). These results possibly may be explained by the fact that these same panelists noted that 100 nM NaCl+15 uM S9583 had an off-taste which was most frequently described as bitter and astringent. It is theorized based thereon that this compound may be interacting with other (non-salty) taste receptors such as T2Rs or unidentified taste receptors.

### Practical Applications of the Invention

Compounds which modulate, preferably enhance the activity of TRPML3 according to the invention have important implications in modulation of human salty taste and potentially other taste modalities or taste in general. In addition these compounds are potentially useful in therapeutic applications involving other taste cell related functions and phenotypes such as taste cell turnover, digestive diseases, digestive function, regulation of metabolism, regulation of immunity in the oral cavity and/or digestive system and the like.

Compounds which activate taste ion channels in taste papillae on the tongue can be used to enhance salt sensation by promoting Na⁺ transport into taste bud cells. This has obvious consumer applications in improving the taste and palatability of low salt foods and beverages. sodium homeostasis, including blood pressure regulation in hypertension (high blood pressure) and hypotension (low blood pressure).

Also, TRPML3 enhancers and blockers can be used to modulate renal sodium balance to control blood pressure; can be used to modulate plasma osmolarity, including decreases in osmolarity (hypoosmotic) and increases in osmolarity (hyperosmotic) via reabsorption of water in the kidney in normal and diseased states such as diabetis insipidus; can be used to modulate ENaC function and/or trafficking; can be used to modulate aquaporin-2 function and/or trafficking; can be used to modulate vasopressin (antidiuretic hormone) secretion from the pituitary gland; and/or can be used to modulate aldosterone secretion from the adrenal glands.

In addition the genes and gene products herein can be used as markers for identifying, isolating or enriching specific taste cell types or lineages.

Further the genes and gene products specific to taste cells identified herein can be used to identify compounds that modulate apoptosis of taste cells, modulate transcription factors that control taste receptor expression, modulate autocrine/paracrine modulation of taste cell development, prolong taste bud lifetime, yield supertaster animal phenotypes for use in screening such as for bioterrorism or animals for use in screening for compounds that induce the activation and differentiation of stem cells into taste cells in vivo.

Also the subject genes, gene products and cells which express same may be used to screen for compounds that affect trafficking of taste receptors to and from the apical membrane/taste pore region to enhance or repress general or specific tastes, regulation of taste cell action potential firing frequency/membrane potential to control the intensity of general or specific tastes, regulation of neurotransmitter release to afferent nerve to control the intensity of general or specific taste, and autocrine/paracrine modulation of taste receptor function.

Further the subject genes, gene products and cells which express same can be used to identify compounds that regenerate taste cells such as in geriatric individuals or patients with cancer, chemotherapy radiation, injury or surgery affecting taste, drug-induced dysgeusia, ageusia, and for alleviating taste bud loss.

Still further the subject genes and gene products and cells which express same can be used to screen for compounds that affect oral hygiene, halitosis, detoxification of noxious substances in the oral cavity, and neutralization/elimination of bacteria, viruses, and other immunogens in the saliva/mouth or digestive tract.

Preferably, modulators of TRPML3 can be used as flavor additives in order to elicit or modulate (enhance or inhibit) salty taste perception, and to treat conditions and physiological functions involving sodium metabolism, absorption and excretion. In particular, modulators of TRPML3 can be added to foods, beverages and medicaments and other consumer products in order to modulate or mask the salty taste thereof. In addition, TRPML3 modulators and enhancers can be used to treat and modulate TRPML3 related cardiac and urinary functions such as blood pressure, fluid retention, urine production, plasma osmolarity, stroke, heart attack, arrhythmias, aldosterone production, and vasopressin release.

In addition, transgenic TRPML3 animals, e.g., knockout and knock-in animals have practical applications in the study of the effects of sodium metabolism and other activities on physiological processes and diseases such as Addison's disease as well as for the identification of compounds that modulate TRPML3.

As afore mentioned the taste cell specific genes identified according to the invention and the corresponding gene products and cells which express same e.g., endogenous taste or chemosensory cells or urinary (kidney) and recombinant cells including these taste specific genes, and their orthologs, allelic variants, variants possessing at least 90% sequence identity thereto and/or genes which specifically hybridize thereto under hybridization conditions denied supra may be used in assays to identify taste modulatory compounds as well as in therapeutic screening assays.

For example these taste specific genes, polypeptides and cells expressing same can be used to screen for compounds for treatment of digestive system disorders. These disorders include by way of example conditions affecting digestion such as dyspepsia, autoimmune and inflammatory diseases affecting the digestive system such as ulcerative colitis, inflammatory bowel syndrome, Crohn's syndrome, celiac disease, and precancers and cancers that affect the digestive system such as cancers affecting the salivary glands, taste buds, stomach, pancreas, gall bladder, esophagus, small or large intestine, anus or colon.

Also these taste specific genes may be used in screening assays to identify compounds that affect taste cell turnover. It is known that taste cells turnover rapidly (about every couple of weeks). Moreover, many conditions including chemotherapy or radiation treatment, as well as old age may negatively affect the ability of taste cells to develop. The result is a diminished sense of taste which may result in a decreased quality of life in cancer patients or the elderly. This is particularly pronounced in patients with head and neck cancer, esophageal, stomach, lung, or pancreatic cancers. Additionally, this may evolve along with another condition, cachexia or wasting syndrome that combines to reduce the desire to eat. Lack of proper nutrition is a serious cause of morbidity and mortality in cancer patients. The subject taste specific genes contain genes expressed in stem cells suggesting that they are markers of stem cells that are the precursors of and which evolve into taste cells. These genes or cells which express same can be used to identify signals that accelerate taste cell development. These signals which likely comprise cytokine-like receptors present on taste cells likely mediate taste cell development and can be used in screens to identify compounds that induce taste cell differentiation or proliferation. The ligands therefore potentially may be isolated from saliva and may account for the ability of saliva to influence taste function. For example, patients with Sjogren's syndrome (an autoimmune disease that attacks the salivary glands) exhibit altered taste functions. The subject genes and the study of gene expression in the salivary glands by use of gene arrays will facilitate an understanding of these differentiation mechanisms.

The subject taste cell specific genes and corresponding gene products and cells which express these genes may also be used in order to identify potential therapeutics for modulating the immune system of the oral cavity. The oral cavity is populated by normal flora as is the digestive tract. Alterations in normal flora may give rise to conditions such as gingivitis, halitosis, gastric problems and other infections that may result in tooth decay or tooth loss. Included within the taste cell specific genes identified herein are a number of immune system genes. These genes and the corresponding polypeptides or cells which express same can be used to identify therapeutics for maintaining immune homeostasis in the oral cavity, preventing overgrowth of pathogenic microbial and for identification of the cell types in the oral cavity that are the key players in maintaining proper oral cavity immunity.

Moreover, the subject taste cell specific genes and the corresponding gene products or cells which express same are useful in screening assays for identifying compounds for treatment of diabetes, eating disorders such as obesity.

Gene products and compounds that enhance or inhibit gene products identified by the inventors can affect: oral hygiene, halitosis, detoxification of noxious substances in anorexia, bulimia, and other metabolic disorders. The expression of taste receptors in the digestive system likely represents a comprehensive system that detects food and different types at different places during digestion. Therefore, “sensing” the presence of food or specific types such as carbohydrates, fats, umami foods, salts, should trigger various signals that may regulate the production of molecules that participate in the regulation of digestion such as GIP (glucose-dependent insulinotrophic polypeptide) and GLP-1 (glucagon-like peptide 1) produced by the enter endocrine cells in the intestine. It is likely that taste receptors on these cells regulate the production of other molecular signals in other cells of the digestive system when triggered. These phenomena may be studied by determining which cells express different receptors and then using gene arrays to study the molecules that these cells produce when activated.

More specifically, this invention identifies and provides functional (electrophysiological) and immohistochemistry data which indicate that TRPML3 (MCOLN3) encodes a polypeptide that functions as a primate (e.g., human) salty taste receptor and is involved in sodium homeostasis.

Also more specifically, the present invention provides the use of these taste specific genes as markers which can be used to enrich, identify or isolate salt receptor expressing cells.

Also more specifically, this invention provides in vitro and in vivo assays which use TRPML3 (MCOLN3) and TRPML3 expressing cells or TRPML3 transgenic animal models to identify agonist, antagonist or enhancer compounds which elicit or modulate (block or enhance) salty taste in primates including humans. These assays use cells which express TRPML3 alone or cells which express the TRPML3 ion channel in association with other taste specific polypeptides such as NALCN or NKAIN3.

Also more specifically, this invention provides transgenic animals, preferably rodents, and the use thereof to confirm the role of TRPML3 in salty taste in mammals and in other physiological functions involving sodium and other ions such as sodium metabolism, blood pressure, fluid retention and excretion, urinary function and cardiac function.

Also more specifically, this invention provides in vitro and in vivo assays which use TRPML3 and TRPML3 expressing cells or transgenic animals in assays, preferably electrophysiological assays in order to identify therapeutic compounds which alleviate diseases and conditions involving deficiencies in the expression of this polypeptide including hyperexpression, hyporexpression, and mutations in the TRPML3 polypeptide that affect its ability to function as a taste specific sodium channel in a mammal including e.g., human and non-human primates and rodents. These conditions include by way of example Addison's disease and diseases involving aberrant aldosterone production or vasopressin release such as hypertension, hypotension, fluid retention, and impaired urinary or cardiac function such as arrhythmia, heart attach and stroke. In addition conditions treatable using TRPML3 modulatory compounds include melanoma and other conditions involving melanocytes such as pigmentation disorders.

Also more specifically, this invention provides a method of modulating sodium homeostasis in a subject in need thereof comprising administering a modulatory effective amount of a TRPML3 modulatory compound. Even more specifically, this invention provides a method wherein the modulatory compound is an agonist, antagonist, enhancer or blocker of TRPML3 and is used to regulate blood pressure such as hypertension or hypotension.

Also more specifically, this invention provides a method of modulating plasma osmolarity in a subject in need thereof comprising administering a modulatory effective amount of a TRPML3 modulatory compound. More specifically this method is used to treat decreases in osmolarity (hypoosmotic) or increases in osmolarity (hyperosmotic) and/or to promote reabsorption of water in the kidney in a normal or diseased individual having a disease that impairs normal osmolarity such as diabetis insipidus.

Also more specifically, this invention provides a method of modulating ENaC function and/or trafficking in a subject in need thereof comprising administering an effective amount of a TRPML3 modulatory compound, i.e., a TRPML3 enhancer, blocker, agonist or antagonist.

Also more specifically, this invention provide a method of modulating aquaporin-2 function and/or trafficking in a subject in need thereof comprising administering an effective amount of a TRPML3 modulatory compound, e.g., a TRPML3 enhancer, blocker, agonist or antagonist.

Also more specifically, this invention provides a method of modulating vasopressin (antidiuretic hormone) secretion from the pituitary gland in a subject in need thereof comprising administering an effective amount of a TRPML3 modulatory compound, i.e., a TRPML3 enhancer, blocker, agonist or antagonist.

Also more specifically, this invention provides a method of modulating aldosterone secretion from the adrenal glands in a subject in need thereof comprising administering an effective amount of a TRPML3 modulatory compound i.e., a TRPML3 enhancer, blocker, agonist or antagonist.

Also more specifically, this invention provides a genetic screening assay for detecting abnormalities that affect one or more of sodium homeostasis, plasma osmolarity, ENaC function and/or trafficking, aquaporin-2 function and/or trafficking, vasopressin (antidiuretic hormone) secretion from the pituitary gland, and/or aldosterone secretion from the adrenal glands comprising characterizing the sequence of the TRPML3 gene in a subject and thereupon comparing this sequence to a wild type TRPML3 sequence in a subject with normal TRPML3 function as a means of detecting for mutations including substitution, deletion, addition mutations in the TRPML3 coding sequence or mutations in the regulatory regions that impair normal TRPML3 transcription and/or translation.

Also more specifically, this invention provides a method of using a transgenic animal having a mutation that affects TRPML3 function in assays for screening the efficacy of compounds for treatment of conditions involving at least one of sodium homeostasis, plasma osmolarity, ENaC function and/or trafficking, aquaporin-2 function and/or trafficking, vasopressin secretion or aldosterone release, e.g., a Varitint Waddler mouse.

Therefore, in conclusion this invention relates to the identification of MCOLN3 as encoding a human salty taste receptor which allows for the design of screening assays using cells transfected with this gene for the purpose of identifying agonists, antagonists or enhancers (modulator compounds) which affect the function of this molecule. These compounds can be used as taste modulators and as therapeutics that modulate sodium metabolism, absorption and excretion. In order to further describe the invention and exemplary embodiments, the following TRPML3 nucleic acid and polypeptide sequences from different mammals including human are provided below. However, as afore-mentioned, the taste cell specific genes identified according to the invention and the corresponding gene products and cells which express same e.g., endogenous taste or chemosensory cells and recombinant cells including these taste specific genes, and their orthologs, allelic variants, chimeras, and genetically engineered fragments and variants possessing at least 80%, 90%, 95%, 96%, 97%, 98%, 99% or greater sequence identity thereto and/or genes which specifically hybridize thereto under hybridization conditions denied supra may be used in assays to identify taste modulatory compounds as well as in therapeutic screening assays, preferably those for identifying compounds for modulating conditions involving sodium transport, excretion and homeostasis, plasma osmolarity, regulation of blood pressure, ENaC function, aquaporin-2 function and/or trafficking, vasopressin secretion and/or aldosterone release.

### REFERENCES

All the references cited in this application are incorporated by reference in their entirety herein.

While the invention has been described by way of examples and preferred embodiments, it is understood that the words which have been used herein are words of description, rather than words of limitation. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

1.-282. (canceled)

283. Isolated taste, adrenal, pituitary, parathyroid, melanocyte, or urinary organ cells or an enriched taste cell sample wherein said isolated or enriched cell sample comprises cells that express a TRPML3 ion channel polypeptide.

284. The isolated cells or enriched taste cell sample of claim 283 wherein said TRPML3 is selected from a mammalian, avian, amphibian, fish, and reptilian TRPML3.

285. The isolated cells or enriched cell sample of claim 283 wherein said TRPML3 ion channel polypeptide possesses at least 90% sequence identity to a TRPML3 polypeptide selected from the polypeptides having SEQ ID NO: 2, 4, 10, 12, 14, 16, 22, 24, 26, 28, 30, 32 and 34 or the TRPML3 polypeptide encoded by SEQ ID NO:39 or 40.

286. The isolated cells or cell sample of claim 283 wherein the TRPML3 polypeptide expressed by said cells or enriched cell sample has a mutation that renders the ion channel more sensitive or permeable to sodium, has a mutation that maintains the ion channel in an “open” or “closed” orientation, the TRPML3 polypeptide expressed by said cells or enriched cell sample has a mutation that is cytotoxic, and/or the TRPML3 polypeptide expressed by said cells or enriched cell sample has a mutation that results in a cell wherein sodium influx and efflux is uncontrolled.

287. The isolated cells or cell sample of claim 283 which comprises taste cells which respond to salty taste.

288. The isolated cells or cell sample of claim 287 wherein the taste cells are selected from human, non-human primate, rodent, canine or feline taste cells.

289. The isolated cells or cell sample of claim 283 which is in solution, or on a solid support.

290. The isolated taste cell or enriched cell sample of claim 283 wherein the taste cell is a human salty taste cell.

291. An isolated taste receptor that modulates salty taste perception comprising a TRPML3 polypeptide or variant thereof that modulates salty taste in mammals.

292. The isolated taste receptor of claim 291 which is a homopolymer, monomer or heteropolymer.

293. The isolated taste receptor of claim 291 which comprises a mammalian, avian, amphibian, fish, or reptilian TRPML3 ion channel.

294. The isolated taste receptor of claim 291 wherein said TRPML3 ion channel polypeptide possesses at least 90% sequence identity to a TRPML3 polypeptide selected from the polypeptides having SEQ ID NO: 2, 4, 10, 12, 14, 16, 22, 24, 26, 28, 30, 32 and 34 or the TRPML3 polypeptide encoded by SEQ ID NO:39 or 40.

295. The isolated taste receptor of claim 291 wherein the TRPML3 polypeptide expressed therein has a mutation that renders the ion channel more or less sensitive to sodium, the TRPML3 polypeptide expressed therein has a mutation that renders the ion channel more or less permeable to sodium, the TRPML3 polypeptide expressed therein has a mutation that maintains the ion channel in an “open” or “closed” orientation, the TRPML3 polypeptide expressed therein has a mutation that is toxic to cells containing the ion channel, or the TRPML3 polypeptide expressed therein has a mutation such that when this ion channel is expressed in a cell sodium or calcium influx and efflux is uncontrolled.

296. The isolated taste receptor of claim 291 which comprises another ion channel polypeptide.

297. The isolated taste receptor of claim 291 which is a human or non-human primate salty taste receptor.

298. A transgenic non-human animal which has been genetically engineered to knock out or to impair the expression of endogenous TRPML3 with the proviso that said transgenic animal is not a Varitint mouse or a transgenic non-human animal which has been genetically engineered to express a heterologous TRPML3 polypeptide with the proviso that said transgenic animal is not a Varitint mouse.

299. The transgenic animal of claim 298 wherein the TRPML3 polypeptide is a mammalian, avian, fish, amphibian or reptilian TRPML3.

300. The transgenic animal of claim 298 wherein said heterologous TRPML3 ion channel polypeptide possesses at least 90% sequence identity to a TRPML3 polypeptide selected from the polypeptides having SEQ ID NO: 2, 4, 10, 12, 14, 16, 22, 24, 26, 28, 30, 32 and 34 or the TRPML3 polypeptide encoded by SEQ ID NO:39 or 40.

301. The transgenic animal of claim 298 wherein said heterologous TRPML3 ion channel polypeptide wherein the heterologous TRPML3 polypeptide expressed therein has a mutation that renders the ion channel more or less sensitive to sodium, the heterologous TRPML3 polypeptide expressed therein has a mutation that renders the ion channel more or less permeable to sodium, the heterologous TRPML3 polypeptide expressed therein has a mutation that maintains the ion channel in an “open” or “closed” orientation, the TRPML3 polypeptide expressed therein has a mutation that is toxic to cells expressing the ion channel, the heterologous TRPML3 polypeptide expressed therein has a mutation such that when this ion channel is expressed in a cell sodium or calcium influx and efflux is uncontrolled, the heterologous TRPML3 polypeptide expressed therein results in the ablation of specific cell types; and/or the heterologous TRPML3 polypeptide expressed therein is a mutated human TRPML3 polypeptide having a mutation such that when this ion channel is expressed in a cell sodium or calcium influx and efflux is uncontrolled.

302. A method of using the transgenic animal of claim 298 in screens to identify salty taste modulating compounds.

303. A method of using the varitint-waddler mouse to assay TRPML3 function or to identify genes or polypeptides specifically expressed or not specifically expressed in said mouse as compared to a mouse having normal TRPML3 function.

304. The method of claim 303 wherein which assays the function of TRPML3 in taste, pituitary, adrenal, urinary organ or melanocytes.

305. The method of claim 303 which comprises the use of gene chips to compare the genes which are specifically expressed in salty taste cells of normal versus varitint waddler mice in order to identify genes that are specifically expressed in taste cells which detect salty taste.

306. A method of using the genes detected or corresponding polypeptides detected in claim 303 in assays to detect salty taste modulators.

307. The assay of claim 303 which is used to detect genes that modulate TRPML3 function, or function as a salty taste receptor, or modulate transmission of salty taste signaling from TRPML3 to the nerve fibers and/or control the development differentiation or apoptosis of salty taste cells; or to identify antagonists, agonists or enhancers of TRPML3.

308. A method of using a transgenic animal according to claim 298 in order to elucidate at least one of (i) the effect of TRPML3 on aldosterone production, sodium metabolism, salty taste perception or vasopressin release; (ii) to assess compounds for their potential therapeutic regimens for diseases or conditions involving aberrant aldosterone production, vasopressin release, sodium metabolism and/or melanocyte loss.

309. A recombinant cell which expresses a salty taste receptor comprising TRPML3 or a variant thereof.

310. The recombinant cell of claim 309 which is a yeast, amphibian, insect, bacterial, reptile, avian, or mammalian cell.

311. The recombinant cell sample of claim 309 wherein said TRPML3 is selected from a mammalian, avian, amphibian, fish, and reptilian TRPML3.

312. The recombinant cell of claim 309 which expresses a mammalian TRPML3 is selected from the group consisting of human, murine, rat, canine, feline, guinea pig, pig, horse, cow, goat, sheep, bear, monkey, gorilla, chimpanzee, orangatan, macaque, cynomolgus monkey, gibbon, gazelle, and zebra TRPML3.

313. The recombinant cell of claim 309 wherein said TRPML3 ion channel polypeptide possesses at least 90% sequence identity to a TRPML3 polypeptide selected from the polypeptides having SEQ ID NO: 2, 4, 10, 12, 14, 16, 22, 24, 26, 28, 30, 32 and 34 or the TRPML3 polypeptide encoded by SEQ ID NO:39 or 40.

314. The recombinant cell of claim 309 wherein the TRPML3 polypeptide expressed by said cells has a mutation that renders the ion channel more or less sensitive to sodium, has a mutation that renders the ion channel more or less permeable to sodium, cell has a mutation that maintains the ion channel in an “open” orientation, has a mutation that is toxic to some cells, has a mutation that results in a cell wherein sodium influx and efflux is uncontrolled, and/or said TRPML3 polypeptide responds to salty taste modulators.

315. The recombinant cell of claim 314 which expresses another sodium ion channel polypeptide.

316. A method of identifying compounds that agonize, antagonize or enhance an activity of TRPML3 comprising contacting a recombinant cell according to claim 309 or a primary cell, acutely dissociated cell, or other cell which endogenously expresses a TRPML3 ion channel polypeptide with a putative TRPML3 enhancer, agonist or antagonist and determining the effect thereof on TRPML3 activity.

317. The assay of claim 316 wherein the endogenous cell is selected from an adrenal cortex cell, parathyroid cell, taste bud cell, urinary organ cell, melanocyte, adrenal or parathyroid cell.

318. The assay of claim 316 wherein the cell expresses a rodent or human or non-human primate TRPML3 sequence.

319. The assay of claim 316 wherein the TRPML3 has a sequence at least 90% identical to a polypeptide selected from those contained in SEQ ID NO: 2, 4, 10, 12, 14, 16, 22, 24, 26, 28, 30, 32, and 34 or encoded by the polypeptide in SEQ ID NO:39 or 40.

320. The assay of claim 316 wherein the TRPML3 is a wild-type human sequence or a rodent or human sequence comprising the Varitint-waddler mutation (A419P).

321. The assay of claim 316 wherein the TRPML3 is a human sequence encoded by SEQ ID NO:1, 3, 17 or 18.

322. The assay of claim 316 which is an electrophysiological assay.

323. The assay of claim 322 wherein said assay is an electrophysiological assay which uses an ion sensitive dye or fluorophore or membrane potential dye.

324. The assay of claim 322 wherein said assay is a two electrode voltage clamping assay or is a patch clamp assay.

325. The method of claim 322 which uses a sodium sensitive dye.

326. The assay of claim 316 wherein the assay measures activity by an ion flux assay.

327. The assay of claim 316 which uses a fluorescence plate reader (FLIPR).

328. The assay of claim 316 which uses a voltage imaging plate reader (VIPR).

329. The assay of claim 316 which uses an automated electrophysiology instrument.

330. The assay of claim 316 which uses an IonWorks assay system.

331. The assay of claim 316 wherein the identified agonist, antagonist, or enhancer compounds are evaluated in a taste test.

332. The assay of any claim 316 wherein (i) the effect of the identified agonist, antagonist or enhancer on aldosterone production is tested in an animal, (ii) the effect of the identified agonist, antagonist or enhancer on vasopressin release is tested in an animal, the effect of said identified antagonist, agonist or enhancer compound on at least one of Addison's disease, hair loss, hair or fur discoloration, taste cell regeneration, pituitary cell regeneration, adrenal cell regeneration, melanocyte cell regeneration, blood pressure, fluid retention, sodium metabolism and urine production is tested in an animal, (iv) the effect of the identified compound for the treatment of a disease or condition selected from edema, blood pressure (hyper or hypotension), liver cirrhosis, primary hyperaldosterinia, renal dysfunction, diabetes (Type I or II) and the pathological symptoms associated therewith including circulatory problems, edema, ocular disorders relating to poor circulation, hypercortisolaemia, atherosclerosis or obesity, e.g., abdominal obesity, as well as liver disease, sexual dysfunction (male or female), cerebrovascular disease, vascular disease, retinopathy, neuropathy, insulinopathy, endothelial dysfunction, baroreceptor dysfunction, migraine headaches, hot flashes, and premenstrual tension and other cardiovascular conditions such as atherosclerosis, heart failure, congestive heart failure, vascular disease, stroke, myocardial infarction, endothelial dysfunction, ventricular hypertrophy, renal dysfunction, target-organ damage, thrombosis, cardiac arrhythmia, plaque rupture and aneurysm or another condition treatable by an aldosterone agonist or antagonist is evaluated in an appropriate in vitro or in vivo animal model, (iv) the effect of the identified compound for the treatment of wherein the disease or condition is selected from cystic kidney disease, acquired renal cystic disease, ocular circulation related disorders such as myopia; nausea, emesis, sexual dysfunction (male or female), edema, hypertension, congestive heart failure (ranging from class II of the New York Heart Association to florid pulmonary edema), periodic idiopathic edema, nephrotic syndrome, ascites due to cirrhosis or other causes, cerebral edema of various causes, dilutional hyponatremia and metabolic alterations collectively known as the syndrome of inappropriate ADH secretion and other diseases or conditions wherein vasodilation and/or antioxytocic activity or the administration of a vasopressin agonist or antagonist is therapeutically desirable is tested in an appropriate in vitro or in vivo model.

333. A method of modulating blood pressure or fluid retention, urine production and/or excretion in a subject in need thereof comprising administering an effective amount of a compound that modulates TRPML3 identified according to claim 316.

334. A method of identifying putative salty taste modulators in a binding assay comprising providing a TRPML3 polypeptide or cell which expresses TRPML3 and contacting said polypeptide or cell with putative TRPML3 modulatory compounds and identifying potential TRPML3 modulators based on their specific binding to TRPML3 polypeptide.

335. The binding assay of claim 334 which is a direct binding assay.

336. The binding assay of claim 334 which is a competitive binding assay.

337. The binding assay of claim 334 which uses a mammalian TRPML3 polypeptide.

338. The binding assay of claim 334 which uses a TRPML3 polypeptide that possesses at least 90% sequence identity to a TRPML3 polypeptide selected from those in SEQ ID NO:2, 4, 10, 12, 14, 16, 22, 24, 26, 28, 30, 31 and 34 or a polypeptide encoded by SEQ ID NO:39 or 40.

339. The binding assay of claim 336 wherein the TRPML3 is expressed by a mammalian cell.

340. The binding assay of claim 336 wherein the TRPML3 polypeptide is monomeric or polymeric.

341. The method of claim 316 which is used to identify a modulator of TRPML3 utilizing a mammalian cell or oocyte that expresses a functional TRPML3 sodium channel with a putative TRPML3 modulatory compound, comprising:
(i) assaying the effect of said compound on sodium transport through the TRPML3 channel; and
(ii) identifying whether said compound is an TRPML3 modulator based on its enhancing or inhibitory effect on sodium transport.

342. The method of claim 341, further comprising (iii) confirming that the compound identified modulates salty taste in human or mammalian taste tests.

343. The method of claim 341 wherein the in vivo effect of the identified compound on sodium excretion or urinary function is tested in humans or mammals.

344. The method of claim 343, wherein said cell expresses an additional gene or ion channel expressed in taste cells.

345. A method of using the compound identified in claim 343 to modulate or enhance salty taste perception, or to modulate sodium excretion or urinary function in humans or mammals.

346. The method of claim 343 wherein said assay is an electrophysiological assay.

347. The method of claim 316 which is a mammalian or frog oocyte cell-based high-throughput assay for the profiling and screening of putative modulators of TRPML3 comprising: contacting a test cell expressing TRPML3 or a variant, fragment or functional equivalent and preloaded with a membrane potential fluorescent dye or a sodium fluorescent dye with at least one TRPML3 putative modulator compound in the presence of sodium or lithium; and monitoring cation mediated changes in fluorescence of the test cell in the presence of the putative modulator/TRPML3 interactions compared to changes in the absence of the modulator to determine the extent of TRPML3 modulation.

348. The assay method of claim 349 in which the cation is sodium, lithium or potassium.

349. The assay method of claim 349 which identifies said compound based on a detectable change in fluorescence.

350. The assay method of claim 349 wherein the test cells are loaded with a membrane potential dye that allows for changes in fluorescence to be detected.

351. The assay method of claim 349 wherein TRPML3 is a human TRPML3 that is encoded by DNA selected from a human TRPML3 DNA cloned from human taste cell cDNA, a codon optimized or wild-type or mutant TRPML3 DNA, or the TRPML3 DNA is selected from those contained in SEQ ID NO:1, 3, 17, 18, 39 and 40.

352. The assay of claim 349 wherein a fluorescence plate reader or a voltage imaging plate reader is used to monitor changes in fluorescence.

353. The method of claim 316 which monitors the activity of TRPML3 comprising: providing a test cell transfected with a functional TRPML3 splice variants and fragments thereof; seeding the test cell in the well of a multi-well plate; dye-loading the seeded test cell with a membrane potential dye in the well of the multi-well plate; contacting the dye-loaded test cell with at least one putative modulating compound and sodium in the well of the multi-well plate; and monitoring any changes in fluorescence of the membrane potential dye due to modulator/TRPML3 interactions using a fluorescence plate reader or voltage intensity plate reader.

354. The method of claim 353, wherein the TRPML3 is a human TRPML3 cloned from human taste cell cDNA, is selected from the group consisting of: a naturally occurring human TRPML3, an alternatively spliced human TRPML3, and a functional variant thereof.

355. The method of claim 316 which is used to identify a salty taste modulating compound comprising: providing a test cell transfected or transformed with a functional human TRPML3, splice variant, chimera or fragment thereof; seeding the test cell in the well of a multi-well plate; dye-loading the seeded test cell with a membrane potential dye in the well of the multi-well plate; contacting the dye-loaded test cell with at least one putative modulatory compound and sodium in the well of the multi-well plate; monitoring any changes in fluorescence of the membrane potential dye due to modulator/TRPML3 interactions using a fluorescence plate reader or voltage intensity plate reader; and identifying at least one putative modulator as a salty taste modulating compound based on the monitored changes in fluorescence.

356. The method of claim 355 further comprising evaluating the identified TRPML3 modulatory compound for effects on salty taste perception.

357. The assay of claim 355 wherein the TRPML3 is selected from the group consisting of: a naturally occurring human TRPML3, an alternatively spliced human TRPML3, or a functional variant thereof.

358. The assay of claim 316 which uses a high throughput patch clamp electrophysiological assay system.

359. The method of claim 358 which comprises an IonWorks automated patch clamp system.

360. The method of claim 358 which is used to identify compounds that open (activate) the TRPML3 ion channel or compounds that close (block) the TRPML3 ion channel.
